SEQUENCE LISTING (21 sequences)

SEQ ID NO: 1 - protein, 512 aa, Marinobacter algicola
MATQQQQNGASASGVLEQLRGKHVLITGTTGFLGKVVLEKLIRTVPDIGGIHLLIRGNKRHPAARERFLNEIASSSVFERLRHDDNEAFETFLEERVHCITGEVTESRFGLTPERFRALAGQVDAFINSAASVNFREELDKALKINTLCLENVAALAELNSAMAVIQVSTCYVNGKNSGQITESVIKPAGESIPRSTDGYYEIEELVHLLQDKISDVKARYSGKVLEKKLVDLGIREANNYGWSDTYTFTKWLGEQLLMKALSGRSLTIVRPSIIESALEEPSPGWIEGVKVADAIILAYAREKVSLFPGKRSGIIDVIPVDLVANSIILSLAEALSGSGQRRIYQCCSGGSNPISLGKFIDYLMAEAKTNYAAYDQLFYRRPTKPFVAVNRKLFDVVVGGMRVPLSIAGKAMRLAGQNRELKVLKNLDTTRSLATIFGFYTAPDYIFRNDSLMALASRMGELDRVLFPVDARQIDWQLYLCKIHLGGLNRYALKERKLYSLRAADTRKKAA

SEQ ID NO: 2 - protein, 513 aa, Marinobacter aquaeolei
MAIQQVHHADTSSSKVLGQLRGKRVLITGTTGFLGKVVLERLIRAVPDIGAIYLLIRGNKRHPDARSRFLEEIATSSVFDRLREADSEGFDAFLEERIHCVTGEVTEAGFGIGQEDYRKLATELDAVINSAASVNFREELDKALAINTLCLRNIAGMVDLNPKLAVLQVSTCYVNGMNSGQVTESVIKPAGEAVPRSPDGFYEIEELVRLLQDKIEDVQARYSGKVLERKLVDLGIREANRYGWSDTYTFTKWLGEQLLMKALNGRTLTILRPSIIESALEEPAPGWIEGVKVADAIILAYAREKVTLFPGKRSGIIDVIPVDLVANSIILSLAEALGEPGRRRIYQCCSGGGNPISLGEFIDHLMAESKANYAAYDHLFYRQPSKPFLAVNRALFDLVISGVRLPLSLTDRVLKLLGNSRDLKMLRNLDTTQSLATIFGFYTAPDYIFRNDELMALANRMGEVDKGLFPVDARLIDWELYLRKIHLAGLNRYALKERKVYSLKTARQRKKAA

SEQ ID NO: 3 - protein, 512 aa, Artificial Sequence
  Synthetic polypeptide
MATQQQQNGASASGVLEQLRGKHVLITGTTGFLGKVVLEKLIRTVPDIGGIHLLIRGNKRHPAARERFLNEIASSSVFERLRHDDNEAFETFLEERVHCITGEVTESRFGLTPERFRALAGQVDAFINSAASVSFREQLDKALKINTLCLENVAALAELNSAMAVIQVSTCYVNGKNSGQITESVIKSAGESIPRSTDGYYEIEELVHLLQDKISDVKARYSGKVLEKKLVDLGIREANNYGWSDTYTFTKWLGEQLLMKALSGRSLTIVRPSIIESALEEPSPGWIEGVKVADAIILAYAREKVSLFPGKRSGIIDVIPVDLVANSIILSLAEALSGSGQRRIYQCCSGGSNPISLGKFIDYLMAEAKTNYAAYDQLFYRRPTKPFVAVNRKLFDVVVGGMRVVLSIAGKAMRLAGVNRELKVLKNLDTTRKLATIFGFYTAPDYIFRNDSLMALAQRMGELDRVLFPVDARQIDWQLYLCKIHLGGLNRYALKERKLYSSRAADTDDKTA

SEQ ID NO: 4 - protein, 512 aa, Artificial Sequence
  Synthetic polypeptide
MATQQQNNGASASGVLEILRGKHVLITGTTGFLGKVVLEKLIRTVPDIGGIHLLIRGNKRHPAAGERFLNEIASSSVFERLRHDDNEAFETFLEERVHCITGEVTESRFGLTPERFRALAGQVDAFIHSAASVNFREQLDKALKINTLCLENVAALAELNSAMAVIQVSTCYVNGKTSGQITESVIKSAGESIPRSTDGYYEIEELVHLLQDKISDVKARYSGRVMGKKLVDLGIREANNYGWSDTYTFTKWLGEQLLMKALSGRSLTIVRPSIIESALEEPSPGWIEGVKVADAIILAYAREKVSLFPGKRSGIIDVIPVDLVANSIILSLAEALSGSGQRRIYQCCSGGSNPISLGKFIDYLNAEAKTNYAAYDQLFYRRPTKPFVAVNRKLFDVVVGVMRVVLSIAGKAMRLAGVNRELKVLKNLDTTRKLATIFGFYTAPDYIFRNDSLMALAQRMGELDRVLFPVDARQIDWQLYLCKIHLRGLNRYALKERKLYSSRAADTDDKTA

SEQ ID NO: 5 - protein, 512 aa, Artificial Sequence
  Synthetic polypeptide
MATQQQNNGASASGVLEILRGKHVLITGTTGFLGKVVLEKLIRTVPDIGGIHLLIRGNKRHPAARERFLNEIASSSVFERLRHDDNEAFETFLEERVHCITGEITESRFGLTPERFRALAGQVDAFIHSAASVNFREQLDKALKINTLCLENVAALAELNSAMAVIQVSTCYVNGKTSGQITESVIKSAGESIPRSTDGYYEIEELVHLLQDKISDVKARYSGRVMGKKLVDLGIREANNYGWSDTYTFTKWLGEQLLMKALSGRSLTIVRPSIIESALEEPSPGWIEGVKVADAIILAYAREKVSLFPGKRSGIIDVIPVDLVANSIILSLAEALSGSGQRRIYQCCSGGSNPISLGKFIDYLNAEAKTNYAAYDQLFYRRPTKPFVAVNRKLFDVVVGVMRVVLSIARKAMRLAGVNRELKVLKNLDTTRKLATIFGFYTAPDYIFRNDSLMALAQRMGELDRVLFPVDARQIDWQLYLCKIHLRGLNRYALKERKLYSSRAADTDDKTA

SEQ ID NO: 6 - protein, 514 aa, Artificial Sequence
  Synthetic polypeptide
MSQYSAFSVSQSLKGKHIFLTGVTGFLGKALLEKLLYSVPQLAQIHILVRGGKVSAKKRFQHDILGSSIFERLKEQHGEHFEEWVQSKINLVEGELTQPMFDLPSAEFAGLANQLDLIINSAASVNFRENLEKALNINTLCLNNIIALAQYNVAAQTPVMQISTCYVNGFNKGQINEEVVGPASGLIPQLSQDCYDIDSVFKRVHSQIEQVKKRKTDIEQQEQALIKLGIKTSQHFGWNDTYTFTKWLGEQLLIQKLGKQSLTILRPSIIESAVREPAPGWVEGVKVADALIYAYAKGRVSIFPGRDEGILDVIPVDLVANAAALSAAQLMESNQQTGYRIYQCCSGSRNPIKLKEFIRHIQNVAQARYQEWPKLFADKPQEAFKTVSPKRFKLYMSGFTAITWAKTIIGRVFGSNAASQHMLKAKTTASLANIFGFYTAPNYRFSSQKLEQLVKQFDTTEQRLYDIRADHFDWKYYLQEVHMDGLHKYALADRQELKPKHVKKRKRETIRQAA

SEQ ID NO: 7 - protein, 505 aa, Hahella chejuensis
MKQSLTLTAFANKNVLITGTTGFVGKVVLEKLLRSVPTIGKIYLLIRGNSKNPTARKRFQNEIATSSIFDTLKASQGSRFEELCETRIHCVTGEVTEPLFGLSEKDFTDLAADIDVIINSAASVNFREALDQALTINTLCLKNIIELSRRAADCPVVQVSTCYVNGFNQGVMEEEIVSPAGERIERSERGYYEVEPLIARLLQDVEQVSAAAADDHSREKDLIDLGIKEANKYGWNDTYTFTKWMGEQLLMKELYGKTLTILRPSIVESTLLGPAPGWIEGVKVADAIILAYAREKVSLFPGKKNAVIDIIPADLVANSIILSATEALLDSGAHRIYQCCSSEVNPIRIREVIGHVQQEAEHNYQTHDKLFYRKPKKPFVMIPGAVFHALMAISFHMLKWSSRLQSLFGRKASGRKLSNMETTMKLSKVFSFYTSPSYTFSNRRLQELSTRLGEYDQSEFPVNAGMYDWAHYLREVHVAGLNKYALRPKVVKMNPPAAKPRSRAA

SEQ ID NO: 8 - protein, 769 aa, Marine actinobacterium PHSC20C1
MTTIRALTTEHVFLTGATGFVGQAILERLLSSHPETRISILVRGKGATTGEGRLTNLMRKPVFAQWMESLGKEQALAEVARRVTVIEGSLTDVGTLPDDIDIVIHGASTVSFDPPIDEAFDTNVGGATGIYTALLASKSRPHVVHISTAYVGGIRKGIVPEASLVHNVDWRAEYEAARTARTRVEFESRQPEALRAQLTAAKARHGKAGPQAVAQFTEAARAEWVHDRLVDYGRMRAESLGWTDVYTLTKAFAERVAEEMWAQAGHRLSVVRPSIIESALHHPFPGWIDGFKVADPLILAYGRGQLPDFPGLPDSILDVIPVDFVVNATLAAAAAKADPKAPRYYHVSSGASNPLPFHRMYENVNAYFTANPLPAEDGEISVPLWRFPGGQRVERALVKRERQAARAERVITRMPTTPRTRRWLDEVKSGQHQLEVLRAFTNLYRAYVQTEIIFDDANTRELLASLPKKTAHSARFDVTEIDWENYFQQVHFPAITTLTRAFANRPAAKTRTAKKLPERTDVVAVFDLEGTVVDSNLVKQYLLLWGGTVPRAKVLHDLANFTFSLRKYMRAERRDRGEFIRTFMRRYEGFKIAEIERMVRGSFGRAMMRRVMPDALRRVQEHRDAGHRTILVTGTIDLMVTPFLPYFDEVVAGRMHERDGILTGFLADPPLVDEARAAWLRHYADQNGFNLTQSYGYGDSHADLMWLQLVGNPSAVNPDVNLYKHAQEKRWNVLDWKRRSPNSRIPRPRDAAAATQEGDGHSSTPSSQS

SEQ ID NO: 9 - protein, 542 aa, Unknown
  Synthetic polypeptide encoded by DNA from NCBI locus EP750655 from marine metagenome collection
MIRENLSGKRIAITGSTGFLGTALVERFLRSVPDCELVLLVRPGRRGAEHRVKRDILKNDAFDRLRDAFKEDPIAAGGLDGETFDEMCDRRVFAVKGDVGQDGLGLDDAGLTLFSTVDIAVHSAATVSFDSALDDAVQVNLLGPGRVAAALRVAAEARTEPTPGGLAPGEKAYLVAVSTCYVAGSRRGNAPEQMVQDSPFFVDVDWRAEAHNAFQARKDAEQASRTPQRLKALEADAIKTLGAAGTPAIAERVESLRQKWVGEKMTETGRVRAASLGFPDAYAFTKALGERSLVETRDGVPVAIVRPSIIESALAEPVPGWIRGFRMAEPVIAAYARGLLKEFPGVPEGVIDVIPVDLVVASILATAARGPHISEQSGEHEPDIIQIASGSANPFKYGQMVDLVQAYFTKNPVYDEKNQPISVPDWTFPGRGRVTRQLNRAKFALTTGERILDALPLRGKQAEIGADLEQQKEQLDRAGGYVELYGAYTECEATYQLDRMYALWNSLDEADQRDFNMDPLSIDWPHYAHDIQLPSTVKMARL

SEQ ID NO: 10 - protein, 523 aa, Unknown
  Synthetic polypeptide encoded by DNA from marine sample JCVI_SCAF_1101670217388
MIKQNLSGKRIAITGATGFLGTALVERLLSSIPDCELLLLVRPGRRGAEKRAEREILRNDAFNNLREKLGTDGFDKLCKKRVKAISGDVGIDGLGLDENGLTELAKCDLFIHSAAVVSFDSPLDQAVEVNLLGPVRIAQTLNELAVSPHLVSISTCYVAGSRRGAAPEEPVDASPFFVDVDWRIEVDAARRIRQETETASRTPERLEEFRKEAREEIGAAGIPALASKTEQLRSRWVDDRMAEAGRSRAHSLGFPDAYAYTKALGEIALRETAHTIPVSIVRPSIIESALAEPFPGWIRGFRMAEPVIISYARGLLKDFPGIPEGTIDVIPVDLVANSIILSLAEALSGSGQRRIYQCCSGGSNPISLGKFIDYLMAEAKTNYAAYDQLFYRRPTKPFVAVNRKLFDVVVGGMRVPLSIAGKAMRLAGQNRELKVLKNLDTTRSLATIFGFYTAPDYIFRNDSLMALASRMGELDRVLFPVDARQIDWQLYLCKIHLGGLNRYALKERKLYSLRAADTRKKAA

SEQ ID NO: 11 - protein, 515 aa, Unknown
  Synthetic polypeptide encoded by DNA from marine sample JCVI_SCAF_1097205236585
MIKESLRGKKIAITGSTGFLGTALVELLLREIDDVQLRLLIRPSGKRSASKRLERDILRNDAFDQLREKLGTEGFNELASNAVEALSADISLDNLGLDDTGLRELSECDIVIHSAAAVSFDEPLDRAAEVNLMGPVRLVETLQNLDAEPHLVMVSTCYVAGNRKGTAPEKPLSQSPFYVPLNWREETEAARRTRSYTEDDSRRSENLEKFRGEAKSELGAPGISVIATKTEQIRERWVKEKMVEAGRERATSLGFPDAYAFTKAMAEQAVQEIRGNIPLSIVRPSIIESSWGNPKSGWIRGFRMAEPIILNFGRGTLKEFPGIPEGIIDVIPVDLVASAIVAVAAQEKPSDPFVVQVASGACNPIKIGILADYVHEFFGNFPILDDKNQPITPSKWEFPGRGRVVTQLTRAKRILQAAENGLHKLPIRGNQAMIVADLEEKRNELDKAMEYITLYGKYVECEAIYDVANLLHLWDSIDDGDRSSFLFDPRIIDWRKYVYDIHLPTVITQGRVKTT

SEQ ID NO: 12 - protein, 372 aa, Unknown
  Synthetic polypeptide encoded by DNA from marine sample JCVI_SCAF_1101670289386
MSNTTVIFLTGVTGYLGSRLLVQLSNLPVKIYCLVRPARSENGAQGRLLKILEDNNITPEEGKYIAVEGDIRKDKLGISDEKYLSLSREVEVVFHSAASVNFLSTKEALKSINVVGAINVMNFAQRCYANNQSFDKFCLVSTAYVAGKTSGVAEEIPVTKARVFNNNYEESKWLAEQRVVEEVDDLPYVVIRPSIIIGSAIDGRAESQNVIYGPFRIMIQYDNKKPQWLPGYKSTRLDFVPVDYVAECCRHIIFEKDPKPIYHLTSGPDNQAGMNNMFKSVSDVFSVTVKLYPYWLFDMFIKPFLKLRKNKDDLKFLRIADAYGNYMKYKTQFDDRNTAELRYKNGIVRPSWNDVFTKSIRYAKDTNFARDV

SEQ ID NO: 13 - protein, 490 aa, Vitis vinifera
MELGSIVEFLENKSILVTGATGFLAKIFVERILRTQPNVKKLFLLLRAGDTKSATQRLHNEVIGKELFWVLREKWASDFNSFVSKKLTPVPGDISCDDLGVTDSNLREEMWREVDIVVNLAATTNFDERYDVALGINALGARHVLDFAKKCVKIKMLLHVSTAYVAGEQSGLILEQPFQMGETLNGTFGLDIEEEKKLMEERLDELQSEGATREAVTLAMKDFGIQRAKMHGWPNTYVFTKAMGEMLLGHLKENLPLAILRPTIVSSTYKEPFPGWVEGIRTIDSFAVGYGKGRLTFFLGDIEAIVDVIPADMVVNSMIVAMAAHANQPCEVIYQVGSSVKNPVRYSNLQDFGLRYFTKNPWINKDGKAVKVGKVTVLSTMDSFHRYMALRYLLLLKGLQFVNTAFCQYFRGTYTDLNRRIKFLLRLIELYKPYLFFKGVFDDMNTEKLRMAVTASGAEADLFYFDPKCIDWEDYFMNIHIPGAVKYVFK

SEQ ID NO: 14 - protein, 991 aa, Vitis vinifera
MAVSTTTTPNTSITNGLGILQFLAGKTYFITGATGLLAKAVVEKILRRAPDVGKIFILIKAKNKEAAVDRLKTEIINSELFECLKQRHGKYYQDFMLSKLAPVVGNLCESDLGIDANLISEIAEEVDVIINSAANTNFEERYDVSLHANTIGPCRLMDFAKKYCKNLRVFLHVSTAYVNGEREGMITEKPFYMGESIAREKVASEFLPLSYPALDVDDEIKIALDSKVAFEGNLEDQKMKELGLERARIHGWHNPYEFTKAMGEMMINSMRGDIPLVIIRPTAIESTLEDPFPGWIQGNRYLISLPFSCPCTKSHIFSYNQMLDPMILSYGKGNLPSFLVNPEVVIDMIPVDMVVNAIIAAMAKHGIAGKPGIKVYHVGSSAVNLLPLGDLFKYSYEHFICSPINMDTEGKTTDMKEMKFFSSMDDFSSHMQTEIVQQRRLAISGNNASQRLERKCKMIVEHAINLARVYQPHMFFRGSSFQEKTYFITGGTGFLAKAVVEKILRTAPDVGKIFVLIKAKNKEAAMDRLKTEIIDSELFECLKQRHGKYYQDFILSKLAPVVGNLCESDLGIDANSISEIAEEVDVIINSAANTNFEERYDVSLSTNVLGPRRLMDFTNKYCKNLRVFLHVSTAYVSGEREGMIMEKPFHMGERIAREKAASEFPPLAYPVLDVDGEIEIALDSKVAFEGNLEDEKMKALGLERARIHGWHNPYEFTKAMGEMLINSMRGDIPLVIIRPTAIGSTLDDPFPGWIQGNRYLISLPFSCPCTKSHFFSNNQMADPLILSYGRVNLPSFLVNPEAVIDMIPVVMVVNAIIAAMAKHGIAGKPGIKVYHVGSSAVNPLPLGDLFKHSYEHFICSPINMDTEGKTVDMKEMKIFSPMDDFSSHMQTEIVQQRRLTISGNKASQRLERKCKMIVEHAINLARVYQPYMFFRGRFDNSNTHNLMEGMSEEEMKRFRLDVENVDWEDYITNIHISGLKKHVMKGRGMPK

SEQ ID NO: 15 - protein, 548 aa, Stigmatella aurantiaca
MSQLPELNVSQAFTGKRLLFAGSTGFVGKVTLSMLLTHYGQALDKVYVLVRKGSAASAERRFFDKVAISEPFQPLRDALGEEAALEFIRQKCHVLDGDITDPLMGLTEAQADELTGKVAAIVNCAGLVSFNPSLEVGLNVNTHGVKYSVDLALKWSAPLIHMSTAFVAGNRSGLVFEDEEVAGYFPKKDELDGRDFSLEQELKDAERIVARLREQADDKALTSLFRKKALDRLAEEGRDSRDEKTLRLAVGRERKLWLTTELVRAGMERAQHWGWPNTYTYTKSLGEQVMAATPGLRYSIVRPSIVETSRHFPFPGWNEGFTTSAPLAFAGIKGQRGIPAGFKTILDIIPVDQVAGATLGITAHSLTVHERRVYHLASGDENPFYASRSVELVGLYRRRYYRNKEGGNALLNEVRARIEPQPITRQRFESLSAPAFMKGARLLKQVIEEVRPAWGAPTVQALLDRAKVKLDDVEEQASSLSQLIELFLPFLWENRYVFRCDNTRSVYARMLPSDRAKIPWDPENINWREYWMETHLPGLEKFVFPGLE

SEQ ID NO: 16 - DNA, 233508 bp, Phytophthora ramorum
acccccccccccccccgtctcttcgtgactgttcaacgtctgcttcgcttattcttccaggttgcttcaaaatctgcaatgagatccggcatttgtgaaagattctccactggttcccatgttggcttctcttcacccatccactccacaaggaattgtgtacgttgttcccggtgacgctttcctcgaagacgcttggctatctggccgacactgccgtctgccagaatcacattacctggtcgggatagtcgagtcgaatccttgtacggcttcaatgatccagcattgaaaacagagtggagcttgtttctggtctggatgttgagctcgtaagcgtggttgtgtaccttccgcttgatcgtgttcgggccaatccacttcggtccaaacttcggtaggcctgagtgctttgcgtcgatgtgcttcgtgctaagtacactcggtacccaatatcgaaaacttgttccttacggtcccaattgtaaatatcgcgcattccctcttgggctttggctagtgcatcactacagcgtagtagaattgctgcttgacgttcatggaagtcagcgccctggcgactcttgggtatacgtgcaagggtttccgccgttagttgaatcgggttcaattggatataacccagatccgcttcgaacggcgcaagtttcgtgctgaagattaccgtcgagttaatggtaaactcggtgttggccaagtgaacgtcccaatcatcctccagtgggccaacaaagcagcggaggtactcctccagcgtacgcttcattcggtctgtttggccatcacgttgtgctcggtgtgccacggtcatgctcggtttcgtcccgatagacttgaagacgtgttgccagaagttggacacaaacttcctatcgcgatcggacacaatgttggatggcatgccgtgtagcttccagatgtcgttgatgaagagtcggacaacaccttcagctgtcaccgtcttactcgtgggcacgaagtgacaacggttggtcaaccgatcgacgacgacccagatggagtcgacgtcgcgcttcgttcgtggtaggtccgtgataaaatccatagagatggatcgccacctctcgtccgggatgaccagcggctcgagcagttctggaggcttcgtcaatcgtggcttgttccgcacacggatcgcaatccgtgatggatcgcttgatggacttgagcatattggaccagtgccaacggcgtgccagtgtcaagtacgttcgttcggcgccaggatgtccgccgtagttggtgtcatggcactcgctaatgatcgtctgccacaactttaaatcgttgggcacgacaatgcgaggtacgctaccagcaggtgtctggtaccacagcaaccacttcacctcgttatagcaccgtggtgctgggtatagcggggctacacagttggttcacctcctaccttagtgcaacacgcactaatggatacattcaggttgaccgcaccacaaggtggtctatattcatagcaaaggcatatgactggtagaaaccaacatctttctaccaatgagagtcattcattagatagggattctgccagccatccaatgacagaacctctcattagaaaaatcagtttaggaggaaaccccatttacaatcaatacagttctcatttggcaccaaagctatcgttcggtcccattatcaaagtttaagtatacttgtggatacaagtatactgaacttaagttaaatacacctacgcttcctcctacacgtgcgcacacgtgtaagcaaaagtagtgggtttaaggagacccattacactgagatacacgcttgggtattacactagctgtacgtcgctcgatcgccgtaaagattgcttggatctcatggtcctgcttgtacgcattcatgatgctgttgattaactcagagttggtagtcatctcactgatcgctaagctaagtgatgcctcatcaaagcttgtcacgctcaagtcgtgtaaaatcttcgtttcgggttctacgactcaaagtgtccacgataccgttcttcgcaccatacatatatgataaagccagctggtactccgcaaggatgtcgtaccagcgagctagccgtcggttggccatctgctgcgtgaacaggctttcaaggcttttatgatcggtctcgagaatcggaggtatgcccaagcagtagattctggatgcggctagagtgtgaacgatcacaaggtgttcctgctgctgggttgggtagttcagctccgctggcttcattttcctgttggtgtacgcaatgggtcgctcaacaccatcgataacttggaacaggactccccctacggcaaacttgctagcgcccgtacccagatacatgggtaagctgaggttcggcagatgtagttctggtggtttgcacagccgtctcttcagttccttgaagttcttaaactgataatcattgaggtagatcttggcgttgcgcttggttctcttcttgagaagattgaacacgcttgcggttaattctccataatttgggcagaaacgctggacggaaccagtcagaccaaggaagctgtgaagttcctctagagttctcgggaccggacagtccttgatagtctgaactgcccggatacattcgcacactagttcgtccgacgaaatctccgagacatggaatctcgttcgcacagaagacacacgtgaagagtttcacatatagtttgttatcacgcaagatgtgcaacgtcttccgtagtgtttccaagtgattttcaatcttgtacggcttcgtaaaaatgtagatgtcttcgtagaaggacttcgtgtccccaagatcgcggaacaatttcgacgtcaaccgatgcatcgtagctggtgcgttgcaaactcccatcggtagcacaagatattcccacagttcgtttggtgcttgggaggtggtaaacttgatgtcttcttcacgcagacgaacatggtagtacgctgacatcaaatccattgtggaaaaccagtacgctcccgccattgagtctaagatgtcttccttctgtgtcattggaacgctctgccgaaccgtgttggtgttcaggtatcgaaatcgtgtacgattcgccagccaactggtttccggacgcagaatgttggagcagcatgtgggctgatctcggtcagacgatctacttcttcaccatatcttcgacccaacgtacgatttcgcgttgctgttccggtgactgttgccactggtgtcggtacatcatgaggctcggatccttcacatcaattcgatgctctatctcgcgcttctcgggtaacccttcgggtagctcaggacggaacacagtatccttgtaacccttgagtaactcgaatgctgggttggtctggaagctgtcccagtcggtttccagatactgttgtgccttcgcacttccgacaatatcgggtttatctcgccgttgcttgatcatctcgttcgacagaaccagcacgtagtctggctcgtcaatcgttttggtcttcaatctcaggaactccgcagcttctttcgcgtgatgtacttcgtctgcacacccatttcatcgacaacccccatcttgaacatcttctccacgacgctgtctttgccccggtcactagtatctcatacttctctagtgcctacgattgaagatgacctcttaccatctccatctaccgctgattcacaaaccaacgactctttcggttgtccgtagataacgcgtagctttcgctctgctcttcagttcgaattggattaactgcagacctcacatccgtttccagcgcggcgcctccacaggaatcaggtctttcaacgataggcctttcgccccgacatatctcggaagctcggaggcaatcaccggtccgccttcctcgattgatccacaggtctcatctgctcgtttccagcgcaaagtccttgttcgcattccttccactagtcgaccccccagccaatatgcgggtgcttgtctacccacggcctggacacgtagatcgtggtagcgccacagtccagcaaaattcgcgatatcatgctcttgtccgccacatcgatctctccttcgacggtggacagcgggcgtggcttgtaaactagagggttttcgcacagatcattgagactaatattctgaaactttaacgtttttgctacaccatcgaccacgtcacggtcctccttcactgacgccgttgttcgttttcctgcttggcttattccttcttccacgagaatcaatcggccttgatgtgaccaggcttcttggagaagtagcaggtgcgtccttcggcacgctttgtattcgagcttgacttgaagcgaccttttccacagaaacatttccttttgacgagttcccgtttccgtgggtcgattcccatgaaagattcagcttgtcaggacgaactgggtgttcgcgtgcttcctctacgaagtgggccacttcgtacttgacggccaaattcatggcttcgctcagcgtctttgggttctcgagtttgacatagctctgagtccgcggcttgagaccgttgcgtagttaagcacttggtcgagcatgctcatgcctccaacacgaaatatcagtactgagtatttgttattgtccgtctcgatgtctgccgtcggcttgagacgctggtgcgtttcgcgtaggacggcctggtaattcaatgtatcgaagtgttggactgaaaatcccccacgtgtgctcctcgactctcgtcgtcgaagaccaatgcaggaaccaccccgaagccggcttctccatagccgacctggcgatccctggtaggagcagcccttgcgtcttcgatcgggatgccgttgatacggcaggcgttttgctctggaacaaccattcgcacacgtcttctcctctcttgccggtgaacttggatagattgatgggcacacgcgtgcctccagccatggcttgattcagccgatgtatatcggcgcttagttgggcgttcgtcggcatggtctacttatgggcaggttgctgggaccaacaagtgctatcaatttatcttaagcaaatagttttgaggggggaaggcgtgctaaaagagaagtgcccccccccgcagttcgggcagatggaagcttaaaatacagcgtccacagctgccgtacgtctgactacttgtattcacgtaagttcactataccatactaaacctcaatgtcagcactactgtttctaattggccaacacagaccgtctggcggcgggcggaggggctgccagattttaaatttggtcgagggtccggcgctgcgcagtacttgcatattaagtgctggcagcacttggctacgacactttcaagaacactgaggaggaggaaatcaacaaccacgagatcgttaagacgctgtagcacttgcatgaaaacgatgtggtgtcggtagatgagttcgttaatcctcacgatgaaacaccattgtggaggagcccccgacgatagcgtaaatgaaccagaacatgttgcggatgggagtgaggctggttcaattccactagaagagaatgatgcggaacgggatactgaggaggccgttaccatcaaggaacatctgatgtggattggtactgtaagctgctcgttcgtgcgaatgctaccgggcgtcgatgatcgagatgcagcgtacattccgctaattgttgaatagtataatgggtagttgggatgaaagtcgtctcacacaactctacagttgggggagcctctggatgagacggtagagccatcccagttgctaataatactcccccacagtgctggtaggacctatgtgctaggagaagcgagttttattgaaaatacactcggcataatcgttcagggagtgccaactctcccagagtgtcttctagcgagaccttctacaaataggaagcacgaccacaaagcagcagaagtcttagctagacttcgttggacgtcctgtgctattgtgatttttcaatagttaagctcaaccggactgcttgtcgatttcgtattccttcgaccgctgagcggtgagagggaatacttacttgagtttgtaaaatgacgatcgttttcatattcttgttgagcgagtattccaggcctgagggtcaccaccgaggggtttgaccatgctgggacgaaatgttgtatagtaaaatgctggatgagactggcttgctatcgctcgtataccaaaagtgcattgacctcgatcgagtagcttcacccaccgtcgattcactcctgttatgctgaaagcgcttctgcatccagcatttacctcagtggggtgagatggtcttgctccagcctaatgcgatgggaggagcctggtgtgccaagatggcacgtgaggctgagtgcagcgcacgcatgtgttccaagcaccgcatgccgagaagcagagcgccgccgccacagctcttgaaagcggccggtgttttgccgagccaagcgttgaagttgcacgtcattcaactgtactggctcgtcttcccattcgtcgactaaccgaagccaacatgtaaatgaccggaagtcagcgtctgcaccaagcagcaatagcgccacatgaagcaacaatcacagcgtcagcatgtatcacaaaggtaaacgccgccgccgccgcgtaaaagaggttgggcgctcaattctgctactagctgctcatacgaaacagcagcagagactcaaatagtactagaggagagttgcactgtggtccaccagcggggtatggagacacccacacagcaccagttcacaacgccgtgaagaagcgcggcgtgcatcctgcctccaaagcgccgagcacggcggcggcctcgagcatctggatggcgtccaacctgccacgaatggcgcggcatgttgtcctgtgtggttcagagcgagactggccacctggcaagcgcccaacgcgtgtcaatcttcgctggtaatcgacccagccaccacgacgccagcagtagcgatgagggcgagcgcgccatgtggcagcgcgacgaaagtctgacggcaagttgactcgacgacagggcggcaggcgtgcggcagtacagtaccgtgcacgagactctcttggttattaccgcggtgtcgtgtcacgctgcccgatcgccgatcggggcgaggataccgcaagcagcagcgctaaaaggtccgcctccgtttaggtggagcggtagaggtcgcctgcagtctggctgcggagacctataccatttcgcccggcgttgcgccgggaaagcctgaggaagtcgccttttggccaattatagctcagcgtcgcttcgcgctctcgacgatacgaccgatagtccgagaccggctccttttcctgctgctgcgatgccgaacaagtggcgcgtgagtagcgctccctcttcattcccaaacggaaggctaaccgaccggaaacaaccaatttcctcggtatctgcgcggcggcaagaggcagcaagcgagtgaacgagcgagggatcccccagcgacgacagcgaacagtgaacagacgcgtgcgtgacggacggacgcgtacgaggcgaaaagcgatcgaaacaggaaactacaggcagctcatgtggacgaaactcgccgtggcgagcgcgctgctgctgatacaggggactgcggcgcagggtacgtccagctcgatgtagagaggcgtagatggctttctgacttgcgctgggtgttgtgcattcgtgtagactgcgcgtacactgtcagtgtgctggatgtggcgggcgtgcagtgcgtgacctcgacgccctgctcggggacgtactacgcgtcgggtctgtcccagggggtgggcgcctgccccaccggcacggcctgcgcgctgctgccgctgacgcccatcatgggctgcgccgcgtctggccgcacggacctcacttacgtgaacgcggacggcacgctgaccaaggatggcaagtcggtgacgctctcgggcgcgtcgtctacgtcctcggacacgaccacgacggcccctaccacgagcagcacgagctctacgaccagctcaagcacgagttcaacgaccagctcgtcgaccagctcatcgacgagcagtggcaacggaagcaacgcgggctccggctccggatcagccaccagcagcaccaatggtaatgccttttctgccaccagtaccaattctgtagatattaatcctggctctgtcattgtagggagctcagacgccagcagcaaagcgactgccgacaacagcaccagcaactcggcgtccatttccgcctcgtcggcggcctccaacgccaacagctcgacgtccaactcgtggaattccgagtcgggatccggcagctacaccatctcacccggcagcagctcagcaaatcaacaggctgagttcaaccccgggtccatcctgagcgacggctccaactcatccacgtcttcgtcgggcgacggcctcggtcttggctctatcatcgccatcgtcgtgggctgcttagcggttgtagctatcgcggccggcgcgcggctgctgaagaagggcaaggagaccgatggcgagctggagacgcctgcgggagcgctcgaggactataacaacggaggtggcggcagcggtatgactcccaaggagaacgtgttgctgctctaagttatggccagcggcagatgatgtttttgagaggaatacagtgactgtgcaaactttgtgaacacgagaaagcgcgttcattgcgtgaacctggtgaatacaagtacatggcgcggtgcattcaagaaggaagcagagcgagggaagtggtggcgttgggtgttcggtgttgtcgtggatagatgcatggagctgtgggcgttggaaggtggggagagttggtagcacgtgtgaatagtagctctctgctcttccactcagatctctacgatcacttatatacgtgcgtcgacgcgcaacgtcgtctagtttactgcgcagctcggacccagcgaaaggctgtccttgcatcgtctttcatctcttccgttgctaaattttgctcatttatcccgagcctgtccgtgacttctgtagaaactacgagacagacacatgaacaaaatacatgaactcttgcgtgttggacagatggatggatggatgccttgcgagcctcatccgtcagtttggtgcgctgacgatgtcagcgacccccacgcgaaaccgcgtgcgctagtgatgccgcgttcttgtcgaaggatcatttgttattactctcaatgaaacctccgatgttgctacgtgccgcttaggtggccttggtcttggtcgccgcctcggacgccgccttggcctccttgagcgtgaagagcttggcgtccgggtccacgacgccgtagttggcgtacagtcggccgacgcgccagaaggccttgaggatcggctcgtcgctcttgcgcacgagctcggggaaggcctggtggaatgccgccgtagcgcggttgagcttgtagtgcgggatgatggggaagaggtggtggatctggtgcgtgccgatgttgtggctcaggttgtcgatgaacgcgccgtaggaccggtccacggacgacaggttgcccttgacgtaggtccagtcggagtctccgtaccatggggtctcctcgtcattgtgatgcaggaaggtggtgatcacaagcatgctgccgaacacgaagacggggccgtagtagtagagcgccatggtcttgagaccgaactggaagctcagatacacggacagcgccaacacggcgaagtgcgcggagagcgagatgacgacggcggccacctggcgcacaaacagcggctcgaatgggttgaagtggttgaccttgcggggcgggaagccctcgaccaggtaggcgaaccacgcggcgccgagcgccagcacgaggttgcgcgacagcgggtggtcgtcggccttgcgctgcgggtagaagatctcgtcgcggtcaatgttgcccgtgttcttgtggtggtggcggtgcgtgagcttccacgactcgaagggcgtgaggatgagcgagtggatgaaggtgcccaccacgaagttgagcaggtggtaacgcgagaaggcgccgtggccggcgtcatggcccaccgtgaagaagccccagaacacgatgccctgcagcagcacgtaaccgcagcagagcgcagcgtccagcgcccacaagctctcgaccacgggcagcgcgcgcgcgtagttgaggccgaacgccagcgccacggcgatggccacgatgcgcaccgtgtagtagagcgacagcggcaccgaggcctcaaagcactcgctgggcagcgaccgcttgatctccgtcagggtcgggaactggtacggctgcttagtcgccatggtggtggagacgtcggtgtctgatgcgccgcacgtcctggaatgagctctgcggacgtgtggctgcgaaaccaccgtggagtcatagatacccttcgccatagttcgggcaagtcgaagttggatggtgaacaaggcaccgccgggattcacgatgacatcacgcgcgtaccgtctaggacgcacacgccattcagcaaagccgtcccgcacgcgtcccgcattatcggcgctgctcaacgcaatgtgtattttattgttttatcatgcaagtagtgtaccgacgtgcagactttggtggggttgaaaacttggttttagcggaagggcagcaccacctcatgctgcaggatgtcttcggcgagaggccgctgcaacgcgtctgggtgcagcatgctggcgatcaggtgctgcagcgagttggagtattgtctgcagtgaacaagtgagcaagcttgtcagcaaaagaaacgaggccgcggggtggcaacagagtatcacgttcgtaccggaacatcacgaggtctccgtcgcggatcttctgccattcttcgccgccactcgccagcgtcgttccaagcgcgagctcatagattgtggcccccagcgcgaagatgtcgccggcgcgcaagttattccggttgccttctagcaattcgctgcaacgaacaaggaagtgacacgttagttaaacagccccttgcctcggcaagtagctagggtagaaagcacgtaccgactaagataccgattgtcgccctctgtgatctccatgctgccgtcaaggtgagcgacggttcccaggtcacccaactgcacgcatcaaatgtgagctccaaaacctagacatttcggcagcattcgagagcagcttaccttgtaaacctcttccgggccgaccagcacattttgtagcttgacgtccatatggaccattttcttgctgtgcatgtcgtaaagagcctacataaatcaaagacattgttagttccggtcaaagaacaggaatctagaagacaaaaagatctacctgagcaatatggcacagcagcttgcacagcgtctcttccgggatcttgtgcggtgcgtacttattggcaaagccagccaacgagcacccctccaggttttcaagctggatgtagagcaaatcgtcttctatccaggcgtcgaaatagcggacaacgtggttgctgctactcagcgctgccagcgcctgcacttctcgaagagctcgctcgctgtggtgcaagcacaacaacaaacgtaagtgcctatttacgcaaagcaccagcaaacaatgcatagctctgcgtacgtgtctgccttcccgcgaaaatggcgcttgctctttttcaccgcgtacacccagccatcaatctttttcatgcacttgtacactttcgagaacgagcccgagccgattaactgccaagcagaacaccacatctcagtactgtgcactgatggcaaacggcaatagatagacagcataccccaagctcggagaaatccgacagatacttggaaacgggcgatccactctgtcctcccacccacgcagacgggaacgaccgtcgtttgcgcctggaaatctttttcttccccgagtcggtctccggcgcgaacgggttcacataactctcgtcggacaagaacgacaccgctcgctctctgcaaaaaaaccaacaaccacatgaaaaaatagagcacacactagtgagcagcacgaagacacgctctcaccactacaatcactcacccgttgggtcgtggtctcttggcattattcaccaccacgttcaccctcttcttcagcggcgaccccgtgtccattgatgattggctcgagaaagacggaggccccttcctcgcgtgccgaacgtccatgtgggtcatatcctgcgtgaggttctgtacttgttcctcctgtaccacatccgctccgctattgagtctcggtctcttcttcaatggggagggagtcgtcgggttggacagcgggatgacgggctggtctctgggcgtaaggaagtccatgtcgtcctggctgaactggtgcatctggggcgtgtagcagccctgactcgtgggcggcaagccgaagcgcacgggcgtgtgcgtgggctcccagtcgggctcgctgcgctggctagagaagtcgctcttcttaggcctgtcgctgctctgggaggccgcgagtatgggttcctcgctgctctcgtcgtccatgatgtcgtagtcgatgcgtttctgcacgggctcgtcgtcgtgctcgtccatggcggcgctgctgaagctattgcccttggcggaggcttcgagagaccgtcgcgccttgcgcagtttgcctccgcgctggcgactggactgggagaaaaagtcgtggatcgagcgctgctttttggctccgctgctgccgctggaagggcccgaagtctgcgacgagccgctgccgcgcgccttacttcgcaacgcatttagcagcataacggcagaggtggacaattcaacaatgacccggatcgacgtttcccgccttttcgtatgctgtctgagaggtgctaggttatgatataagataggtagtggtgaaagagaaaaactatgagcttcaacgtggaatggctacactttcttcttgttgagcaagttgagcccttggaaggtcttcaccaagcgactttgtttcttggtgcgttcctgctcttctagtcgttgccgctcggcttcgagctccgctgcttcttgagctgtttgggcgcgctgggcggcttcttgctcttgccgacctcgctcctgctgctcggcttccaatgtgccctcatccagagcgacctgaacgagttcctagaaaggagagactcggttagtgcagggagttgcgttgggattgttgcaacacataccttgagccccatgcgatcaccgacgagccggagtggaagcgctttctgctcaccgctggcaatcgcgcgccgactgtacttggcggaagcgtagaattcgcagatctcacaatcgtgaaaaacgcgacagagacgacaggcgtcatcgagtgtttctgcagtaccacgaccaaacttgcggaacgatcgctcgtacgcgtcatattggcgcagaatggaggcgtggtcggcatctgcttggatcgcacggaggtagtaatcgtcaaggacgctgtatttagcggcagcagtatcgagaaatgttatacactgttcaagtcgtttggcaatgctatgttgacgtacctttgggtaggaatgggaggcatgaccactagcgtggcgtccgcgctgcactggaccgtaccgacacgacctacatacttgaatgccgagtagacgccaaacacctcgacctctgcgaaatggaggaaattcctgttttgtaattgaactcggacgaactgacccacagtgttagctgtagggagcacccactccgtcattcgtttgttggctcggaagagctcgaacgcgctcgactgaactttagcggcccgcaagctacatcgacgcaaggattaccagagatggtttcgtaagcacttttgtacgtggtaatgtgaaattactctcgagctcacccttctttgccttccacgtctttaaacgcaaattcactcacgaagatccagcacgggaaaagacgaccgctgtactcactcctgctttttgatgggttcaacggctcgtccgttcgattccacaactgaaagagtacaggttagtttcgcgcaagaaatgtatccaattggcctgctgcatacccgaattttttctatgacggcaggctggccaagatccacctcccaccacggcttgtcttcatgctacaaagcaattatgcccaaaaccagtgagtatggtaatagcgagagagtaaaaaccctcagccttacatgagtgtggatgcaagtcgttaaagcacccccagtttccccgttgacagccaggtgcggtccttgttcgttgtagacggtaatctgacgacatggcttgtgaaatgcaaggttgtaaggttcaacgtgcaggcacttgagtagctctggtttttccagaaacgcgcgctgtgctcaagacacacaaaccgatcggggttagtatgttaacatccttactactatcaacaaatatgtatacgtacgctggcatcgtgacctgtacatccatgattattgcgaccccaggtgtaaagttctccgtatgaggatacagcggctgtatggcttcctccacacgccacctgacgaattccaacctcatcaagctctggaatacgctcccacgacaaaacgtaccggctaagtgcagtagcacccccacagatatacacctgaccaccacgtagcttcttgcttcctctattgtattctgacgtgacctcggtgcaaattgcggagtgtgctgttccgcatgagacctgccataccgacactttcttgttcacaagatctcttaccagcgttggaacaacgatcgtatcgaccacgtgaggcccaagcccaaggcggccaccatttgcggatccccacatgaacaattcccctgccgttgagactgctccgctgtgcgaggctccgcaggacacggatcgtattcttcgtttaccagggaatttgaccagcagtggcgtcaaagagtactgcttgtactcgtcctcaacaattccgacacccagttttccattgcatgccgacccccaagtgtatagcgatccgtccgagtcaattgctgcactgtgcgaaaaggagcaagatacctgacgcaggctcacatcctaaaagcacgtggaagatgcattagtttacagattgtcagcgtacagacatacgttcgcacattgaaagcttcgacgcgctgggggtggtttgtgtcaaaacagatagctcctcgctcgttcggctgaagacccaggcgtccacttgcaccaattccacatgtgtaaagggatctaaaaacgcaggagagtgtgggttgccaatgccaacaaattcaagaatccgtccgtacccatcttgcataagcaatgccacgtgggaaccccctgcagaaatatttgctacggcctgcaaaatcaaatcagcacccgtcttcactttttatagtatatacttaccacagctcctctgggtgagtgaagcggtggtcctcttcgggtaccaacaaaatacaaagatcagatcgaggcacttgcacactaccggtacgttattttcctgtcttaccttgaagtaactccgctcatatcaatccgggaattttcacctcgagctgtaaactcagggattgtatcttccaactgaacaaccctccgagcagccctggactcgtatgctcgatcctacaccaaggttgtccacaaggacttagccgaaggggcctagtaaaatgatccgtcgctttactcaccgcgcggtcaactttttcgatgattttggcttttgcatcctcttcacgtttggcatgtcggttgtcgcgcgatattttgacatctgcccactgcaaggaaacaagttaagtttactccatcagaaaacgctccattgatgattaagatgaggcatcacatactccttgtagtagtttctctttttcctagcacagaaacacaaaagggccatgagttatgacagctaagccaactctggacaggcaaaaatgtaccacttggaggaaattaatctgtttgtcttgagtgcgctttttgtctgcactgtattcctgttcatgcacatgtacgttttgtgattgcgagggttgtagagaatcacatttttatctttgtaccttgatcatattttccgattctttgaatgcggtgtgaccctcttcaccttttacctcgacttcaagctcgaagcattcccccagcgtgagtgacaaatccattttggtcagatgatcgagcttaagcccacgcagttggccagacgtaaccagctgctcatattcaatcttgggtaggagcacttgctccatgaacagcagccgcgtcgcattactgggaggtggttcccaaacgtcgtaatacgtgacaactcgtttcaaccttcaaagaagcaagttatcgaattgaaatcacgccagtccgtttatatgggtatccacctacctttcatagtgttgattctcttcgttttgtcgagcttcatgctccagctgttcttcttctctcgcttccacaccaggatcatgtagcccttctagctgtagtcaaaggcatcagacaagttttgactattgctgtattgctgaaggccatacctgtgcgggggtacacatcttctgaaacgctgaccgaggggtactcttcgactgtacgccattatccgattgtttctcttttcttttcttcgtgttggtaccttcgagatcatcatctgagtcgctgtcaaatccagttaggcgccgagctgatacctcccacgtattatttctccctccccagctataaacactccccgaacttgtcacggcaaaagcgacactatcgctgattgcaccatgcacgacacttctaccccacaaaggccccgtatttgtcatcaaagatgttgatcgaaatcttaagctccgaacaaattcccggttttcaaagaagagcttggcaagttcttcctcgccttgattgctctcgtcacgggtgaactcttcaatttcggatgtttctgtaggtgcgtttgagtcaacatgttgatctccctcgttacgttcattcggcacctctctctcacgagacattgctcggatatgcattttcgtctgggaaattgtctgtttgttcaatttcggtggactctggaacttccagcgtttattttctaatcttcgacgaacgtatttggatgccgaaacactgagctcgttacctgctcgaggcggaagagcattctttggctttggcaggacttcacttatttccggcattgggtccactgctttcttttgtttgtttacctttgctgctgcgcgtgacttgttttcctgggcttgatcagttggatttacccgataagaccaaatctcgctcacagccttaaaccctgggaacagatcttcgtctctagtgacaggatttcctgcaaactgatcgtacttcccacttcccataacgtacacctgccccgctgcatcaagcgcggctgctagctcgtcttcagctcgaataagtgctttgcgactcgacatttctcgaaactctttgtctcgctggcggtccagaaatcttttcgctgcctccagcgcaacagcctcgcgagcctcctcttcgtctaaaatcgtctctagcatttgctgcatcttccatagccgtatatgcttcggtacttctatcccacggttcgtaagctcttcacgtacatctacgagacgaacccacatcgagcgaaactccttgtaatcaatgtatccgctcttgtccttgtcatatttcttgaaaatcttctcctgcttttcatcgctcacgtccatgccaaagtactccagcacatcctatttacacatccctcatcagtcaaaaccatgagtagggtttcgccccacatcactttgaccaagctcgtaccgcaaactctaatgcatcaatctgacccgtcccgtcctcatcaaacaactcgaaagcgtccctaggacccagcaaagccgacggagagaaccccagggtgttacctgacacaggatctacagcaaacatggccatcttgaactcggcgaggtcaatttcgcctccgccgtcagtatcacatctacgaaaaatccgcagggcttttgcccccgagatggaaatacccagatccggtagctacacagcagcaagcacaaaaaagtcaacaataacacatttttcaatggtacattttgaatcctccgtaccattgccagaaactcggatgcagatatgtttcccgacccatccgtgtcgtatttcttgaacagctcctctggcgataactacgtggcacgcggcagttagcacctatttccctcctctacctgacttgaatgagtcaccctcaccttcgagttcagcactgcaactttcctcttctcagcctcggctgcttccgcctcgttcaaactctcgtcccccccgtccttcgcgtatcgctcgatggtcgcctcgtcgtcgctctcgtcgctgtcggctccgaacaagtccaagtccccattcttgcccttcgcagacgaagctgccgtagaaggagacggtgcagccagagatcctcctcctccttccgctacgcggctgctagtgcgctgccgctccttgcgtccgaagagccccatagcacgagccacgttgaagtggcgattcaaaacagccaatcatcgttaagtgatgtggttcacttaacctgggtacacttttgaatccattctgtacccactgttgaagacccacatgcatcagtctcacttccgcaattgcgatcgcgagcacaagtggagcaggagtgatggtctcgtcggactgggaggcgcaactgaccagagaaggcgcctcgggtagtacagagagccaactggtgttccaagaggagttcgcgctggcggagagcgtggcggccaaggcgcagctggtgcatcggagctttgtgcctggctcgccagactttttgtactacggggcgctatgtgagctgctggaggtgcagaggctagtggagaaggaggagcacgggaaggcctggcagagactgactgaggtgaagacccagctagggatggcagagagacagcttcagaccaagagctgcagtaaacgcgcacagaggatccttagtcgccaactgctgctggagctcgacctgcagactaagctcagcaagccggaagaggagattgcggtgagtggggtgaagtggaggatacataaagggaaactaatttggtttttgtggttgtgtaggtgacaacacagcgtatctcgacgtcgctgcaggtgagcttctcggacccggaacctgctggtatatcaacagaggccaggaaggagacgtatccgacagtgttgaggtgggtgaggtgctatgtggggaggttctgtgtgtgaatgcggagatctcatcgtgtacttgactgcgatttgtatatattatagtaatgagttgctggaccttgatgtatggattcaaacgaagctggattccctgtcgtttcgcactgcaaaagatacggcgttaatggagatattgcatgacctcgatgtcttcgggcgcgaaaagactttccaaaaaatatgtgcttgggaaggatctgattatgatcgctgggtgatgctcgacgtgttcttggacacctacgcgtttgaatacgcggacattccggggtacgttgcttctgtacaggaaaccatactcatgagttgaccattttcctagcaactgacttttattaaaacattccttgatctagatatgtggatgtgatcgtcaaggatgttgagcgtaagaaggatacgtacaagggctacagctttaacttccgttcagcgcatcgcaacttgtcctttttgcaactattggaatgtgcaaagcgatcgccagagatatttcgtagcaacagcgagtttgcagttcgcgggatgcaactacttcaggctgctgcagaggtggacagtgctcacagcttggacctccgaagtgaagctcggaacgagaaagagctgcagcatatcgagacctatctagagtttttgcgtgattttacgccgactgcagtatatggacttcggatgaaaactctgtaccggaagctggaacttttgaatatggcgagctggggaagctctggagccttggtgggactattgaacgcattggtggagtacctgaagatcgctggtggtcacattgtaggtcttaataacgttagcgggtatggtttcgcgagcgtttctcagatgagccacgatgagatcctccaaaagagtttgagaacactatggtcatcccaattggatgatgaagtcgttctcaatacgatacgtcctcgcttgaataacgacttcgtggaaattcagtatgcgttggtcatgatcaaattggggaagggcgaattctcccattggacagcaaagctcccggatgagggtcaagcgctatcgagccaaacgagctcgtcggaggtgattttctgtgagtcaaacccaacttattttttacccgaggacccactgcgggtttacgtgcaggctcgcaatgttaagtcactgaccgcccatctgtatgagatcaaaaccatggagtactattcacgattgcgccgtgaaatcaagggtgatatatgtctcgatggattgctaccgacggaagagcaggttatcgatttaagccatctcactgcttggcaagaagtccgtgttccaatcgaatttcttgctaaaagaaatccccagcgtggtgtttttgtggtagaagtgttcgaaaaaggggttacgtgcagggctatcttgcgtaaaggttttctccgccacgtggagcgaatcacgacgcaagggcacgagttcgccgtgctcgatgaacgtggaaatcttctccgtgatgctcgtgcgcttgtgatcaatgtgaagtctggcaacagtaggtcccaggtcggtcgcgagtatgcagcggacgaggaagggggcatatctattccatttcggcacagcgacgtgcattcgagagataagttcgccatcgtattctgccacgggtcatttgggaattttcatggatcattcagctatttaccagaatcatttgccgtggacgttgacatacacatcgattccgagcagttggttcctggctctgttgcccagcttgctactcgcccacgtctgctggttgctggtattgcgaccggcgaatcacttgagctgcttgttgatgtgaagttgacgattgagttcgatcttgtcaacacgagcaatgttggctccagctctcacaaggaagtcctgtcattttcgtccatgcagtcgatcgtgaatcaccctccttgctttgagattccgatggatgccagaggcttcaatgtcatggttagtgcttgtgtttctccacccaacgtgacaggctccgttaaatcgactgaactgccaaaagtgagcgacgcaaaacgatttaacgttcagcgagtgaacaactttgacagcacatacaccgctcatttggttcgaaggtcgcttgagtcggggaacccgtacagttcaagcgagttccgggtattagtgctcggtcataatggagaaccggttccgagtgtgcgtgcgttgttcacttgtaagcacgtgcactccaaaaacgatatcaaggtgacacttcaatcagatgccacaggtgagatcaaactgggccaacttcaagatgttgtgcgccttacggttcaatttgaagctggtggtaaccatactgattcatatagtgggagttgcccaacttgcgttcatatcgacctcaaattgtgaattgctcggtggatgaaatcgtggagattcctatcccgttcgcgtattcgggtaaagtggagacttgggtcaaggataaactgatatcagtttgtgaaatcgttgatacagtactacaaaatgcactacattgctcggaagtgacggtggtaaagagtaatctgaattgccctgtcggcattgctgttcaaataagacgagctggtacatacgtcgtctacctccggtcgttgaatctcaagtatcccatcaccgtgtgcgagaagaaatccatgcacacatcgctaccgcttggtttgatcgtgcagccgacgcaaatccttctcgctacccaaactctgcctttagtgatttgttctcaacagctaaaggtggaagataaacagaagacagtactggagattcaattgcggaacgcttccaggaattcaacgcaagtattggtgtttttgaagcactttttggacactcgaaccagcaaaataagcgaggtatttgttggagatggtctgaagcctgcttcgagttctggaacccgatcaccaagactgtcatttccctccaccccactcgaaaacgactttttgaaaatgcgcaagatcagcgatgaatatgcttacatacttcagcgacgcgcgctggtgtccgctaatcaaaactctctgagttttttggggacctcatcgataccaaaaccatcgctacttcaaaatccccacgttcaaagtgaaacagatatggaggtggtaaatgttgaggctggtgagaaagtaactggattcaagtctgtggccgcaaatgatatccaatcgggaggaacttctcggaaaaatatgttgcagaagaagatacatcgaggatacggaggatctacagcaggtcgtggtggtgggatggtaccaagtatttcgttccttgggcagcaatcgcaactcgtagcttcgtgtattgtagatgagaatgatcttgtccgcatcgagctaagcggtctccctttcttttccgacggaatgagatcttttgaagtttgcacggttgcatttgactgctcaagtggatacatgtgcacgcaggaggtgccaatgagtcttcctgctgtggaagataaagtggtgataccgaagcgcgacatccgtctatcagttgaggaggctctttcgccatccgaccattttcatcaggttgacagccacaagtgcgtgcatagtggcgaaatgaaaaacctaccgcgttcattttccagcaagtatgcgttgtatgaaagtcttgaaagcgctatgaacttgtggccaacgcttgcctctgagggtaatgtcgaagagctagcgagcaaactgaaagaatggtggggtctgagcaccttggagaaggaacatttctactattcaaatgcgtgtgatgatcttcactttttcctttcgcgcaaagacccagccttcttcaagcagtatgctgagccgttaattgctgcgaagatttgcaagtccttggtggactatcacgtgcttgatgatgaagctagtttgcggagcttctatcttggccctgctgtatttcagcacttaaattgcatcgagaagttactggtggcggaaaggatgacgaatatggaggagaaaacgcgcatctgccgtgctgtcatcagcgagatcgaatctatctatccctcgggctgctcgagtgtactggcgcgcgtgtttaacactgtgttgtccctaggtcaagttgagccgtctgccgtcccagctccaccaccttacgcagcattcggtgcacgtccagcgatgtctgggtaccaaggtggagcacaaatgcttcagcacgcctacgctcctgctagtgaagtctgttatagccccacatcaccaatgggtgcgccatgtggatcagcaatcgatctttcttccagtgcgctacgttttggggaggcttgtggtgctacatgtatagaaactaccgagattgcttctttcctggaatctgatgatgactttggtgtaagaagcctgggcagcgagagtgacgagagcgagaatacagatgatgacgatgacgaccctcaagaagaaagcaaaaggaaaaggaagcctaagcaagaggctcagtatatcccacctggcaaggttcgcaaagtgcaagaaaagcgctatttctctggccaacgtccacctctgacaggatgcaacatgttctggaaagagtacgcagagcacatgttgcgccgcctagaagatggtggtcggagacgctttgtgtcatcatatttcccggaggcactagcctccatctctgaagggctattcgcgctagcagtgctggacctcgacaaggacgcaacacccagtcaagtgcaactgacctcctcaatttcgccagctagtgaagctgtcctctatcatcgcagcattggccctgctgagagctcgcccacagccaacacaatgttgattttgaagcaacgcattcgagacgaaaatggggactgcagcacagagctactggtcaacaaggtttacaccacgagcgtcactttgagcaacattggctcggaccaccttaccaatgtcaatttattgctgcaaatacctcaaggtgctcttccaatggggccgagtgggttttacacgataaacgagatcggaagcgtggcgcctaaccatacaaatgagttcgtgttttcgtactacttcccagaagaagggctattctcacaatatcctgcgcgtgcgtcaattgaaggtgttgtggttgcttgggcaaaacttgaagagaacacgacgacctgcaaggtggctcgcagtgcgacgtgtgtgaatctagcttcttgggcagatgtcagtgctcggggctcgctggaagagatcgtacagttcatggagtctgcaaaacctggtgtgaagactgacttccagaagttgttctggcggtgccatgatgagttatttttccgccggctggtgaattatcttagaaataagatgctctttgtgtcggggatttggaaatacgggcttcttcatcgtgatcaacaaacaatgaaggagttcttcgcaagctcgaatgatttgtcgcagtcggtggggagtggcttctgctcctcgtatgtggacgagacacgattgtatcgtagcgagcggtttggatcgatgtttgatcagttcgatcactgtgagtttggacctttccttacaaggcgcacgcatccggtagcgggcagagtggatacacaaatttcgtgggggtctggcgagacaggtcaaacgtcaggcaagcgtatactgaatgctgaggctcgaaaatactatggggaactttgtcagcgtctgggaacgcacacacggatgaatgcgcagcacttgctggtgatggcgtactatatgattctgtttgatcgtatcgaggatgcagtcaagacttttgctcggttggaaaatcttgacacgccaaggaagttggagcttgagaatacggttcagtatgcatacctctctgctttcctcgactttttccgtagcaacggcgagcatggccgagcatttggcgtcgctcgtcgtattatttcgcgttatgctgcgcatccacaacctcgctggcaacgtcggttccacaaaatggaggagtttctggaagagtttgacgccttcgaggcacaaagcttaggccgccttgaggacatggaagtggtcgatactcctatggagactgaacgagagagccccttcaatcaagatacagcccatggaaaccaagttaagctagaagcaagtgtggacaatggcctggttacactgttgtcacggtctctaggagcgtgtgagctcgcgttctatccgattgatgtggagcttatgttttcgaccgagcctttcaacacattttctgactccgcagcatctgcatcaagtcttctcttggtcgagcctcggcgacaattgtctgtcgatcttggtactctaccaaacgatgtaaacctgtcaaagacatcggtgccaattcctcaagagcttcgtgctacgcaaatgatgattagaatccaagaagtttcgtcgtcgcgcactgtcagtagtgtggctccgcctatcgacatgattcagccatacttcaattcgagtttaactgtcgagatcatgactcagtgtggtgtactccaagttttccacgacggattgccagttcgctcctgttacgtgaaagcgtacgccaagctatcgtctggaagctcaacccaccacaccaagactgagtttttcaaggacggctacaccgacttgctcggcaaattcgactatgtgggcatcaacggagacctcatttcgaatgtcgagaaattctctatcctgatctctcacgacaagtttggtgcaagcgtggaacaagctgacccacctgtacttgcggctaccgttggcgatttcaccagccagccggagcgcaagctgcttctctactagggcgctcgaactgctgagcacggtgaagcagaaaaaaaagtactttgacgcagggagtgcagttgaatcgatattcattttcctgtctgactttgttgatcacgttttcgcagccgactaacatcttcatagcacagactcaagcatatagggcttctcacctccagacagcagaggagtctcaatagtagctacagccgcacgtcttccacttcctgccgcacaggaaacaaaactcatggccgttcaagcacgccatgcggttgcaccccatcgacttctgcacccccactccacaaccaggacactgcaccccctggtgtagctgcaccacacgcgtgtattcacgatccgactgaaggtccagcgcgcgccaaggtttgtcctggagaaagcgttcataagttacgaattcggcaacattcaacgcctcgcgcacgtagtcggaggggaactctctcttgcagcagtgtgccggtaccatggagcgatcgcggacacccatacgacaacgagtcgcaacacacttggtgcagtacagatgaccacacgacaaaatgcgacggataccctcgtcttccaggggcaacagacagcatgcgcagcttggaggtggtttggactcgccatccaccaagccggagacgtctaccacagccaacgtctcgtccgcactgtccgcttcctgatcagcctccgcgggatggagtaacttgtgttcctccttggcgtagaagctaagagcaaccgcttcatcaagtacaatttgctctgcacgacggatctcttcgctgtacacctccaacgcgtacgcttccgccagttgggcttcaacagcagcttcctcgcgaccggcaacttcatcgacaaagtcatttgtgtcggagtttaggtagaccacgtgttcgaagtgaccatcagcagcaggctcttccagctccctcaacgtcggaagaacttccatggcgtcacttaagagcccaagtcgcttatgctccaacagggaaattggaaagtgacatcaccaccaacacgagtttccactacaacaacaataagaagaacttcttcgccagttagagtcaccccttcccagcctgccgttgagtcttgacgatggcgtgcagacagcaacatatcaatcaattacatcaagaagagagagaactgctagttctcgaaatgcatgattagtagattagttagttaccaattcaacactatccaaatctgcagattgcacgtgcacattgactggagtcagtcttactgatagatctttccgcaagtacactgcacgtgcagacagttccaacagaaaacgtggccgagggtgcactgcatagcattgcaccccgagatcttaacaactccgatcccgcacacaggacactgctttcccttaacttgacgcaccaccttcgcgtagtcctggtcggactgaagcgttgatgaacgtgggtcacgctcggcaagaaagcgctcgtattgagcgaactggcccttggccagtaccagtttgacgtagtccatagggaactctttcgagcagcaacgaatcggaacttgttcacgattcgcaagactcgtcttcgccatcttcgtcaagcaggatcggcagtagccgtggccacacgggcccaccagccacttggcacgtggtgacacagtcgacaagcaggaactacatttacgcgcgctcagctgcttctggttgtttatcttctccacctttttggtaggtcggagctgcttcacggatgaagcacgcgctttcccttttgcgggcgctgcggccagcacggtctcgggatctgccttgcgcttggttccccgtgtgtttgcagtggtggtggtggtcgttgcgccggtgtatttaagcatgagcttggcatatcttgtgagctcgccgcgtggaataacgcgattcaccagatgcagtggcagcacttgatcacaacagctaactggtgccccactattgggagcatttagatagctgcggacgccagtttcaacgcaatggtgacaaagcgcgtgaccacagtgcgctttaatttgtgaagtggacatcccgatgccacacatatggcacggttgactggataacgccaccgattgttttccgggcgctggaggcacggtgttcccactctgcttcgagttctgcacctgaacacgtcgaaccttagagccgcgagtgaagtatctgcgctgaggcggttggggtggtgagttatcagctcggtcatcaagaaatgaagatagcgcagccgacgctgtcgtggtgacgttgtcctgcaccaacttcgccggttgtttcatctcggtgtcatcactcacgacaggaggggcgggaagaggtagcgcaggtgtactcttaacgttcattcgggtagcaggcggcttaccactcccagcttcgtcgcgaggttgtttcatcaggaatgagtagtaaatatcttcttggcacgcgagtaccatacgaatgaagtcgtgcggaaccactttttggcagcactgcaagggcgcgatcctgcgtgacaatgcctcccgagcttgccgtcccagacatgctcggcataaaacgtctccacacaacagcgggaacagttgctgcgtcgactccacccccgaccacgagaactctcctgcacacccagaacattgagcagagggcgccatctcaacgtgctcttcggatcttttgccagaatgcaaagtgtgagtgtttgatcaaaccgcgtaggatctgggacgcggcactcgctttctctttggccaaaagcgttggattccataggacgggccaagtcgagacgcatcaacgccatacagtcgtgcaattacaggaaatacaacgttgaatgcaacagctcctattaaccgacaccaagccgaagatcactcgccaagaattgctcgcagttcatcctctgggatcagcgggcagttgcaggagccccagcgctgcaggcaggtgtagcaaaactgatgcccattagggcatgacatgtgcacgcaaccgaaggcacgctgcacgccgataccacaacccgggcactgcttcgctccgaaggctttgacggttgccgagtactcggcatcagacacgaggtccgatattttccagtctttctccatcacgatcttctggtactttgcgtaatcctcaggtccagtcaacgcttcacgcacgtagtcatcaggcagttctttcttgcagcaccgcagtggcgccagtgcgcggtcacccagcgctagacggcacatgcgcagaagacacggcgggcaatagtagtggccgcaaggcgcaattcttagctcctccgtcgctttccaaacacaacagctcacacaatttgtacgtgtcggggttttctcacgctgcctcatgtacaacgcgaattctgaccggctcagcacaacctccacttgattcagagctaattcgtgtctgcacttccagcacgttgctggagtaccgttcttgagagccttcgagcacgccttcgaaagacacgtaaagcagacgcgatgagggcaacgggaaagcttaacaacccgggttgccttgttgctgcagcacgaaacagtcttcttctttgccgaccgtccacgtgcagcgaacttgacagttgatggcatgcccaagctgctatttgcgcaggtctctctccttcaaatatgcgcccatgatcgctaccaatcgcttcttcgcctcgtcccagcgtttgtcaatcgtgttcaacgcaccgtctcgcagcagaagaaacgagtgaccaccttggctactcgcgtcgttccatggagcagcgccaacgccaccaacaggaacccaaacgccgttgtgtacaccgcaaacggatgctgcgctgtcattgcatccaaatgaaccggaacgcggctgtctccaatcaagaagccaagcacaccaagcaccagcaccaccgccagcatctggccagcgccaatcaacaacttgaagcacccaaagtacacccctgcaagcactgttctgggcgacgacgtgcccaacgacctctcgaatcttggacacggctcaaaagcctgtccttgtctccctgcaattggcgggggcactcgaaccggcagcagcgggtgtaattcagtgctagaacttgagaccccccactcggccaagtccatgtcaatgcaatccgctgccgttgcagccagtgcattgtaaataaaagcgtctatgcagcacaagtagaagaccgtgtgcagcagcgcgtgaaagaccgcgacgccgcagccacagaggggcatcgtcacgaccgccagcgctgcccccgcccacactaacaatgcagcgacaatactcagcactgcactgcacccgttgaagagcagcatcctcgtcacctgactggccaaagtgcagggccgtaccaagttgcagcctcgcggttggcgcaacacgctcacgcgatggcgttccggccgataggcgggcggcgacggaggccacgcgaggcgttggcggggctgcgaagctgtatacagtggggtgaagtctgccacggcctggtacatgtgctgcgacggcggtagcgtctccgtggcgcggtacgaagcgacgaacggactgcgcggcggcgacgacaagggcatcatggccggccagaaatcttcgatttggtccgattttttggtccaatttggtccaaatttgagctgatgcgaatagaggggatcccctcctctgttgcatagcagacaattccggccacaagttgataatcatcatgggagactcgcagaactggtcgtcgttcgagcctgaggcgcccaagcctgccaagcagccacagacgacggagagcgacgacatcaagagtagcaagggactgggcgcgctgacagagcccgtggcggtgcgcaacctgcgcaatcgcctcatcttcggcagtttggtgggtttcgtcacaggcgccaccttcggaggcagtacgtacagttaacgtcgctgcaatatgttgcaatagcttctgcattgctgacttgtatggctggtagttgacgcggccaagacgtaccacaagacgcacgggaagctggcgcctgcagctctgagctccgtcgcgcgtggcgctgcagtgacgggaggagccttttcagggtgaggaattgcagcgacgatgcgtgtgttgagcgcgttgctgacggtgttttgtgtggcttttggtgcagtttcttcttcatgtactgcgggatcaagacggtgctggcgacccagcgagaggacgatctcgtgggtgcgggcattgctacagtggcggcgggtctgccgttcatccgctcgacggtcatgaagcaaaacttgccgtacgcactcatgctggtggcactcgaccacttccacgaggagctcagcgaataccgtaaatagaggatcgatagggtcttgcttttccgttgcgttaatggtgttgtagtggatgaagcaggtttgcagttgtgtgcagctcgatcattgacggtggcttaccaaaatgacgcaggattgtgtcccatgatgaacttaatttgtgtttctgtgcattacgctttgcgtgtgcaatataggtctggctagcgccaagtttcttcctgacgccatggagacgatttaacttcacgcctacagcctggcgccgacctccttggactgcgcaaagagctgctgcatctcgacgaggctctcctcgagcaggtggcccatgcgggcgccccaactgccgtgctggcggcacgcgatgttgaactggatcgactcgttcttgacgctgtaggcgataccaagaccctcgggcacgacctcaccccagccccagccgtcaaagagctcgttggtcaggtgactcgtggaaatgagccaacgactggccctagtcatcaccgggtcctcgaagaacggcacgcgctcgccaggctgcaccagcagcttcaagccgagaatgtggcggtccacaccacgaccagcgccagccttgcgcatgtacgcaacgtgggcattggctgcagctagcagcagcttcctcttctcttccgcagtgacggcaccggatacgtcttccatggcgtcggtaaacttcgcgctggccgtggagcaactgcgcgtcgtctcggtgcgtccgtgtaggaagatgcgagtctgcgatgcctcatacgtggccacgttcttcttgaacagcttcttataggcaagttggatcgcaagctgcacaaacgcgtcggggctgcagcggaatcccttgatggagcgtgcgccgtacccgtagaaggcctgcacgaacacctcgtggtcgatcacggtctgatcaaacgccttctcggcttcaacaatgttgcgaagcgtctgagtcgtgaatcggaacgtgagttgcttcggagcaacgagcgagcgctcaagctgtgccgtcgtctgaccacgcgggcccaggtcggtctggctgtggtgaagacgcgacagcagcgagtccgtgtagcgagccatgggcatgccgtccagcatcgagtgttcaccgagcagtccggccttgccgttctcgaacacgacaatctgcacgcatttgtcgaagaagcggttgcggccgttgccatgccacaatccgcgtgcgacctcggtacgggacacaggcgcttcatcatccatattgaccactagcaacgagctctcaatggcacgcagcgattcctcgttcttcggggacgcccggatcagctcctcacgagcatccgcccaggagtcgcgatcttgggacgtgagcgcccccacagccgactccttggctcccgcagcttccaggatcctggccagctggaagcacagcgcttcaaagcccagcggctcctgagtgcgcggatccagcagctccagctggaagaacttgttgtgccgcatgaccacggcgtgctggtgcatcttggggtcgtagatccggtacgagtcctgctcgcgccgcgggatccggcacgcgttgaacatgtacttgtaggctgtggcacacacgggcgtcttgttcttgccgagttgctcgggctcaagcgatccgtccgcaacttggcgcgcgaacagcaccgagcccctcagcagcgcggcggcccgtccaatgttggagcgctgggccgggtgcacgctgtctgcgaaatggaagaagtagctgacgttgaacaccacggggtcgcgcacctgcaggtagccgagcgtgttccaccactcagccaggaagctcgagtcgccgcgctccttggctctggcaagcaagcggttctgcagctcgtcacccacgcccccgggttgcaggaaggcggcggccagctctttggcgcgcgtcaattcggtgggcgtcacgagcggccgcagcgtctccaggtacagcgccaccgtgtcacccagcgcgggcacaggcagccgcggcagcgcgcgctggaagcggtagagcggacgcgcggggtcctccaggtccgcctcggcgcggtagtcgccccagcactcgcggaagccctgcgggccgaagctgccgtgtggaatgagagccatggcttgggagatggtcgtgtgtctttcgcggtagatcggagactgaggctgaaagggcacaggttcaacgtcaacgtatgcaatatttccaaacgaagcgcaatatttccaaatgaagagcaatattgtgggtttgaaaagcgagcagtgacgacagcgacggaccttagcttaactggaagatgagcggttcctcgctggtgcctgtggccgtgacggcggcggtgggcggcttggcgctctatgggtttctgcgtattgctcgcgcccgcatctacgacgcgacgatcgtgaaattgaccactggatggtacgaggaggtgctggagcggctccccgagggcaggaagttcctggacgtgggcattggcacaggactggcgctgatcaacaatggggcgcaactgaagcgcaagaatatcacagtagacggcgtggactatgacgaggattatgtgacgcgctgtcaggcgctgctggagcaaaagcagctgacgacgcacgttcaggcgcaccacgcgtccatctacgacttcgcgggcggcccctacgacgccgcgtacttcagttcgtcgctcatgctcatgcccgacgcggtgaaggctctacagcactgtgtggcgatgctaaaacccggagagggcaagatctacgtgacccagaccatccagacgcgccactcgaagctcgtggagatcggcaaaccgctgctcaagttcctcacgaccatcgactttggcagcgtcacgtacgaggaggagctgctgagcacgttcaagaaggcgggactcacgctgcgggagcatgtgccgatctctggctcgacgatgacgtccacgcggtccttcagactcttcgtcctggaaccgtgaagcgggagtgagacctgctatatggaaaccgctatgactttcgaacagcgttggcaatagccaaacaatgaagctttgtacatccttgtgcagttcaataacacgcttcgggcttctttctacttttgtgcgatcttgaaccgacaccactcactgatttacggcgggggactcaaagggggagaattcgagagatcttggagagggatgactcgctcgaggtggagcaatgtgctccctttggtggcgacgggtatttgcctcttgcttgcgctaggtttcggtcaaggggtggaagcgtcggttgctaacttgaagaggaacgcggagcctcatgacccggtggccgccactcgaggtctcattcaccgacgactgggagacaaatacaatgaccaggttcgtcatctctacgcccggccttgttgtaattgttaaccttgtgttgtacaccagatcgcacttcgtgtactgccatcagatgccgatcagctggacgtgtttgagcttggaagtgacggtgacaagctcgaactcgccgcgaactcggccaccgccatggcgtacgggctgcaatggtacttcaaaacagtactacgcactcaaacagattgggacaaccacaagctgcaacttccagacaaactaccgaaagtggatgaacgagtgcgccaaaagcgaagctcgaaattctcgtattaccagaacgtggacacgggtagctattcactgtgggcctggaggtggccgcagtgggaaaagcatattgactggatggcgatgaacggtgtgtgctcatatcaatctattgaagttgtgtaatactgattgcatatttactatccaggtattaatatgccattggcattcactgggcaggagaaggtgtggcaaaatacttttcacaagtactacaacgtgagctatgagggcctcgggaagtttttcgctggctcagccttcttgtcctgggggcgaatgggaaacctgcgtggaagctgggtgaaaggacctctaccccaagccttcatcgacaaccaacatgagctccagctacgtattctgaagcgtatgcgtgagttcggcatgattccagctttaccagcgttcgctggacacgttcccgaagatttgaagctcacattgccaaacgcgaacttcacacggtcgccaaactggggcaacttctcagacgaccattgctgtgtctacatgattgagccaaccgatccgttgtatcgcgagattggcaagatcttcctcgaagaacagcgcaagttgtacaactacacgtcatcactgtaccagtgtgatacctacatggagatggcgccagagttttctaatctggacgaactgaagggtgcagctcgcgctgtgatcgacggcatgtcagcggcagaccccgatgccgtttggctaatgcagggctggccatttgtggacgatccaaattattggaccaggccacgcgtgaaggcgtatctcgatggtgtgcctacagaaaagctgatcatcctcgacttctacagtgaatccgtgcctatatggaacaagatggacaactacttcggcaagagctggatttactgcgtgctgcacaacttcggcggaaatactggaatgcggggagatttgccgacgctgggcacagcgcctgtgctagccaaccgtgccagcaatggcactttggttggtgtcggcttgactatggagggcattttccagaactatgtggtgtacgaccttacgttgcaaatggcttgggtggataatcctcttgatatgcagaattgggtgccaaaatttgccgcgcagcggtaccattctcgaaatgagcatgtcgaacgggcttggaactatctctcgcggtcggtttacaatcgcacacttgcatatggcggtgtgaccaagaacttggtttgcttgattccacactggagacttttgtacgacaggtttcagcctaccctcatcaagtacgacccgaaagatgtcgtgcttgcctggaaggagcttcttctcgttggagaagagttgcatggtgtagacacctaccgacacgatctagtggatgtcacgaagcaattcctgagcaacaaactcttggagcagtatgttcgcttgaaggatctctacagcgcgaaagtcgtgccatctcatgaagtatgtgggttaaccaaggccatgctcactacagtcgagcgcctagaggaaatcctcgccacaaacgaagacttcctgcttggaaattggatcgctgatgcactagcacttgctggcgacttaaatgttggagacgacaggttgacgaaatctaaactgcgagaatactacgaatatgaagctcgcaaccaggtgacgcgctggggcgatgacaacaacgaggctattcacgactatgctggcaaggaatgggcaggtctcgtgaaaggatactacttgcctcgctggaccatgtggctcaacgaggtctgcgaggcgtatactgaaaaacgtgacatggccgtgaaggcgttgaagaaaaagcgtatcgcattcgagttgcaatggcagctcagccacgaggtgtatccaaccactacagtcggcgatccgcttgcagtgtccaagcgcatatacagcgagtatatcgacagctatgccgtggttccttgggatttctttgggatcaacacagcgacatcgtagtgagcttacacgacaagcttgaaggagagcaacggtctgtacggccagcgagcgtaacctgcatgtttagcatcgttttgcatttcatcgtactcttgaatactgcttgaactccatggtacttcgaagtattacttagccttggcattgtcattagtcttgttgatatactgctgaaatattacagtaaagctcagtaagcgtttggttcgttaagcttgttttgggtttgtagactcatcttgtatgtgaacaaataagcattacggtcacatttgagctttcgtcccagcccgcacatagtgctgtggttggcgacaatgatcccggtcttcgaaatacttaagtgaaacaagctgaagccaatgacatctcttactgtgaagcagcattgttagctacccccatttaaccaactgaaggataaactgtagtcgtttcctgcaccagagttttgtcagactaattctggtaacttcaatattccttggtcaacacgcttaccactatgctcgagtgcgttttccactgtgaaaaagatccagaaaacaaatttggctgaaccattcgctagccccgatcgagcacaaatgaaagcaggcgtagcatgaagattccaagtttcctttggatcgtttatcgatttatcatgaaatatagtaattctaataccgcgtaggtgaggacaccaccaacctaagatggcaagaccttctttatcaagcttctttaatacattcggttgggaagataaaagtcggcaacttcggtgtattccacgagaatcacggaattccgtcgtgaaaagaacttgagccgttttgtggacgaaaacgcttcgttctctcgaagtataaagattctacatgtagattcctcgagagactttttttcgcgaggtttaatcttcctgtgcgctgtgtatttgacatgggcacgagcaacactcgcttgacgtgttttttgcagctgtgtcgtcgacagcgccggatgagtttaagctaccctcgctaacatcattgtgtttcggaacggctagtgcggagctgtaccgaaggcgaacgtcaaggtttgacttcgtttggaagtcttccttgatgagctgtagtagtgcaacatcaatgatgcaagctgatcttaacgggcggctggccggaattcggcgtcatagtacagcgatgctcttcatcaagtggctggtatccagcatcgacaccaacgcttgaattgaatcatcgctatccatgccttgaggattttcgcacaaaatgcaattttcagactaacatttcttggcaacagcaatggaactcttcatgggctagaaaatgatgagtttcagttcggtatgcgagagtttgatgaacaataactcaagtagaccaagagagccccctcgtagacgtgctaacaataccaataatgttcactctaccagcaagtagacgtgcttgcgtcactctgttttcatttttggtaatgatttcccgcttttacaaataaattatttctaaggaatgtatcgtcattacgaaaatgttcacagtggaggtaagaaaagtctgctccaagaatgagtcaagcgtacaaataaggcgctgctaaatgcagctgtgtgcgtagtacgagccttgagcggagacaatacagcgatgcgcagccctccaagtggaactgcgcggacagaccgcggaagccatcaactatgcagcaatgcacaaaaattcgagtgtagccaaaagtcgaaaatcaggagggcgggtgtccctccagcctctgcataacgtctccattctccatcgatctctcgagacgcgcgaggtgtcgaggtgctgtctctcgcattcttttttgagtcggggactaaaactcttgcgtctccacttgcgctggcctcttttcctcgctgaccaccggctccgtcctcataattggaaaacgatggcgccgtccgagtggctggcggcctggcgcgccgtgcactggacgtgcctcggagtcgcgggcgcgagcgcgctcttcctgctgctgcactcggccgcgttccaccgactcaagcggcgcatggccgacttgctgctgagcggcatcgcgctggccgacgcgggcttcgtggtcatggaggcgcttaagcagctgctgcacgcgagctacttggctcgctacggagagggacacgacggacaacgcggctacaccacaggggactttgtgctcacgctgctggggcgcttcagcttcttcatgtccttgtactggatcgccaacctgtcgctgctcatgcggctgggcaatatcgaggcgctgcacgtcaaacgcagcctgctgctgtcgtcgctggcctcgctggtctacggctgcatgcatgctgtcctgccgacgttgagcgacgcacaggcgggggcgtcgtatacagcgacggctgtgctactgctgatgatgcaggctacgccgctgctactcatcatgacgaacctccgcgccgtgagacggtcgagactcaactcgtccatgcaggggcgtaatgttatcaggaggttgacggggtactgcgtgtgcgcggccgtgttcacgctgccgtacgcgctggtgctcatcgtgtcgcaagatttggtcgggatcggcgccgtcacggagacgtgcaactactttgtgcccattgccaacgcgctgctgttcggcacaagtttgagctgctgttgctgttgtgctacagcggcagacacggcggcaggaccgccacaagacgggaaacggtgtgacgatgataccccgtcgtcgtcgatagatatttcggctggtgggcttacagacggcagcgctactcctgggagtacgggttgttcggggggtggcgtgttaggcgtccgagacggactgctggcgggggggcctattgctgaaatggtgacggaaggaccagcggtgaagatcggcgagggatcgagcgccgaggtgtacagagcccagtggctgggcatcacagtcgcgctcaagtgcctgcgcttccatgctggtgctagttccgaagcagatctgtacatgacgcacctggccgagctgcgtaccgagttcctggatgaagcagtgctggccgcccagctgcgtcacccgaacatcacgctgtttatcaaaatgggcacctacaggggcagcttgtgcctcgtcaagtacgtgctggctatgaagtgtataggacttggaccgagagatctgacgtttgttgtgccttttgttcgtgttaccagcgagtattgtgcgcgtggatccatgagagatgtgcttcgtgcgaatccgctgatggagtggagcactaaagttcgactagctttcgagaccgcaaaaggactggccttcatgcacaaccgtgagcccatctacctgcaccgcgacctcaaggcctccaacatcctcgtcaccgcagactggacggccaaaatcgcggacttcggcatcgctcgggttgcgacggattttactgtgaggaagcagcacatctcgcacaagttctccgcgcagtcgcttcagtcgttgcagtcaattggtgagagcgatgtcatcgtggacaacgcagcatcggagctgatgacgacttttgcgggcacgtggcgctggaacgcccctgaaatcatgatgaaccccaacgagtgccgcttcaaccgcgaaacagacatgtacagctttggtgtgtcgctgtgggagattctgaccaacggagcagtgccgttcggtaatgtggactttgatcatcaggtgcggcagctcgttgcagttggcgagcgcccaacgcttccccctgcttatatgcggcgcgtgccgcccgagttcgtcgaggtcatgtgtgcctgctggcaccagcggccagagaagcgacccagcgctcaagatgtgatgctgcgacttggatccttgtcgtactcgctgtcgagcggctcggagttttacatgtcgtcgcagggaacagcgcgctacatcttcagcgacaactactaccaggccatggactctggccagagttgcccaggactgtggtgaggacttggaacggcatggagtggcgtctgtaatttaagagaagtctgcaccaagattcgcagagcagcaaatatactgagcaacgtagataaattacgtatcaacactgtcacaaccttggtgcagtttaccacgcaccaccatcggcctccaccatcccatgtgtcactccggaatcatccttctcactgctgctggtcgtggctgttgcttcctcggacgctgccaggactcgagaccggcagaatccggtcgatctcctcgtcactaattccctggtccctcaactggttccgagacagcgcaaggacaaccttggcctcgtactcgtgcttttccgccaacaacttctccttcctcgcctgagcctctaaaatctccaagttgagtcgttctcggcgctgttcccgttggtctctcttgatctccagctgcagtcgtttctcctcgatctccagtctccgcttgttgaacgcagaggtcgttgccgcttgcgccgtggtcgcaggtccttctacgatggaattgagccacaaatcgtcctcgttgagtgcagcggcggcactggtggccgtcgtgctccaccgcggtcgatcgcgcgtcctggcgggcgattggcgctgtttcttggccggtggcgcgttgctttcggccgaatttgcgccagtcgtcgttgccgtctcagtgcacatgctgtcggcctctatggcgtgatcagcgagctctgagggcgtcggaatcgcgtcggacagcgagctgtggcccaagtcggttgacggctgctgtgcttcgcccaatgaagagcgcgcgggacacacacgcagatgcgactgcaggcggttgacggcagccgacacttgcttttgacaggccttgcagtccaccctgtcgaccttggcgccggccacccggtatctgtgcccgaagagttcccattccttattgagcggcctgaccattttcgaggaagtcgttggttattatttgcatgaaattaaccggtcaatgaaaaggaatttgctcgacggtgattgaacgagagaagaataaaaaccggggattggtggaaaagagtagcttcaacctgggtgcgaatcactttcatcaccttctcagcgatccatcctgcgttggatgtgcaacgtgcggcagcgacggctgcaggaagcgcaaacttaagttgagactctcaaccagaggctctgactcagtctggctgactgtcactgttgcacgcaggtgatctggtatgaacaagacagtgaagtgactctcttcacatgctctcggacccgagtcgggctgctgccattcctccttttcccaccaaacgctcacaagctccggccgccggtaaagaagaaaatgtctcgcgtggagtgcaacctcaaagtcctccctcacgtcttcatggtcgacaacaacaacgtcgtctgccaggcggctcaaagccccgacgaccacacggtcatcattctcgtcactccgcaggacgcgtgggtcaagaaaatgctcattagcgccaccaacgtgttcgggtacgagctccagtgcggcgacaaggctggcgagcccatgcaggggctcaacgccaacatgaagttcagcaaagtggacctcggcaagggctgcggagacgtcgccatccagttcctcaagccaaagttcctcggtgtctataccgactacgggacgctgacaatccccaactcgctgcgaggctatacgatcgccttcttctggccgaacgacgccgatggagaccactggaacatctttctagagaacttaacccaaacccaacgcgctgtcggtgggattggcggcgtcgcgacggaggtcgtcaaaatcggccaaggcgcggtcgacttcagagctctgtttgcctaattccagtgcagcgaaagaaggattcggcctggttgtgtcctgtacagacaggggtcgaacttgctcctaaatgaccaaatgaaggtttcaagatatgcttgtcttgggtactcggattctcgttgcacttatttccaaccggaggtaacgttagtacgctgatgacagcattgcctcaatgtcgaccccaacagtgatgacagccatggtaatgatgctaagctaaggttaacgatgttgaagtcagccggttcgttcttgcctatgggtctcttgttttttgtgtagttggtggcttaggtaggtacgccaatttcaaaactgcgctgttcgcccgagtcaaacctttgccattgagcacaggtgcacgataaacgatggaagagagacacagttaattctgatgaaggtgacaaagttctcgagcgaacaatgatggcattactggaggcattactggagctgcccatgtagatagctggtggtcgtattcaagtaccgggccatgcacgcgttcactggtatgctgccgaacttggtcgttgaaattgctttcctgaacgagacgttttgatgcgacggcgagttatcgtgcaagtgtagcaccaaagtaatcgtactgagatcgagttcaaggttcaaagcgagtacgctgcactgaggcgtagacatccctgaccagcatgttcttgagaactggttcttgatgagatgtcaggttaagcaatgcaaatcgtccttatgtccttgagcgctaaagataacggatcgaaacgacactaacttttagtgtcagcgttcgactcgacgctaggtagtgcggggcggggggaaatggaaagcgggccaccagtgttggacgcgttagccaagttggagctcgcgatgcgcccgcaaacggttccgtcatggctcagtagcgataccaaccacgctcccaaggtcaccgctctcggtccaaggacgtccagcgtcgagttggtcgataacgcaacggtgcacggagctgtggaagctgcgatcggtcgtagaaatgacagcccccacggaatttctccgcacaaagacaaaactggtcccatggcccccagttcttcagccgttcgacctcgagctccgattgggatgcacaaggatgcggttagtgctgtagacaaggaatttctgtcgaaagggtcggaacacccgtctccgaagagatcgaggcagcgtgtgcgacaacaagcaaccgagtcaaaaagcaggtgggtactgatgtcttgcagtgcattatacctggcgattggcacgagctcactcgtgcgttgtaggcgggtcaagcagtcgcttgtagaatcgctgcggtcgctggagttctttgacaagacgaaccgaagccgcgaactcacgactaaaatctctgtcatgcgcactcatgcagaccctggaggttagtattaaacgttccctgagtgaagctgctgttgctgatctttggctcttgtggtgctctctacagacaatgtgcatttcgtcaatatcgacgatagcttggaagacagtgaagatgacaaagcgctagccgacaatgcagagttcactttgtacgacaatggccgccatactaaccacgaggccgtggaaaaccgagcatcgaagtatctcgcaaagccactggggtcgaagccgtcgccagacccaccgcaagttttccaaattcccgagctgccgcatggacaaaaccttgtcatcaacatcctctcaacatggggagatccgttctacgtagggcttatgggtatcgagatatttgaccacacaggccatgccgtgtacctctctgacgtcgacaagcagctttcagcagatccacctgacctcaatatccaagatcacgatcgcctcgaccctcggacggtcgataagctcgtggacaaacactattttacgtgcgatgaactgcgttcgtggcttgcgccgttctcacgcggccaaaatcattttatttacatggacttcgactatccgttgtccatttcgatgctgcgtatctggaattacaacacaactcgcattcactcgtaccgcggtgcacggtacgtggagatttcgctcgacgggaagtttatcttcaaaggagaaatctgccgcgctcctggaagtgtcatcgaagacatcgacgactgtaacgagtgcatcctgtttaccatgaaccaatcgattttacagttgattgaaaaatacgaaaagcgaaacgagacactcattgatcgtgattgtgcaggagaagcaataccatcaacaaatacagggcacctgccttccgtagagggtcaaagcagtaccagcgccacaattggctcccccatcggctcggatccacgtctacttgagagaccaaagacaggaaacaagacatctcgtactgctgccagagggaacatcatctgctctcctgacacgagtgacgcagcattccctattaatcctgtgttcactcccgtggccccgctgcctaaagggatgagttaccaaacgtcggcgaaggcagctcaagcttctcgtccatgcacagctccagtcgtgggagggccgatgacagagcgtccactgagctgcaaaacgatagaactgatattggaggcgaactggggagatccgtatgagattgggcttgcaggtctacaattcttagacgccaactacgtacccttggcaatttgcttggatagcgttatggtctccgctccggtcgctgctgacaagatgcaaaagctcatcgtgactcatggcgcccgccacctctccactaatcctgaagacatgtggagtgccccgctacaggatgtcctcacacttccccccgagcgccgtgcagtcatcgtagattttcccgagaaagtaagcattcgagcgctgaaagtttggaactacaacacaactttggaggactccttcaaaggcaccaagcagatgtcagtgcgaatagatggcgtccatcatacgcgtgtatcgctgcggaaggctccagggaatctcatggacttcgactttggccagttcctgtatctacgcagcagctcatcgttgcatggtacagctgcgatcactgctacttttgaggcatttccaactagcaaagaaggtgacccgctcgcctcgactgcagaagaagtcgaaagcacaaggtcatcgtcacctgcggcgagtatgggagcgccacgtaccgacgcctcgagtggattcgccgagggatctgcgttctgcaacgcgactttctcgctgtcgcagtcatctccattggtacgaaagaagatcgccatgaagcccatttcggaggttttccagcagtaccagacgccactgttcccgaccgggtacatcatcaagttcgtgttcacgtccacctggggcgacacgttctacctgggtctcaacggtgtcgagctctacgaccgcaacaaccagctcatccctctctgcagtgagatcgtcgacgcccaaccacgcgatattaacatcctgaaagaaccaggcgcaacacacgacgttcgaacgctggacaagctctacgacggcgtcaacaacacgtacgatgaccgacacatgtggctagctccgcacgcaccgtcgcacccgaacaaactcgtggttttcttcaatgagccggtggccatctccaagatccggctatggaactacagcaagacacctgcacgcggcgtccgcgagttcgaggtgtttctagacgacgtgctgatctaccacgggatcctgaagcaggcgccgcctctgaatctgccgagtcttgacggaacgtccgacgagtctgcacttgacgcgtacggacactcgtctatggagaaccgccgaatgcgcaaaaagtggcacgaacaagctgaagtcgctgccaacgcggtcaacatgacgcagacgctgctgttcacggatgacctggaggtgatcgaggccgaggccaacaacattttcatgccccaggacgagctcgaagacagcgtcacgttctacgacaacagtcaagtgctgtccctctccgacctgcccgggcacgagaccatcatgcgcccttcaaccagcgcaggctggtagaagcgctgctggcagcaacatactataaacttcaacctacacccttttaaatcgatatagatacgtatttctaacttgatatttaattcccggggttttagccaatgaagtgacggaatctgctgcgcatcgtagcatgcgactgccatgagcagcagcggtctccacgtgcgcacgggcgaaagccccggcgcgggcggacgcagcgccgggtccacaatgcgaagcagccaacagtcacgccctagcagcagccgcagcagtatggcgtccagtcgcgggggcaggaagctcgaggagcaagtgcgcctcgtgggcacgcactacgtatgtcttaagcaagtccaggaccggttattgcgcggtacctcacagttgctatgatatgtcttatcctgtgtatgaccgcatagcaattgggggcggagatcgggcgcggaggctttggtatcgtgtatggggcgctggatctacgtaatggacgctccgtcgccattaaacaagtgtcgctgcgggatatcgacaaggacgagctgctgtcgattgaggtacaagctgaggtggtgtgttagcgctgtgctgttgtgtgtgacgactctatcctgtattattcaacataccagaccgagatcagtctgctgcgcaaattgaagcacgagaacattgtcaagtaccacgacacgatcaagacgcaggggaacctgtacattgtgctcgagtacatggagaacggctcgctcgcccagtttatcaaaaagttcggctcactatcggagacgctcgtggccatgtatatcacgcaggtgttgaggggacttgcgtatctgcacgagcaaggtgtgctgcatcgagatgtgaagggggccaacatcctcacgaccaaggacggactggtgaagctggccgacttcggcgtggcgatcaagctgaacgagacgcagaaggcaaattcagttgtggggtcgccctactggagtgcgttgctgctgaatggtactttgtgtagcaccgtgctaatgaagtgtgctgttgtagtggcgccggaggtgattgagatggctggctggtcgtctgcttcggatatctggagcgtgggctgcacaattattgagctacttacgatgaagccaccgtacttcgacctagcgcccatggctgcactatttcgaatcgtacaagaagaccacccaccgcttcctcagcgcatgtcaccggtagagacactttgacttattattgcaaactgtgtgttgctgacttgttgtttgttgctgcaggccttacacgatttcatcatgaagtgcttcatgaaagagccgcgactgcgagcgtctgcagaggaactcctcgcacatccgtggatcgcacagattccgaaaaacaaggtggagcaaagtacgcagctcgttgcagagagcgtaacctcttccaacgatcgtgacgctgtgctgaatactattaaactatacgagaaaagctcgtctacaacggacatcccaccgccaacttcaggcaaaagctaccgatctctcagtgtcgcaaatgagcagtccgacgaggatgccgaggattgggacgatgagttcggtgtggattctaaccccacgccgttcgtgcttagagctgacgagaacaagaacgatagcatttcaaaagcatcggtggaagctgcgccgagcaagtcaaagttccagttatcaaaagaagatgcgaacgcactatttgacgacgatgtgtgggatgacgaggggtctgaagcaagagttttatcggaggactcagccatcctggatgctagtcacggtggtagcggtgtcaacatgaatcagagtaagggcgaaaagtcatcgatgaactcgtgggatcgatcatcgcttattcctgctcaaaaccgcatcgcaaagctacagcagtttgtcgaggatccggaagaagacctcacatttgatgatattgacgagaagcaacttttgcaagcagctgcaaagcaacaacgtgctatggagtcagatcctatcggtgctacagtagttccagccaagaaacggctcagtgacttcaaggaggacgctgatatcgacggtgacttcgactttgaagagcaaccggggagtttggtgttacgtgttggtggcaaccgtgatagtaatgtggggtcatcttctgggggaagtgcgcaggagaatctctttgacgatgagttggacttcgattattcgactgctcgtgacactaaccaaaaggcaacagctcgagtggtagagctactttcgttactggatccaagcatggaagaccaggtcatccttgatgcttgcaataatttggtacgttacgttgaaataacctgcactacgcccatcttcgatggagaagggggtttgataccgtgtgctcactataaatgcgtgccattctacaggaagaaatatttgatcagaacgtaactctgaggcgggacttgatgtctcagcctggtgtggtgcccaacatcatggaggcgctggaaatgaagaagatggacgtgctgcatgccgtactaagggttatcaatatcgtaagatctgtttgttcgctgtgctgtgtttggaatagtgcgacttactctgattgctattgcttgcatccgcagattgtcgagggtaacaaaaagttccaggagaacttggcactggttggactagtgccggtgattatcaagctcacgaagcagcacaacccatactacttgccaggtgaaagcggcagaggattccggatggataacccagaggacaacgaattctcaatcgcggtgcggatggaagccgctaagtttgtacggcagtgctgcaaaacaagctcgctcactttacaaatgttcatcgcgtgtggcggattacccgtattggtggactttttgaccctggaagataaaccatccaatcttaatgacgacgtggacacgcttcgggtcgctctggatggaatcttcagcgtgttcagcatccagaccatcccgaagaacgatatatgccgtctttttgtgaaggcgggtctgttgaaaaagtttgtcgtcgtcttttcggagattgttgcgtcagtgtcagcgagcgatacgcgctccccgaaatgcgatgaaagtgtgccgaaattattgaaaacaaacgcagaaccgaccaaatccaagcgtggatcagtggcacagtggacaatgaaggagcttcacaagacttgtgatgtcttcgtgctattttctcaaggagatgctgtggtgaaagagcatatgtgcgatggtgctgttttggaaggcctactggaagctatccatcctttgccgctttttaactcaggcgaggaacaaaacaaggggtcgctgccgttgatacggcattcggacgaatttgtttccgccatgctgaaggttctcaagtgtattcgtaatctgagcatggagcccttgacactcgagaaactcgaccgagccggagcaatcccgacacttgtgcgtctattaaacgaacaagagacagaaggcccctctatctcagacgcgaagcggaaagaggtcgaaaacattgtgctccaatcgatgtattatctctgtcggattaatcgcaaccgccagactcatgccgcacaggctggtgtgattccatcgcttattaaggtggtccggaattccagtccgctcaagcagtttgcgcttccgatcctgtgtgatctggcgcatgcctcgcctacagcacgagctcacctttggacgtacgacagcgtgtccctttttctggagttgctggaggataaatactggcaaattgatgccattaagtccattagcgtttggcttgtgcacgacacggtgaagatggagaatgtcttgctcgttccagaaaatctgatgaaaattacggtgtacttccacaacgctttggacaccgagttagagaacctactggagccgctgttggagattatgagtcgctcggtgaggcttaaccaggcactcggacgaagcggcatgtttgtgacggagatcctaaagcgactgcgtctcattccgaaggcgattgtgcgcaagaacttgctgaagatgctcaagagtcttttcgagtctcatacatcgcccatccagttccttgtggagtacaacctccgcccaatcgtgtatgcgctcgcacaagacgagaacagcatgattcttgtcaaggaaattgcgtcgcagctcctccaagccattctggtggctgcgggggtgttttagatggagtgtacgtgtcagtcctagtgaggacataactgcactggacaaggctcacaaaaaagtggtcaacgccagcgacaagcgcgccgctcgaggggtaagcggcgagtggagcagtgtgctttatttattggaaatggacgtcacctgtgcttgtttgtatgcttgtcatttgtgtgcgtgcttctgctgttgcagcgacgacagaagaagttccctcttgaatgcttctaggttgcggtggttggaggcattgaaggttgccagagacgatggtggcattttctgcgcacgagacttggcgagttcttggtggacttcttcccgaacgacatgttggatcatggcctttgttggtggcaactcaccttggcatgaaatgcacttggctcctacaagcggtgccgatttctgaactgcatcttcgatggtagggctggcttcgcccctcagcagatttgagctggaatccgtccgcccaggttggccttgcagtagcgccttcagttggcttttatctaacttgcggcgcaatcttcggtggatatcaacgagatcgtgctcatcgagtgacgcgttggcaacattctgcacctggcgttcgagtacctcttgggtccaaagcaattcgtccttgtccaattttttgctgacgcgtgcgataagctggttcatctcctctcctagcagtccgtcgcgctccagtgtctcgctgcgcgtgttctcgagactcgttgtgagcaactccctcatctcgttgttggaggtcatcagctcctcaatggacttcttgacgtcctcctccttagcatatgcaaacgagagctccatgaaggtatcgaggcgtgagttcatcagcttgaccagcgcaatggtcgagtcgaggtgaccaagcgcgaaaccgtgttcctcgatcttgctttgaacgagagtctgcgtggagcggagatcctccacctggtgacgcgagttcatcgcttctgagtgcagcagaagcagaaagagaacggaccgtacttgaaccactacgtcgtgcatgaagagggtcaactccggaatctggttgccttctcggtgagcttctcgtgtgatgagagccccttccaattctttcagcttggtggtcgtcatgaacacaagatggtcgtcaaagctcgaatgttggttagaggttgcggtgccaccgccataggcgtcattgtttccgcctgtgtcaagagcaagtgaatgagcagccgctttggccatagctgcttggcccttgagcttatcgagttgctgttggaagtccaacgatgtctccaccaagaagtctatctcggacgcagcttcttgggagagaagcgtgttgcagttttctatgaagtgcgtgaagattcctcgcagtagctcaatgccaacatcaagcgctgttcgcgtagagctctttgtggactgctcaattttgccaagctcaaccgactggagcatcgctccgctcattccgagcagcgtgtggtagatgcggttgcaaaattcggtgatgcgaagaagcacgtttccatctgaagttcgttgcgaggtcttgatctggtccactattttctgaagttccagcgcagcctcggtggttcgctgctcggcttcttgtcgtagcgtgtacagcgaaagttccaacttctcgtggagagcaggaatttctgcctcgctcacctgcgtcaacagtttctccgaggcctcaaaggcagtaacttggtgattgaacttggaggatacttcctgtagctgcactgatatggcgctcgtcactgcgcttgtcacgctcgaagttactgagctagtgcggcgagcagaagttcgcagccgctggatctccacatcaatctcctgtaacttttcctctacatcgacgatggtgcgagcctgtgtcttctgcgtgcgctcaagcttttggactctgtccacaacaggctggagctccatgatactctcatccagagcattgatggccgagctgttatcctgaacgtgccgctcgaggtactccagcgatccatctaggcgtgcggtgttttcctctagtcgcttcacacgcgaacccatggacatttgctgtcgacgaaagagcgctttctcagcattgatgccgttcaattgtcggagccgctgcaagcgtacaagcttggccagtaagataaaccaaagctctttgactctctctttggtgactggtgggcgtggctcaacaacgacgggtgtttccggtcttggaagtggcaatgaacgcgacaccagctcgttcccgggggaaaattcacgtgatgtgacgtctatatccttacggttggtggttgagcgacgctggcgccgtgctcgaacgactgacgtaagcacgtttggacgtgagccggaagatgacgaggaccggaatagtgccacctgatcatccctttccttttgctgcgtggccaactgggaacgatactgccgtggtaactcctctcgtgtgacttcttgctcttcttctttggcggtactttcactcgccgctacgtcttcctgcttttcttcttctttgctgtcttcaacgactgcaaccggtttgggggtgataggagtgctgctaggaacttgctcaacgacaacagtctccgtagtgcgttcaccctccgactgctgacgggaagactctggtacttgttctggcagagctgtatctaggacatcaatgggttgttcaacaaccacttctgggattggtggatcgaccaagtcacttgataccactggggtggctggttcggtagcgtggtgacgtcttcgtaatgaacgccttacgaacttggacaccaagacggtggctttcgctctaagtggagggcctgagggtgatagtggatgttgcgtagctggaacgtccgtcgcgctgtcttgctcggatacatcagggtgggcaggcaccacctcgacatcttgctgctgcggtaatggttcttcggtcggagtttctctcgcctcagcggtgacatcgagctgggaattctctgaccgaaacactgggacgaagtggagagctgcttttctagcaacaggcttggctgtctcctcttcatctgctggcgcctcttcttcctctgcagatccatcgccctcctcgctggtttcagagtcaacgtcgtcgctacccagcgacttggccagcatttcgatcccaacgcgcatgtcctcgatgccttgttcttgtcggtcggatcgacgcatcagctccttcacctccatcttcattctcgacaagtaatcaaagcgcttttcgagctgctcgagctgcgtctggaccgcttcttccgtttgtcttgttttctccgccaaactgtccgtcgcctcgcttcgcagcttcccaatctcgagactaaccttctggtccagagctcgaagcgtctcctccagcaaggccctcgctggtccgtcatcaaccacttgcgaactctgtagagccacgagcgcctgctgcatctgctcgttaacctgctgctgcacattatgctgcagctgctccatcttggtcgtcagcgtggtcaaagcgtccagcaccgacgtgtcgggcagcgccggctccaacgagggtacctcaactaccacaggagtcggcgccggagcgttctgcagctccctgacggcgagtcgcatcgagagctgcgtggattcgagctgcgccatccgctgtgcctgccgctcgttcgccgctgccaaatgctgcagttgcgtggtcagtgtggtgatcacagacgttagcgcgccttcctgtgcggccagaacgtcgcccatgccaggcatgaactcatccagcaagtccatgggccttcaagatgcaaaaaagacatctgagcttttggaggagcggccaatagcgtaacggttttcgcgccacttcaacatgcacgtgattggctgattcggtcctgtatactattggttttctgtgcaaagaaaaaggtctacgatacaggaagtcatggcgtcgtggcagcactacccgggctcgtggacgcccgaagaggaggcggctcgacttggaggacgcacagcggcgaatgcaccgcctatgcgcttcgaagacgccgaagaaacgaagatggacccgtttccaggtcgggatggacgtgcgcaacgtcctccctcgtctcagtactatgctgtcaatactatggcgccgcagcctccaattcaacaggaagagactcgtcgccagcaattccacgcccagcagctcccagaatactcgcatccacaaggttacgcaaggtaccccgttgtccaagaacattacgctccgatgtcttatcgagaccagttgacgtaccaagctccagctccccgtccgcaatcgacgtcagtgcggggccagtgggtggcggtggacgagcaacagactcgtcgtgggggttggatggaaggccccgaagctccaggagcattgcagatgcgacctgttgcatcgtcgactcgacgcaggacgtcagggtttctgacgactacgctgctactttgtgcgttccatgtggccaacgtcttcttcgccttggtggcgtgcggcgtgctgtgtgcgggcatctgctcggcggtttcgatgatccccgtgtgctgcattggactgctcatcttccaggtgctcgtgtacattggatttgcgctagcacaatgggatatcacgctcgggaactacatcgcgccggcgcatcagtgcgcgtactcgaacttgctgccacccggcaggttcgtgggcagggaggggctctcggtctaccgtatttctccaaatttggcgttcttttcgccgctgtcgctcatggcgatgctgtacttcgtgctggtgaagcccgtgatcggcgctctgagcttcctgtcggtggtgctggttgttgcgccgacggtgtcgttcatcacatcgctgacgtcgcaataccatgataactacgactccatccagatctacggcgttggccccacgtcactgagtcgctatccagggctgtcgttggtgggggccatttgcgtgacgctcgtgggcgtggcgctaatgcagctcgtggccaagatctcgctgggggccacacggtttttctgctgcgagaagctcgccgtggcgaccaggcatttccaataccaaccacttgcgagcaatgggtacggaacagcttcaacgaggagctaaccagcctggttgccaggggagtaccgttaccggtatcctttcgtgtggccagaaaaaggcaacatattcacaatcattccactcaaaatgtatgctgcgccacgaatgcctctatggcacaacactttccaacttgctccaacccacgcagcggcagcgatcaaggtctccaaacatggtcgagtcgtcacgcttcagcacccgcctgtaccccgacatcgaggctccagtcgacccgagatacggcgcagcgtaccctgcagccaactctcctccgatgacagcggccgagtcggagcgctggccgctgacagctgcagtgccatcgtacacccagcaacagcaagagccgtacaacgcctactatgtgcccccgtcctacggagcgccgcctgctgagaatcgccactgccatcgccaccaccagaacgaccgccaccaccgcagctaccacgtgcaagctgactgcaaccacgaagtcttcgtcctcttgaccctgaaactcgggcttttccactcgctcaacgcgctgctggggcttgtcgctttcgttgcggtcgtcactggtgtgcacgtcgctgtcgggctgatcccgctgtgctgcctcggccttctggtgttccgcggcgtggtggctttggtgcgatggcttgcaacgctagatgtcaagctgagcaactacgtcgcatcgcctggggaagaacgtattctcattgcggatgcggaccaaccgctgggagccttcgtggggctgcgactgtctccagagctgtcgtacttctcgcccgtgtcgctgctgggcgctctgtatttctcgacggtgaagtttgtactgagcatcgtcagtttgatcgtcgtttccctgtttgttgcgttgcccatggcgctgctcgctttcagcagcgacgacgacaccgagtggactatcaaggtgcaccaccacaagcgagtggatatgcgtgagcacccatttgccttctacgttgtgtggggctgcctctacattctcaccatcgtggccatgcacgtcgtagcgtggctatcacgggctatgaccaagttcttctgctgcgagcgggtgtccgcgtccgagtacactatcccaatcgtccactacccagacgccgccacaatgtacggctccagccgaagcaacttgtaacagaacacgcagtaaattgaccatgcctagtaaatacatgtagcaacacaatgaaaaagaatgtagtaatatcaagtgcagaaggcagtagaacgtcgcgacaaccaatacatgcattgtcttgcattgtccctatatcctaagctgtaacgcagtgacatcgcactcttaggatatatccccatacggtactggtgtacagtgtcgaggtgtcgcatttcacaactggagcgtcatggtggacgcgcaggccgtcgtgatacaggctcttcgggtgctcgtgttcctggtgctcaactatgtgctcgcgtccacgggcttcatggtcattgtgtttggcgtcgcattctcgctcgggtcgatcgcgctctgctgcttgggcgtcgtggtgttccacggtctcctgtacctggcgccgctgctcgcgcggctggacgtggcgctgcacaacttcgtggagcccgtggaaaacaaactgtacggccagatcccgcgctacggggagaacggggagtacatggctcggccgtcgctggccgtgctgctgtacttcagcacggccaagctgggcgtcggcgtgctcagcgccatggtggtggtcatcccgttctccatgcccatccacgcgctcacgtcgccgccgtttcgagccgagtactttgccgacggatggttcaacctctcggccttcattgtggtcgccactgcgctgctggtgagcgggctcgtcgccatgccgcacgtggccaaactctcgtgcgtcaccacgcgtctgctgtgtcgtgaggtgttctccaccatctacacgcgcgacttcgtgctgtccatctcggaagcgcctccagcgtcctacggcaccaagcagcaagtctgaatgaacaaagtgaacgacaattaactgagacctcagcactagaagaccaaaattgtactacaaccgttgcctgataaatcaattccagctctggtgccgcaccagaatttttgcctgcaccagaaattgggtatgtcgggacttgatggctggagccacataaatacgtccaccgaaataagtcactctttcgggaagcttcatcgcactatagtcactcttcgcaaactgcacatatcatacttttttcgcgcatcacccttgcacttttcaacttgcacttgccagcagagctcctcgcaaatgccacgagccaaagacgcccacgtgaccttccagtcgccctccaagagcccagcgtcgcccacctacatgttccattccaaaccgccgccgcgtcactgctgccccgagccgcccgcagtcgacgccgagggcctcgttcacgcgttgctgcgcgtcacgctcaagctcgtggtcttccactggagcaactcgatgctggggggcgccgcctttgtggtggtgggctgcggcgcgctgctgtcgttcgtactgacgccactctgcggtctcggcctcgtgctcttccgtctcgtgctgtgcctcgtagcgctgttgtcggagctggacgtggtgctcgccaacttcgtatcgctgccagaggagcacatctccattaagaggctgagcagcggaagccacgccagcccgcgcgcgtgcggtgagacgagcgtcgagcgactcgtgccagaactcgacacgttctcgccccaggcgctgcgtgctacgctctactttgtgtcggttaaggcgctgattgggacgctcagtagcctcgtgctgtccattgccttctcactgccggtgggtgcaatttctcgaggcagtttaggggacaatttccatggagttgtgggcctgctcgtgttcttgctcgcgacgaccttgctgctgggcatcggcatcccgctcatgcagtatggcgcgagactgtcgcgagcagcgacggtctacttttgctgcgagaagtgcgcgcccgagcaccatcaccacggagaacagggtcattcggctacgtacggctctacggacatgtacgcagcagcgtgaggcagcaacaacacgaatgcggcagtgctcaggtctccagaactttgacgtctgtgtactacatgttttgatggagatgtggaatggttaccatgcgtgccgattaatgtaagaccacgaagttctcaaacattttccgtgttattaccacttcgatcgtggcttacgttacactgtactatgcgcagcaactcgaccaacatacaacgagcgggttgacgcttctcaacaaaaacgactggggtgtctgggacctttcacaacttacagcacaagacgacacagcagcacactccagtcgaagctcttcccaaaacaccaattgtttatcatcacctcttcactcgccagcttactgctgggcgccgaagtcctgcgtccagtaggtgtcatactcgctgcaggcgctgttggtgaccagagcaaatccgacgttcaccacgtcgtccttgaggatgttggcgcggtggcccgcagagttccaccaggacgtcatgacggccgagacgctcgtttggccggcagccacgttctccgccgccacgctccagtcgtatccctgcgcctcgatgcggtcccagggctccgagccgtccgtgccctcgtgggtcatggtgcagtgcgtggcctggtcctgcgagtggacattggccgcggccacaagttgcacgtcgatggtcaacgcggacagtccgttgtccgcgcggtacgagttgatgcgcgccagcatctccgccgactcggcgcttgtcagcccgttgtcgtacgtcgtggacccggacgtctgggaggaagttgagtccgagttctgatcggtttgcgatgcctggctggtctggtcgttctgagcaacctggtcgcccttttggccggcgttctgtccagcgctttggtcggccgagtcggagaagcggtcgatgacgtatccgcagccagcgccccagtaggccgaggcggtggccgtctcgccgctcttgagccagcaggtgccgccgttgaagtaggtccacgtccagtgcgtgcacttggcgtcgtccgcgcacaggtctccgcagacgtccgggatgcccagcaccgaagacgtgtcgtcaccgtagaagtcgcagttattctgccacatgacgcggccctggctgccgacttggaacgcggaggacgagcccagcgccatcgcagcgacggcggcggacagtgtgagcacttgggagcggcacatgcttgcgggatgtctatagagcagggagagaggattgtttggtgcccgatgaattgaaaagtgagagaccgagcaccggcgtcggactcaaggtgaaaggcaaaggatagactgcaggcagagcttcctccgcgctggtgaacatctggcggccagttgacgagtacacgctgccaatatcgttgaggcgctgtcaacttggcgctaatatcgttgaggctgtcaaattggtgttttgcggtggaagtgccgtagtagcgagtaccagcgagcctgagagcgtcggtggcctgctggtggctgtgcgctccaggactctacatggcgtgttccaaactttggaatacgctggcactctcatcgggcttggtggtggaaaggcggaagtcggagcgcatgcacaaacgtgagcacgtcatgttgctaaggcagcgaagcacgaaagaatggagctcgacttgcgagagctgaatgcgttcacagcgttggttgtcgtggatggcacagcgtgttgcttggcgaccaagtagtgcagagtccctgtgctgccggcttctacgactcatccaccgaacgatccggctcactcggtctccgttgctgctgagcttttccgcagcaccactattatatctatacagcagtggcaaggataatggagaccagccgttcccgtcccggtacttgcgttgaagcggttgttgaagccacatggcatcttcatcgtcgcgatctttaccaggtggtgagtcaacacacactgggatagcgtgcatgctaaaggcgagcggctccaataagactaacccgccagcggatccagttgttgctgtagtaagtgggcgttgtgcttcagcttgatgtgtaaaagttctcctcaacggtcgtcaactctacagggagtaaaaatctccagggcaaggctagaagagcgatggtacatacggagctctctcaatcctggcgtttgtgcggcaaattctaccgatggccagcttccagcgaaaacggatatgctgttgtgcgtttgtagttttgttgaacacttgacgtcgttgacaattactacggcatgttataatactcactgctacacatattacacgctacgcttacgcactagagtacatcgagatcgttgacatgaattggctttccactgagccatttactgctgcatatcgcttcagccctggcggccgaagtcctgcgtccagtaggtgtcgtagctgtcgcacccgccgttgatcaccttggcaaagccaacgttcgtcgcgtccttggcgaggatgttggcgcggtggccgggcgagttccaccacgaggtcatgacctgctctaccgacgtctggccggcagccacgttctcggcagccatcgcgaagctgtagccctgtgccttgatgcggtcaccaagtcgggagccgtccgagcccgtgtgcgtcatctcgcagtggctcgcctggtcttgggagtgcagcgcagccgccactgtcaagcggttgtccatggtgagtgcgccgaggccattctggacccggtagttgttgatccggcttagcatctcactcatttccgagctcgagagaccgctgctggaggcgacgatagctgaggcttgaatcccctgcgtttgaacgcgcacgttgcgagacgtcacgtagccgcagttggtggcccacttcgacaccttggcggagcgggcgcccttcttaagccagcaagttccgctgttgtagttggtccacgaccagtgagtgcaagtcgagtcgtccgcgcacacgtcaccacacatagctgggatcccgcgcagtgagcggtagtcgttgccggtgaagtcgcagttgttctcccacatgacgcggcctccgctgccgagctggaagccggtggtggttggtgcgaacgaactgcaggttatggcagacgcagccagcagagcgagaccagtgaagcgcggcattgttttccgaggtgttgttgatcggtccaaatggaaaatgaattccagggcctcgaactcgcgcactcctggattgcagagcaatgggccctatgtgcacatctattcgaatgattcagtgaataccaacagatgagtgtccgcctcccgccgcgatggaaaaggttggagcaaacatcggttgtggatcctgatgttcatcctgatgttgggtccatgccacgtcgatcgttaagcaggaatcgccatctcggtggctaaagttaaaggagctatgcagggatggggaataagggcgactctgtttgtccgtacattttaggtagacgttttgataccacaagcatgcatgggggcccatacaagccctgtccatctttacgggcgtgcaggcaaggcgttttcgctgtgcagacccaatatctacatgtactggacgagtgcagcaatgcgtttgttgtccacttttctgttagaaagtctaacttgacggcgttgccaacttcaagatcccaagatgtgtagtactgccactcgcattactcagagcaatagttgataacgttgctcgagctgtatctcgtaacgtttccacttgatatcctggtcaacgttcatgcgtgcgttcaacatttactgcacctgctgctatttgctctattcaacgaataattggtggcggcttgacggcctgctgtgcatcgtagaatcctgaggctgtcgtgctgttcacgctggggtgcagtcgctcgcataaaatccttctttaccaaaagccactggctttgctgtacatctgtgcttcaacacatgctctccgcttttggctcgcaacgaagcaacgcagatggcactcgcaatagtctacaactatacatgtaatacaccaccatgacgactattcctcatatgtcaatcgcacgcaaacccggatcttcaaaggtatgggtactctactgcaagttaagcaagtggattggttcatatttaatattgatcttgatagtagtctacatggactaagtagctgcgttcaatacagaggacgcccgaaatcctgcgtccaataggtccggtagtcgctgcagcctccgttcgtggccaaggcaaagccgacttgaaccacattcttgttgaggatattggcacggtggccgggggagttcaaccacgagttcatgacatcagccacagtggcttgtccggctgcaacgttctccgcggctgtgccataggtgtagccctgcgccttgatccgatctccgaagttaggaattgtcgttcctgtgtgcgtcatcacgcagtgactcgcttggtcttgagagtgaagcaacgcagccttcacgagccggtagtcaatggtgagtgcgccgaggccattttccgcacgaaccgagttgatccggcccagcatgtcggccgtgtcagcgatagatagaccgctggtgctggtagaagtcgcggtgcgtgacacaacgtagccgcagatagcaccactgctcgttgccatggtgaagcgtgtgttcgttttgaaccagcaagtaccgccattgtagttgttccagctccagtgagtgcacgtcaagtcggccgcgcagaggtccccgcagatttcggaggaccctttcagtgcccggtagtcgttgccagggaagtcgcaattgctgtcccacatgacgcgtccgctgctgccaagctggaagtcagcagcgaccggcgcaagagaaatggaagcgatgaaagatgctgcggcaatcgtgaagctggttagccgaagcattttgacttgagagggggagttggctggaacgaggagaatgtaaagtgatttctctactacaagcaagatccgtgctatggcggtcgttgccccctgatcacatggatggagtgcaaatagatgctgctagatggacatcctcaagccattgaaaaggacgcgaagaagcttcgatggcggatcctgatgcccttttcactgcgagtatttgcatggcgttttctgggggctgcgcacaacgcagggctggggaataagaccccgataaatactgtacatcgttattatagagtgaggacgatatataaatgcgtcgtcgccttgaggcgtggtagttcaacttcagcggaactgcactccgacaacaggtacacgtagcttcaagcatgggacaactgaaccaaaacaactaacccaaaatctgtgaatgatcagcacgagaaagacacgagcgtctctcacgtaaaatccaaagtagcctcaagatccacattgacaggatgcaggctattaacgcacgcctggatgtgcagcgtgcgtcgatctgtagacgaaaaagacaacaactttgtgtagccatttcctcccccgctcccattggtagaaactgcgtcactgacgtgtcagacgtaacgtcccatcgaaatgcaaatgatacccagtttacaaagcgtctgagtatggagcaggtctacgcgggccagagcctcttcgtcacgggcggcacgggcttcctggccaaggtgtgtctctgcactgcttgtccttcgtcttctgcggctgctaacccctttccctccctcagacgctcatcgagaagctgctgcgatgcactccagacgtcgaaaaggtgccatttgctccacattccctctaaatccgctgtaaattggctgaaaattgctgtcactttggcagatcttcgtcctaattcgtcctcgcaagggcgtggcccctgccgagcgcctacagaaagagatcgtcgagagtcgcgtcttcgatcgactgcgggccgagcgtccacgggattttgaggcgtttgcggcctccaagctgcacgcaatcgccggagacatcacgacgccagacttgggactcagtgcagaagacgcacaacttttgagagcctgcgtgcagatttctatccattcagcggctactgtgcagttcaatgagccgctggaggtggcggtggaaatgaactgtctgggcactctgcacgttgcaagattcgtgcagagctgtccgaggaaccgctgccatttgcatgtgtcgacggcttatgtgaatagcaaccgacgtgatacgaggatctcggaggagctgtatccgctggactttgacgcacatgatgcactgaaggctgtgacgacggcgtcgccgagtgaattggaacgactgagggtgaacctcatgggcacgtatcctaacacgtatacgcttaccaagtccatgacggagcaccttttggtgagagaggtcgcgccggacttcccgctggttatttaccgcccgacgatcattggggcgagttggaaggaacccatgccaggctggacggaccagatcgctgcggcaggtgctatctttctggctgcagggatgggtgttttgaccatgctgcctggtgatccacgaaatgttgcagatattgtccccgtggatctcgctgtcaacagcatcttgctgtcgatctgcgcaaagattcaccagcaagagaggctgagcttatcgcaggagccaccttgcccactgatcaagggagtgtcggtgaacaagcctatggtggttcattgtggtacctcagatcctcgacagaaccctttgcgctggcgtgtgccgtgcatgctggtgcctgaatacttccgcaagaacccgccagtccgtggactcttcccgtccaaattctctatgattccaactcaccagagcttccagatacaatggttcctgacgtatgcgctgccgtcgtccgtgtactcgacagttgccaacaagagcgggcatcctggtcacatcaagaatgcagccaggctgtggcagcttacttggcgtgcacgcaatttggtagagctgttcaagccgttcacggagaatcaatggattttcgccgctgatgctgcagagaagacgctgaagccttgggcaacgaaagacttttggattgattcgcatgaaattgcgtgggagcggtatgtggtcaactactgtgtcgggttgaaaaagtatatgctgcacgaggatgtaattgacgtggacatcgagggcgtcaaccaaaccaagctcgcgctgagcacggggcgcattctggactgggaccctgatcaccacgctatctcgtttcctggactactgtccgatgtggcctgggcgtacacgtccagtcgcaagcctgggtacaccaaatccggactgctgggtcgcgtgatggggattactgggtggaaggaaggcatgaatcacgaggcatcgcacgtccccaggcctcatgttgagtcgatcggcggccttcggaacagcgttcttgaatcggaagctgtccgcgcggccatcgaagaacgcgctgtggcacagggaatggatctggacgacgtggagcgtcaggccgccagcatgctgagcaccatggctgcgcagatggactacaaggcagtgcgtaaaatcggctggctgctgcgaaaggtgtgccgcaacatgtacgaccaaattcacgttgacgagtctggtctcactcgtattcgcgacttgctggctgatcgacgtgggagtgtcgtgcttgtgcccacgcaccggtcttatgtcgacttcctgctgatgagctacgtgtttttcgcctacaacatccccgttccttacgttgcagccggcgaagacttccttaacatgggcagtctcaccaagcttctgcgtgagtcgggtgcgtacttcatccgccgatctttctcggatgatccgctctactctgctgtcttccgcgcctacacacagtacttggtctcccaaggccacacgatcgagttcttcatcgaaggctctcgcagtcgtagcggcaagcagctgcacccgaaattcggcatcctcaacacagtcgtcgactgttacctctccgagaaagtcaagaacgtaagtaccttcagctcccagcacaggctgcaattcgatatcggcgagatgcatgactgattgttgtgtggatttgtctcccagctctacattgtgcccgtcacgatcgactacgagaagccgttagaagtgctgctccaccagaacgagctcctgggcgagggcaagattcgtgaggtaagagatctctgtgtcatctttcattgctgcgtactcataacgcgctggtggttatggtttctagtcgatcagcgctctggtgaaggccatgcccgttgtgcgcaagaaattcggctccatctcggtcaagttcggcgtgcctctggatataaaacagcacgtagacgcaacgcttgcgcgcgctgaagagcaggaggtgtttgttcctacctcggccatcgtcgaagacctcggctacgctattaccgacgcattgatcacgaatgctacatgtgccatgtcacacgtggtggctaccatcctgttggtgtaccgtcaaggaatctccaagcaagaacttgtgcgccaggcagactggttacggttggagattttacgccgtggtggccaagtcgttggcactcagggtcgctcacctactgtcgtcgtggaccgcgcactcgagctgctacacgaacttgttaccatgcggcgcaaggatcttgttgagcctgccattaccagcgtaagttgtcgttgacgtgacgttcatgctgctattcgagtgtaatcttaccttcgttgtgaatgctgggctgcagcgcgagcaataccccaacatgattggcctcggctactaccgcaacaatctgctgcactggttcaaccgtgagggcgtgctggcgtgtgcctaccatgcgttggacgctttcaacttgccggctggggccagcagcacacgctcaacgtcaagcaactcggaagacgcaggtgtcgaccgccaggagctgctcgatggcgcgctgttcctgcacgagatgcttatgatggagtttgttcgcaaggacgactctgaggccgaccgggctcatctcgcggaggccctcgcgcagctcgagacgcgtaacgtgctcgtccccgtgtccgcttcatcgtcctcagcgccgcgagtggaggcggcggacggcgcgatgctgtcgctgctcaacacgcttgtgtggccttacatcgacagctactgggtcgctgtcacgagtctattcgcactgcgcccacacggcgaagtgacgacggaggacttgctcaagcgccttcagtggctcgccgagaccatgtaccacgagaagctcatcagcttctacgagtcttgctcgcgcgagacgctgcagaacgctttggctctgctggaacgctggggcgtcctttccagtcgtcgccgccccagcaagcgcagcacgaacaagcccgctgtgcagctcctgagcctcaccccgcagttcgcaaaggatggcgagctcgaacagctcgctctgcgcgtctccaagttccgcaaactgcctcctggagtgcaacctgcaacagccagcgagactgaagtgctcgctcgactgcctgcactctctcgcatgtagataacaaaccacgttaaggcaaaagttaaaaagacaagctggcattccgatcatatcaaactcacgaggcctgcaaatggcagcgttgtggagccgcacagatactatcgtgctcctccgacgcacccgctgctaacagcgagaagcgagcaagctgcttacaggttgccacggatgcgctcctgcacgtcgccgatgctgtggtgggcagcagaccaggcgcagtggaacagaccctcgcggtccgtgcgctcgtaggtgtggccgccgaagaagtcgcgctgagcctgggtcaggttggccggcagacgctcgcgacggaacgtgtcgtagtagttgagcgagcccgagaacgagggtgcggggatgccgctggccacagcgagcgacacgacgcgacgccacgagtactggcgagcctgcagctcggcggcgaagtccgggtccacgagcagcgagatgagggacgcgtccttggtgtaggccgacttgatgcggtcgaggaacttggcgcggatgatgcagccgcccttccagatgcgggcgcactcgcccaggtcgacgttccagcccatctgcacacccgcctcacggatgaggttcaggccctgcgcgtacgaacagatcttggacgcgtacagcgcctggcgcacgtcgtcgatcagctgctgcttgtcgacggcggggatctcggacgggcccgagaggatcttggacgcgaacacgcgctcctccttgcgcgcgctcaggtagcgcgcgtccagcgacgcggtaatggtgggggcggcgatggatcgctcggcggcctcctgcacggtccagcggcccgtgcccttcatgccggtcttgtcgaggatcttgtccagcacgtagccgtcggcctccaggtcgtccttcttggcgaagatctgggccgtgatctcgatgaggaacgactcgagctcgctcttgttccactcgtcgaagacgctcgcgagctcttcgttcgtgaggccgcccgcgatcttgaggatatcgtaggcctcggcgatcagctgcatgtcgccgtactcgatgccgttgtgcaccatcttgacgtagttgcccgagccgatggggcccaggtacgtcgtgcacgcgccgtcgtccacctgggcggcgcacttggtgatgatgggctcgagcgcgtcgtaggcttccctggggccaccaggcatgagcgacggcccgttgcgggcgccctcctcaccgccagacacgcccatgcccacgaagtggatgcccttgggctccagctcgctggcgcgacgcacgctgttggggaaccactcgttgccgccgtccacgatgatgtcgcccggctccatgaactcggacagcgcggcgatcgtcagatccacgggcttgccggccaccacgaggatgatgaccttgcggggacgagccagggacgccacaaagtccttcatgtccttgtggcccacgagcggcaggttaccctcgtccttggcgcgctgcaccgtggcgtccaccttgtcgggcgagcggttgcacaccgacaccttgaagccgtgcgacgccatgttgagcgcgaagttctggcccatgacggccaggccgtagagtccgacgtccgagaggtcagccattgtactggaaggaggcgggggagtgatggtctgggagctgctgaagtgccgagctctgctgcgcaaattgcggaatgacttgctcggggggaagatcctccttgccccacgctgagctgcccgcatgcgtacaagaaaaccggtaaatgacgcggttcgaaacgatgacgtaggcgcccaagagacatgaaagactcagcaagggacctgcgtgaggcacttgcaatgaggacatgttgcccgcgggagcaagcgcaaggcatagccgtcgagcattgttcaaccacagcaaccggccgtcaacgcgccgggtgcgagaccgcccagttggcgaaatgatgaagggggtccactgtcgaagtactggggtgcaacgcagcgacctcggctgtacagtacagtacggtaattcggtatatatagtgacacattccctcgacacattagtgtcgagaggaatgccaaaggactaagcactacataacgtagaggttgctgctgctgcagctccgttgccatggttgccatcgcggattttgaagttcgagctgagtgaggtgtatctttaacatgaggcgtattggcttttctcgttgcatgcttacttcgcgactcgagaaacgggcggtgaaaagcgtcgatacctatcagcggagcgtcggcatgcctgagcaacaacggcggcaacttaatcgtcacgagcatcatggcgccaggattcgtggagctgttgtcgtcaatgcggcttcgaaatgccgcctgcgtggccgcagatgatttcgcggggtcctcgtttacgtcatgatctacatgtagcacaccaagcaccaatattacggggaaagtctacatcaaagtgctatgacgtaaaagaggctgagtggaaatagtgaagaccaaaaaaaaagcggagcatggtttaacaagattatatgctacaaacgagtttatgccggctcgacaagtgccaatcctcaatgcaccagcacgacagctgccagttctcctcacctgcttaagctggtacttgccctgcatactcaccgcgaagcagccagttgcgcagaacttgacttttgtatggttttcatggccagcatggtatttatatagcaataatcccttcttgtgacaatacagtcgacaatatcgactgtcgcagttcacctgcttaagcagattggccccacaaacatcggaaatggttgtgcgacaacgtaagctctggtaaaccagcttggcgcgtcaacttcccggcgggattcaggccagcgacagcgctgattcccggctgcctttggctcgtatccgtcgtcggcaacttgccctcgacacgcgctgcactgccaacaatgtaggcagtgcctactaggcaccactctaaactctgtagtttacacgtacactaagcttgcttggtttgggagcgctcagacatccaaggaaaattggagcctcaagcagcaagtcaggtggcaacccgaccgctatcagcaaaacactgcgaatgtttaagggatatgtttcttcatcaacgcgctccacaggtataatcacaatacaaatacacagccatccgtacgagacggcagcaagaagaaaagtgagcactatcgtagtagctcctgccagcaaaagtgcagccttgtcttcgcgtccatcggttttggcaagccggttctggaatcttccactgcaatttgattcgccaaaacccattctttgaacgggttttgacgaatgagccgccgttgctgacgactggtgcgcagtgcgtcttgcaccgccgcgccttccttcaagctcagaacacgatttagaattgcgaagtcaagtgggcccctctcggggggcggcttggggaaattccccggacaccagggcagagagtgccagcgtccaagcgcagactccctcagcgatgaagcttggcggagagggcccgtctcggcgggttggagtcatcggcggattcgcgaggactgatggtcatactaacagttatgcaattggctcagcggcatatggcggacgtcgtgccgtcgccgcttggagctaacccgacggggattactcatgttgtgttctccacgacacccaacgacgaagaaattgccacgggaggcggtcaggcttgtgtttgtcaacgcagatgacctttgcatatctggatattgtgtactgtatatgctgtatatctgagcgcgttgatgaagaaacatatcccttaaacattcgcagtgttttgctgatagcggtcgggttgccacctgacttgctgcttgaggctccaattttccttggatgtctgagcgctcatagttttttattgtatgtttgtttactgtggagtacaaggattaaatttgacatgcgcgtcgtttgaagtctgtgctgtagtggcctctctaatgaaacgacaacctagtaatccatttctaatgagtactcccagttcacgattgatgtagtcataggttcctgtagattgaaagaggtaaccactccaacatgatccttttgcaacctatctgtccatcaagtcttcttgcttcttcttgagttcgtcttcatttgctgtttacccgtggatgcacatgatgccccgactgagcgctcgagtgcacccccagagctctcataaggtgcagacaccgtcgtcgaactttgaagagaaagttgcaagttatacctgcttgccgtcgccgtctgcaccttttgaagatactgtgccaccatcggctcgtggagtgtgcgggctagatcgtcgtcgcactgagtggctgtacaactatccgcgcctcagcttcgtggtcctctttattgtctgcacgtacattggaggcatctacatctccacggaagtcacgtcgtgggcactggatgcactgggtgttcatagcgagctttcggccgcgaggcaagtgtatttggcaacggctcacgcgttgaactccatgtcagagtgcgtggaccggagctcgctgcagtatcttgcaggtgctaagcttcagtttgaggccgatagcaagcgcgttcaaaagataatcgatgccaacgacgaggttcttgccgcccagaggaacgctacgatgacgtgtgagttccatcttgatgcttttgctcctttggacgatcactcacacgtttcttgtgccaggctcgagcaccttcttgaagactctgcagaagcaggtgaagacagcagcattgggcgatgcaaactcgacgttgagttgtttctccgacaacttttacgcgaatgtccccgacacgggtactctttcgtcctcccgggcactggtgcttgctgtccgtgcaatgctgacgactcaagctgtatcggaagcgcacgacaacgtgtccgaacagcaaaaacaatttgaaacccagctctttggtatgtggtcagccgtcgacacggtcaagacatccatcgagaaaacccttggagccacaaacacgttggcttcgaaacagttgagctcactagcgcccatcttgtcagacaacgacggcagtggtcacacgcaacaatctgcttcccgccttggacggttaagcaaaggagtatcgtctaaactgtcgggcccactgacctcgacatccacgactggtctggagaccatacgaaaggctggtagaacgcttcacaaagccctccaagttctttcaggcttgcacgacgggttttcggatgtcatggacaccgtagacgaaacttgggagttgttggagaagcagctgaatgccaccgtccagcaagccgcacaacttcaaaaggagctcgcttacgcttcagagtcaacagcacagcagatcaaccgcacgagtaccgctatcgcaacgagtttcgaacaggctcaagacgaagtaaccgcatccttcagcatcttgcaacaacagtggcagaatgcagccaaaaagctggtgctggcaggtttcacgccgtggcaacgccttggagatcagctagtagccgagctcactgctaatcagcgtcaaagtaccagaactgagaacgccattcagcgcagatacattttgccggacagaacgacaaactcgaccttggggcaagaaaaagaaagactggccattgctgctcgtgacggtctttcagcaaacacaacgtcttcatctgacagcggcgacgagttcgacattgacacggtcgtgctgcgcgcatcgctggtggacgtcggtgctttcatcacgcaattgatcttctacgtcgatattggccgcctcactatgatggttgcggatctcgctgtcggattgatcaccgagtcgtattcggacatgccgatgctcgacatacggggaatcaccaccgccgacacaattgggagtatctgcgaggtcttcctctgcaagcacagcatatcggctgtctgctacacactcgtgacaaaatcttcggagctgctgcgcgttctggtgacgttcctcttactatttttcgtggcctcaatcgtcacggcagggttgttcgtgtggaaacaagatcacatcgcgcattgcggaagtgcgggtgtttcgcggttgaattcctcccagacagcatttcaaagtatcactcgtgcattcttcgagaacagtggcaataccagtaacagagtcgtcgatccactcgatgagatccagaagtacaccaccattatcaacgactccatccgaaatgactacgcagcactgacgttggactctgctgccgtgtggaagaatcagtcgacagcattcgaagacttcagtgactgcacgacaactacaagcagcttggttcgggtgtttcaagactgcactgagcgcggtagcagcggggaattgggtcttgctaacgatgcttctctttcaagtcagtgcttgacgagtagcctacgcagcccctcagagctggtgaccccagtgacttctgcagagcaaatgagcagcgatgcgccgttcctcgtgccatcctctgcctttgcatcgtgctttccacaagaagagttggcgcatttgggccgggaagaagctgttggtaagctgcagcacagtctggcctgtgcgaccgaaaaggccgtgtatctctcgtgtgcatcgtggtggctactgattgtcgtttttgtcgcgaaccgctttacggtgaggatggtcatcaaggccgcgggtatctactggtggcgcttcttgagtgctgaccgactgcaattcgtggcgttctgcagagaggacggagatctcgtggcgaccgacaaacttcaaggtgctatccaagaacaattgcgcgaagccaaatggagaatcgcgggtcgcttccttggcattgtatactccatcacatgtgctattaccgtgatcctcatcacttttcaaggaattctgtagagaaacaccggaaactacgtgtagcgttcgtagctcaatatatttccaacaccaggtcaaaccaggtcgccgtgatcccgcacgctcattacacttgcttcattgcattacgtgctctatccgatcgatgatgcccttccactttcttactcaatcgcgatcatctccgccaagtcttgcagcgcgtagccgaacgaatcgccctcctcgtcccagctatcaacgtcggtaccgtcagtctcaaccaagtcagagtcgtcctcgaccacggtagcaccaacttcctcctcttcacccttgccaaagcacacgcggaacgcacgcacctcgatcgcctgcaacgtcacagacgtgcctttcacaggcaaagaccgaggcgaccagccgtacgcttcgtccgtcgtcttccactccagtccgccgatctcctcaccctccaccgacagctccttcgtgccagtcaaagtcagctccgtcacctccgacaccaccgagttcttcaccgagaacaacgacgagaagtccaccgtcgccggctgcgacagcgtcgagtgctcctccactgcgtacaagtgcgacagacgcaccagcagacactgcttcgacagctcctgcagcgtcgtgagacccacgttcggagggaactcacccactgctagccacggcaccttcgcctccactccagatgccacgggtttgcggaacaccgccagaggacggaagaactgcgactccatcttcgaccgcagactgcgcatgccgtcctccgcagagtccacgttgacgaagaagttgccgcgcaccgccagacccttcgtcaccagcttcttgttcgccgagtcgtacacggcctccgtctcgttcaggtgctcgcccacgcccttgtcgtcatccgccagcaaacgtcggtgcaccatcacctccacctgaccgtcaaccagactcgccgcaccctgcgcacggtccgtcaccacgttcagctgccgcttcgcgtccttaatgtacgctcccgtcgtgatggggaagtagttggccgccaccgtctcctgattgtcgtgcagcgtcaggttccacgtgtcgcggtggttgcgcacgcgcttcatgaactccagaccgttcgagtccgtgtacagcgtgctgtcggacgcgatgctgttgcccgcgtcgaagcgtacgatgacctccttgcccttcttatcgtcgatggggaccggcccaacggtccactcgatctccaggaactcgtcatcgtcgttcacacggtactccagtgtgacccacttgccgattttgaacgccacgcgtggtacgctacccaggcggttactaccagaaacaccactcgtctgcagctccaccatcgtcacgtccggcaccgcagacgctgccgtgccgctaccgccaatcacggggtacgtctggttcgagtcgggacggaacacgtacgcgccagacttgtgtccgtcgccttggaatgcctggtagtacgccacgtccagcgatagcgggatctggatgttcttcttcttgttggccaacttcgtgatggaccccgtcttcttgctcacctcggcgcgcagcaggtggttctccagcacaacaacgtcctcatcgtcgctgtacttgtcgtcgtcgagactaccagcaccctcgtttccacgctcatattcgacacctcctcgaacgttgctgtcgtcgctggcatccttctgcttcaccaggaatcgagccgtgctcaacggcttcagctccacactgaatacgaacgagttcggagccacagacgtctccgggtgcactggcacagccacgtacacgttctgctcgcggagtctcgcatcccccgacaaaagctgcgcctccgccgacctgtgcgtgatcggaactgacagcgtttgcacactggtacgcgccaaggcgttgtgcaccagcacctcaaagtcagtgttctgcgtggacacatcacagacgctcgtgttggccaacagacagaagtggtatggctccttctcgccgatcacgaagagcacctcgttcagttccttctccgcctgactgatgccgtcgttcagccgcagcgcgtagtcgtcggcaacgctttgtttctccgtgccggagagaccatcgtggtgctgcacgagaccaacagcccgttgtagtgccaccaactcgctcgagtggtggctttggtacacggcgtccaattgacgcacctgctgcaggagagtgttggcaacgcgagcgaaccgcttcaaggttggccgactcgtgaagaatcccgaccagtagtcatcttggtcggacgcgtacggcatgaagtcgtccgtcttgacgctccaagttaaaccttctgcatgcttcacgtccgtgtagtacgacaggttcgagtagagcacgttgactcgcgcgtcctggttgacgtagtggatgagtttgtccatattcttgaaccagtgtctcgagttgtcatactggaagtcacagcccatggggatgaagacatggttgcccttggacgcgccaccacgcatcttggcgttattgacgaactgctccacttcatcgcagacatcatagtcgtgcaggtctgcatcatcctgaatctcattgcgagtgtcaccgtactcgaatttgccaggagggcagtacgtgtcgatgatctcacccgtgaatacctgcgactccttaccgcgagacttgctcggacgccaaatgaactccagatccttgttcttcttcctcaggccgtagtcctggtagtccatgcgcgcgaagtacagagcgtcaaagcccacgccctgcgacaacaacgagccctgtgtggccgagtggccgaacggatcgatctgccacccgatgcgcggactgatgccgaactcgtccatgagcagctggtggccgtacgccgtctggtccaccatggcgatgtagtgcggcgtggcctcgtcgtgcatgcaccagccgccgttgaccgtgaggtccagccgtccctccttcacgagctgcttgacgacgccttgcacctcggagccctgctcgtgccaccagcgctggaagaaggactgctccacgaacatgaactggcggtctgggttgcgcacgagttcctccacggccgtgtccaggatgtactgcaccttctccatgtagtactggtccacccccatcagccagcccctagcggcaacagagtcaacatgttagtatagataatatagttttagtatagataatatagttttgcaatgtgctggtgtgggcgtacgggtcgtcgtgggagtgcgcgatgaggtgcacattgagtttgttggggtcgatttggctggaagtgttgtacacgccgtcgctgggtggcgtccacttgctcgggtcgcaggccgcgtgggcggcagacgccagcagtgccaccgcagccatagcttgcacgcgtgccatcgttctcttccgccttcttcgttcgcgcaactcgatggggaaaattctgatcaacgttttgccagaaagatcgcgtaggtggcgattcaaatttgaatcgcaaacgtgcgtggttgaagtggttcgaccaatgagagtggtccgacggagcagctcgattaagtgcgaattggccggttcctgtgctctgagtcgcaccgtcgtcagtgcacggccggagcccctcgccagcgttgactccatgcagggcattgcaggcacgcgggctttgcagcgagctcaccacgccgccagggccacaactcgccgcgtgcgatcgcaggaaatatccacccgggggggttggatcgtttagctggtcgaggcgccggattgatggggggggggtaaatatagaaggaaacgtcacttgccgcagcaatgaccggatgaatcaagcagcggtgctggaaaaacggcgttgccggcagcgctcggggcccgagtcaggccgaggaccaacaacaagtgcatagctctggcgagctcaagcaaatggcgcgcagagcgcggcggacatcgccaattcgtcgaaccgcctccatcaacttgcgcgagacccaacgcagctgggccttggcgctcagctggaaatgcttcgcaggctgaagtgttgccgccactataatggactcgccgtcagcatggggctttcgcagcagccagcagcttgagacgcacgccaagcgccaaatggctcggacttttctacagccaagttgaaaaacgccaaccatgaagccagcgctatgtccacctcattcccctttccctaccaaaagtttccccatcggttcgactcgctcacttcgcgattttcctccgacgtcggtagcaggttgatcaggactcgaaatgccgctcctcttgcggccaacggcgcccagcccctccatcgacgagctcgcgttcgcggtgccgctctcgtcgcagcgctccgtgtcctcggccgtgtcggaactgccgcgctgcgccacgcacggacgactgtcgctgccccgccgccgccgcagcacggccggcaccgagaccgagttcgcgggctgcacggacgaaacgtgcacggacccggagcgccaccgccagcagagtttgcgcgtcctcaagcccaaggacgcgccggaagtagacgcggacttccgcgactcaaagcgctcgtcgcgcgagacgcaacccgacgacgaccacctgtccagcgacggcagctaccagtccgaggccgagcgcgtgtccagcgtgctcagctgcggctgctcgtgcgtggacgccaacgagctggccatcacgtcctcagccgtcgtgattgaggaccgccagctcgccgtcgtggacaaagagctgcgcgacttccgaaagaccttcgtgcgcaaattgtactcgcgcttcaccaaaatctccggcctcgcacggaggaagtctgctaccaaacccgtcttctgagctcgtgcgtctgcaaggacattgggaatcctgtaggtgcaacaagttaggacactctgccgtcgggcaggcggagtggccagaagatgatagattgtgcttgggcagcacagccaacgtcgctaatatttataaagagttgttgtgtcatccgtacgccatgcaccttcaacaatcgtgctcacagggaaacactcttcaaagcaacgcgtgcaacaccagcaggttgtgcggaagcaaacgggcaagtccagttgtagtctatgagtcattattttccaaaagagcagtgtcaacagctaccgtcaacggcttcttccaccgctaaactacagcggtagcgaacgaacttgcactgaatcagtcgaacggcttgcggttggaacggcggacgtgcgcctggtacatcatggagaacacgagcgccaggacgaacacgagcgccaccacgaacgcaaccatctgcgactgcattatctgccgcaggaacaagaagcacaaggtggagcaacccacagcgtcagcgcaattgagtcacgaacggtaggcctaacgctacagacggcgaacgtacccgcttcgcgctggcgtcggccacctcggacggtttcttgtgctggcgcttgagccgctcgctcatcttggtgtagcgctcgccgctgtagatggtgggccccagcgtcacgtcgaggccgctcgcgccctgcttcgaggcgccagtggcgcggttccgcaactccatggtcgaaggcaaggtgacgtcgcgtcctactcacggaagtgcgggtcaaaacccgtgaacccgatgctggatttcagcgaatccgcgaatttacagctcttttcagcgaaccacagcagctgaaaagcagacccgcgggaatggcgtgataggtcaggctcccattggttaatactcggtctcgccaaatgtcgcgaaggggtcacctcgagcattgtgcagcttcctcgcggcagttcttcactagcgactgcgtcggtggcgaccctcgtagagtccgtctcctcctccctccctccctccccgtatctaagcaagctaccatcatgagcgagtggtgggcgaccatcggccagcccttcgcggatgtgcgtggcgtttctttgcgttccctgcgtatcagagccagactgaacgtgatgctgttgtaggcgctgctgagtctggataccgtcaagatcaaggcggcgttcgcgaatgccccgagctcttactacgagtacatccgctccatctacgagcgcagccccgagcacgtgattatcgagacgttcctgattgtgttcgtaatctacatcacgttcgtcaagcgtgacaagcccaagggaacggcctccaagctctcggagcgcgagatcgacgacttgtgcgaggagtggacccccgagcccatcatcccgcctgaagccgtcgtggacgtcaacaagtccaagcccattggcatcgtggaggacacgcccggcacgcatctcaagctgcagggctatgccaacccgctgctgaacttggccacgtttgacttcctcggattgggatcgcgacccgagctcaaggaggtcgccgtcaagacgctcaccaagtacggctgcggttcctgcggaccccgcggcttctacggaaccatcgacacgcacgagatcctggagaaggacattgcgcacatgatgggcacgacagactcgatcacgttctcggacacggaggccacgtcgtcctcggtgctgccggcgtttgctaaacgcggtgacctcattgttatggacgagggctgcaacgactcgatcctggtaggtgccaccctggcgcgttgcacggtgctctactacaagcacaacgacgtggaggatctggagcgcgttctgcagcgcgtgcgcgacgaagacaagaggaagaaccgtggctcggactgccagcgccgttatgttgtgacggaggctcttttccgcaaccacggcgacatgatcgaactgcccaaggtggtggagctgtgcaacaagttcttcttccgccttttcctggatgagagcttttcattcggtgtgctgggtaagtctggccgtggactcacggagcacttcggcatggacgtctcggaagttgctattatttgcagctcgcttgctggatctaccgccagcgtgggtggcttcagcacgggttcgcaggaagtcgtcgattaccaacgtattaacagtgctggctacgtgttctcggcctcggcgccgcccttcacgtcggcttgctgctcggaggccatccgcattatgaaggacgagccgcagctcttcaccaagctccgcgacaacgccaagctcgcgcacgacgctctgtccgctggcgtcaacggcgtgttcgctatctccaaggctgaatgctcgcccatcttacacctgcgtctgcttcccgaggtcgtggcccgcgtcggcagcgacgagaaccagcgtgctctgcagcgtaaggtgtgcgacaccgtcatgcagaagtgtctcgccaagggcgtggcgatctgctcgccacggtacaagactgaccagacgctggagccgctgcctagtctgcgtgtgtcggtgactgccatccactcgcctaaggatctggaaaaggcctgcaagaccatcgcaacggaggccatggctacggccaaacaggtgctcgccactttccctgtggacgatgctcctgctagcggattgcgacagcggaagcattaaatcgttagagtttccaaactccgtcgagagagaggccgccacttttcctaatgaactgcttgacgcttactctgtatcaatgcgttgccagtagatagtcttcaacggtagtgatgcattggctttaggctgtcgatcgtctacatagcctgcttgtcctcctccagcatctccttctcgcgcctctggcggtagatctcgaactgctctgcattcgtgtactggtgcagacaggcgttgagttccctgttgttctcgcggcacttgaagacgaccatgaggccgttggccttggcgcactcggcgaacttctcggagacgttgcggcacttggcgatggcgatgtcgctcagctcttgtcgcagtttctgcagacacgtcaaaccaaaacagttagccaatggggtaacggcagtctcaagcggcgggaggacgcacgtgctcggcctggttgctccacgacagccgcgacgagcggaacttgttcttggctgtctccttgtccacgggctgctccatgatgtcctcggtctcaattaagggtatgcggtgggcatgactccgcgtttccaatcgcaatgtggtgtgcaccacttcagtaatgctatcatttcgatacggtgtgtatcaaagcaacttcaacttgtctttttgcttcggagcagacctagacacacaatggatcactctaccgccgtctgaaaggcgtctcgcgctagttttgatgcccagggccattgtacggtgtttagagcggcaaactcgtcgaaaatgcgcttcgaaacggcaagcgtgtcgcccgtcggcatggttgggtacgactcgtggctgagttgccacttgagctcgaacgcgatgcgagctttcttcaagacgtcctcattgattttgcgtccttgggtgtatgattgacatacctctcctagccacatgcgccagcgaggcaagtagtagctctttacgaggccagcccactccttgcctgcgtaatcatgaatggattcggagttgttgtcgccccagcgtgtcacttgattccgagcttcgtactcgtagtaatcctgaagattgtccacagtggcaatatcaccttcgtcgacaaggccccgtgcgtcggcgatccagttgccgagcaggaagtcatcgctcgtcgctagaatctcgtccaggcgctcgatcgtgataagcatgcgacccgtccaagcacacaattggtctgctggcactccctttcctgtgtacatatccttcaaatggaggtactgcgccatgaaatggtcactaaggaactgccgcgtgacatcgaccaggtcatggcgatacgtgtcgacagcatgcagctctgtccccgcaaacagtagctccttccaagcgcgagcaatgtccatagggtcgtaagtgatcaaagtcggcataaagccatcacggacgagtttccagtgtggtatcaagcacaccaagcttttagtaacaccaccgtaacccagcgtacgattgtataccgactgcagcaagaagccccaagcccgttccgtgtgcgcgtcttgtgagtgataacgttgcactgcaaagcttgggacccactcgtccatgtctagtggagcatccacccatgccatctgcaaggtgagatcgtacaccacatagttctggaaaatgccttccatggtcagacccacacctgccatcgtcccattgctggcctggttggccaacacgggtgctgtgccgagcgtcggtaagtctccacgcatcccagtgttgccaccaaagttgtgcagcacgcaatagatccagctcttgccaaagtagttgtccatcttgttccacactgggcttacctcgctgtagagatccagaattatcattttgtcgttgggcacgccgtccaggtatgtcttcacacgttccttggtccaatagtctggactgttgacaaagagccaaccttggatgagccaaactgcattaggatccgtagccgtcatgctatcgatgacagcgcgcgatgcagcctgcagccgcaccggatcagtaaattctgggtccatctcgttgtaggtatcacactggtacagcgaagacgtgtagtcgtagagagcacgctgctcctcaaggaaagtcttgccgatttcatggtagagcgggtctgcggggtccagcatgtatacacagcagaactcatctgaaaagtctccccagttaggcgagcgagtgaactttgcttttggaaacagcgacttcatctcttctggcacatggccagcgaaggctggcaaggcaggaatcataccgaattccctcatgcgcgtcaggatcttcaactgcaacttgaactggccatcaataaaggcttgcggaagtgggccctccacccaactgccacggaggttgcccatgcgaccccacgccagaaacgctgatccggcgaagaacttgttcaaaccagcagcgctaacgttatagtgtttctggaacgtgttctgccacaccctctcttgcccagtgaacgcaagcggcatattgatacctgagcagcagtggacacacgagtgtcagtactaagccctaagtctcatcttgcatctgaaatatatataaaagatacgcaccgttgagcgccatccagtcaatatggttctcccacttcttccacccccacgtccacgaggagtagctcaccgtgcacacgttctggtagtacgagaacttgctgctacgtttgtgccgcacgcgctggtccacctttggtagcacgtagggcagctgcagcttgtggtcgtcccagtctgtctgcgtgtgcagtgccgtcttcaagtaccattgcaggccgtacgccatggcggtggccgagttggcggcgatctcgagtttgtcgccgtcgttacccagctcaaacacgtccagattatcggcctcggaaggcagcactcggagcgttatctagtatcaatgcattgttagtttcccattgcgatctcctacgtgtaagtgatcgtagacagcagaacctggtcgttgaagcgtgcacccagtcggcggtggatgagcccgcgcgtcgtcgccacgacgtcgtgcgcccccgcgctcttcttcagctggtcgatggaggccgccgcgagcagcggcagcagcaggagcgtcaccaagattcgcttcatcgaatctgttgttttgagctcttcaaccacgtaattagttgcgtaattaaacgacaaattaataaaactgaagtaaatcgctgcgtgtcatgacgtaacgaagcagcgcgaggtaatgtcctcgattaattgaaaaacaccatattctgcgtcgtacgcactcaaataatcacctttatcaaccaataagataacatctgaacgcttattggtcgtaatggccgactcattgagggttttaacgaagaattgggcagctcatttagcctcacacaagggtgctgtcctcgctgacgtacacttcccccacgcagacaaggcataaacgctgtccgagcgcgcagagcacaagtaggcaagcagaagacacagcaggacgatggccttgcgtaaacgccgctcgagtcccgaggggagccccaagcacgcgcagaatgcgttgccacccgcggtcaagcccgcaggaagactgaccaagttcatgacgcgcgtcgttgtgggcttcgccatgatcggcggcttcatcgccatcctgtacggcggccacatgtacgcgtgggggctcgtggtactgctgcagacgctgctcttccgcgagctcgtgaacgtgcgctaccgggcggcggccgaaaagaatatcccgtggttccgctccgtgcaggtgcgtgcagctgcaaatggagagggggaggaggagggaaggtatagactaatgctgactcttgtggctgcagtggatgtggttcgtggtggcgctgttctacaactacggcgactcgttcggcgccttcatcgagagcagcaagattcgcttcgtgccgcctgccatcgtgcactacctgcggtaccacacgtgggtgtcgttcaccatgtacgctatgctcttcgtcatgtcggtgctgagtctcaagaaaggtgcgtgactgtagatttgatgttgagctcgttgtgctgtcgtacggttgcttcaacgactgacgctgtgccgacgaacattttggtgtcaacgatgtggttttattgactgggtaggttactacaagtaccagatgggtaagttggcaggaatctcgttttgagtttgcagacattctaacttgtgaatttctcgcttgcagggcaatacacgtggaccattgtcacgctgggtttgatcgtcttccagatgaaatacgtcctcacgaatatcttcaacggtacgaatgtaacaaatatcccgaccttgtcacaagttgctaacgggattcatatgcttgttcacccctctaggtctcttctggttcttgttccccgtgtcgctcgtgatctgcaacgactgctttgcgttcttctgcggcaagctttttggtcgcaagttcattaagacgccgttcctgcgattgtccccgaacaagacgtgggagggcttcataggtgctttcgtttgcaccgtgatttatgcctttttttccagtgccttcatctcgcaggtacgaagcaaacacggtaccgtttcgcgtattttgtttattgattgtgttgtcttttgttcgttttagttttcttggttgacctgccccgtggagagcttcgaggtgagagcacagtgtttagtgctgcaaagcactggtcttcatgcagcatacgtgcttatggtaacactgatgtctttattttgctacagttcaagctgattcccgacccactcacgtgcacacctcgtgacgtgttcctccctcattcctacggcgtaccggtctacctggcgggtctaattggtcgaagccagatccagcttctcccgattcaggtttgatgttgtggctggctagatcgatgtatggcgattgctgacgagctgtctggttgtgttcgtgaatgcagtttcactccatctggttcgcgatcttcgcctccgtggtgtcacccttcggaggtttctacgcttcggcaatcaagcgtacgtacaacctgaaggacttcgactcggtcattcctggccacggtggtgtcatggaccgaatggactgtcagctgatcacaaactgcttcacgacggtctacttcaacaccttcattcggtatgtcgtttctctagtgttcacacggcccgctacatcgtgttgctaacagttgttgtgctttgtgtgttgctccctctattgcagttcgtcgacaccgtccgtggcgctgattctgaacctcgtggcgcagttgacactcgaccagaaacaggaggtgctgcgagccatccaagagatgctccaaggctgagaccatctcagcattagcacaaccatctgactgcttagaaggaggacagaaagagaaaccattgccaacggtaacagagaccatttgccactcccgttcattttttctgaagtcgtggagaacaacctaacagcgtgatctcatcaccccctgcaaacagcgatgtgaagtagtgcatgaagaaggcaagcacaaactattcgccgggtatcttgggtgagtacggtggtcagtcctctattagtattgggtattgtatggcatttgttgcacaagtagaaatcgcattaccataacctggaaataacccgccagtacaataattacttttagctcattaccgtaattcgctaaatgttagaggtgagcgacaggatcaaacgcaggatgctctacgaaattttgcaggaccaaatttcgggtgcaggatgcctgcataaaattcttcccagctggtgacggatccttagcgttgactgcccagaaccataacattgcagcacatgtttgacctaaaaacgatctggaagagattgaagagcatacatcaagtcgacaacctgcttctagcacatcgtagaacgaatagaatttttttttatccttttcctcgcgaacaattcttgagtctgcgttgtcctcttcacctcccgtgtcttctctgaacgtcgtcctaataaacgcacgacgtaagtgatccacttccgggggtaggaaaccttggacccgcctctctgagagcagtgatagcccaattccttgtgatgcggacttctatcgacttctatcacacatggaagtgtgaaattgcgaatttttccaccttgaacagaaaaatcggtactccatttcgctatccgaaaagttgtagagcaatctccccagctttaacaaatccatcttttccagctagaaaataggcagaagtagacgccaaccaactcttgtggcgacaagtttgcttcctatgacgtcaatcctttcgtgtgaacagtttactttttcatcgccaatcacgggcttcaatacattcttcgagacaaacgtttcattttgttcgtaccatataaaataaacaatttccaatttccaaatggtagtaaacgcaccggcagacgagcgcttctgcctgcgcatacggaacctcccggccgtgctgtccaccgaggcgatcacgtccttaatctcccactacgggtctactcgcgtccgcgtattacagcgccgggtacgtaatgaccagctctgcatgaagggatattttcatgtatcaatggccaatgtttttctgatattttgagcagaatgcagcgtcgagcggcaaggaacctggtgcagcaccatccaagcagctgcaaaaggctgttgctatatttgcaacgcgggaagcacagcagaacgcgcggcgacgtctgcattccctggtattggctggtcagcacctgcaggtggaagtggttggcgacgaagctgcaaccgcctcgacagagacgcgaccgcagcctgtgcagactggggatgagttgccgctgaaaggacaaccgccactgccgaagggtttgcctccaccgctgccaccgacaccgactcccatgcagaatcttctctacgcaccagcaccattggctccgcatcttgggtaagttgatacactattgagctgaaaattatcagtactcatgctggatggggtttatttcgattgtagtctgcattacgccccgtcgccactgctggagtacaagtaccccaaagcaactgaaggaatcgtacgcaatattgcaaatggtttgtggtgctgtgatgatgatttgttggagtcaaagagttggatggatgttcctaactcgaaagatatgttgttgtggttgttttcagctcttattgcgttgcctcgattttatattcaagtgctgcatcttatgaacaagatgaatttaccgccgccgtttgatgaagatgcgatcccaggaagattttccactcaccgagatgttacaccacactactgtgaaagtgagggtatacgtaagcgaactctaaagcgccgcagaagcaacgaaagctcgaatgagcaaattgttgaagaggacgaagaagatgatagcgaggaaggtgggaatgatgcagtaaaaagcatggatcacaacaacacccttggcactgaagaagtgcatttaccgtcatcacggctacaaagtgggcgtgtcacggaagattcaaagtctgttcaaaaatcttctgaggcctcgatccaacctgaagctaattcttcacggcccgtgcttctcggtatgcccttttcgaaacagaatagaggcccagaaacgaagaagagtgcggcgctcaatgtggcgtttcaacttggaactgatactggaaacactccgagatcacgccgattgacgcgtccgggcgtgatttcagagaacgaggtgaagcggcagcgactcgcacaagggggtacgtcgtgcttcagtgggactgctcgtaagtattggtctcacacgctgacaccgttgcgtgcaaacagatttccagaaggaacctctcatggcaacgtactcgcgtggatcaccatcggccgttgttgtggtggaaaatatcgcccaaacagtggatgagaaagacttgcgctacgtattcggctacgttctgccgctggaggaggctttaacgtaagtgttgctgctatttcgttgcggactcgggtgccaggaacgcgttcttatgtttaccattctgtggttagctcgctgaagatcaggctcaagccaggaaagggttctgcagtaataacctaccctgatgaagcggtagcagtagcagcagttgatgaactgcatggtgtacaactggaagggaaagcgttgcttgtggtacggtatcgattttctaccccatcgcataatttgcgatattgatgctcaattccgatcgcataatttgctttttgtgaaatacagagtttccagagcacctctggtgtgtccgaagtgatgccgagtgtacggcattggactatagaggaactggcaagcaaccgcctacacgaatcgcaacttgctctggagaagagcacaaagacgtaccagcgaggcgagccttccgacacgctctatgtaaagaaccttgccaaggcagtggagctggcagacctctgcgctgtctttggtgcagtgctgcctcccgaatctggacctgagtacggcacaacagcgcatttcagccatattttgatcgtgctgacactaaccaccttgcggtattcttgaacggtgcagcacgctgaatattcgccatttcacaactggtcgaatgaaatgccaggcttttgtgaaatactcaacggttgaactcgcttccagtgcgctaaaccaagtccatggtgtcgttctcaaagataagccgatgatcgtggtacgtaaacacttttttcctcatggtgtgtgtgtgtgctcattcgattcacatcgcattactttttgcagtgctttcgtaaaccccaaacaaactaaacacgaagttgctatcgcagtccacaccttcacctctaatgcccctctcttaaataacgatagggaagattcttcacaacttcagggcagcattcagggaagctcacgcagttttgcctcgctgatgctgttgatcacccaaagataattcacaatgatcgctgcagacaccgctttcacctgcgacttcgctttcagatgccttgtttgctggcgcaatgcgttcggattgagcttgtcggagacaatgatgagcttccgtgtacggttttcgcgcttgtgcatttcgtcgggaatgtcatccatgaatggaatcaactttccaccccccatgcagatcaacgagtttagctccgtgaccggtggcataggtggaagaaactccccgatcaagtggaaacagaaatctgcaaacagcatcgtaccaacgttcgagcgatcgacctgtgttggcgcgtacaaaaaacattataagcacgagttagtgtgcatgcagtgccgcggctgagcaacaacgaatcaagatgagatcgtacgtttagagaagtcagctggagcaacttgtcacgtgttgcccgtgaccgtttgactgcgtcatggatccttcttcccttccagtgaccatcggcttcataatttacttcactcacatagccgccatggtctgcgcaaactggaaggtagacgcataataaagaaggtgacaccccgcccatagtgacgccgtcagtatcgtaaataatgaccttatacagaaccatgattgccgtaccttgaagccactcatcgctcacgatccaccgaccggcaaccaatgccttcagatacttcaaggaccggatcttgacccatctgccgggcttcgggtcactgaacaacttccgcttaccatccttacgcgcctggggaggagactttgaatccgattcatccatcgaacgctcctcatccttgacttcttcggcgctaacacatttcacaatcaaatgtgtcacgtgattggaccagtactggacaacttcagcaccaagcagctcggcccattcgtctatctgttgcattcggttttccttaaatcctgagcacaggaacaccatgcctgaatgcgttgtcagcgccttcgttttaggcttgaccttgttttccgaatggggcgagggccttgttttcggagctccagcattcggcgaagccatgatctcagatggtcgtcgacgttggcgctttcttgcagaatggcttggggtcgagtccgaggctatatcctcggcaggtctgcgaataaaagccccgggcacgcgtctatccattccagtgccaatcagaaacttaacgccgtaaaagccgtctccgtgaaccgtgatgatacgcgccgcgcccccacgtttattgattccactccactgccgctcaattacgtccaccaaggcgccgacttggaaagtttcgatttcaagcggaagctgcgtttggccatcaaaaaccgaggatgggtttgccgctagtccgttggtaggagctatttttccattggattgatatgttcgctcagatgcccccgtttgcgcagtgtcgtcgaatccgtttgtttcagatcgtcgccttttgagcggtgtcagctgctgcttcgcttcaatagcccccatctgtggttctttggcgacttcaactgtagctgattgtgaacgcagcgtcgcctggtgttgtgattgcgcttgtaccacatgagttttctcttgggctgcatctgtagctgacttccgccgccgtggctccagagcttgctgctccgttcgattttgctgaaatccatcagaaatgtcaactgtagctgaccgtcgacgttctcgcaatgtcagtgtccgtggcttcggctctacagcttccacccgcgcttcatctgtggctgcatcggtagtagaccgccgccgtcttgtagatacagtttgttgctgcaagtcttgcagttgtgtttcgcctgcggctttatctgaaacaagtggatttcgggaagaccgccgccggtgcggtgaagatgatgctcgctgcacctcttctggctgcatttcggattcaatttcgtctataaggattgcatttcttgaagaccgccgccgccgtggagttatagcctgttgcatcggttcttctaccggtgttgatgcttcatccatggcttcatctgtgattatatctgattgctgacgtcgcggagataatgtgtgctgctgcttggcttttaccacttccagttgtgtttcatccacgtctgcgttcggagccgtaatttccaaggtacactgtggagataatgccttagcctccagttgtgtttcatccgctcctgctgcatccgcacccgaccaccgctgctcatccacattgtcaatctgcgtcattgtatcctcacaatgaagtctctccgcactagaacctgcaatcgcttctgccacttgcgtcttcgctgctctcggcgctgaaatctcaaacagcgctggggacatgggcgacgccatctcggagtacaagtcccgtcgacgcggcgacgctctcctcactggcgtcgtttgcttctccgtcttcatcttgaccaattgaggccgaagcgacgtgctgccgccagtgatccaagcgtccatcagcgtctgcgtctgcgatcctggggagtttttcgtctgcgctctccgtcgcggaggtgacctcctctcgggcgtagtagcagcaactttaacaggcttggctgctgcaattttaacgtcgctcggggctgtcgtcacctgcttctccgtcgtctgcttggtcgtcttcatctcgattgactgcggctgcttttccgctgcagcatcctcgggcgcagcgcagagcatctccgtggcgcatagtagctcctgcactgtcgtatcgtagcgcagccgtcgcttcttggccggcgccttgcagatggggcacagcgtcttgagctccaacgcacggtggatgcactcctcgcagaagcagtggttgcacggcagcgacacggggttgtcgtacgcacacaggcagatagcgcactgcagttgcgccgagaaggtctccacggcgcgctccagccgcttgagcgtccacggcggcatctccgccaggcgaaattgggtcgtgtgctcaaactttgacgttttggtcggatcaattttcagcatttttggtccgaagttttggtccgaggttttattacagtatctcaaagcaggaagcgattgcagcaacgaggatgtctctggatgagtcgtccatcctgatggaccgcgcgctggtgaccatgtgcccggacctgcccgagtcgctggggctgtacaacacggccaaagaccagttctcgctagcagccatgcgcaccgtgccgctgctggagcacgtggagccggactcgcgcaagctggcgctactccgcgattggatgcgcgcgtacgagccggcggaggaccagccgctgacggcggagatcagcatggactcgctacgagtgacggccaagcgcgagcgcaaggagcgcgagcggcagctgcaggaggagaagcacgcgcagcgcacaagcgagagcatagccgagagcgacgacaagaagcgcaagaaacgccagcgcaggaaggagaagcacgcagccagggacaaggagagcggtcgaggcaagggcaaggacaaggccaaacccaagaagaagagcgcaggtaaggagaagaagaacagcaggaccagggcagcaggccgcaaacgtcacgtgaagctcgacagcgacgacgacgtcctgtcaattgaggaatcaagcagctccagtgatgaatcgtcgtcttcgtccagcgagtcggacgacagcagagcgaagaagcgcaagaagtcgtcggggaacctgccgcccaaggaggtcgtgctgtcctcggacgaggatgacgaagagcccaatgacatgttcgacacggacgacccggacgtgtacgaggtggagactatcatccggaagaagacgggcgagagctacggcgacccggacttgtacgaagtcaagtgggaagggtacgatgagaccacgtgggagccggccgccaacatctcgaaggacatgatcgacgagttcgagggccagccggtgcgggaagacgtgtacacggtggaggaaattgttgatcgccgcagcaagcgcgagccctccacgaagctcaagacgcaccagtacaaggtcaagtgggttggctacgacgagctcacgtgggaacccgccgacaacctcccgcacaacctacgacgaaagttcgaccagaagtatgagagtcggaagcgccgacggtcgtgagaagtggccagccgtaaagaacgaagtctgagaccgaagatggtcaaataccaagtgcaaagtgcaacgagccactacacttttctagtatgatggctacattatgtattccacgctatgtattccctctacaagaacagatcagctcatatccctctagaagagtgttgctgtcagggggacttccctcaaagagaacttggctcgcaaaggtcaaaaagctgcggcgatcgttgcgcgaggacggctctatctgctcgggcacgttacgtacagcgcatcaacacggcgcatcaggatccccactcttccgcgcgtcttctggtaccttccacgcgccaatctgcaacgtagatcatgagcggcgtggagagtttgctactcgagagcgatgatgaagacctgggggagctggatctgtccgccatcacgctagaggagatcctacgtgaggaggaacaggccacgaagggcacgcaggcccagggcggcggcgcgcaagacggagacgagctcttcttctcgcccgcgaccttcacattgagcatcgagccaggcccgggcatggacgtggtccccgcgcccaaggcggcgcctgtagccgcgtccaagacggagacgcacttccgctcgtcgctggagatcgcagaggctcgggagaagcagctgctgagctgcgtgggcgtggagctcatctccccgctgcaggtgaagcgtcgactgcgcgcccacgcgcggtccaaagccctcaaatcccgacgagagggactcgtggccatgaagaagaaccaacggaagaagcaaatcgcagagggcgcgggtgccactgctgtggctgtggctacggagaagaagtccacgggcgtggtcaaggtggagcctatggaggctattagtcgccagctaaggaagaacattgagttcaaggagtacggccccgggtcccccacggtggtggcgatccactctaagttcattgcaattggaacgtccaaggggctcgtgatcatcttcgaccacttccagaatatccgacaggtgctggggaacacgaacgacgcagacggagatggacccgttacggctgtggacgtgtcgccaggcagtgactatttggtgtgcggctaccagagcggacgcattgtgctctgggatatgattaagggtacgtccctcaaggccgtgtcggatgcacacgagaaccctgttgtgagtctgcgcttcctcaaggaccagaagcccgtgctcgtctcggtggacaccaatgggctcgtgaacaagctgaatttctccaaaatgatgggcatggtgtacgttgtggatgtggatccgctgtatgacggctccgcggggaagatcttgtccatttcggtgcttccacagtctgcgggaaatgccaagatctcatacctcacggatcagtattgtctcgctgcactgagcaccgaaaccgttacgtttatcattgccatcgaaccagaggtccgtgtgatctaccgctgggcgcgacctgatgacattgcgccggatgacccggtgctcccgtcgctggccttcgcgtggatttcatttccaggaagctcacgtgctctagcgccagtgcttgctcgtggctggggtaaccgcgttcagttcttggaggtggttttcccgggcggcaagaaccactcgcatgggcgccatggcttccctactttcgacgagcacgaccaaatcgagtcctcgagcgctgttatggctgtgcagtggctaggcgaccaagtggtggtgtatctcaactcgcacgatgaaatctgtgtgtacgatgtgatgtctcgccaggagctggaaatcgtggacgtttcgtccctggagttggtgtttgcgtcttatcgtggcaagaacgctcgcagtttctcaaacagtttccgtggttgttacaacattttgtacctgctgggtcttaaggaattgcagacagctcgtgtgctgccgtggacccagcgcatcgatttgctggtggatgatggcgagtggctggaggctctcgcacttgcgctggatcactacgagggacttaagatcgctgcggcggaccgtgcggccagagatcgattcccgcctgtgttcttccgtgacaagcagaatgatcagtgccttgttgatatcttacatatgtgccagactaaccagcgtactggcgagaaggaggatgtgtttcggcatgaagagagtgcggacgaggtgcgttgggtctgtggtgaagtgccatacccgcctgacattgccaaaaagctagaggagacactccaaaaagcacgtagcggtgaggtgacgaagaacttcgtgcctattagtgtggctgagcgcgtagcagatctgctgatggaatacgtccgtcttgccatcgctaacgcgccgggttctacagctgccgctggaggcgagctgagcctgaacaagatcggaatgaagctcgacttggcgaagagccactaccagatgctggctggtgtttgcattgagtactgtgctctcatcggtcgaacagaccttctctttggagagatctacacgcgattcaaggatgccaacaagctcagtgtattcgtggagttgctggaaccatatattctatccgagaagctgcgtaacctgtcgccggtggccatggaagagttcgtcaagcactttagtgatcagggcaagcttgcccaagtggagcagtgtttgctccacctgaatgtggcagagctggacctggataccatcctcaagctttgtcacgaccacgaactgtactcagctctcatctacatttacaacgaaggaatggatgattacacgacacctatcgatgtgttgctggaagcttgctccgacgcaaaggcgtcgaagtcaaagcctgcaccagagccggctcctcgagctactcgtgcaccttctggtagcaaagtgagcagtttggcatctgcgttcgggggtactacccgcacggctggctcaaaggtctcaattgcgaagcctgctgggttgtcagcacgcgaaaaagctgaagaggaagcgctcagtggtcctcgacgccgacggttgtatgggtacaaactgctgttgtacatttcgtacgcgttgagcggtcgctcgttcccgaagcatgaaccgatctcggcccaaaagctaggaaaggtgcgctcgcagatctgctaccatttgttcgaacaggcggtggcggggtcatcaaacccgcgaccatacccgcgactcgagacgcttattgatcttgacgcacgtgagctgttcaacatcatgggacgtatgtttgacacacccagcgttgagtttgaaggcgaaaagaaggacggttcgggccgtccaacgagccgatacgactcggctcgcaatgctgaaatgaccaagtgccctagccgtctgtcgattgtgctttcgcttgccgaggtaatcttcggcccgaagagccccttcagctctgtggagcacgcacacttctttatgtttgaggctcgtcttctcagtggcggctccatcgaaccacaggaatacgctgatgcacgtgccgaggccattggtgacgccggctccgctaacagtggcgaatctatgatggattcgctgatgaacttcctggcgcttggtcctgcgtcacttctttcgaagggctcatctgcagtcacgcctgctacctctcaggaggagggcttcgacaaggctggacgtgaagctatgcttgtgcgactactgacaaagctgagcaaagccacgtacaaccacgaggctttgttggcgagtgtcatccgagagaagatgaaccgcgcagctgtgttgttatacaaggataagggtgactttactcaggcgattgcctcgtatctggctgatgaagaccacgagtaccaggtgaatgctttcagctacatccgcgtggagaccgacaaggcagtggatggtgaagagatggagatgcgcgacagtaatggagctgagcctacccgtcgccgccggaagaccattgaagaggctgtgcttgcccatgcaccggctctgatgaagacggacggctacgcgttcgtgacgttgattctgggccagttcccgaacttgaacaacaagataatccaaaaattcctgtcaatgggcaaggagggtgcggagctcgagttcctttacctcaagcaggtgcttatggcctcatcgaccacgagcggcggcagcccaagcgacgaggccgatgtcgtgaaagacctgctggatcgttcgaagttgcgtatcgccgacgaccccgctgtccaggagcgtttcgttcgccttctgtgcgagttcgaacccgccggtgtgttcccgtaccttgaatcccacgacagctacaaggtggatgcgtgcctgcggctctgcaaggagttcgcaatcacagacgctgaagcctatctgctcgaacgcacaggcgatgtcaccggtgctcttacgcttatcctgcaaagtctggagcaaaagctcaagattctcaagccggcactacgtggatacaacgtctccgctgtgtcttcgtcgtcgacaggagcttcggacatgctagtaagcactggtggtggcactggtggcgctggtggatctagtgggttgacttcgacgtcaagacaccaatcgtctgaccgcatcattgacagcgttcaggagggcaaggacgcaacgaagacgctggaagtggccttgaccatgtgtcagcgtaactcgctgcgtaaccgagatgagcaagcagaaaagctttggttcacgctgctggacaagctgctgcgtatccagaactctgtgaagcgtagcctcagctccaagacctcatctcgcaacgttcctgtcactcggactcacagcagtggcggtaacagtggacatggtgctatgactgccttccaggtggcgttgaacgaaatgattcgtttcattcttgaacgcatggcttcctcagtgtcgctgaagtcgatccttttcaagatcacaaacgagcatggtcgtggtgcttttggtgacttccggcccactatattcggtatgctggacacgtacaactacgagcagaacatttaccagacagcgaacgcgctcatcagtgtggatctgttcgaccagattacgacgctgaagcgagccaagtctcgctgctacgccccaccgtcgaatgtgtgcggctactgtcacgtcacgttgtccaagcctccttttggtatgggtcaatctggcgctcctggcacggagaagtggcatttgcacacgtccatggtgctcgttacgtcgggtcaaaccttccacgagtcgtgtggcaaggcttggcagcagggtgtggataccaagactccggcagcgcgcggagctcctcagagcaagctctcgcgccaccacagctcgatcagtagcatggccagcgatggactggccggagaagatgaagcaggcagccgtctccagcgtaagcagccgagcacaagacgctacttgacacgactaaagacgcagcgccgcgcatcccgacgacaggtgtcgcctcacgttgtgctcgaaagcctcatccgcgaggacaacgggcgcaacaagtacctcaagagctcgcgcgcagttttctcgctgcgcccggacccggaggtggctgccagcaaggtgaagaagcgcctggggacgcgcaagcctggttcgctccccgtggcgcccatccagaagggcggcatctaaggcagcaaattgttgaaatgagggagctggcggaaatgtgtccaaacatcgttgtgcatgttttaggggggtaagcaagaatcaaatggacccgcggactgcgggtcaagtgtgtttagacgttgtgtgttggcaggtgctgtggggggccgcagcttggatctattttgctttgtgcgcttgcagcctggggtcggttgcgctggagagcatgagcgaaggcaaacctgcagccaaagcgacaagaagcgcgatctacagcgttggttcctggcgactcggcgatgatggatccatccaccggcgcggtcgattgaagtactcaaccggtgacgtgtacgacggcgagtgggtggatggcaagcgacacggccaaggtgtgctcacctttggtgcaggtggcagctacacaggggagttcgcaaacggcctgttcgaaggctttggcgtgcttcgtgtgcccaagacgcagcacccgttgaccaagcagtggctgcgtggagagaagtacgaaggcgaattcttgcgtggactcaagcatggccgcggcacgtggcagactcgtagcggggatcagtacgatggcgagctgaagcaggggctttatgacggccgaggtgtgtgcgtctatgcagcatcgggggatgtgtacgacggggagttcgttcgcggtctgcgccatgggcgcggtgagctgcggtttcgtaatggctcaacacacagtggtgacttccgtcttgactactttcatggctttggtcgggtgtcgtacgggcctggaggcattcatggttcctacgttggagaatttgccgatggcaagcggcacgggcgaggagttcgcatatacggcaacgacgaagccaatagaacgaacaggagacgttatgagggtgcgtgggaggatgacgaacctcagggcactggtgtgcttgagcgtgatagctgcactgctgtggggatgtttgaacgtggtcttcagaagggtcctggagcaatgcggtttgcaaatggcgatacgtacgatgggaacttcgagaagggagattttcatggagaaggacgcattgtctaccgtgatggcggcgcgttcgaaggcacattcctgcgctctaaacgccatggtaaaggcaccagagtgttttccaatggagatcgatacgtaggcgactgggctgacgacttgatgcacggccgaggagtgcacacgagcacaatcgattttacgcctcgcggtaagaccaagaagggtggaggaagtggcaaactggtttacgatggggagtacttcaacggacaccagactggagaggctaccattgtctacgtgttcataccgtatatgcctgacaccgaagaacggcctgaaacggagcttgcatccaagacgcaggaggattcagcaaaccagcctcagaagcagtggccgaactgggaatggaacggagagttcgagtttccggagggctctggttgctggcatcgtgggcgcgggaaaacgacgtatcaaggtggtgtcctgcgtggtcgcttccatgggcagggcgcactgcgctcgcctgatgggaaattgtggcgcggtgagtgggcccacgggcagcttcaagggcgtggcgaacgcgtttaccttcctttagagtttgacactctgacgacaaagaacacgctggaaaaggacgtcccgaccatcacgttggcggatagtcggctggggctttatggcgtggtgcattacgttggtgagttcgtgaagaacgtgcggcatggcgaaggtgaactgctgtacacgaacggatatcgacttcgtggtcgttttgcaaacggcttcgtcgagggcgtagcgtgctatggcgttggcagacttggaggagatgctcgatggagatacggtgagtttgttcgcggtgagcgcaagcgatggctcagcgaggaagaggaggcagtgattctgaagcagaagcaagacgagcaagctgctgaaacacgcaagcaaagcctgctacgaacgctgatgccgaggtgacgtgctcaaagacctgacaactaaaacatcaacgcgaacatagttctattctatcatttgacaagctagagttcttcgtggcgttcaggtttactacctgcctctacctttgcgccttgctgttgcagttccttggggaattgtttcacttgagcgatttcgcccaccaatgtgtcttcttccactgcctcgcaagcagcaggcgtctcgaagcgaatctcgtactcgcacatttcgcgctcttcgacggagacgacgcggttggcaacaccgcacgagaactcgacgcgtgtctgacgctccttcccatcggcacacggctcgccgtggttgtactcttggacacgcgcgaaacgagccgaatcttcatcgctggtcagccactgattccagatacccagaccaatgggacggggaacatcttcctcttcattgtcatccgcagcggtagccccgaccttggtttggcgttctgccttggtccacttcgggtagcctggctcagtttgggagacattctggaacggacacagcaccatgacgtattcgcgcgggataacgttgggcgtcccacctttcagctgcttctcgtccaccgtgtgcgggaagcactgacccacaagctcgacgaacgctggtttccagctcgcgtccgaaaaggtgctccgatcaactacgcgactctgaacatacgtgacatactgcactgtccgcagcacattgtacgactgctccaattgtccctgcagctccgggttgtgctgcactcgctgctgcaagtcctcaaatgctcgttgccctgcctggtacgcttctgcaagtcggtcaaagtcggcattcagctgcttcaccttgtccactgcctccgtcaagtaacctttacgaactctcatgcctttttgcaccgcacccagcctcatcttgagcgccttcagtctcttttgccattggatctcacacgtgtcaacacacacgccctccctctcgtcgctgccgtcacaacagtcacacactccatcgcccacgaaagccagcgacaccaacctgtcgtctgctttgcagccaaacttgcgatcaattggcatccttctggagctcaacagcacgcgggaacaagccgaagtgttcggctcgtcgcttccgtcttcgcagtcgcaatagtcgtcgtccacgcgatcgaccggcaaggcatcgacgcagaatccgctctgcgcctctagatatgcggtatttgcattggtgtggtgccctgcgagccccagaattgcgtcgtcgtccatgaaatcccagtcgtcgtcttcttcgtcctcatcctcgtcgaaagcttcgccgatcccgaagcgtttgaagtcaccggagctggcagaagccgcggcgacactcggcacttggatggccagcaccagcatgcacgcgatggccacgatcacggccaggaagaacgccagcagcgagcggcgcacggtacgcggcgccatactcggcgattggtgcaatggcacgagcagctacgaagacattgattggtggaaaatctgctttagttacctttgacatcaatgatgtcaaccttcttgtctgagtttcaccgctcataaaagcgttgtggtcgtgctggcggtggagacttggtggggcatggagacaggacggatgcgtttcactggacgaagagcggcgattagactgggtctctcttctccgtcgctcaactcgatcaactcgtcttcagtagtcgactctgaggcttcgcggcttcttttgccgttctcatccagcaggtttcgtcctccagtctcccacttgtcctcttcttccgccacggacacccaatcgtctgcgaggttcttttgctctgcgttatcgatgctttcggtccccagcaactgctgctccccattcggtaggtcgattcgagacacccctggtaaatggcgactcatgtcaacgaggcgtgcggcccccattcgcagcagcttcaccaccatccgacccacaaacaccatcaacgtgaagaacttagccataacacccatggcaaacagcggactggggagacttgggaccaggtgtcgtcaagaagaatccctcaacttcaacgtccaatcccatagtgacgtcagtttcttgggccgaaaggacacggtgtcagcacttcattagttgctggcacgaacatgacacgaagccgctacaacaggggtgggcaaggcgctggacactactctagtagcagtgaaagcagcttcggcagcgaaagtgatgtcgaaagtagctgtgctagtgacgcttcaagtgatggtctagagctcggactgagcgggaagggcgaggcggagcagaagttcaagcggttgcgccgcattttccttaatcgcccgacgaaggtaaagaaaagggagagcaaccaacagcgacggcggagtcccagcagtagaagatctggactggagcgaagaccactcaaggttttggcgacagaacgatcacggtcacaacgtccaagggacagcgtctcaagtaacagcagcagcacaagcgatgaaaggacgttggaaagaccatgcaaaccagtgaactcagcgcggccactccaagcggcgattgatcaacatatgcattcgctgtgggaacagatgaagcggcattctgcgcagcgcgactccagtacgtcacgtggctccccatcttgtcaatgctacatcaatttctaacaccagtgcttgctagctgccggccaggttcagagtatgctcgagcaggccgtggatgaggttcaatttctaaagaaacaagaagagcgcgcattagaaagccaagtgaggcttatccgagaggtgagcgcgagtacactcaaattgtaaccatgcattttctcatcagtcttcgtggttttgatacagagctacgctttgcgactggaggtcttccagcagcagtacctgagcaaagtagccgaaattgaagatgaaatgtacgcttcaatccaaaaacggcaagaagaatgcgatgcaactgtcctagctgcagtcaaaaagctccaagatcagacccaacacgcgttattgaaggatttggctacgatgaatagcgaaccgatagaggaacagttcgactcaagctcattctcaaccggcagcagttcagcgagtcagtcaaaagtgcgtcgacaccaaagcggaccgcttcgccatgacccactcagctgcaattcttctcacgtaccgccaatggtgaagcacgtatctccagtttcgaagcagcgacagaaattgcaagcactcgaagagaagcttgcagcattttcacctgattcgctaattcgtgtcaagaacgaattggccacatcagtccgtgctgagcggaccgcagcgaagcctaaacgagaatccgtaccgagacgcaggcactcgcaacgcaaacacgaagatcaagacacatttgctgacgaatggcaagtctcgccgctatcaccgcttcgaaatggaccatcatactggaaaaatggcatttcacttgaagcacgagatgtacgagaagtagacaatgctggcaacccgggcaagcatgagcaagcagcttctgcaacagtgaaaagacgagcacctgcatctttcttaagccggcggttccttagaccggaagaggaggaagagctgcgccatctccgaacgagcattggcctcgcaaaagactggatggcgatcaatagtcaggcgtgagcgagcagagaggcattcacgcagtcaataaaggagacacgagcacggcaaggtcagacgcgagcgctaaatattctaattacacgttgatcattgccgagatgctggccaacgcggccttgtgcttttcgcgctcgagcagcatcacaaccgtggtcttgctgctcttgtccacacgctcgcgctcggtcaacggagtgtcgaacgagaagctgagctccgcctcctgctcgaggaactcgtcctcaaagttgtggaagaacttggctgtctgctcgtacgccgtcttggtcttctttccctttttggcgccacttgcgccgccgctcttctcctccgtgcaggtggccagaatcaaaaagtagtccatcttgaagctgttgcgcagctcctccgtgtcttcgttctcgattgcccactcaatgtcttcgtgcagcgccgagtgtagcgctggtacgagctggtacggcaggttcaccatgcgctcattgagcacaagtccgacgttcttggtgctgaggatctgctgcagcttggactggtcagcgctcggacacttcttggccacgtactgcaagatctgctgcacgctggtctcgttggcgaagctcttgaggttcattgccgtgatgaagccgtatacgtcgtcctccccttccacgcagaccatagtaccggccgtgacttggcccacgacggcgttggccaggccagacacgtcgaagggttgcgacggcggcaggtaggccagcaggaactgcttgacactgtggaaatgagcctcgcgcgggtccgagaagagaaagtcaacgctcacgttgccaccagcagctgcagcggcctctccgccgtccatgtcgtcgtccgagtcctcgtcgtcatcggctccagcgggcgccgcagcgaacgtgagatcgtcctcactgtcgctttcgctgtccgagtcgtcctccatcatctccacctcatggtcctctgcgctaggggccttcgggtccagcggtgccttgccggcgttgcgggtgcgcttattctggggcggcatgaacgtagctacgagcttgattagcaatggagctgctggttggccagcgtcgactttttgagccaacaattgaaaccggtacaaaaccggttgttccgttgttgacaatggacggatcttccttcatcccgccgccattcccgttcccagaggcgtggccgcgagtcttcgcggagcaacagcagcgacaggagcagcggtggctcactcctaccgcacttcctctagtgcaatggccggcagcacaatattatccctaccctgctgaccaaccgattccagctcaagtgccaccgactgtgacggttgacgcgcaagacgaagacggagaggacgacggagaggacgagtacgaatacggatacgtcctgagcgacgagtggcgagagcggttccagtcgtcggttcaattgcagcaaccacagccaaagcgcagcaagggcaaaaagaagaagaaaacaccgctaaagaagaccaaatcgcagcggcagacagtcaatgcagaagctgttgccgctgccagtgcgagccggtccggccacttgcagcgcgagattcaagcagcaaaaacgcgcgaactggcccggaagtggaagcggcgaggcggcaccaccgctgcaactactgacccgcaggctgcagcactggagacgtcgctgaacatgctgttcgacgagttctgcgacgcgttccagccagtcgtgtggccgcacgatccgctccaacgataagaagtgcaccacctaccggtagcgttgtcgggcagaaattgtgactacttgctaattgcatcggtaacccatcattcctccaggggcgtcctataggtacgaagcctacagagtccaaaaacgtatcggatttccgacttgtcgtaccctctgacatccgtgaataacatcgttaaatgtgaccctctgcaaagtgatcatcggtaatttgagtgtggattctcactttttgtcgggaaaaagggggcaaaaaacataaagaaatttgagctgtacatgcgccacccggacatctcgcaatgaacctcgggcagatgaagaacgacccctccaacggctcggcgcagagcatcccgtcccttaagcgcacgctgagcggcggcgcggccagcaacgtgggcggccacggactgggcagccacctgggcgtgggcatgaacaacatgaactccatcccgtccgtgctgcacgcgacgcccaacacgaacccgccgcagatgctgcgtgccgtgacatcctcgggtgtgtacaaccgcccgtcggccaaccaggccttcccgggccgcagcggaggcctctcggccgtgggcggcggctcctcagccagcaccaacgtcaccgccacgacggccacgcgcagcgtctcccagtcctccaccatgcaggactggaggatgtacctgaccattgaggagcgccaggctgtgcgctccaagatccgcgacgcttacaccagtcgctgctccacctacgaagacctgctgcaagtggtgaggccccggcagacaactagaggggttgcagtgctgtgttgatgttctaatgtggtgttttttgttgttgctattgttgcaggcctgcgctattgaagaggagctgctacacatttcggcgccgtctcgtctggactacttcaagagcggcttcgagttcgagaaccgcgtgaaactcaagaaggaccagttgcagggccaactggcggccttggacgccaagcggcggtcgcccagcgggcaacagcagcagggaagctcaagcagtggcaacagtggcagcaaacaggtaggatcggccaagaagcagaggaattaggacgtcctagtggcgccttgcactattcagcggcgcttctgcccctatcaagaggctcagcggtgacgaccggaggcccgggctacgtgtgggaagcgataagctttcgtgtatatcttgagcccagacgaggagcgacagggaaccagacattaggagagcgctccttgctgagctcctcatcaatcaagtaattttcgggatcagcggtagaagctccagcaaacgagtcaagacaccgaagtgatgtgcatcgtttagtgtatttggttgctcttgctgcggttgtgcaggtctacaggtctagtatttgctcaagaagaaatgcttcaacacctccaagtgcagttgtagcatgctactttgctgtagctctggggcgaaggcgttgaagttagctgcttccagctctttcaggtacaaagaggagctcacgagcggcaggaacgcacggcgtgactcacgtggtagatcattccggagcgagcgagcttggtggaggtgctccattgcacgacaggcgacatcaaacactacaggcgccaccttctctccgagcttgggatcctccaccgcagcgaccagatcatccacggtgacgccatgctgtacaggtacaaccaacaccacaagccaacgcttagcataaggattccgggaaccaaatccaagccgagatcatcgatccgtaccttgttcatgaggtcctcgggcaagtaagactgctgtcgtgagctgtgaaacgctgttcctcgcagcagcgtcgccaacccgatagccacaccggcgtgtccggctacgcggtctgccgtgtcgtcgcgaacaccgagacactccagcgtgaggtacagcagcgacgaggccgtctgctccgcgtagacctccagctcccgcagactcagcacgtcctcccggtccagatcctggtcgcgcgcgtccagcagccgctcgaaccacctccgtgtcaagtcgtgttcctgaatgccctggtccagtgcccgcagcagcgctgtcgactgctgcggtcgccccgcacccgccgaggctgacacttcatacaaattgtagatacgctcccgccaccattgcatgcggatcttgcccgtgatttggttgctgtgcgcagagtccttgatggtcgcgatctcagcgttcagcgcgcgctgtacaacgcaatatcagcctttgctccagcaatccacccgcaactaatcaagaccagggacgtacgatggcgaagaaggacgggcgagtctttaccggtagcagcagaccgcacagatacccgtcgtagtccaatttcctgatgggcattgcaatattagatatttcacatatttgcagccgttgtagcgccttttaaagccatcatcttaccggactgactcacgaagctccaggccttccttgttgtgttgcccacgcgccgcactgaacgctctgcgctgcagcagcgtcgccggacgtcgtaaggagctcgacatcatacttcaggagaccgcagcgtaaccgccgctcgtttttcggctaccaccaccgaaacgaccggtttctaccggtatatcgtagccatttctaaatggaggtacaccacacaatcgcattgatggctacctgtgtgtacaccacataatacgcggcctgccagtcaattatggccaatcgcgtctgctggccaccattggcccgctgcctgtcattcggcgcgtatcgctaccgaagacaccgcctcgctcgcattgcccaccggaaggtgctgacttccatctaccagagccatgagccagctgaagaagcgcgcggctgccgagttttcgggtgacctgaagcgccacacggcgcacggccagaccaatggcgaggaggacctggtgttcgaggacccgttcggagacgacatggaggacgaggagatggtcgagagcgacgaggaggtcgtggacggtgaggctgccaccgacaaggacgtagatatggacggcgatgagcaggctggtacgcaagtgaagaaggtgttcatgccgggcgtggacaagctggaggacgacgaggtgctggactacgacagcagcgcctacgacatgtactacgccatgacagccgagtggccctcgctcagcatcgacgtggtgcgcgacaacctcggagctgtgcgttcgcgtttcccgatgactgtgttcatggtggcgggcacgcaagcgaccaacccggacgacaaccagatcacggttatgaaaatgtcggagctgcacaagactaagcaccacaatgattcggactcagaggatgaagagtcggaggatgaggatgagacggagggcgacccggtgctcgagtcgcgttccatcccgcacccgggcggtgtcaaccgcattcgctgcatgccgcagtcgtctaacatcgtggccacctggtccgaccgcaagaaggtgcatctctgggacattgccaagcagctcgagtcgctggacggcaaggccggcgccccactgccggccaagcaggcccccgtgtacacgttctcgggacacgcagacgaaggttttgccatggactggtctccggtgcaagctggcagtcttgtgacgggcgattgcagcaagtttatctacgtttgggccaactcggagggcgcgtggagcgtggacaaggtgcccttcacgggccacaagagctcagtggaggacctgcagtggagccccaccgaggcgtccgtgttcgcttcgtgctcgtctgaccgcactgtgcgcatttgggacacccggcggaaggcgggcagcatgctggacgttgctgcccacgacgacgatgtgaacgtgatctcgtggaaccgcaacgtcgcctacctgctggcgtccggctcggacgatggctcgttcaagatttgggacctgcgcaactttaaggcggacaacccggtggcgcacttccggtaccacacggcgccggtcacgtcgatcgagtggcacccgaccgacgagtctgtgcttgcagtgtcaggcgccgacaaccagatctcagtctgggatatgtccgtcgaagaggacgccgaggccgccgtgcccgtccagggagagagcggcgaggcgaaggtggacctgccgccgcagctgctgttcatccaccagggccagaccgacatcaaggagctgcacttccatccgcagtgcccaggtgtgctcatgagcacggctggcgacggcttcaacgtcttcaagcccgctaatatttcgtaataagacctgctatcgtttgtgtcaagcccaatacagtagacttttcaaacgtcgagttgtaattgtgtattgtacaccacccgtagctttatttcttcgaagggcggaccacaacgttcccaggcgactaacgaatcggccgcaacgctaatcgttccgtacacgatactcaggccactcgtctgcagcatgacaccgctcacagtttccagagcgtcgtgccttctcttcctgctctctctggtcgccgtccaagcagagcctctggccatatacggctacgggggctacggtggggaccacatgcgccaccagggtatggagtactacgccccggcagaccgcggctgtgccatgtgccgcaacggagagaactgctccgtggctgtgcacaaccaatcggccggcgtgttctgtggcgacgtcttgtccacctttcagccgtgttgctgcgccttccgcaacgagtgcatgaccaccatcttctcggacgagtgcgagtgcttcgacggcgcacgcgaggagcagatcatgaccacgaggttctacctgttcgtgggtctgtccgtgatagcgtgggctctactggcctacgacaagatgtgcgcagggccgtacaaggtgatgaacagcaatagcaatcaccagctactggccagttcgccgtctgcggcccgggcgagaggagaagacagtgttgtggacacggtggacagtgactcggaggatgaggaaacagcaaggggccaggatgctgctactggttcagctactgctgcagtcgcgactgtgagtgttgaagttgcagaggatgaagctgacgttgacgatgatacagccccgttgcgtccagagtcatcttcaccgagtgttgacgttgaagctgtgatcgacgagccgcagacccacagcggaaggcaagagcaagatggttcttcgattcaaaccgtctagttggtatctgtctcggatgttgctggggattttactggtgggaaggtgagggggttattgtgaaatgtagtttgatattatggtacacacgatactctgaggacgacgaggacggagaacttcactctaacgagatttcttgaagctctgccagagattccggcagactagtactaacttcttgcacagcatcaacaggaacagtgacaactacgacttcctctggcaacggttccttctgttcgtcggccacaggaatatctgcgacctcggcttctgggacattggcgaaagtgaacgcaggcgacgaggttggtgctggagactgcaacaccagcagcggtagattgtcgtcagcgtctgtatccacttcagagtctcgccgccgcttgccgaaccgttggcggaaccgaagcatcttcatgtcgacggcctcgccgtacgccaccattcggttccgtcggaagtcgctgaaacactccacccagtggtccacgtacgcggcgaacacaaagagcgcaagatacgccgacaaatggatcagaatctctgtggtcacggacatttggctgtccttcagatgggcctcggaagctgcgaagctgtcttgcacctgctggacttctgtttcgttcgctgcttcggtgactgaaagcctctcgtggcgttgggcttctagttgcgcggaccacgccccgccgcactcgcatgtaagcgacttgctggttacacggcactcggtgtagtacggacagcagcacggctgcgtgttagcttcggcatgcagatctccgcaaaatactccggacgctgaatcgttcgtggccacggagcagttcccgctgtcccgacacagcgagcagcccgcagcgacgctaccaacgctggaagaaaccacgcgtacggggctgagtggcactgcgttctgtccctgtgcggacaccttcgcggcgacgtgaggtacactacagactgcgccgagcaacagcaacgcgacggagcgcctcagcaatgactccatggcgaggtgcaattgtagaatgcaacgtggacacggacacgccgcaattcgtcaggatctcctaggatggggtcaaatatcaacgctcggcaaaggcgccgtgtgtaccggcgtgcgtttccacgagtgccatcgtgcacgattgccaggtgggcaaccgcactgcactgtattgcccgtctacacatggtgccactgtccgcgatttcacccaatgcgcgtcgggcaaatgtagtcggtgcaaatactgaacatgggtcaaggtgagtgtcaggttgacactgtcaaccaccgggacactaattttgctcctggcgatgattgccaaggctcatcggccgacctgcatatggagctacagtacatgtacaattttgatggccataccagtgcatacattgcaaactcacgcgaaggagttatggcgctttaatccttcagtgatacacactttgcgatcaccgcaaaactgttagcaacgcaggcaacaaggccgctgaagtgaaacccgatcgcgacgcagcagacgaagatgtcaactctgccatcatggctatcatgacagttccaatgccagcactcaatgtcagacgacgggctcctatccgccagcaatgccatcttgtatacatgtatacacaattatacatcgaatcagtcgtgtatacactccattccccattcaccttgacgatggcgtcgacagctcggcaggtgacgctgttactgctcctgacggcgcaggtgggtgtacaacccgtgctcatgggctggtacgcaagcgaggcgcgcgacgtgcgtctgcgcgtgggggttgtggagttccttaagctgctgctggcgctggtgccgttgagcctcacgcgtggcgagggcgggctgctgaaccagctcaagacgtggaagctgcgcgccgccatggccacgacggtgctcccggcgctcatctacgtgctgcagaacctgctgaaccatgcagcggtcgtggcgctggacggcgtcacgttcaacgtgctgaaccagaccaagatcatctggacggctctgctcgtgtacctgctgctgggcacgcgccagtctccgctgcaggtcgtgtctctgggccttctttgtgtggctgcggtgctcatgacgtcgtcgtcggacgcagagccaacaagtgcagagtcgacagacgccgtagccttcacaggcatgtaccaagcactactgggcgcggtgctctcagcgctggctgggagtatcatccagagagccttgcaacgcgaaaaccgtaatcagtatatggtgacggtggagctcagtgtgctgggcgagatgacgctcctggctctggcaattgtgcagggcggactgaaggctgcgacggcggacgcagactcgcaggacagcatgtgggagggctggagtttgctcacactggcggctctgttgtgtcaagcactgggaggagttctggtgggttttgtgatccgagactgcggcaatgtggagaagagcttcgctgtcgtgggaggaatgggccttacggctctgttggagactcacttcaacggaaaacccttcggtcacaacgcgctgctggctatggcgctcgtggctattagtacggcgttgtacactctcaacccacctgcagttgtgtcatctgacgagaaaccactcgcatcgcttatgccagctgaagttggcgttgctcctgtgccagtggttgggccttcatcgaacgtgcggcattcgaagaagaagatgagcgctgacacgagggagctggagccgttcctggtgcctccaacggttgtcacagtcagtagcaatagtgtacatacaagcagcacttctcgacaggacatgcggcggtctgtaaggcagttgcagtctgcagagatgccaagtactcaacgggtagtggaggcgatcgtgtgagcgactaccacttcgatgtaccaacaaaatttactctatatctcaaatcgatgagatcccgtcgagttctgctctgcgtttgcggtaatgtcttagctgagctgaacggcacgctgcagtgactgcgctaaagcggcactagaaaatttgtcaagctcatcggtcttccataatgccccaacgtggccgtctgggcgcacgagagcagcagccttggtctccgtaaagctagtccatgccttgttgttggccttatcgctgtcgtctttgacaacattccatgagagcacggagctgaaggtcgccgatttctgggcctcagcgatagcagcatcaaccatcgcagaaccaccatcagaacccgatcgcagcacaaccagggatacatgtctcgacactgtcggtgaagcagacgctgaacatgaactgatcacacgcgcagcatcgcgtccggtgacgaccagaacgaactgcacgctgccggcattctccataggcttttcacgttgaacagcaagacgcatcatatcgtgagtggacaccttcgcagcacatggactctcgctagtgcaccaaaagtgcggaaagcgctcgccaacgcggtaagtcggagaaaagatgatgctgtctccatctccaacctcggtgcggaattccacggactggtccaacgctgggtcttgcatgagcttcttggcgcgagacgcccacgaaggtgcgtcgtacgaaaaaccgatatcaaagtggtagaacatcatgccgagcgttttacggcgtgtcacgatatcttgtacgttggcacgcatacgattgcccaaggcgctgcctgcaccacctgcatcctcagcctcatcaagcagtccaagaggcaatttaccgacacgcatgatccgctgcgcaatttcacgctgcgcgcgcagcgggagcaactgcatcggtgccgagttgatcattttcgcgagtgtctgggcgttgtcgtgggacacattgagcgcacgcggaattttcatggttcgagcaacattgctcagactgaactgcgtgtttgtcttcgcaataagctggcgctcacggccgtacgaacgcaacaatgcttgagtgtcgacgtcgttgtcagtagcttggctttcagcatccatctgaatcgcaagtgcgagcttccaggccaggttgtgcgtgtcctgcagtcccgtattcatgccaaatcctcctgccgggggaaactggtgcgcggcatcgcccacaaggaacactcgttccttgccggagtcatagtgtttggccacacgcgcccccattttccattggccagctgacaagatattgatgtcgtcgtcgttgactttaacgtcgttaggcaggatgtggcggacaatttccgtgcactgggcaaccgagaaatcccaggggatcgactcttgaggaggaaagaagggaatctggaagacccattcgcccttattcaggtcgtgagcgatcagaatgccaacaacctccttattgaactacggacacgcaaatagcagcgttgggttagctacagtcatatgtgtcatgtcgggggaagaaattgcgcttaccacgaagtacaacatagcagggttctcgcgcgccgcctccgacaacgccttgctggtgaagtgcacgttcgcgatactctgcaggttactcgttccagccatgtcaataccgcagagactgcgcaccagactgtgcgcaccatcggctccaataacgtagtcataggacgcttgctcgaccgtgttagtatccctgataaacactcaaacgcaaacggcgcaacctgccatcagtccatgccccgaggcaaactcccacactcaaccccactacaactcacaaatggcgcagtgtgacttctggctgttgcattgcagaaccttccgactgcaacttcaggccctccagctcaactcctcgctcgacacgcaggtcgttttcagtcaggaactcgtccaacagcgtctcgaaacgattctgagggaagtgcagaaactgcgtcggcgacaacgccgccaatgacttcctaagactgtcaccattagctgccgcctgctcaatcgtatcacgcgggatgcctgcacaaacagcgaacgtcagtctcacgtcgatggcagttgccagttgcaatcatttcacctacaaggaccaaactggtcgatacgcgcgatctcccgagccttgcccacccccgtgcagtaaacgtagtcacgccactgcaacaaatgcatccaaaactgagccgtcactcattgacacatgctccacttgcagatactactcacgagcacgctgggcgccgcctgagcgagcagccggtcgtggaactccggcatcgtagcgtatagcacctccatcgtgcgaaggttgatgaaatgcgcctgcgggtgcgacgtagggctgcgctgacgctccaccacgcgagtagggacgttatagagcttctcaagcatgaaagcggtggccagccccacgggaccgccgcccaccaccagaacgcgcggcttctgtgcctgcaacaattgcgacgccagcgtcatgaatcaagaagcgctgcaagccacgagtagcggggaatgcagacagcgcaatgctacggcgcgttgatacgcgccgttcacatggcacacttgccctctcgaatagaggtccttcgtgtggaccacgttggatgagactcgttaaagtacgaagatagttttttgcggagtgattgtcacttcaacgtcatggacctgttccccgctctaccgctgccttctacgggagatgaagcgaacaagcggttgttgcaggtgatgagagcatttgaggaccacttcggacggctgccgacgggcgttacgcgcgctcctggacgcgtgaacctcatcggagagcacgtggactacgaaggctacgcggtgcttcccatggccattgagcagagtgtctacgtggctttcgacgtcgtccaaacggaagaaagcagtgccaatagtgcaggggtgaggacgttaagtgtggcaaacgctaagtcatcatacaaaagcatttcgttttctatggaggagaaagagcaggaaaacatgcagacactggagaaagagggagcggcctgggccaagtacgtgctgtgtggggtgctgggggttcaagatgagcgcccggggctgtttggcggggagcggagggagctgcagatgctggtggacggagacatccctgttggttgtggtctctcaagctctagtgcgttggttgtagcatccgcgttggcgacgagcagcgcgttgcagacaacagggataacacctttcagcagaatggagatggcagagctctgccgacgagcggaacatcgtgtgggcactatggggggcgggatggaccaagcggtggcgtgcctggcgcaacgtggtgtcgccttgcacctcgatttctcgagcgttcctgcagccagcgatctcgtgagtgtaccagatgatgcagctggggtgaccttcgttgttgcgaacagtctagtggtggcagaaaaagcagttgatgcagccactcgctttaacaagcgggtcgtggaatgcgcgcttgctgcaaaaatgatagcaaaacgggctggtatcgagaagtggaaaagcgtacgtctttgtgtatcctgcgtagatattcgggcgctaatgatgtggacgtttggtgaacagattaatcgcctcgttgatgtccagctagcattgaacaatgctcgcggtgaaagcattagctatactgagttgcaggagttggcttcgactgcttgctctctgagagagtacaatgttcatgagctcgaagacgaattcggtgtaccagtcattgaacttttcagcagttcttcacttgaggcagctagcaaggtttggatgtggatgtgcgtacttcttctctgatggtatcactcactctgtaacgtgacacaggaagttctcatcacagcgacaccacttaaactgcagcagcgagctctccacgtctggagtgaagccaaacgtgtggaggagttccgagaaatctgtgtctcactcgcgaataatgtgcagcagtcgtcagatcagtcgcctttcacgcagaagcaagttgtacaactcggagaggtcatgaatgcaagtcacaacagttgcaagacgttgtacgagtgcagttgcccggagctagacgctctggttgatgctgctatatctgctggtgctttgggagcacgtcttactggtgctggatggggtggttgcattgtgacgctcgtcaaaaaggatgaggtatccaacttcatgaaaagaatccgctccagttactaccatgatcgaggaagagcagcaacaaacactttggacgcaatgtttgagtccgctccggcacctggagcggagattttctcgccacagctattgtagtttgtactgtcagtaattgtttgattattctcacggagttagcaacgagcggagtttctctaaatacacatcctttgcctgtacttcgacctcaataagcgcaagctatcacacagaaatgtaaaaaagtcggttagtcaacgaaataacaactgtaataatgttgtacggccgcacctcagccttacgctgggctttctctgctggtgtcattgaaaactgaagaaatgcgaaatcacgtttcagattggacaaggtttcccgtcgctgcgctatctcctgctcgtactgctcaatctctttccgcatctggagcaggttaatacccattattgtatcagttattagggaaaacgtacgtgagcaataaacgcgtacctcaacgttggcagcaaagcgatcgagatctaatatgaccctgaaatacgtatcactcctctgcagtagggtagcaacaatacagtaaggtcaaaacgagaagcagaccgcaagcagcacagagcgactcacatgtatcttctcaaaggcctttttgacagcaggtcgtgtctgaacccagcatgaagaaacaagcgcggcattcttgcacggaatcacgccaatctcgacaacatcctgcattgcacagtaactgtgagtactgcctacgaaagatatcttgtgtttggccgaagcgcacctttggccgaagaaacagtacttgctgatcaatcagattcaagaatttctgctcgccttcctcgcgatggttaaatggcagcagcgcctctacagggtcgacgcagttaagtggtgtcttgtctttctggttcagtagctgttgtggcgatccaatcagagcattaccgcctgttactgggctcacatcagccagcgtttcaaggcatttccacgtttccaacgcgttgaaaacttgtgcaacatcggtaggattcgatgccctactggagacttccatcgataagtctggatgattgcttgcagcggatttagcaaggggctgcagacttccgtctaagtgcaatacttcaagcagtccaacctcggtatcttgcacaaaggtattgcacacttcttgaataacggcgaatcgctctccgtgagacgccacgtcattcagattactgtccattagtcggcgacaaatctcgtcgatatcgctccaccaattccccacagtttccaaaattactccaacttctgctgcacgcttgctgttttgcgcggggtctgcatcatcttggctgtccgaaagcttgctgcgcgtttgctctggctcagataacagcttcaggccgtgcagctcctggaaatcaacaaccgatcataacaaggaaaaataatcattgtcaacttcagcttagtcgaagcaagcagggtcaacacataccgatatctcacgtagtttttcctcagccgaggtcaaatctactagtccatttgcagggcgcagcataatagcaacaaagtcctgggcgacgtcggaattcgtagcatcgagcccactctgatggcgtgcttctagccactgcactttggatggtgtcactgttgaggtggttagcagaaccacgtgagccaacccctcactcgagagttgatgcgtccaatggagccatctctcagcgtcttgatcgctgagtgtgtcaaaatcccgatgcaagaagacgggaagcgtagttacttgagcttgatgactatccaaacttgtcggtgcatctgtttggctctcttcatcagtcttctcactgtgctgttcgttgtactgaaggtcacgaagcgcatctagcaatgcgtccatcatcgattcctctgctgaaactttcaatccgacgcgcgggtcaaaattctcgggtacttctccatgcagcgccttcttcagttccgaataccactttgcaatcgtatgtactgcaatctccagcgtcaaagttggtgcagtgagtcgctccaaagtttcgtgtgttggttcttcgtaggacccaagaacctttgcgccgccaagaaggctgctcacacaaacaccaacaccaattcgtgagtgacacagtccaccaattctgaacagtgactagccaaaccacgtacgtgttcgtaacaaacgccggattgtcaccggcagaaattgacagcaaatacgggaatttgggcggcgcactgcccagccaggcggatgcaccggtcattttgggcggtttagccgtcgattccgagatgtttgcctcattttcggaagatctacgccacctcaggagatgctgctttgcgtacacgaccttgttgctgtcgtcgtattgttgcgctgcttcaccgattgctaaagctgctaggggcgtcagcaccgccacagtcacgccgatagatcgagccttgcgcagcattttgccgtgtcaattttgtattagatgtagcaggtgatgaagggcctgttgagggagcgatataaaagggtatatgtgatagaaggttttgggatgggtggaccgcactgcaccgctttggattgcatgctgctgacagttcagttctcattttgcattcgttcgcagcaattgctctctcagctgttgtcagtgctttagaagtagtgtgcatttatcatcggagtggcgtgtgatgtctttcatgctggggaggtttcggcctgcccagagtgaattcctcgtttctgacgaatccgctgaacaagcggcctcaatgagaccaacctaagactaaacgactgatgctctccatcaaattcattctgtcgtgcagtcaaatgcaatgcagtctctctaagaagtgcctccagaaagaaagttgtggatgctggatagaacaatcagctcgaccacgtcatgcttaattaagcgagcaccacgataagaccaagtggtttggaagctgaatactttagctttgctacgtacagtacctacttgtacgaggtcttctccaacacctccgtaatgtcgagttgctctgcgcgatttactcgctaccactctcgcttgtggctaataaataccagcggattcgcccgcttatcactcgcagcttgcagttctcctagtactggaagtggcgtatcttgccattggcgcctaacgcgaactcatcgccatctccgcttggtcggggatcttcacttggtcgaacttcttcacgcctgccatttgtcttgtttgcctgacggaacggcggcagcatcgaggaactgctctctgaggggcttcggtcgtaggtcgtggacatgtccgacggactgccctcatccatctcgaagtcatactcatccagcgacgaaagtcgtcctccaaccaggcccccaccacggcggctgccactaggcaagcctacgatcggtaaattcgacgaatcaacgtttgctgggatcaactccactgcggcactgcctcgagcgctcagcatgctgatggtgtccgtggtacctgcatacggggatgcgaacttgtagaattgctcgttgtcctggtgatcatggtattcctcgctcatcccgctgctggtgctgctacgagcggaaaaccgacctcggaagaagttcgtaatgcgctgacccaggatagagctcggccgatgctccagatccgaagtaccgtcaccatttcgcagcgaagagaagatgctgttcgtgtactcggaacctgacactgacgccatcgtggtaaaatcgttcgagttcttcgacatgttgtccagaattgtttcgagtatttccgcagccagaggcgctgacgggcggactgaagggttctgcgcccagcactcctcgataagctgagcgaagcgcagagggcaacgttgtccagggttaatcggtggtcgttgctccacaagcaccttctcgcggacctcgaagtcaaacttagtgtcgctaaaggggaccgcgccatccgaagcaatttcccacaaaaccataccaaacgagtacatgtccgttccacgactgtatcggcagttgtgcgggtcctgcaaaatctcgggggcgttccatcgccaggtgccagcaaacgaagtgagttcatccgacacgctagggttttgctgctcagccaagctgacagtacgctcgctgtatcgcacacggttgacgaaattcgataccttggaaataccgaagtctgcaagttttgcttgccatgtttcagtgactaaaatgttggaagccttgagatcgcgatggatgtaagtcgggttgcgcgcatgcaagtaagttagacccttcgaaatatgaagcgccaacgccactttgcgccgccaactcagtgggaaatggcgcttgagcacgtccttgagactaccccgtgagcagtactcggtcaaaatgcccagcttaccctcgaagtatccggcacgcatgaacagcgtgatgttcggatgccgcagtcgtgagcaaagtcttgcctcctcgatgaaagcttcttctgagttctgaataatagtgcggtacaactccgcatccgcgacatcgcctgcgcctccgttgggaagcttgatgaccttcattgccacttcaatccccagccactgagctttgtatacactgcccgctgctccttcagcgaggaaggccaaatttgtaagtccattcagctcctctagagtcaccatctcgttgccaacatacgtgctcttcatcgactcttttccagtgttgctactggctccactgcttccaaaaacggttcggtacgacgaggctaaaacgttggcaacgccgttgttgctgctgccggccaggagacccgtccccattcgatctcgctgccgttcgccatggcgtgagctgttgctgcttgattgcaacatcgtctggttgttacgcagatcgttttcgtcctgaggaaatctcgcttctcctaaatgcctccagccgaacacgatagcgttgaacagcggtacgctgaacagcaaacaatctgccacttcgtatgtggagtagctcaccacatcatgacctagaacgtagcacagcatcattggaaactgcagcacctgaaccacgaggaagtagtgcataagcttctcatgagccagtcgcagcgccaattgagcctgaatctcccacgttttcgtagacgccagcgctgcatggtccgcgctgtttccaagtgacggctctgtcatggctgtagcattgggggccagatcgcccgctctgctcaaccgacgtcgctggatcaacagcactgcgttggcagcaatcacgcagcctgccgacagcgcgtacactgccacaaacaagtagcgcaggcagttggtgagtactatcgtggacttactatctgtgatgaccgcctgagttctcgccgtcgatagcagtccgcttgcgagtcccaccgtccagataatcaaccaacttgctcgcactgggaagcgtctcatgggaggactggtcccttggtgtccccttgcccagcgatgtgtgcgcaggacgaaaattgcaagcacgacaccccacagcagcgagaccatgaagctcacgtggaagacggaggcgacgctggcgccaatgactgtgcgcactttgacgcctggccccatgaccatgtccatggactggctcacggcgcccgtgacggagaacaaccagtcggtcatgaccaccgaatagaagagcgcaggtcccgtcgcgtggggcggcggcagcggcggcttcctgctgcgactgcgtccccgtctcctcctggagctatagcctgggtccggtatcctccggaagagaaccaccgccacgacggacgagatcacggccgcggtggagatgatgacgagccgcgccgtctccagcgcgtcctcggtcgccgtcatttacagtctgcgcgggcactggtgtcttctcgttagcgcagacttgatatacagggactccgtgttgcgctgcgaatgaacctgccgccgcccagtgcaggaagaagcatcgccagctggtcccattgctccgcctctactctttgaccgggtctcttccgctaggagcatgctgcagtgtgggattacctaacatacagcatgtgctgacttcttgacccgcgtgacccatctcccttttggaccaattgtgactaaacgacacaaattttgtgtcggagtgtacttcacgccgtttgattagatgctcttcaactcagcatcttgtatggctggagctcgtcattctccaagtcgctgagccaagatcaagactatggacgccgtcgcgtggggcccgtacatcgagacgctgcgctcaggcgcgcagagcgtggaccaggtggtggagaagctctgccgggacaacggattgagcgccgacgccgcctcctcgtcacagacgacggcgcaaattgtgacactcgtgctgcaggcgctggcccaacaggctgccggtcactacgtggcgccgtcgctggagcagcagcagagggaagactccgctgtcgcctttgaccatcaccagcaacgctgcgccgccgtcacgcgtgtcgtggaggcggccgtgagccagaagctgctgcacgacgagagcccgctgatcaacctcttcagctgggacgccttccagaagcgcctcgccaacctgcacgccgccttccccgagccagagttcaaccacgcgctggcagttaaaacgaatcccacgcgcggcttgctgcgaggcgctcgggccaaggggttcggcgcagagtgcgcgtctattgcggaggccaaacacgcgctatcgctcgggtttgaaccgcgtaaagtcgtatatgactcgccgtgcaagacactgggcgagctgcgcgagatgctgctggctggagtgtacattaacctggacaacgaggaggagatccgcaaggtgaacgagatcttcgcggagttgggactctcggaggaaacgaaaaggaagcacgcggcgcagatcggactgcggattaaccccgtggtgggcggcggaacaattgacgctactagcacggcgacggtcacgagtaagtttggcctcccactcacggcggagacgaaaccccgactttttgcgatcttcaagcagaattcgtggctgcagggcgtgcacgtgcacgtcggttctcagggctgtcctcttgacctactggcggcaggagccaagaaagcagtggcgtttgcacttgaggttaacgctcatgttggtaacaagcaggtttccgtgctggacatcggtggtggcatgcctaccgtgtacgacggtggagagcgtgaggcatacgatttccaagaatacgctgatgcagtgcgcgaacaagtgcccgagctctttacgagtgggttctcgtccattatcacagagttcggacgcagtatttttgtgaagcctggcatcaccgtgtctaaggtggaagccgtgaaggattgggcaggccagcacatcgcggtgatccatgtcggagcagaccagtttccgcgtacagcctatttgccggagttgtggtcacactgtatctccgttctcgatggccacggccgcccgaagacgcatcctagcggctcctacgtccgccaggacatcgctggtccgctctgcttttcaggcgacttcctcgccaagcaactacttctgcctccaattgaagtcggagattttgtggtgatccatgatagtggcggctacaccatgtcgatgcactccaagtataacagccgccaagtttcgtcgaactttgcgtattcgagctctgccagcgacgtgaatttttctttgctgaaggaacgtgagacgactgaggagacactagcttgctggggcctcgaccgtgacgtggagctgtagctttacacaggttagctttccgtcccaacattgtgctagctttgacgagtgcaaggacggggtttctctattttccatgtcgatttcatgcttattcgtctcggtagatacacagcgaaatgcggcaatgagttgcatttcaagccttaaggttccgcggcgtattccatcagagttgagtgatttaaagacacaactagtagccgctctcgagcatgctgcgaatatagatcacctggccgaacgcaatacccacgagcacgacgttggtgatcatagacatccacttcacacgcgagttggtgctctcggcagctgcaagtaacacaaagcagcacgagttccctcatcagtgtttcgctacgaacaagaagcaacagcagcaacagcagtgcccgtaccatcgcgactcagccgttcccggtgcttgacaaaggaaatgtcgccacgcaaggtgaacactgactccttgaagaatgccacctcgtgcatcagttccagtccaagatctaaatgatacaatccacaagtttgttagtccaagcaatatcaatccccaccggtcacagcgatggcaaacgaaccttccacgacgatctgcgggaagtagtcctccgcgacgtggtggatgccgaaaacaacggcatcgctgttctcgtgagcatgaatcgagaaggcgaccgtttccgtcttgccgtcgagtttgcgctcgaagatctccgtgatgtcgaagccaacgtgactgccaagctccgtcacggcattatcaatcacgtgatcgcggtagccgccggtcgcgtagccgttcagcgtctcccaagtgacaggatattgcatgcgctcgggcagcagcgccagagcgatgtccacctgctgtccgtccgtaatgctgtccaccgtcagcagcagcttcacgcgcgtgttgccgcggataatgctacgcgagacaccgaccaggtcgaattccaggatgcccatctttgtggcctggccgttcgtcgtcgtcaccgccatctgagtgtaggctggtaccgtcggggcgtccttgctcaccgtggcagccacattgggcacgagcgttaagctgcgagcgcctgtcgagatgaagtcgaaggaatagaagagcgtcatggtggacgacgcgcccttgagcttgcggaagcacaagtggtagagtccgctttcgcgcaccgtggccgtgccaaagttgttttgctccgccttccacgtgagcacctctcgctgggatgggttcctcacctgcattaacgcaacgaagtgaggaccaacaggccgcggaaccgatgcagtaccacgaaacgtaccacgacgtcgacgagatcgtagctcttgggctccaggactccgaagcggaagagcaccttgttgtcggacgcgcgggcgttcacctcctccatgaagcattcctgcgcaacagagttagcggtggattgaggagaccgtggggttgggtggggggccgaacctccgaccgagacgtcagcgtgaactggaagcgggacgcgtgtgaactcggcagtaggcacacgcacaacagcagtgccagtgagaggaagctgcgcatagtcgagtgtgtggtggcgatgtggagcaaggagacgtcgctttggcacgatggaggaggatattggctcgcaaaagtgtccgattgttgcagcaacataagttggttggagcaactttatgtgggctcaagtcagctttctcgatgcgcgatgcagcatcgtcgatgcttgcagtgcggtgaggagaggagaggagtccattcggtctggcccggactaggggggctcgctgccaacgctggagaggattttcatcgtttgtggttgtcttcgctcgcatttgtaagttgaatgagctcgtctacaaattgcaagcagaggacagcatttgtccggcgcatgactacgatattgtcacgaggagcttaccgccgatagtattgctattcgcttatcaaaaacgaccacgaaaacgcggtaagctgtctaatgactggcacttcgggctttttcatccgttgcaagcgcattgctgatagagagtcacggccgatttggtgagcagctctgcagtcattggtattgccgtctccagctccacaccttccagcacttcttccccgtctcaggttgcactaacctcggcatcgtcgtgttgccaggagttcgtcgtgtttgccaatattcgcttctgaaacagggtggagaggaacggctgcgcgaggagagcgaggcgtgcgtggcgaaatgtgctcagagcgatctcgtggtcaagattcagtgggcgatcacgcagcggcagcacttcaagcggtatctgggtgcaggacttttggattccgccatgacttatgctataaatacatgtattatacttcttgctactgtggcggtggagctgcaccgagcttgctggcgttcagccatcgtgcagccaagcacacctggtgacttgcgcacggcaacttaccaggctgtggcgctgcgaccggctgcgatcccagcagcatcggtgaggacgcactcactcgtaggcaccgctacgtgtcaatccttctcgtcctcgaggtccgatcgggcgtgcgtgtcctacggggtgttgaagcccgcgccgacttgattcgacgcccaaagccagagatttcagacctaccccaggaaatgccttaggataggtcggttcacacggcgtttgtataggtgcgcgtcgacactcgtgaggcccgatcgcaggctgaactgtctttctcgccgttgcggcattgtagcctaccggcatacgtgtcggccccagtctctgcaaggcaaagtgtgcacacgcttccagtatcctcaagccctggcggttgtcgctcacttcccatttcgccttgccatcggttgagcacctcgcctatcaatagtgaggacagaaccccttcccagtgggagataacgacgtaaaacccccgctacaacacacgatggtcaagttcttcagtccgctggcgctgtcgctcgccgcctctgccgctgcggccgccgctttcgccactcacttgcgcttccgcccgcagccgcccaagcagaccccgtccgagcccgccacgatggccccgcgcacggcccaccagcgcaacatcgacgagatcaaccacctcgccggctacgatgtcatcatcgtgtgcacgtccacgccgcaccaggcgcgctactggcaggaccgcctcatggccacgcgcggctccatctcccccaaggatgccaaggtcatcgccgtgtacgaggactgggacggcggcgccggcaacggactcggcacgctctacgcctacactaaggcggtcgctgccggcaaggagctctacggcatcgacatctcgtcgctgctcgcctcgggcagcgtcagcgtcgcactgtaccacacagccggcaagggaacgcgtctcgcgccgctgcctggcagtgaaaacaacaacaagtctggcgtcaagctgccggccatggtggaggtgaatgacaagttcgtccccatgaccatccttgaggctgtggtgaagcagacgggcgtgtacgcttccagccgccgtggacgcctctcggtcttctggggcgatcaggtgttcatcccgtcggcggaggtcaagtacgtggccaagcatcacatcgacatcctagctgcgctggccccgatgccctcggagaaggagtggaaggagaagggccttgagaagtacggcctgattgtcgtcaaccagcaggacaatgcagcgcaggtggacaaggtcactcacgagaccgcgatccgtctgttgtcctccttcggtgagatgaagtcggtcggcacgagtctcgggtcgttcagcgtggacgccgacatgttgcgtgcgctcctgagcgagttctcgaaggaactggacgccaagtccggcaagcttgacagcgacccgcacttctggatgccgctcacacttgagctcgatgcgtacacggaggtgatggcgcagaagggagtggagaaggcagagtccaccacgcactaccagcgtatgcagaagatgatggccaacttcgaggagacccggacgaaggagcttggactgtttggctgtgtcgacgtcggcagcgatgtgtactggtgggactacggccagctgaagctgtacctgaagaacaaccgcctggtgaccaagccgggtgtggaggcagattgcctgcgctcgttcctcggcatcagcaacagcctgcagcacagcaacgtcggcgtggacgcgaccatcgaggatgctacggtcctgaacagcgagattggtcacggcgacattaagcgcagcgtgttgagcggcgtgtgcgcgaaggaggtgaacgcgcacggttcaatcctcatcaacgtgactgcgcgctccatcacagcgcccaactgcgtggtgtacaacgtcacctctgatgaggccgagggtctgtgccttgaggagggcagtgtggtcgtgggcgtgctgctgcccgatggcaagaaggtcgtcatgcgctcgagcatggatgtgtgcggcggcaaggcatggaagaccgtgctggaagagaaccagcactctttcgagaagatctatgagctgaacgcggatgcgaacgtgtcaaagctggagcagctgatccaggctgagcacttgaagatgcgcgaggtggtcatgtcctaagcatggcctgagctagtagtaacgtagataattcccccccccacccaccgctcgccggtagaaactcctaaatgcatttgatttcttaccctgaacgcacgcgaaccaaagcttcaagccgcagcagagcttcaattgtggtcaagttccatgccgtgtagccaccgtgacgcaggcctcgtggcagaccttgttgctgcagtttgattgcaaatggcgctgcacttgctcttcgctggtcgtcggagattttcacaaccccgcgatgccacgtcagaacgcttttattcggagtggactcggagtttttgtactcgtacttagtccaccctttgaaccacaccagcgtgacacaagtgacattcgtcatcctgataatcgctagtcgaccgccacacgttccggagatgccagcggacgcgaagcttcagctgctcgtagcggccctgggcgcgatcgccctgcagcaattcatctctcgccgccagcgccaagctgtgcaaatggagaaggtgaagcagcagcagaagcaggtccagagtgccgccgcggccgacgaagacgaggcgtttgtagtggagatcgagtactgcaccggatgccgctggatgctgcgcgccgcgtggatggcacaggagcttctcaccacgttccagcaggatgagagcagtcgccttcgatccgtgacgctcacgcccaattcgcgccagggaggtgtgttcaatgtgtacctacgcgaggttggccccaacgccgaccccgaagccgagccagacatgctgtggtcgcgtaagatcgctcgccgcttcccggagtccaaggagctcaagcagctcgtgcgcgacattgtgtgtccagagagaggcctcggccactcggacaagaagtaataaggcaggaaataggatggaaaggaatcttctattctctactgaggcgctcggaatccagccatgacgcttggaagtgatgaacgggtcgaccctccgggaaacactgcactaggcctgtttgaacgacaccatcgtcagccacgagagagatcgcagcagcaaatgaatgaaccaaactcaccgtgcaaaaagacttcgcatgctcttgccgatagacgcaacacctgccagactctttggcatgttggagaacgatgttcgccggccgtcgggatgggtcacgcacttgatagcagatacttggaattctcgccatgcccacgggtgcgccttcagacaagaccgtacagcaaacattagcttggagttgctagcgttcatgctaccgaatgaaatgaaatcatactcggatctgttgactcgtgaaacgctgcttggcgtccttgtcgagaatctgccagcgttagaagtgatgtgagaaatgcatcgacgaagcttaaacaagaggaattacagtctacgcacgtgtttcaagagatcgataagctcactttccaccacacgatcgccaacgctgtccgggtactcaatggtgtcgttctatgcgaggttagcccagcaattagtactgtgctgcattgtaacaacgctggcagttttacatacctgaatcctgccgtacaactccgtcatcgttttagcgacaaagggcggatgtccatagataaacatgtacaaggtgactccgcaagcccaaatatcaacgggcttcccttgaaaattttctcctgtcagcatctccggcgctagagaaagatacatttacttagcacttctgaaaacagtatgccaacacggtctacacacgtacccatgaagatagcagttccggcagtttgcctcatatcctcattgtcatttgccatttcctggatacaacgtcaacggtgaaagttaggcaatattcacgatggctttcgcacttccgtatcatcttacgtgagaaaccccgaagtcggcaatcttgaggataacctcattttcgtcgatagttttcagcagcaaattccccggttttatatcgcgatgaattatctagcgacagcacatccgtattagaagtgctgaacgaatgtccacccttgaggttaacaagcgtaccttgttctcatgtaggaaatcaataccttcaatctaaagaggaaggaagcgattagaaggcaaagttgttgcgctgtgttgacgcaacacggtcttaccaggtgtgtgaaatacgtctttgtaagttccgcgtttagtggagtacactctctgagacacaaagtgaaatgatgtcagtgatggtagcaatttactgcaacggtagggcgataaattcgtacatgtcatcatccagtacgggcccaccatctacaagctcgctgatcatgaagagctcttcgctgtctacagtcaaatttaaatgagtttcaagtctgcaagtatcactttggcgcgcgtgcgtaccgtccgtatcgatgacttcacgcagcactacgatatgccggtgtttgaatcgcttccagatattgagctctttcttgacggattgcagtgcattaccaaaacgcccaacgcgaattcgcttgagagccttcttatctacacactttgctgcgaaaatctcttttgtgtcgttgcattccaccttgaagacaataccgtaagctcctttgcctagctggtccatgaacgtgctgtaaaaatgaaaccacatcatagatagaaaagtaagcacagcgcaatgcccaggaggagcaagcaaaatcgttctcacgtactactggttcagttgcctccgaccgtcagcatctacggtgctaaccattctcttggtttctcggaaggactggcctcgcttccgtagtggtacgattggtttcattcctgtcaactgtttatcgcctccactgccttgcatgctcatcgaagctcgactgagagcgacgataggctttacaactaccaaagcaggaggatccggcgatggacctggttcgactttttcttcagcaatcggatctggatcgggactgagagcgatgataggctttacaactaccaaagcaggagggtccggcgatagacctggctcgactttttcttcagcaatcggatctggatcgggactgagagcaatgataggctttcatgacaaccgggtccggcgatggacctggctcgactttttcttcagcaatcggatctggatcgggactgagagcaatgataggcttcataccaaagcaggagggtccggcgatagacctggctcgactttttcttcagcaatcggatctggatcgggactgagagcaatgataggctttcatgacaaccgggtccggcgatggacctggctcgactttttcttcagcaatcggatctggatcgggactgagagcaatgataggcttcatgacaaccgggtccggcgatggacttggctcgactttttcttcagctactgggtcgggatcgggactgagagcgaagataggctttaggataaccaaaccatgaggcttcggcgttggacctggttcaacttttccgtcagctacggggacgggaccaggaacaacagcactgatagttatttcgtcgatagcattattgggtggggatactggagcttcagctgcagcctcggacgatccagaaaggcctcccacctcagtgacgcgttgcagcgggtctagcctggctgcaggtccatgcctgttgttgggctccaggccagtcgacgacgctttttccatcgggaatacgctcaccagcggtggctgtatttgataaagctgggcagctggcaccagagcagctttgctaaatacaaccactacacaaaagtgtttcgcgcagacaaagatagcggcatcattcatgtatcacacaggaatacttacgtatatatattttttgctagaagtggtggacttttatacacatggacgccatacacctccggcgcccccaccccacaacaatccgggttgactgaagtctagtagtaatcataatacaggtcttggcagtcgagcaacgctttgacattgtcacgaacgtgtaccagccacgagatctgttcgtcgaggtggaccagcatttcctccagctgcgggatcggcacggagacacgagcgtactccacaatctcgaccttttggccacggaaggcgctgcaaaatgacaatgcgtgatagagggttagaaaactaaagtcagcaattaccaccaaaccatggcaccataccagacgcagccgatgaactctttccgtgacgcacagccctccgggatgtttgtgatcggcttctgcgtagccatcaaactaaaggcttcaaccagctattgaaaatatgaacagcgcgcacatgttagttcaacgtctccagtactatggagcttgacgcattcacacaccagttcgccacggtgaatcgagaaatccacaataacttcggacggcagtgccggcttgaagctctgcacaaaccaatgacgctgttcgtcagtatctctcaccccctacaccttctctgctgaaaaccaacgtacgtggtcgacgtgagagcagtaagggaaccgacgacggctgggcagccgcaggacgttggcggcgttgcgcagctcctgacagatgcggcccaggctctggaaagtgcgctgcacgatctccacgtacacctcgacgtcatcattgctgggctcgtcgtgtatgagctcctgccgctggacgtgcatgaattccttgagcgccgcgagcctccccacttggctctgcacgatgccgtggaactcgaggatctggttcagcacgcagtactgctgcagtctgtgggcgtcatgcagacatggacacctggttataatctgcagaggaagctgcatccagtactactactcgtacgtgctaacgatgggcgcattgcgctgggccttggtcagcttgagctccaccttggtgaacaccacgaggtggccctgtcattaggcgacgcgtatgtaacacagcacatgccaatgccgtcagtatccatcctaaatcagtatgcgttcgaatctgaacctacttccacgcggatgtcggccttgatcgcgggcgacgccgcgccacggtccgagcggatgcgtccccagatgaagtcgatgcgctccaactcatgcgccgcgtagtcgatggccgagttcacgtctttgagcaggtagtagcgcgtctcgcgcttctgctcggcaggtggcacgaacgtaagcatgtgtactgtactaggttagacgaagtagccctacccacccatgcgctgagctcgtcgtcggcttcctggagcgccacgggcgccgctgcgtgctgcgtgaggctgctggacatggctgcgatcggactgctcgtgctctggagtggcggatagaaggaagaatggcaggcgtctgcgctcactcgcgtgcgcctcgtgtggtgtcgtccatgttgaaccgcacggaaaggacactctgcggacagccacgattcagatttgaattgtgcgtgtcgcagacacgctacctttttgttcactacagcgatatccctgtgatacacgatggattcacttacatttccaagtcctcccactctctgaaagcgagtattgcaggactgtcaccagataatctcatgatttggctcaggcttgctgcaacgtttcctcgccacaattttgatcgccgtggccaatttctcccgagcgtgtcacagccagcaccagctggcgcgatcgaaagcctaacttgaggccctcgactcgagcgtagcaagtgatggctgtgcatgcgcattggtatccgccgaaaagttctcgagaagttctcgggccagtcgcacctaaagatactttttaagccctctacggtatttatatcgcttgaaatggctacagttcttgcttcaacatccgagctacgagcagcatggcgaccacggaggcggtagcgctggccatggccatcgaggaaggcaagacgcgcgacctgtacaacgcgctgcggacgggcgagctcggcgtcaggtgggcagccgaaaactgaagcgttttaatcgattgacgcgcgtgagacggtccgttctgattgttttgctgttggtgctgttgcagcctcgaggcgctgatgcaggcgctgtacttcctgcagcacgccgagttcacgctcttcgatgtgaaggacgccttccgttcctcgattggtgagaagtgcagactttccaccatattgccgccttgtcattaacagcgctgatctgacgctgcacaggggcgagccaacgcttcctggcctaccgcgagttccaaatcgcgctaggaaagatcgcccgtgtcaagtattacggcaattgcggcggggctgacgagtcggttgggacttcttgggatggaggagctccctcgccaaagcagcgcccacgacctacggagctggggctcatgtgctcgcttgtggatcagctgaacagccatcgccaccagcttgacacgttacggagccacatgacccgtcccagtactcttgacacgctcgagcacaatttgccattgcttgtccagagcttccagctctacggcacgcagaaccgcctctcaacgaacaccgcgctatcaatgctggcaaccataacgctggacgggttcgtcgactttctcaatgcgtacttcgagtatgaggagttcttctcgttcatcgatcttgcacgtgtgagtatcagccacacaaccaggttgtttggcgaggttactgatattcgctcttgttcgtggatctgttgcagatgtcaaccgaagtgctttctggcttcactagcgctagcgctagcgcaggtgcaaaagccgactcaaaagatagcaaggaccagctcctcaagggggagctgaactttgcgcagtttctggagttgttttgccgtgtggcatctgcgtttcaccacaagctgctagtaaaagagggtgcgcagcttcgacgggctgtggagtcttgccgtcttgagttcagccttgaggtgctgttggatcacatgcatatcacgcttatttcgaatggtggctcgagatcgacaagtgcctctactccgcaacagcaagaggtttcgaccatcacctccagtatgaaagctctcacgacagaatcaccccagtcctctcgtggtgatgaacttccagcgctgattgcaatcgttcagaatattcgagatgtgcttcggctcgatgctgctgaccatgctatatctaagaagcctaaatatcgtgtgaagcgcgagggtagcttggtaatgcaaggtcagctgtccagaaggcctagtagcaacccccccgagcaggaattgtcaggtagaagccccccaagtgtgcattcgaccagaacgcaacgccaggcaaaagtagaggaaagcccgaggccgcttccgcaggtggctatgatacgagaagttgtgatgccaccagcacttcctgcagacgtgatccagcagctcgaaagtgccctaaaattccagaacttgggccagtacaacgtatgttgctcaactatttatcggatatctattgtaccggtgacgctagttttacttacgtacattgtgtatgcaacgcagatggctctgagtgctctggattcctgtgagcaacgctatcaaaagcaaatcttgcagcccatcgacgatgttagcggcatactcgacccgattcaaaccgaagtccatctcttttttgctctccaagccgcaagcatacacgatagcgcccaccgcgactcacacgctctgataaaatactatgaggcaatgaagttgtcgcgccaacttccagcatcgcatccaggtcgattgctcgtcaaaagctgcctgggtgtcactctattttacgctggagagctaccgctagcacaacagtgccaccaactcgttttggatgcacgaaaagctacaaaggtcagtggagctgatgcccgccaccttccagacaaaagagcagcgtccaagactgataacaactcgaaccgtgttctcgtcgacactgcttcggcaatgaacaacctcgcctgctgcttatcccaagaccagagtgattcggcctcgaaatcttcggacagcgcgtatcttttgttcaaacacgctcggcaggtctacgtggatgcttttgggcccgcgcaccctcgtgtgggactgatttcgcgaaacttggatcgcgtacgtgcttgtcaaaatggtgttgtgagtgatgcggctgatgccctagcacgtggtgaatacgcacatgtaattcctggcagcacattccagattcaagcgtttgagttcgtagcgaagcctgtgaagagcagcagtagcaaaagcagtaaatccggaggcaagaagaagaaaaaaggagctaagtaggttgttgcaacgtaaatccaaccattgttttctttcagcggagattccgatgtcttcgacttcaagaagagaaaatggaaatgacttcaagcaacaagtacgacaaagtggaggaaaaaaattatgcacagaataatgcaagtctattcgtgagttggtacgacttagtttgtcttcgacttgacaggcgacgtgagcagcaggccaagaagttctcccagcggcttcttccagtacttgtccaatgcgaaccaagccacgcagaagtatagcacgcgagcttccaagattcgttcctccttcgaaacgaaatggttagcaacgtgagccacgacgccaatgataccgctggtcgcaacaatctgtaggatgaatccaaaggcgcctatggataattccaccacacggtgcgtctggccgtagttcgtcaagtagcggagcaccataccgctagcgtacaagttggcagtagaaatcgcaaggaacccgagtgggtgtgcatttgtattcgtctgccattccatcaccagcttagggatcgtgacagccttcaaccagctaacactgagcccaacgactgctttcagcagaggaatcgggatacggaggccgtcgaccactacctcaacaaggaagtaaatgggcaccacgagtctcacggtctcttcaatctgcggcgcagcgccgaacacagggcgaatgatggtcgttcccagtaaattggcagtgacggcagcgaacagcgcgcgagcgtggcgaatcatcgcctttcgcagcgacggcggacgacgtgccagctcggggggagtcacaaagtccgtgagttgggtgcgcacagccaagcagacccagacgatgaacgaacgcttgaggaacgactggagctctggggactgcacgtagatgactcctgtcgtgaactcgaaccaaacaccggcgagggccacggtgatcaccagcaaattcggcaaaaagctgaagcatatggacaaccacaatggggacgaaccagcaacgccattgcgtttgcttggggtggtctgggggggagatggagtcttggcgacagcacggcgcttggccatgtcgattgagggatgcaatgcaacggagactgaaggtcggaacttcgagtcggacagttgacgcgacgacacgttgcagaatcgggaaacggaggaaactatatatagcaggtttttgtaatagacattgcgtggagttgtgttttcgtgacggttcagtataaactatgaaaggctttatggatcctacgacgcgacgagcatgtgtcaaaatggaaatcagtgtgcttgccacgccataccttggcttcctttgtacatttcaccgtcgttgtctgtagtaccaagtggtaaaactgtgcaatcgccaaccacgcgaagatcggttgctctgggcaccgaaactggaaaagaaggcaatacatacaacatctattacaataactccactgcgtttcactaccaagtcacagttttctttttgatagactcgatgagctgcgctggcagcaattcgcgactgaagaaggaaagcgtcccgaattcctctatcacttgcgagctgtacatgtacgccacccactcccacaagcacccaccaacttgggccgcatggaacagagtctcggcttcttcctgcaacgctgatttcgggagcggtggcgcacactcagccgcctctgacagcaactcgtaaaactctgtggagagttgagtcctcatcatgctcggcaggagactcacgtattcgtcgaacgtgcgcagcgtgaaggtactatcgaacttaggctcgttctgaccgcgattgcgatgcttgtcggtcgctttcgaagtcttcatgtatgcaaagcgctctttctcgctgcagctagtaatccattcgtgaccttccgtctcgatacgctcggcgaacagatcatatttctgcttcagctcttcaaatacagttgtgaatggagattgccactcccagtctttcttcatcttcggccactcacggtcaatgcgggtgtgatatgtcgaaaggtacgtgaacaaagattccttgacgtcccagatagccaagaagggattgcacagccccagaccaccaaggttcaacggccaataaatccacgctgctggcatcgctggacctggactctgacgcctcctcatgatctgctcacgcaggtaacctagcgcatcgccgtcagtcttcggaaatacgagcttgtgaacgcggcggagtgtagataacgcctcgtcgatgtgacttaacccgaaaattggggacgggttcccaaagttgcgcaggaagaagaacatgtacttgttgtacacgttaatccacgcgaatgctgaggttgcagcagccaaacggtctgccatttcggttgcgaacgcctccacctcatcttgacggatatccactgacccatttgaccgcagctcaagcagaccccacctaattggagtcgctggcagcggggctggacccacctttgcttccgtggtgttggatccagcgttcaagcagattgacccacttttctcttcattgaatttcagaccgacgcaccgggcatagcgctgcatttccttccaggctcgtagcaccttcgcctcatcggcatcaaagaagcaaatatcgtcatgcgaccggtacaagaaaatatccgatgacgcaagcacgagataatccatcacaaacaactccacctcagcgagcagcatagtcatcattcggctgacagggagccctcgctgcatactcttgggtttcgtgaaaccagggaaaatcagcggaatttgtgtgtatcgccggaacacgctcaacatatcatcatgcacgcccaaaaagcgaaggcacgcaaagacagcttcgcgcgcgacagacggcccgaagaactcgaggtctgtcataacggccacgagaggtgacggctccttgccctcgctggctagaattttctgtaggtgcgcttccgctagcacacgacgaatgatatcctgtttgtctgatttctggtcctcgtcgtcgccgtcgtcggatgccgagtcatcttcatcgtacggaccgcgccctccatctttcatagactccggaagagcaaacaactgaaatcgtcgcatcatgtcggcacgaacagactccacggatgtcaacccgctcatccgtgggtcatcactcagtgagtcgagtagtgctcgaagtaccgccttaatttcgatgctccacttgaggccgatgtactggaagagcagcagcgtaattgcatcttcctgtagcagacagcggaagcggccgttcatgccacgttggatgtcgacgtgaactccttcttcaggccatcgccactcactcacattgctcaactcaatcgtaagcacttgggacagctctttcataacctgcttgttgcgctgggcatcggtgaaaagcgccgtaacatcaggattcaggaaatccttgttacgtagcagcgcggccacacagcacttgacatcttcttcggtgatagggatttccccacgcagcatgctgtcgccaaactggcccaaacgactgcgcgcctggtgcagcgcatcttgctggcgctgcgtcttggcccgggagaattcttcgcgctcctggaacacctcggtcgttaaaaacgcgtacacttcgtcctccgacacgccgctactctggaaaaagtaggactccagtcgctgctgtgtttcttccctcgacagctttgcaagtggttcattgtcggcagcccctccaccgacagcagacgacttcggttcaccgccttgtgggcggtccgcctctctgtctttttggtcgtgttccgtctcctccagcgactccatgagcagttcgcccagcagcttgttgtgctgccagagactgcgacgctgccgaacttctcgcaggagctgctgcttgcgctgttcgaccaccgtgtcggtgctccaggggtcggcggctgcgaactcgaccagctccccggtgttttccaacccctcgtgcaccttggtctgcacgacctgcaggtcctttacaccctcgtacagccctcccagttgttctcggccggtcgaagctgcgccagtcttggccaatagcgccttgtagtgcgtctcgagcttgcgctcgtgtttggtgagttctcccaacttgatctgcgtcgacagcttgagcagctggaaatgcgctgtcgcgggcttctgtgaggtcgccattgcaatgagagaagaaaacacgtgagcagtaggttgcaaaatgaagagtaaggacgaagatccgcttttgaattttgtacccacacattttaccatcactcacgtatattgtacaaaaaggcaacttcaacgctgtgatcatttttatcgctgctgtacagataattcgtgctgaggttagcgatgacctcagcacattatctccaggatctgctcgctgcggcctgctttcagaattttcattggaaagtcgtttcctgatggagtcgtaatgtccacctgaatcgaacgttgttgtactgggatcgggtaggggggacctggggcgcgacaagcaacgctggtgaagattgctgctgaagtcgtcgcttgcggcagctcgtgtatatgctgatacccagcacgaccgccatcccagcgatgcgtgcagcgatctagtctagctcagagcgttgcttggcgccattccacttgagctgcttcgtgcgctactggagctcctgcctgagggatagttgccaacgactctcccagcggaggttaaggaccctgcaagtgttatgggggggaagctgctctgacgctgaaaagccaggagcgtccacagcactattacagctcttcatctcagcgccatgctgctaacgttcacagtttcgggcggtgtcgcatggtggagtgcaagttttcgtgcgtgtcgagctctccgggacgtggaccctgccatgactacgacaccacggctcgcctttacttcgccttttcacgaggcaagtaatgccatgacttgtctacttggaaaatcgagctgaacggagaccgaagctatctactgctcgtaggagtgaatagaatctaagccaacagaatagtgcacgagagcgaatagaagaagtatcattcttcgctgacactttcgtcggcctagaagtctcctgcaaactcgccaccaccgaaatcgccgccgccaccgccacctgcatcgcccagagcttcaccaatgagcagcccacccagcaagcctccagcagcgcccattgccacggcaccgcccattcctcgatttccgccacgtcggtagtcgcctccatagcctccatggccataacctggttggccgtaacctggctggacgtaggtaggctgagcatagcccccctgggcgtacaccggctgcgcataggtcccaggagcgtatccctgttgggggtagccaccctgggcgtaaactggttgctgggcataggtcacgcccgcttcaggtgggtagtcaccgtgccgctgttgcatgcgccgcttgcggatggccatcgaggcgccaccgcctatcaaggcgccaacgacaaagacgccgaggcctatccagatccagtaggacggacgcttgttgctcttctggcacgtgcactcgtacgtgttatccgaggagcagacatcactggcggggcagcagcaggcacgtcggttgttggacgagtccagccacgagttgcagaacttgccggcggctccgttgtaggcctcggagcagtcgcccgagtcgcggcagatttcgcactgtgacttggacgaggacccggatgcggatgagttggacgcgacgatggtggcagccgtcgagccgctggccgactgtgccgtcgcgacagacacgagcgccaccaccaccagcagctgcactgccagaatgcgcaggacactacggcgcataacgaacttgacgaggaggttcttccagcggtgtgtgtcgagaaatggacaaagtttgtctaataaaagaggtgtgctgaacggttttgacaggatatgcgtaagtaccccgctcgcggcgacatggagtccacctacaatggatacccggcttcgtccagaatatccatccgcggggggaatcacacgtgatgcggcatcatgtccgacatgttggtagcggatttgtgctagggcaccttgctgtcgaaggtgtcattaaggtgttgaaaatagcgaaaccccttaatttcagatagcgcgcaatatggcgatcgcttggggccggcgcgtgctgacggcgctcgtggggatcccgacggcgctgcacctgctgagctcggacgccggcatgctgtgcctcgccacgacgctctgctgcctgtcgctcgtggagttcagcgccactatctgcccgcagatcgtgccgcagcccaaggcgacgacggagaggatggcgcatcgcctgctagtggtcgcgtcgggcgcgctgctgtgcttgggcgcgtggaccggaaccaaagtcgtgcatggtgaggagattgcagttgctgttactatgaatatggaggctgatggaagttgtgctgctcttctgttgcagacgccttgatgctcggagtgacgctgacaatctccgcctttcacattgcgaccgccaagacgatggaccaaacaggttaagtcctgcaggtattgcaggttgtagcctatactaatgggtggtcgtgcgttgtgcgttttcgtgttgcagcgctgaccaagttgctgctggacctgttcgcgctgctatatatcgtgagcggtttcagtcacgctattctgctgcggtacagctccggcaaatatggtcttggactgcagatcctggggctctgctgcgcctgggtctgcgacagtggcgctcttgtggcaggctccttcatgggtcatgcgaagctggcacctgtagttagtccaggcaagactctagtgggggcgatagctggagtcacctcgagcatcgcgacggtgctggtgatgtttattctcccgcagctttccagcgcgctgccagttttcgggcaatggacacacgacctgctgcctcctctagaagtggtgtctcgtacgcaccaggtagtacttgggttggtgcttggagtgttgtgcatcgttggcgacctggtcgagtcgtacgtgaaacgcgtcgccggtgtgaaagactcgggagcttttttccccggccatggtggctgtctcgatcgcatggacagcttcctgtttgtcgcgccgtgtctgtacttctacagtcaatttgcactcccctaagcccaacaattaggccagagctgctgcttcgcatcaatagagactcaaaagactcccaagcttactgtaatttgcgattacaatgtggcgtccacagcctcctccacgatggattcgacgggcgctttcttcttgcccttgccactcttcttgatctcgggtttcgatggttcgcttgttggtagagcctttgcagcttctgctgctgccacctgtgcgggatcgttaatgtcgattgtatccaccgccttcgtcttcgagttcctccgcaggaacttctgtcggtccttccgggacccgcccaacttgctcgagtactggcggatacggcccctgcacgccgatccacaatataaaacatttaagtatactgcaagccaaacatagcagtatgttatccctcattgcacacgtacgtgatgacggctgtgtcgggttcgctgagcttgaaggacgtcctgcgcatggccatgctgaagtcggtgacttgtccatcaccgacgccatgcagcgcgtccagtttggcagcggcttcagcttgctggcgggcaatgtcgtcatacacgcacttttcattctccttggtgcgccagtagtagatgcccacgcagagcagcagcaggatggacatgttggacccgaaggcagactacagcgagaagcaacgcaagaaagaggtcagcaaaacagagataccgggattgacgctgcttgacgcaccttgaccatccccttcgcgatgatgccgaacacgaagagcagcagcaggtactgcgtccggagctccatgatgctgcctgccttgccacgtcgtttcttgcgctgtcttgggcttgaatcggacttagtaagaggctgttatcatttcgacacagttcgtgtacaaaacaccaagctgcagagctcttcgtcttgcaagtggttcaggtcgcacggagctctaccgcaagtctcatgcctctacgttgcgacaatgctcgagcttttcggtggtgctgcgattactggtcgccaaccacggaagttagcaaaatcgcaaacattttcaacgtgcattagtaagttatgctccatggcgcgccttatacatacaaggtaggtgaggctgttggaacgccagcctcttctactggctcggcggagatctctggcgctacaggtttcggatgctcctgcgaatcggatagtccgttcacggctcgtcgaagcccaggaactcccataccaagtgcattacgttctcgtctagcggctcgcccagcgaaaacctctcgaacacgcgtagcacggccaggcgctcgaacaggtagttttcatgccatgtagcacagtcgcccgtgaactcaaagtctccgagttccggagggaactcgttgccgtactcgatggctaagtgctccaaatgcacgttgtactgcagggcctccagcagcgcacgcgtgccgatagcgtcgatgtcgttgaacagcaggctgagatctgtgagccaagagtcgagaggggactgactgtcgctgatcagcagcatggcaagatcgaacgcgccgcggaaaccaagcatgttcgagtccagattgagactccggatgcggcagcggcccttcttgtacgcctccagcaacggggtgatgttcatgagttggcagtcggacaggttcagcgtcgttaaatatggcgagaaatagataatccggtgcaggatctccgtggctgggatgccgaactggtcggggctgccgatgcggttctcgctcagattgatgtcctccagtcggtcgctgatacccgtcatctggaagaagagcgtgatcagtggcaccaccatgccctcagcgtcggaggcctcgatcatgttccagaacgatactgagcgcatgagcgtcgccggcgagcaaatccgaggcgacatgccgatctccgtcatctcaatggcgctttggcgcggcacccatccgagtccgaagccaggaacaacaacacacaaccacttgcgcagaatacagaacacctctagcagctccttctccacaagaaggtcgctgtatacttcgtgctgtgacagcttggacgtcggcagcggacgcaccgtggtgcccttcttgataagggcgaacacacgacggtgcggaatgcccttgagcagcgcgcgcttcacgtcctcgcgcaggatcttatcgcggttgctcttgctgtaacgctgcaaggctgctgcgtactcggggatcagcagaatcagcgccgggtggcccccagccatgatcccttccacgacacggatgtccttcatccgcatcatgttgcgcgataggtccagcttttcaattcgggcggtcgagttcgggtggaacaggccgaacaccagccagccccagcacaggtgcaggtcctcctccttcacttgggcggggtccatcagttcctgcaagtcgatgttctgcacggtgttcgagtagtgcatcgccgagccgatgctggcgaagaaagtgttgtccatgggacactcacgcacggcgatctccttcacaaggccgcgctgctcctcgggcagctcggcagccatcgagttgtacttgatcacgggatgcacggcaaagatggcggtgcagaaccggcggaaggccacacgatgctggtagatgaagttggcgtccagcgtgtcgatgaggtggatctgtcggacgtgcgccatcttgtcggtgatgaggcgcgagagcgaattgagctgcacctcgttcatctggaagtggcgaagcgtgagaaactcgatgaccttgcccacctgcgtgccctggtacacgtagcggcgcacctcctgctcctggatggtgcgcagcaggtgcttgaggtccacgaagtcgtgcgcgagctcgaagctcaggccgtggtgctcgtcgcggtggtactcgcagcgcacgaagtagtcttcgtcgcaagagcagaacgggagcggctccggctcgctctccgtctccgaataggtgcctgtggagtcgtggtatttgcgcccggaaccacccgggggcgagctcgtggggccgtagtacgctgtcttgccgctcaccatgtcgaggcgcggcgtggtctgcggctgcgggtagcgcgggttggtctgtgggaagggctgcatccagcggcggacgcggcactggcggctagcaacagcgcgagcgtgggggagaatcgaagtgaccgccacgaggccagccgcgcgtctcgcttcctgtcactgcgtgcgcgtgcagcagcaattgaagctggccgtatcgttgcggcttttttgcggcctctcttttgattggatcagagcggagatgccagtttggttcgaaagtgacgtaaacctcaaattaaagatctgtttgaggtgttttcgacggtgcgtgtacgcgtgagactttgtgctagattttaatgctgcaagggaggctttggatgctggccgtgtggatcctgcctatgccgtggtgctcgaccgtgtgcacgacccccacagacttacaatcgcaactgactgcatccgctgcagtgacacacatctatgcaaaacaatagttttgaagaaaaatggcaggttgaggcagcaatacctcccagcggcggctctatttgagtgcagtaccctcaaatagatgcaatacgctgctttgcaaggctttacaatgccatggcgttacgcacgcagtgaccgggcgaatgctattgccgccgtacctatcatccgacggcaagcgatggagccaccgcggcggcgtggcacaggcatcgcaacgctgccagccagccagccatgtccaaccaaaccatgccagcttcgagtccagcgtccatcactgcgagcctcgctgctggcggtggtggcgtgcttcctgcactgcaagcaagtcaaaatgactcggaggcacgcgtgtctcttgcgccccattaagtgcgaccgctcgatatgtcggcgatcttcggcggtcgcgactggagatgtcgcctcgtcgccgggttcaagccctgccgctgcgaatgaacgctggccgcgaccaccgcgcgctcctggttatgccccagcgtggtggcagcgcactcggctgtacgtcgccacacctacgctcgaagagggcgggcggcgagcaccgtcggaacgattgcaacggacgcttcaatggtggaacatgaggatagctaataggcgagggtgctgctggtcgatcgcctgccggaagcataacttgcctgtccagtgaattgtacagcgtgccggagccaagctctagcacttttctagaactgcttgcccgcggttggtggcctcttcgaagcattgcgacgccgaattcactgtcgccctcccgagggtctcatatccgccacgtagtggtctagacggcaacgccgtttacttaaggcagacaccaaccaccgcacaaaggatacacgcgccacacttcactgccttctcgtctactcgaaccattgttctcgcattgctcgatcccacgtaccctcaggcggcctcgcggtgacgttgcagcgccgaccctctgctgccgttcgtcacttgcgtcgtcgatcaccaaagtctgcccttttcgccgacgtctaccacgatcgctttacgacggtttcctcttgttccctgcggtgatgtataacgagtgaaggcactcggacaataaggcagcgctgtcggggtccccgactcggggggtcacggtcaagcaagaatgagctctttgcagctgaaggtttgccaggacgatgggtagcttcaagcagagaaatcaggcagtttttcgcgtctccacgcaatccctaccgctgcggcgctgagttgccagcgatccctgcacgccctgcaatgctcgcccaagctgtgcagtgattcactgccactagcagactgccagtggttaagtgactgccccttgtgctgtgcagcaatgttacactgctacgttttcagacaatttgcctgaggcaaatacactgatatttgcaagaaaaaacttcaaggagcatccctaccccttccatgatgttcaaattgaggcggctccactcttaaatgagggtatgtgtgggtgggtattttccctatccgtaccggacatatttcggaccacttgccgcacatacaaattcaagccaataagcaaaaagcaccacttcagcgatcggcgcgaactgccaccaataatagtctttgggcgctgaaaatcgcgcacaaagtaagaggagaaagccagctttcgaaaaaaaaaacacacacacgctttcctcagatccattttgacgttacaacatctccccaaaactcccccaatgaccaaggtcccagtcgagacgtgggatctgctgccattgccgctgtcgctggcggcttgagtgagcgcaaggccaccaaggcttacggcgtctctcgtgggcctcttcaccagcgcatcaacggacgtgtaccgctcgaagctcgcacagggactcagctggactacactactgaaggcgctgaccgaggcgtcgtcgagatggtgcgctaccgcgcgctgcgtagaatgtgcgtggagtttgaagagctgcgcggcatgctccaagtacatgggggatgcggcctttgactaatgactttcctaacgacaaattcattcagcgctggcttgccaaacactacctcgcactactcagcttttggacattgaccgtgcttcagcttccacagctgatgtcgtctcgcactacttcaataacccccagcgcgtcatgaagaagctggatctgtcggacaagcccaatcgcatctggaactgcgatgagactcctctttccaatttgctgcaagttactgagcgtcaaaaccgggtgctgcgagaactagatctgaatgtggacgcgctcaatgtcgtgaatctgcttcgagcacggcggtagcaccacaagctaaaaagcggaaaagaggagactgggttgacgaaaattactctggagggcagctgctgagctacgcgatgatggttgttgcggctgctgcaaaatcagcatcaatagcccaaaaaaaacgaacaaggagtctgctccgaagcaaaaacgtgcccaggatgcgatgaacctgcacgacaaagccgaccaccaaacagacaggtcgaagaagacactcgaaaagtgggaacgtgctgccgtgaaacgggagcgaaaaacagaacgagctgcagcacaacgtgcgaaggcaatgcgaataactgccgagcgcgcgcagcgactgaaccaggagaaggtgggggagcaagcgaccgatggttgtatgctcttggtagttgtgtgagcagactggcatacaaaactcaatagtaaagctgggtggtccgaaatctgtccaggggtgtatcgacagatatacaaaaatagcataattcgctaagtggtccgtaatatgtccagcacggatacagtactttcacaatagactacaactgtagacatgccaacgttggaggcgtgcgaaatctactgtgggaacataacggccctcttcagttgttgttctcgacgtcgggtgtatccgcggttttacacctcggacgtccacggccacaaacgacatcgcagttactccccgtcggcacccgccgtttagtcactctttcgaattaagaaatgaccacttgactccacatttgcggctttaccctccaccttcactgagtagctgccacgcgcagtgcggggtgaacggcaaccgcaacgctcgcaacaacagtaaagtctcaccacaaaaccctgacgtattaagtaccagcaaggaccagcacaacttcccaagtcacacttgtgaagcagatggataggttatacttggattaatgttacaccgttgacaatgacttagatcggcgttgaagtttaaaatgatcgctgcagggagctgtgcgctggtcaagataccgctggatgtacaagttctagaacgacgctgaggggttacgagcacatcaaatgctgctgcgacgtatacgttttagcacgactagctccctacgccgtaaaccttaaagctggcatgtttaccgaaaatatcgggaagaacagaggtttgtctgcacgtggtaggataaatgccgtgtgcattacgctggtggatcatcagaatgcacacagcatcggatttgatggagcaccaccgcgtcaacgggttgcagctcgatcaagttcagggacggcgatgacagtgaccgcacgttcaccttaagcggaacagtgcgagtttagtgtttgcggctctgaacgctatactggtgaagtgggcatctaactgcaagagatttggccagaacgcgtcgggccccacaagtatctatgaaagctcagcgagtaccattcacacgaaacttggacctgtaataatgtgaggtagaagagtaaatactgaaggcgtcgagcaacgtcaaaactcgtgtattattgtaccagtggtcttaactatgacaacagtgatagcctgtaaaaggagtatacaaaatttagcgccaagctccccctatagccttatgctggcgcattttcaacaataaagaggatatccctgaccgtatcagccttgagggcaccacgtcgtttaataaccgtattaccggccgtggaaaatgcgcgctccgaaggcactaacttggcgatacaatccaaccacttacgaacaagaggagcaatagtagcaaaatttgaatggttctgtttccaccacgacaaagccggcggatattgttttgctgtatagtaacaactgcctcacatgaatttcctatatagtttgagaacaccaggccacaatctgcaaataccttctcatctgtcaccccgacctgttttttttaatattttatgcgtacaacagctttagtaagttttggttgaatctgtctcttgggttgttcagcatttcctccgagagcgaagaacttggtatatcacagccaaaagggataactttattttttagaatcaagaacatttggcgaataaagacaattttcgccttctcaaatgtgtcgtcagtgaaaatctgggcgttagtcatgtagagatggagcagcgaaatccagagaaggtcactctctttctccttatagatatgcttctttagtagtgctaaaaggctctgacgaacggaatgcaacgttcggagtattcgtggagcgaaggacgcttcattcacgcttgtgtatagcacgtcaaacatacacttgttatccaacattattttgagcaactcaacgacaaggtgaagtgatatcaaagttggataagtttcaccatcaaggttttttgtcgtgacgctaaatggcgccaatagctcaatgaggcactgaatggtgaaccactgttcagcactagtgaagcttattttttttcgatgcggcatcatcaaattccttcaacccttttaccgacaccaggtaggtaaaacaaatctttcaccgccgaatggagcttgataaaccgctggagcatatccaaagagctattccatctggttgggccatcaatcttcacttttaggggttccttcggctcgattggagttcatccagtcggttgcgtcctttttgagagcggtgaaaatactcagtaagtttgtgaaaattgtgaacagatttgcgggtcgactccatatcgctgaaattgacttccgtgtcaccaacgctcgcaacatccgctacaaactgtgcaacttgtgcgaaaagagatcaatgtcagagcgctaactaaattatctagttcaccataggcttgcatgccgttttccagaatttccaaccgatcatctatccaggaaaactcaatttgattttttgaaacagattgcgaatagggaccaatatctaagacacttttaattgttttgccactgttaactccagcttgattcccattattaataacagtaccatccgcatctaccgtcaccttgtttatcatatcttcactttacatcaaaagcgcagctacaactaccaaatgcagggaatgagcagtacaaaatctgtgcgtaagaccaagttttgagcatgcactcgtaatatttgatgctccatctcttaaaaagcgcacgcaggttttgaggtcaagacgccaccacgtaaaacaatgagagagcgtacttgcaattgagtccccatcgtgctttcccggaatgagtcgtacttcgagagttcatgatttcatgttgaacaacgcatccacataatgaagcgtaaagctgatgtagctttcacattttactgatgtccagatgcttgatgaaacagagaaaaaagcgcacttctcgtcacttgacagcatatgtctgagtgtcaaccttgagtcagaggcttgggaatggatccgatccgtaactgtttggcgagatggaacggcaagaatctcctgtacactgttggtgaattaaccagtcttgatgaattcacgatcaccaacagcctcctcatgtcacgactgacccatttcaccaatatattctcgaaacaacgctggtcggattcttctgctgattcagatctgtttctcaggctttgacctcttcctctcgccgaatattttcctcaaacattctcagggcatcaatatgaagcttcctcatgtggggacgaaccttatttttatattttatgaatggcactccctcattgcagctcaatcaaaaaaaagcaccgggcctggtcgcgactgagcttttctcttttcacgcctgcttgctgctcactcgccacacataccatgaattgatactacaccttctccctttggcttggcactggatatgttcgtccggtgggacattgctgcacatttctgaggactggatatcttctgctagcctgagcgagtgactccgacgcaaaagttgctgatggtgatgctcgcaacgattttggtcttaatgggggtgatttcgtacacttccgcgtcgctcgcgaaggcactcttgaaggagttgtccctgatggtgtcattcgatatgatttggtcgagcgtgtccttggtcgtgatgttgctccagactttttttgacggtgtcgtgtgtacctgcgttgctgacgacgtatctcctgcacgttttgatttcttgcttgatggttgggagtgtggattggttctgcttcttcttgagggagtcgtagaacagctcgcatctgctacggtctgatcatccatggcggccgtgtgtccagcctgatcgatgggaggtctgatcagagaatgagccagctaccactgagtgaagctctcgaagcaaagatttcgaagaaggattggtatgggcacgaaacttgcgaatctttctgcccaaagctaccgcgaagcgcagtcggtcttgtttgccgactccttcgaaggctttcaatataacagctgatgtggcatccaacatggaactgctttgattggctaccttaaagactttttgccaattaaacgactacaatagccggggtgtcggctgtgtgtatccgcgacgtcgtttgcaaagctggacgtccagcccgtcaacgtccagatcgacgtcaaaggtcgggacgtcgacagcattcctcttcagtgatggcctcagacaccgccgcttgttgcggttgctgtgtctgtcctcgctgaatttcagaatgaggggctatgtacgttttcgtggcacctccgaaatggctacatacccatattattgggaaacagtgtccaagctttcatagtaaccccagcatttcgcctgttctgaaggcgttgcttgacaccgacctgcttgcctgcaccaaactgcgggaacgacgccggaacttcgtcaccacgacggcccgtcagttttggggtccgaagggggatacatagaggacgggagcggcaaaaatggccaaaatgggcactgcgctccgtctacgaacccatttctgaaagatttctggaaaaaccaaccgacacttgctcaatctctctgccactattcagcctgccccgagactgcgcattgatcacgtcgagctgctcacgccgcacgccgccacaccacaggtgctcgcctccatctttgtgcctttcttcgcgtgccagtttcgagtgccacatcgccgatcgttcgccgctctcccgtccagtctacgcattcggttggccgccagctgaaagcagcggcaacagctctcctctcttcctctcgcgcacagttccgcctgccgcgccgccgagaagaagagcgagcgaaacgcagcgactgagcagcaccattggccaggccttcttctgcctgcagacgcggccgcagccggtggccccgttcgcgagcgggacagtgctgagcttccacgcgcctggcgaacccgacgccgtgcgcgaacgcctcgcgcgtctactgagcctctctgcagccatgatggccgaacccgacgtgctgaccaaagccgatagcgacggcgatgccagcgagaccgccgatgccttccgccccgcggacgacgacctgcagcaactcacggtgcagtcgctcccctacgtcaagtgccgcgagagcgtgagcgccgtgttcagcttcgactcggcggcgcgtcgccgtgtttcgctggcgtccaacgcgacgtccgacacggccaattcgtcgcctgaacacgccgacaagttccccacgtcgctctcgtactcagtcccgtccaccgcgatagtgaccaacacgccgcgcggccggcccgccgtggacgtgatcgctacgtacatgaacacgtgtgaccagcgcacggtgaaatcggagctcatgcgtgcccacgcgcactttgcctcacctcgaggtacggacatgctcttttgctcttgcctgctgtggttgaagttgaaggccattgcggcgcagacgaaagaactgactgtagtgatttggtgttttgtcgttgacacgtttgtgttgcgttgtttgttgctgctgttggtgaaggtggcccgccgtcgcgcatccgttcgtcttccattatgagcaaggagggcagtgctgaatggggctggttcgccgacatcgactcgagtagcgaaagctggggtggcgatggttccatccactcgctgcggcgccgactaagcgctgacttgggactcatcgacgtgggcaacgtccacgtcctgggtgatgaacagaccaacacacgcatcacagcagcgcacaagacgtttacaatagtcacggaaggcaacagcaaggtggtctcagctacagtgtccattcccaagttccgcattgtgcagtcacgctccggcgccgaccgccacgcgcagtatttaatcaccctcatgctcggcaaggagctgtacgcagactggcgtcggtactcggagtttggtgaactagtgaagacgctggatgaaaagcgctacacgcggacacaagatgcctgggcaggaatcgagacgcgctggttcaatcggctagaaccgtcgtatttgcaccagaaatgcatcacactcgagaacttcatgcgcgagctcatgtacgagtcgaacgagccgaatgtgctgatcaacttcctcggcggctatctgggtaaggttaacgcgcggcctacggaccccgtggcatatcgcccagcagctcagctcccgaaggagcttcgtccaccacagccgcaacacgagcgagagctgttcgagaagatgtgggctgagaactttaaacggtcacatgtggagtactcgacaagtgggccggcgacccaggcggctagctagacagtgagctggctggaggagtgatgaaggcgtaaaggctccgtctgatgcccgttatttgattattcgtttaagaggttgcccctgcgcccttttctagtggactctttctcatttagtcgttggcagagctattgacagagctacaggaacctgcttttgaaggcagcagcggcgtccgaatagcagagctcgtgttgatagacaattaggtcggggatatgggagatgtcacgcaagtagaagaactgggcgagtcgcgtttgcgaaacgagcgggaacaaccacggagagacctgtgtgcagcatcaccaccacaacataagcatcacgtcgaagcataccaggccgactaacatgtaaacgaaccgtcgaaatcagcacatgccaggcaaagccgacgatcacgccaacaaatgcaccaacagccacttggtccttggtgtgatagcccaagcgcactcgcgagtagcaggtcgccgctgccagcaaaatgcagccgacaatcgtgatccactgctcgatgtagcggtgcgagttcaacctgcaatggagacattcgtcagttttcctgtgatccacggcatctcagcgtgacggacctcgaacacgtgtaggcgacagcattcgaagcgaagtacgagatgaattgcgagtgagccgagggcattcccgacccgctcatgcgtgcgccgtcaggccgttgctggttgattgtcttcttgagcaccttgttaatcacttcgctcaccacctgccaccaccaaattgagcgttagttcggtaagaattggctggcaacgcattgattgagatacctggccgaaaaacatgctgatgctgtccaagtcgcgctgggaggcgaccagcgtcacgtgcatgagggtcacgaacctacgagagcgttttcgcgttagttagctagagagcatgatattgacggcggaagtggatcgtacactggcgagagcgtgaagagcgccatgatgaggccaaaccggtcggtcgggtcatacatgacccacgtaagctcgaaggccttgagcgccttgcccttggactccgcaaccctggtcatggcgtcgtgcggtggtcgctttctgaagttgttggggagagttcgtgctaccggatggtcctccacgatttgcactgagcattgcagtgcctggtctcgtggttgctattgatattgcttcgccaacattatcggccgtgactttggtcggatggagcgtgcctgaattcgacaatggccctcgatgaagaaggaaaaccattgctgcactttcattacaattgattctgagtaaacaaagcacaacttctaggggtgtccacgtaacaagttactgagacttcttgctaggagaagcaacaggggccttggagcggcagatttcgtgttggtagacggtcaggtccgagatgtgtgatatgtcgcggatgtaaaagaacttcgcgaggctcgactcggccacgcgtgggaacagccacggagaaacctattcaaccaaaaccacagcaacatcagctactctcacatttgcagtcaccggaaaagtcaaaacagagccttctcgtaccgaggagaccaacatataccacacaactccagtgagcacaccaaacaccgcgccaactactacttggtcggtggagtggtagtttaggtgaatacgcgagtagcacgtcagcaccgccatcatgatgacactgaagatcgtgaaccactgctccagtcgtcgacgcgagttcatcctgatacacgacagcaaaggtcacatcatcgcaactcaatcaacttaagggctacagtccgcgcacctcttccacgtgtaaatcacgacgtatgccgcaaagaatcccatgaactgagagtgcgcggagggcatccccggtccggtcatgtacgcgccttccggtcgcggctgattgatgaatttcttgagcaccttgttgaacacgacgctaaccagctaccgagccacgttaccagcgtcagcgactgcatcaatcaagcaatatattgcaatagcaattgatatttacctgccccacgagcatacttatgctgtccaagtctcgctgcgaggcgacgatcgtgaggtacgtgatgaccaggaagctatatgtacaacagggggtcagcataggcaacccagtcgcatcgatggcagtaaacacacatgggagacagagtaaagagcgctaggacaacgcccagcgggtccgtcgggtcgtacacgacccacgtgagctcgaactcctgcacgcgcttgaccgcatccatcgtcgacgtcacgtttgctattcacccccaagtacgcaaaccggatgtagccatgtcacttttacgagtgcagttgtagtgaccgtcagagcaatcatgacttcgcaggccacgagactcgagagcctcttcctgctcgtgcgaggtacacaacagtcaacagaccgaacttctacttgcagcagctgcctgtgtgcttcctctatctaatgcggtgcatttcgtgttgtttggacagacggaagcagcgcgcagatccgcgagaacgcggccgagaagctgggcgaagtggctgcgcgctctccggagttctgcgcctcgattctgcagcagctgcgcccactgatagtgcactcggaatgggacatccgtgtggctgcatccaagtgtctcgatgtggtggccagatccctacatcgtgaggacgggaacgtcgcggatctcttcgcgttggtctcccgtacgtgaacggcgtagaaatgatgcaatagagtcgacgcattaacttgttatttatattgcagttggcagtcacgaagcttccagtgtggcgctgaatctccagacggtggatatcaagaaggtggtgcatgaaggcgcgccgttgctgcggagtggaggagaggtgagttaactgctgcctttaaacaggtgattgaaactaactgagaacgtgttgttctacgtaggaataccaatacgcgacgaatctgacggaggaagaacgacgtgttcatgcggcgaggcagcgacgtctattgttgcggcgtcttagtggagcgggaggacctatttggaaaacccgagaggactcacttaccaagcagctgcttcccagactcaacagagatcgtacgtgttggagtggaggtcttttgtgcgtgtttactgattggtagcgatgactgccgtacgggtgtggttacctgcagatgcacaggaaatcgctgatgacattgaggcgagcgaagagcaacccaacgttggtgagaaccctgccaaaggtatcaatcggaagcaagcgagtgaaatgttatcgatttcaacaagatcaaaacgtcagctagctgatggtgcagctgagtttgggagcaaagacaaacgggcaaaagtagatcacaacggcgatgcaaatgatgcggacatgaacttaccagttgggacgagctctgacgagcaattggacgagtcggaatcacagaccaagggttcgattgtcgcaagcttggtctcggatctttttgagagcatgtttgattccaagtgggaagttcggcatggtgcactcttgtcgttgcgggaaatgttattagctgcccatttcaccgctgcagttgaagcagtccgacccgcgacggacgagagcatgcagcgaggcttcgtggacaagtggctggaagaatgcctaatacggtgtatgtgtgtacttgctttggatcaatttgtggattactcggcggacgggtcgatctctcccgttcgcgaggtttgctcgcaggttttcggcattttgcttggaagtctttccagccaagatacacttgtcgggtatttacaagtggtgcgtacactgtttaatggatcgacatggcaggcttgccatggtggcctgcttggactgaaatatcttgttcgagctcatagcagtcacgcccaagcgctaattcctcttttctttaacgatatcgtggcagcattctctcagctcgattcggaggaggatgttctagttctggccgcagaaatgttcacagattttgtcctatacttggatcaggttgaaagcataaacatcacgaaggctgcgctgttgttgtggggatctctgaagtttaacgaaaaggctggcctggttactgccagtatagtccaagcactaaatgcctggtacaatcattctccagtcgcagagctcttgcaaagtgacagtgcagtcaggtcagcgctttgggagaatttgtcgtacacaatcccgatgctccatcaccactcgcaatctgttcgagcgtccagtgcaacatgtgtagcggccatcttttcaggggggtctttattcttgagggaaaactcggttagctttgctcatttttttctgccgcatttgctgcttcaaataattctggaaatgaacgagtccgtccagcaaagcttgcttgctgcctggaaaaaggtagtgagcagtctgtctaccaacgagctgctgatgactgtaattgctggaaaaattccattgtggatgaaactgttgtggtctacggacgaaatctattgtctaaatgttgaaatggtgaactctgacggcactgtatccggtagtgtcgttagcgatcctgccggccggataatacgagacaatatgtcttctcgtgtggctttcggagaggcgatcggttttgctatttcccatgtgcctctttccagcgcgtgtatgactgaattagtagagcttgtctgcgagggtctgtgtggtagctccggtgaacagcagtgcggagttctcgtagccctgtcaagatggggtttctttgaaaaacaactgagacaggaaagtccagaggaacaagcccggcgagctcggcacttgcaagatacaattgggccctttctgagctcgttcgctggaaatcattggaggacacttcagcctgatgccctgagcgaagaaaaattagtgctctatagcgagcaagctggatcgctgaaacgtgttatgcagatggaagctcgaattattgagatattctcctctgtgggtattgcgctccagccctcagaaagatcgggctcgtcatctacagcggaaatttcacgccagatcgctgagcacattgctctttttccctacgagaagttgcaagaccatccgaacgaatacgaattggctcatttcaagcggcaagacctctttcttgctgatgatctcgtgcagcaaagtttctctcgctgttatcaccgaatacaaggtcttggtagcagtgcactctgcgaaatactgccaatccccaaaaagagtgggtttctagtcaaagcactcatgagttcgatcaaagaagaagatgattcagcattccgcacgatttcatcacaaacggttgcagcctttgtggtcgatcaagcacaagcccaaaagaagtgcgtcgcgaaaatcatttcgaacctctgcaacagcgcggcggcattggtaggcgtatcgggtggccctttggataacattattggattcagtagttcgccggagaacccgagtgattctgtttctcctggtgttttcaaaaagacaaagatccgagctgatggggctgaggctgtgctgcgcaaaatctgcaactacgcaggagattctcttttcgatgtctgtcacgcattggaggatgccatttctaaggcttggaaccaaccaaacgtagacgagccgacaattcaacggtgcatgtatttgatcgtcttagtggcaccgcatgtgaagagcagcgcaatgacaacatgtttgtcatggctttgtcaacttgcacaactgacacagaagccatttgttgaaaatcaaacgcgaggcatcgtggctcatgccattgctattatttgcaagtatgctcaaggacatcatcgcaaaaacgcgatgctcgtcgtgtatgaatccatcttcgtgatattttcaaacactggtggcgctgacgtcttaaaatcggaagctctagaaggcgctgtgatggtgctcgaccgcattgtccgctcgcttggagcggagttgacaccgtatgttcccagcttggtacactatgcgatgaaaaccatgagctcgcaattcaaacttgttcgaacgtttgctgccggggtgtttgcggacttggtcccgttgattccacttcaaatggatcttgagcttcataaatctgaccagctacttccagtctcgctgaggacgattgtgaatcagaatgctgtttcacgtagcttcctggagagcttcacggaagggaaggctgttcagcataccaatgtgaagccctggcttgctccagagacctctttgcgcgtataccagcagcatggcgtagactggctatgcttcatggccaggaacaatcttcatgggatcctcgccgatgatatgggcctcggaaagacactccaaacgctttctgccatggctgcaacactagcaatgacatctacagcccaaagcaaacgtccagcgaggccttgtctcgttgtatgcccaccgattatcgtgcatcactggatccaagaagccaagaaatacattccaggatacttcgcatctgtcatcgactactcagtcccagcaagtgaccgaaaagtgttgggtcacggcaaaaatatcgtgatatctgatcacggcacgactttgatcgtgactacatactcgatcctgcggacagacatcaaccgcttgggagatattgactactcttttgtggtgttggacgaagcacatttgattcggaatccatcgacagcgttattccgtgctgtatcgaaccttcacgcatcacatcgtgtggccttgagtggaacgccgctgcagaataacgtcacagacctgtgggctctgttcgagttcctaatgcctggatacttaggcgactttactttattccgtcgcgagtttgtccttccaatcacaaagtcaaaagagcgcaatgcaacgtcaaagcaaaaggagctcgcagcgattgccatcacaaaactgcaccaaaaggttctgccatttatactgcgacgaacgaaagaccaggtgctggaagagcttccaccgaaaatcatctcgaacgtgctactgccgttaacttcgctgcagaaacgtttgtactcgctggcgtcgtcatctgagagcgatcccacaagctctgtggcatcttctcggtctacgaaggtggccgagccgaaaccattgacgaacgtgctaacaaatcttcaattactgcgcaagatttgtgtacatccagcgctcgttgctgacaacgcaatctctttggggcttaatacgaaggaaacgggggccctgcttgactggaaaagctcaggaaagatgactggattgcgcgatctattggttgagtgctgcgatattgcagcatggactcaggaaggtcgccaaggacctgcaactgaaggtgatgacgatagcaatttttctcctcatcgatgcctggtgtttgcccacctgcagaagacgctcgatctcaccgaggaaatgatgcgggacgcattacctggtgtaacttatcgacgcctcgatggacaaacacctcacgccaagcgtgcagacattgtacagcacttcaatgccgatccgtcaattgacgtcttgctgctgactacgtcagtgggcggtttaggactcacccttactggcgcggacacggtgatcttcatcgagcactcgtggaacccctttgttgatctgcaggcgatggatcgtgcgcatcgcattggacaaaagcgaaccgtgcgggtattccgactaatcatggagaagtcacttgaggaacatattctgaatctgcaagagttcaaagaacaagtagctgctacggttgtgcagaagagtgatgctcagagcagcatgaatacgaacacgaaaggagtcctcaacttgctgcagacgtcctcatcagcggtggctgccaaggagcttcgcgcgtcagttacagcaaaaacggcaggtcaagatgatccaagtgtagcagcagcccttccacagggagcccaagagctgctagaccagattggtgagctatgggatgagtcccaatacgactcgttagcgtttcctgagcccatccacgagtaaccatgcgcataacgattcagactcaatgatggctctacaaaaatatctcctatgtattggatcatcgctgggatctccagaatctggagtccaggtgagcagaccacgccgagttggtttactgggcatcaataaggctttcgacgtcgagctggattatatccgcgtcacagtcctatggcagaaaagcaaggaggggagacaacgtgagcgaacctttacgcgtaatagcacttgcacagctgtacaacataccaaaattgacgcgacgttggggttggtccgaccacggtcaccgtgcacgaactctttaacgtaggttcctgcggatgtggtgaggcgcaacatcatgtaatgcttgttcagcacctcacacttggcagcatggataatcttcggcctcgtcatcagggttcggcgatgcagcacgcgaataggagtttgctgctgaatggtgaggtcttgaattgcatcaatcttcgcaacggactcgggggtcaattcaccttccgaccacacaacgcagctgataatggaagcaaatgcgagtttgtcagtctaacctgaggtagtagtttgtcccaaggtaaaacactcaccagtaggttttcttctttgagtctgctcctgcctgcagcccagcgaaatagtccttcgtcgagctcttaacctggacaatctcgacagcaccattgtttgcggcattcacagccttctgaattccatcgtactctgattgattcaggtacgctttcttggcgtctgcaggcaaaacaaatacggttgagataggccaagtctaataccgctacataaacgtaaagttgcagcttacccaaaatctccaagatgaaaggccttccgtttcctagcatgcgcacgtcgacatcttcacgacctgctgtgtgaaacttgtagcctgcacccttgaagaatggcagggcgatgtcaccgatgcactcttccacggaggattcgcccattcgctcgccgtcaagaacccacggggtctggctgagtccacgctggaacttcagatatcttccctgcatgtacaccggatcgcgttcaaacgtggcaacagcgtgcggggcagtagtgatccgagtcggtggactcttaatagtctcgggcatggacgaaagcgtccccaacgcgcgggaaactgcaccgaaaccatcaaagaccgggggagggccacgaccgcgcttcttcgtttctagttgtttctgccgaagaggaagcagcgaacacacgtgagttattgcacccaagcaccagacaacgtgctcaccataccctgatcgacggaatctggcgcacgtcgtcggcagattcgtcatggttgatgtcgatcagcactgcaaactcgctgccgttcgcgcactcagcctggttgagcatcttcgcgataaaaggcgccaacaaagactattcacgacattcccaaccgcaatagacgtaagttcataccatcgatcgcaaacgcccgtagttgtttgtaccttgagcacatctttcatatcgaacggcgttttgcgcgggaagtccgccacatccgagcgcaagaagtgcagcagcgagtactcgcgcagaagcaccacactgggcaacttgatgttgagtgcgaaggccttgacgtcgtagtcgcttttctctgccatttgcttcaggtcttctaggatcttcgaatggaacgccccgttcaagacgcccacgcagcatgtacagcctagagaaataaaacacaaattcaattgcaaatggtgaataaattgcaacaaaacccacaacagcctcatttacacatgctaacgaaccggaaacttcctgcggctcgaaacccgagacgccactctccttggcaagctcatgcaccgcatccaccagcttctgctccgagcatctgcagcggcaaacatcatcaaagaagcgcgcagggtcagtcagtcgtgtgtcactgtgcagctgggcacatcatggtgatctccggccccacacgcacgtgtaaatgtccaagtcttcgacgtaggcgaagcgcaggcagcagcgcacacagacgccgagctctctcgcgcgcaaaagtcccgtggcggacagcaggccgccacgcagcgcacccgagacgtccatggcgatgctgctccagtcaagtgttgatgttgatcgtcaacagagagcagggactttcgaccaattggcgacgtggtatcgacccgttttcgaccaattgtgaaggtgagctcgccacgtttacgacgcatacaaatcgcaagtgtgactcacgcgctcccgctctcggtcacagtcgccgatcgccgtagcggcatcgcaaggggggaaagcccagtcgcagtcagtctcggacgtggcggacagaagaaagcaatcgcagccatgccgagcttctgccccaagtgagttcgtctgcctctagtctgcagttatatgccaatgggagctaacgaacgcgctgtgccgtgaccagatgcggggactttgtggaagcggacgcttgcgagaagtgcggcgagaaggcggtgcgtgacgttggtttaggcgcggttttaggcgcaggtgctgacgttgtagctgcaggtgacgacccaagagaatccgtggagggcggcgtccaaggttcccggcggagagcccatgagcgttcccgcggtgcccacggccttcaccgccgccgcgcgtatcaaaatgatggggtgcgtcctcattagcagtcttagagacttttgactgctatcagtgcagcgactgacgccgtgtgtttgattccctcgcgtcgtgcctatgtgtgtgcgtgcagggtgcagcccctgccggagcccgcgcgcaaagagcaggcggaggcacgcttcaacttcgtgccgcaagatccaagcacgcagctggcgctggccaagggcgatgaggtggatgtcgtgcgcaaggacagcgacggctggtggttcgtcatcaaggacggcgagaagggcctcgtgccgggctcgtacctgcacatcgagaaccccattatccccgtcacggatgcggcgatcctcgcggcacgtgagacctccaatcagcggttcatggagatgcagcaaagctcgggagcaactggacagggtgccgagccgggagtggaataccagtcgccgctgggactcgaagcgctcgcgatcgcagactcgtgcgcgcccaagccgcccaatgggcctgcccgcgctgatgccaagtctggcggcaccgagtttaagccgggcgaagtgcgcaagttgagaaaatgctacgcctgcaaggagacgattctgggccgcacgaaagtgtgcaaggaccagatattccacgaaatcgtaagtggcagtggtcggcttgcttaaaacacagtgtgtatgctgattgcgatggtgtttatacagtgtctgctgtgcaaaggctgccgcgagtcgatcgaagagggcgaggacttcaccctcatcaagaaaaaggttcactgcaagcactttctagggaatgctactggctgagcgctgaccattgtgttggaactgtaggcctaccacgctgaatgcgctaaagacgtcaaccactgcatggtttgcgataaggtattgctcgctgctgcaagcgtttcggtgttttgcgaaaagttctcacgttgatgcatgatgtaggaaatcttgggccgagtgtaccgagctggcgatggcgcattccacaagctttgcatggtttgttcagtttgcaacaaacaagctaaagaaggtaagcaacgccgttaaactggaactgaaggatggtggagtgtgcaagcggatgaaactcaaattttgatgttcacttaccgtttgacgtgacagctggatcgaagttggaaaacgacctcctcatttgcggtgaatgcgtcgcggagaagggtaagcgcatcttacggtgcaatggcgagtcgaacaataatgctaacaactacaatgtggcatgcttgcagagagaaaagagagggaggagagagaagccgccctcgaacgtgagcgggctgctgccaaagcgaaggctgaaaaggaggctgctcttgctgcagcacgcgcgttggaagaaaagaaggagcgtgaagcacgcgaagcacagaaggctgtcgaagacgctgcgcgcgcaaaggttgaggcagaagccgctgcggctgcggctgctcgtgcccaggctgagaaggaggcagcagaggcggaagcgttgcaaaaggctgctgcagctgctgtggcaaacggcaacgggcggtctggaaccgacagtctgcgatccagcacatcaaatcttgatgagtttgatttggaagctacacccatcgacatgtcgggtcagaataatcgtatctcggaagtatcggatctggactcgattcttagcgacggcgattacgctggatacatgggaagtggccaacgggagtcgctacgcttgtcagaggttctagacctcgagtcgtatgatgaccgtttgtccgacttgagcggcatttcagagggaggtgctggtagaccacgacgctccacaattgaaaggaccgtgaacgaaaacgcagaggccttctcggaagatgaagacgatcgtgagctgtgtggtggttgcggacttgtcctggaaggagaagccgttggtgcattgaatcagtacttccactatgaggtacgtgtttgcacactgtggagttcatcatgttagaaatcgtttgacgaacaacctatgctgcttgtgctgaacagtgcttcaaatgcagtcactgcagtcgtgttattgcggaagatgatggctatgcagagaaagacaaccaggtactcttcttcttgaatttggtaccataccctgccctggaaactcatctcacatcttgctttttgcaggctttccaccaagcatgctaccaggctcgattcggaaagaagtgccatcggtgcagcaaggttctcaagggaaaagtcgtcaaggccctcgatcatctctaccacccggattgctttgtgtgcttcaattgtagtgcttctctttcaggtacataccataatagcccgctggatcgtatgtgtgaactactcaatactaacacagggcgtgtgtgatgttttatcgtccagcagagagtttcttcgagcacgaaggtcaagccgtttgcgctaaatgcaaacacgaagcaattgctttggacgaaccaagcctcccgtatgtcgtgtgctttctgctttaactagcactgcctaccaactaaatttgtgtcatatgttttgctctgcagagagaacgtggcaccagtgtctaaaggcattgctggttatcggttcgacgcccaggatgacacacaattgactatttatccgggcgacgaagtcactatccttcagaaggtttgttcatttagaaggcggctcttgctgtaaacgcttcctaacgttggcatcttgcattgtgtgcgacaggacgacgatggctggtggcttgtggatttgcgaaacaagcgtggttacgtgccaggctcctacttgatcgagcgcccaattgaagagccaaagccccagccgaagaagaagaagagtgccttcaagagtaagcccaaacgtacaccgggtctttgcagctcgtgctcgacgcaaaacccggcggaggcacgcttctgccgaagctgtggcaacaacctgaaggcctaactggcggatgggtaagaatgagaaagtaacatgcaacaagcgacagtttgtttagagtacgcgggcggacgagaccaaccggccttatgagccgtgcgatggagtttcttttgcgcggcagtggctgttagatgcttcgtgtttgctcttgtgacaagagaatgtcgctctcgtcccctgtgttcttcatcatgtaggtgtttgcgctaaggaaattaactgtcacttcagcgtcccagttttcgtgatgttgtcttcacttgagtattttccaacgagaccaccgagcatatcgaggctggaggcgatcatgtggagtaaagtgacgaaccgctcttctgcagctttcctattggcgagcgcggaatgcagtctcggcgcaccgttcgcatctgggatcgggtgcagccatccctctggcatcccttggcatcgttggagcagtcgcttatggatgtggaagtgatggctcgtcgagtgttctcacctcactttccgacgtttgcgccgtttgcgtccaacgcgatggcggaagtgtatgacagtgacgacgagtttttcaaggacctgccaatcaagagaccggagcaacagacaaaagcagtgttagaggctgcaacgccgcgtttctcttaagagctactacagcgctcaacgccaagatgcagtgcagacaacgcagcaatgaaagaggaagctgctgcgacaccttcgactggtgaaggcaccgctgcgccaagcccatcgtacagtgccgtactcatacagctacttcgagtgtagtggccaccgtatcggcactgctcgtcgacgctatgaagactcgacggcgaggctgaaggcgacgcacgagcgcgagatcgacgtcaagaaggtcaagatgttgtggcagcggtccaatacgcaggacgagggcgggcacgagacgatttgctcgagtggcaatccggaggagttcgagagggaatggaccgacacaccgcttggccgcgcggaggacgagaagctggccaagtggatggaggagcgagagcgcagcgtgtacgtggccttcggcatcgagtacaagccagtgatggagcccaaggtgaagcaaaatgacacgaccccagaggagcgcaaggtggtggaagagggcgggaaggagacgatccaggaaatatccaagaggagacgacgcccccgcagttcttgtcgtgaagcagccgaccaggcatgaagagagtgccgctccctcatcttgaacgatcgggatcatgttgaagtctcaagatttgagcgctgaaaacaagagtaaagcttcacaaaaggcagcttcggtatgttacaaattaattggtgtctgctctcgttcgcgtcttctcctccgcttcgcttgtctcccccgtctcgctcctctccttggctcttttcctatttctccgtttgtgcgagcttggattttgtgatacttccgcttccgcatcaagctgtgggtccccgtagtatcgaaatgcaaattgttcatgttcattctgaacgagatctgcaaggcatcgcccagtccagacgtcgacaggatactattagtgcagcgagggactacatccttgccacgatccacggacgcgagtgtcagcgcatgaagacgatagcttcgctgattaagaagtccaagacggagtgcgagagcctcacgtacgttggttcctctctttcgctggtccctatcgtggttaattcacggtatcttgttgtggtttgtagtgtctgtcattgctcaaaaggttgtccacgggctggagaaagttgtacgttcagactgaagtccgtcttgctcggtcgtccgcctcatttggagtgatttctgtgctgctgcaggaaatgtgttcgggttgccaagatgcgatgcgccgtccaaagctggtggcgttggccgacgccgacagcaggcgacgcggttcccttggtgcgtgttggtgtcagatgggagactacatgtaagcttatagccatctgaccgttgttcgtggtctccatcagccccgagctcgaaccaactgcgcgaggagcaaaagatcctcgagatcgacgtcaagcccggctggaaggacgacaccaaggtgacttttgaaggtcaggacgacgcgctgcccggtcgaccagctcaagacatcgtgttcgtcatcaagcagaagccgcacaataagttcacgcgtgatggcgacaacctgctgtaccgcgcctagctgtcgctgcgagacgcactgctgggcagcggcacgttgaccatcaagacgctggacggacgcgaggtgcccgttcctctgggaggcgtcgtcgctcccggtacgcggatggtgatcgcgggcgagggcatgccgctgcagaagagaccctcgcagcgaggtaacctcgtggtggagttcgacgtgcagttccccaccaaactcacagaggcgcagaagagcatggagcgccaggtattgtaagcttaccagttttgtcaggcgcggttccgaagccccaggccgcgccgcaagatcatacgccagcgccagctgagtgcgacgtgcttcggtgtgcgagcgtcatccgtttagaagcaccaagatgaacaacaaggacgttgcttttgtccaaatacgtacacgctggcacatttcacaaatgcttacgatgcaccccacccttttaagcgcgtggcggcgacgcaccactaccaccacagtgcagcaacagcagcagcctagtcgcgcatcgaacacggacagcagcagctcgcgcagattgttgccgaacttcttcttgtacacgggcttgatgtcgcgcagcacgatgtggtagcgcaccacggcccttacgcatcatagcaatgccttcgttgacttcgctcatgggcatctgttgaacgagttcaaccacatcctacacataaacaacgtgatgcttggattcgtactttacaatctcaaaattgtattttagctacccatcatccgagctgtgcctccagtcattcccccttccaacgtcatggacgagcagcaggacgagcccatgctcaagccctgcggcggcggcgcctcagacgccagcgctgcgccttcgccataccagcttaacggcttcggcgtggacgcggctacgggcgagtccaagcgctggctgcagagcctggagaaatgcatccgcgccatcgtgagcatccgcctgctgagcgtgcgcgccttcgacggcaacggcgccagcttcagcgtggccacgggattcgtggtggacatggagaggggcatcatcctcacgaaccgccacgtggtgacgcccggaccggtcgtggccgacgccattttcctcaataaggaggaggtggatcttgtgcccgtctacaggtgatgactcaccctgaccccatgaacagaggggagatgggctcattgcttttttctttgctgtttggccgtagggaccccgtgcacgactttggcttcttccacttcgaccccaagaaggtcaagttcctgcagctccacgagatcccgctgcgacctgaagaggccaagagtgggtgacgattaacgcacgagtttctgcattttaaggggacgtattgacgggttttggtgcttgttgctgtgactttcttagtcggtgcggagattcgtgtggtgggcaacgacgctggcgagaagttgtccatcttgccgggtattttggccaagttggaccgcgacgcgccgtcctacggctccagcacgtacaacgacttcaacacgttttactttgcagcggcgagcagcactagcggaggatcgagcggctcgccggtgctaaacatcgatgggtgcgctatcgcgctcaatgcgggtggagcgaagaaggcggcgtcgtctttttacctgcctctggaccgagttgtgcgagttcttggacttattcagcagggcgtgcccgtgccgcgcggcacaattcagactattttccgtcacacggcatttgatgaagtgcgtcgactgggtctttccggcgaaacagaggcgctggtgcggcaacaatttccgcaggagacagggatgctagtgatagaccaggtcatccagggaggaccggcgcatgatcagctgcggacaggtgacgtgctgatcaagtttgccgatcagtacggggcaacgtttctaaacattgaagagttcctggattctcatgttggagagaccgtcgaagctcagtttcagcgcggagaagaacagtatacagccaccctgagcgtccaagatttacattctattacgccagacagatatttggaaattggaggtggcatcgtgcatgcactttcctaccagcaagctcgcaacgcttcactacctgtcggtggcgtgtatctggcccaagcaggtcacatgttcatgaaagcacatttggcccagccctgcatcatcacatccgtggcggggcaacccactccgacattagatgactttgcgcgcgtgatggcctcactgcccaacggatttcgtacggtattgcgctacttcatgattcgcgatcgccatcgtgtccgcacagccttcatcatgatggatcgtctttggttcccaatgcagatgtgtacgcgcaacgacgaggacgggctgtggtattccaagtcatatattcaggatgatacgtcatcacttgtggcagctacagactccaatccgacatttgccccagcgccattaagctttccgggaggcaatgcactgggcaagaaaacgttgctgtcccttgtaatggtgtcatttgacattccttatatgattgatggtatttcgagctcaagctaccacggaatgggagtcgtggtcgatgctgagcgaggctttgtgcttgtggaccagaataccgtccccatcgcgttaggcgatgtgttgattacgattgctgctaccgttgagattcctgccaaagtggtttttgtgcacccagtccacaacttttcgatcgtccagtacgacccaatggcacttggtgctgtagccagccacatcaaaaacgtagtgctggcggataagccgttggaggtgggcgagacagccgattacatcggcctcagtagtaactggactgtggttaccatgaagtctgtggttactaaactagaccgtcttgtgctgcgcgatttccagccgccgcgctacaaggcgtgcaacatcgaagtcctgcatttcgatcgtatcacgaagtctgtaggcggtgttttcattgacgaccaaggcgctgtcaacgcactctggctttcatttagctaccaggacagtgccggccgcaaggaagtctttcgtggcctgccagttgagattgttcggcccatcattgacgagcttcagacgtctcgtatcccggcatcaatcaacatcctaccggcgcagttgttgacgtactcgctctcgaaggcgcgttctggtcttggactctcggacacatggattcaaaaactcgaggcctgttacgaagacaagcggcaggtgctcggaatcaaaagatgtgctgctggtacggactgtgccagtaagcttgagagcggcgacttgcttctagctgtcgatgggcaggctgtggtgcgcgatgcagatgtagagcgcgcggttgatggtaaaacagagctgaaggctttggtggtgcgtgatcagaaggaaatgatagttactgtaagcacatctcagctctcagcgatgggcacagatcgtgtaattgtctggtgcggtctcgtcattcagagtccgcactacgctgtcgcaagcctgggctatattccggaagaaggcggcggtgtgtactgttcgcggtggtgctatggatccccagcgcataagtacggattgcgagcaaccatttggcttgtggaagtcaacggtgaacctacacgcacgttagacgatttcttgcgtgtggtagaacgcctggaaaaccgagaatcggtgcgtctgaaaacgatttccatcacacgaagcctaaggtattcacgctcaaaacggactatcactactggcctacgatcgagctgcacagagaggaatgggactggaagtatgtggagcaccctgtggtcacataagtgttgtcctgagtgcagtgaagaccaccgcattcaatattcttcatcaagcccccaacccagaaatcaagaaatgagaagaccccgaagaataatacatagagcacactacacagagtgcgaccgcaccgccatcgctttgaaatatacacaggtgcgtgtttgcctcacatcccgatgactattattaggaaaccagcttggactcttacttccaacgtcgtgttgcacagctcaaaagcagatggggacggtgggtgttcaaattgaagatttaagcgtgcgttgtctaccggcaaggacagaagactgctccgagagtgaaggtactgtgtacattgcccaacaggagcagcagcgatccaacacttcagctcaagacgacgagaagctcgatgttatccaaacaaagatgttcaagcgacgacgacttgcaactgctgtcctcgatggtgacccgggacgtgccctcccaccaaagtgttgcaagcgctgcaagagcgatttgaacggaaaacttatggtgcggtgcgctacgtgcccattccggtgtcacttgtattgcttctcacctccactcaagcagcaccctgggttcctgatccggcggcaacaacagcaaattggtggccaaaatgatgatacttcgccaatttggaagtgtgaaaagtgtgaaggcacgtcggttgtatttaatgtcaaccccagaccttcgtcagtgacgacatcatcgacacgtagaagctccctcgtgaagcgcaaaaacgacacacaagcgcagtcaaccactactacgacaattacggaagaaaaggacgcaggatggatccaaccgcagtggataaaaaatgccgacgttgtgtttgattggcatcgctttcgagcagaaaaggccaatgtacttcgccaccacgagactgacgctgatcaaaagcgtagtggtgttgacgaactggttttttattcgaaatcctatgcgctgatgaaaaaagttgcagcaatttggcgcggacttgtattaaagcgacgatatcgacaccagcaagaactcgaagcgaaagccatgaattatccaggtgtgaagcgcaaggtaatgtgtgtgcgaatatcaaccgagaaagacacatcagactttagcaaggaggagttgtgtgcagaagaggaagacgtggatctggttgaggacatgaaccggtacagaccatactctcggagacctccacggcttccaattctgtgggaaggagaacctccaagcgcattaatgtgttggactacgggtgagagatccgacgttgctgatgtcttggctgagatcgaagagcgggtgtgcttagcgtttgaggatgctgatgcacctcccacgaaagaaacgataattgctaccctcgaagatgttactgacacaaactcagcgggatgcaccgttgctaacgtcaaaggtgttaacattcgcacagctgctgcgattattcagaggatattcgctcgccattgtatttggcgtcgaagaactgatcgagccaatcgacaacgacgcgtgatcgaagaagcgaaacgcaaagcgaaggcgcggtcatcggttgctctcttacggacgtgcatccagtttattgttctgcttgtcagaatgcttagtcaggctcaaaagaagaaagcaatgctgttggtgctacacgatgcagctgaagaaacccaagtaaagaatagcacgttgacgaaggagcaggatggaaaccaagatgcagcacgtttagctcgagagcgtacacagaaaatgtctgttgtgcaaattcgtcgttttttcattcggcgagtccacccgtacgtgaagctgaagaaaactgtgatggcacggcgaatacagcgttggtggaaacacaaaacatacctgtgcaagtggcgggagactgcaattacgattcgactttctcatagaagtcagtcatgcacccgaattcagcggttttacaggcagacccgggctcgagctcaatttcgcgttttgatcgaaaaacacgcactgaagaaactacggcgagccctaacaggctggttgattttacgtcttgctagaaaggaagctaaacgtagtgctgcgtacgaagctgcacagctcacattgacagaagagagctccataccgcaagacgcgcctctggatgaaatccttgagaagcgaggattggacctctacgaacagggcgatttctggaatgctgcatcgatacttgagcgactttgtgagatgagaacagggaagttaacccgagcattgcaacaagcacttgcatattcacaccacagggcatggtacacatcttacgaccagtttaacctgcatcgagcgtatgagttgtactgcgactcacttgagggctcaccaaatggtgacccacgtgtagatccttccgtactacatgaccttgcgatcgtaatgatgcacatggaacactttggcgactccttgcgcttgctagccaagctgatcgagtacttcgcacgacaacctcagttttcactttggctgctacttgccgctgtacaattacagcaacgcggcgagtgggaacaaagtgtcgggtatctgacgtatcttcacgacttcccccccactccgtatttggagcgagatatactggcattgtgtgcaatcggatacgaacaattaggaattgcagcggctaaatcgtctgagggacgcaatgcaaactccaattttgctcaagaggcatggcgtgctgctttacgacaatggaatctcgagaaagttactgctggagaagctgacggaaccgcgcgaagtcgaggaaatgggcgtacgttgacgttacgacaaaagtgggaactgctaacaaacctggggcaacgagctctagagcagggtcattatcttttggcttgcagggtttacatctatgccttagaacgtgaatcaccggcttctgacctcacttcaccttctccgtacgccagctactacaaacaagaaaaacatacagcctggtggaatcttgccgatgcgtttcgccaccttgggcacctcgatttgtatttaaccgctgcacagcgcagccagactggcagctcagctgaagaagtcgacgaaaacctgcaagaaagctggcgagagcttgcgagccaacaggcatccagtttcgagaccgaactaaggacactgacagtgcttgaaaaactgcgacaactagctggtatcgctaagtaatgtgcaaaatgaaatgtgtattacggagatgtagatagaatttaatcgtcagttgaatgttgccggacgttcacgtcgtctgtatcttcaattgtagatgtcaactctgccatgagatgttcatcgtgcctgtgatgtcctcctgcatttgtagatgcggtcattcgcctgcgttgagacttccgcctgcgtggaacggcatcttcgtttgatggctgcgcctgaatgaaggtactttgacgttgtggcgaattacttttgcgctcagatcctcgtcgctgaatatcttgtaaagctgcgagtgcatcgcgttctttagctcgcatcgctagaccacgggcgccagccagcgcacgattaaaatgcagcggattttgtgtcatagtcgcaaccatctcgagctcgtcgtcgtcgtcatcgtcactgtcatcatctccacgctttccaccctgctctttatctttttggttgcgatgccagtgactaaaaactttctgcacccgagtaatacgtttgtagtagtcgggtcgaagcgctacaacaagctctgttatcagaactagtgcaaagtacacacaacttgcggctacaagaagtgtggtggcaacggccagcatttgtgccgaagtactcgtgaatccagtatcgttacttccaagccgaccgctttcaaacattacgccggccagtaacacgagagaagcgctgaacaggagacacgcctccactgtgttgtgatcccagagatatccgaccacgttctgcggttcttgtgatgcgaggatggcttcattagctccacgaagtcctgaagtgggcggagcagaactaccagtcacaccattacccccatggtcaccatggccgctcttccaggcagttaccgtcgaagcgtgcatttcaagacgcaggtgagctggttgaatgaatgcctctcccttggcttttgcacgccgagtttccaacaggactcgtttggcatagtccgctaagacccgctgtgtctcatagacactcatgtatgggcggcagcgtacttgtagcgcgtagttcacaaacaatacaaaggtcgctgttgccagttggaacactgggtcttgacggaacagaagcccgatacctgcgagtaggaacttcctcaggataatgagcaccatccagtaccagtagcccggcttgaattgataatacaattttgaaaacttcttgcggaacacccagcagtttggattggttcggcgacttgttcctctctgcatagcccgtagaagctgatcttccatgaccaacgctcgattacgagacaacgtcagaagtacaaagagtggatagcccagcgaatataccacaaatgccagcactgcgtacgggaagagctgcatgtgcaacccgcctggtttgaagcactgagtataaatggccaccatgtacagctttccgtccgacgggatcgatccgacgcagttgaagatatccaatgtcgttcgtgtgagatacaagaaaagataatacatcatcacaagcgttgatccgaaaagctgcggcaggtgcgatgttagacgtgtgcgtcgtttctttataaagcgtttgtaagcgtaaagagctacaaagaccccgagcgccgtggccgccatgacaagtgggaacacctcgatgatgaaccacttattcacaaacgagacctggagtgaaaaacactcgggtgcaatcagctccaaattgaggttgaatgcagacagagtcgcgaataaattaatcaacgacgttggccacgaaatacgagcgttgccgaagatggagagtgtctggaagtaatcgataccgatagatagtagaccaaggctcactccttttcgagtaagaagataactcacgatggatgcaacactaatacagacgaccacaattgcgaccgacccccacttgttctccgggcacacgttgcactctgaattgagcttgtggtagtttgtctcgcattcgccgcaccttatgtcagtataaccctccgcgcatttgttggcacctaggcacgcatacatgggatcgcatgcgaggacaacctcaccttcacgccagtagccaggtttggctaacggcatctcgtccccaccagcacaagtagccccctctggacactcgtcgcagaattccgtcgaagttcggtagtaacctggactgcattgaaacttcagctgtgtttgactggtagcagacatcacgttgccacctaccgacagaataatgctcgtaacaccagccttcgttttgggtggataccataccaattctgaggcactcgtccagactacggtcccggttgagttctctgttgatacgtttgtggaggcaattgaactgtcggacactaactgccgatgagacgtgtcttcaaaaatactcgagatacttggcaagtaggagatgcctggggaactcaatttgcgggatattgactgtactgtatctgacgtatcgtccgagccagcaaaataaatggtaattgcatcattggggaaacaaccaaagtcggtacccttgatcgtgatagcgttggctccatttgcatcgggctgatttggcatctgagcagtgatgacgggctgattgtacgaaaatgttaaggcgttactggattggttgtcaactataacgtagaccaaaacgtcagcaccatacccagcaggcacacttgtcacaattacaaagtcagagtggcttgtgaaatcttcagacacatcgggtgcaggatctgttgtctcacacatcatttgagcccctgtagtgtcgttacagacctcaaggaaaccaaacgtagggctcacgacgtacagcgatggatagcagacaaacgtatcagtatcaatacagggaccgctggtcgagtattcacatgtgtcggatccacaagccctcgggtgagaactttggctatcaacgcgtgcatcccggccaccaataagcactttagcacgagttgatccgaaatttttaccgtaaagagagatggtgtaacctccgcaagtcgacaagttggtcgtattggtttcgtagccgatcagctcagggctctgatagccgaatgcgattgatttgttcgcaagctgaagccctgatacagtcatattgacaatacgtgagatgccttgtcctggtggcaagttgaatttgatgtttgaatggctgaagtacacgatgtcatcgctcgtaatattcaccgattccatcacgctatcggtgctggccagctcgaggtgccaatcaaacgtctggacaccaaagtttttgccggcaagtgtgatagctactctctcgcacagctccgtttctgcaggacaggcacgtgaggctgcctttgagcaagtgcccgggttgactttcatagccaccgcactaacgggtggatccaccatcacattgagccaacctgcaggtgttgggttcactgtcgcggtactagcacgataaatacgttcatcactttgtaacaattgccaaaccccaccggtgaatagcagctgtacatttgtcgctacatcactcatgtacactggcttgttgctggagtacacatctgcctctgcagtgactgttactttttgcaacaccccatttagcagtgtttccacgcaatagctaccaagctcggtggtagttgagcttgcttcgaggaaattgcactcaaacggtgccaaaagcgttgcgcagggacggtctggatcagtgcagctgcaggaatacccagaggtcgggccattgcttttactaatgctgtagacgagcggggggtcatagctgaaagtcacattcttgtcagcattaacttgaccactgacagtgactacgattgtatggccacttccttggccctcgcttgtcatgcaagtaatcactgcatgcttaacactgttgattgtgcatgaaaaaatatcaccaattaaaacggtatgagcgcctgatagctcgcttgtcccgaaattctcgccttggatcgtcatcaaggaaccaccttcggtggtagcgtcaaccacttcgacattataaatcacaggggcgttgtaggaaaacaccgttgcagtttcttggttccccgcggtcaactttattgtgtagttctgcccttccccaggaggaatagtgactgtagcacgcgagtgatcatacgacgtgatttcaagtgaaacagttctgttggaattcccagtcatcgaaatgactggcgtggtaccgaagtttgatccaacgagagaaacaacctttccggttgtagaagcagaagtggcagagcttccgtcccaatccacgtttggcggaatataactcaaataccgtggactacttagagttatacctcgtgttacaagaagtggaagcatgacgccttgaccactcggcactgtgcacgaaatcaggcggatattgctcacccctgtgacccccccagacagaagtatgctggaagggatgaccggagtgctcaccgcgacattggtacaattgtttccacccactgttacccggggactacggccaacatacttcgtgtatagacttagtgtagctccaccactagttgctgattggtagctaaacacccccgacataacgggagtagttgctactgttcggaataagctcgtgttgccatccaatcggtgactcgttgtacaatccagtaccccgcaggtcgagctaagtgtgtaccagtcgagaatggacggatcatcccagctgaacggcgcagaattactcagcagcttctctctgccaatcacaatggtcacgtatcctagacggcctacgtactgcgggattatgatggtgttatgagaccaagacaaaacctcaactgaagattcatagctagcaccttctaggttgcttgcggtgataaggtactttgacactatcacgcgccccgaggtatagaaattgtttccagtaatagtaagtcggatgcaagaaggtagacactcagtggggccttcgtccgtagataccgagtcaatcaacggaggcccatagctaattggctgtggtgcacttctttgaacactcgacgtaccgaccaacacttgcagcgatccaccatagccataacccaccggtacacgaatcacaatgtactcgctgctgttgctgtcactaaaatctccaaacccaattataggaatgcgataacttgacaaacttgcttgcggtggagtgaactcaacgcgtgctgctgcgaaaggcacgttcgccccaagattttctccgataacagtaatctccgtagtcccatcagttgatgcaacggaagggtacaatgagtaaaccacaggcggtgcatatgagctcgtggcagtcgacaatcttgattcttgatcaccgactgacaccatccaggatagattcgcccctacaccaggaacagatgtgcaaatgattgtggaatcattgtgctcgagcacttccttcacatgatagtctcttcctgaatttccatacgaaattccatcgatgactacagtatcgggcccaaaattgcctcctcgaagaacgactgcctgcccaccgcgaactagagcgttggttgccccattaccctcaatcgaagtgataaatggtcttgaataactaatcgtgggcgttgcgctggttaaaccatcgattgtgactatccaagcaaggcgattgcccacaccaggtgcggtcgtacagaggatccgttcgtggtcgaccacgatggcacaatcctcagccgtgaactcgtcaccattgacgccatatgtcacactcgaaatggtggatgcattgatggtgccaaagttcttgcccgtgataacaacttgctgatccccacttgtcttgatttcggtttctccatccgggcgaaacacttttgtcacaatcggtcctgcatacgatgagttgtgtgcagtccacagggatgcttggccttcgatggtgacacgccacgagtggaacgagccactaccagccacagacacacactgtatcaaattatgattggtcggatcgttgtattttcgtttgcagttgacactgcgaaggatcgtaacattgtcgaaagagtactcgacaatggggtccgtatagacagcaccgtcaggtccgaagttttccccgcggagttcaaagaccgttcctcccgcggtaggaacatctttgaagcccatgacttcaaagagttcgggggccttataagaagttgtgattgtagtagtcgagtaactcttctgatcatccacggaaacctgccatcggaataatcctccagctccgggttgagataaacaggataactgcacgtgatcaatgacaaccttacagtcatcaagctcaaacgagtgaacgttgttactataccacacagtgactttgccacatccctgacacccgtatccagctccgttcaccattacgatgtcacctccggtggtcaacattccggcagccacagttacatcagaaatagtcggaaacgcatagtgacagctagatgcgaaagcggtgctggcctgaccgcgaactaccacaacgaatgcgagatcagcgcctgtaccaacaacactgcgacacgcgatataatgacctgttgcatcttcgcgtacctcacagctttgggcttcgaattcggagccatatacaacgacaagatatgcatctgcgcttgttatctgcgttgaccccaggttttcaccataaatcctaatattgtttgtgttgctaccatcggtcggtagcgcactgttgcagacaacagagcctataaccggaacaccataggatgccaggggaaccgaggaggatgcttggaatacggtttctccaatagttgggacctccattaccaaagtaacgttccagtatagatttgcgccaacacctggcgttgacgtgcatgcaattccggtgttcccattaacgagcccacagcttgtagcgcggtatagtgcagtggctccatacgccccatattccacgatgagctgcttgattacaactgagttatcagctcgttgcttgaacccaatgtttgaaccagtgagtaagaacctttcaccccccttcgtatcgaggtctgaggagctggtatccataagtctgaacgtatcaatagtagtatctagaacgtcaaccacttcgaccgcgacctgttttacaatcgtaaacgcattgaaaccactagaatcacgagcttcaacgctcactgtataggcttgaacagcctcgtagtctatcagcccatctactatcaaatcaccgcttgcagcatcaatgcggaatacattcagtcctggtggatacacatcgatcgcgtaccgaattgtcttgaattcgtcagagtaaatatctgggtctgccgtcttgatttgaagcaagaccgagttaattgccgtattttcgaaaacagaatatgtcgctggaaccccaataaatgaatgaatctcatttacatcctccacttcaactgtgataccagccaccgcagtcagcttaccagcatcctgagcaatcaaagtgaagctgtagacttgtcgtacctcgaagttaaagctttgtttgaccacaatcctagcacacagctcgtggctatagtcctcttcaacttgtaccgcgaaaagagagcaatcagctgtatttgcacacgaaatcgtgtaagcaagtgaataaccatcgccttcgtcaggatcagccgcactgatacaacgcggaagggctagattcaccgcttcattctcttttatctggaacgcgtaagactggtcactgaatacaggtgcctcgttgacatttatgacctgaacagtgacgtcttgtgtactgcacatgtgtgctggatcacatgccgctactgtgcatacgaatgcgcgtttcttctcgaaatccaaagtgcgtcctgcacgaagcgtcagagcgccatttgccaatccaaggataaacatgtctgaacagtatgtgtcggctttcgactgcaatgtaaactgaattgggtcgctgtctggatcactgacaaaatgagatagggaggcccccgcttgcattcctgcccctgaattttcagctatcacgagtgacgacggcaacgtgcttaaccagacaggtgcaaagtttgactttgtcgttagaacttgaatgagtccgttacccgaacagtgatcatagctggtgtcggtaacagcaactttcacatagtattgagccgggtataccagctcggcgtttaaagggttccgaccaagtctaagttgacctgtcagcacgtctattagaatcaaacccgctgaagcatttgtctccaagatgctgtacgtgagagtatcggaaacatcttggtctttacccggaagtggatagccaatttgagtggtgacagaggatcgctctctcaccgtgaatgttaaagaagccagcacggggcagtcatttgcgttgagaacagtcacagttatacgactctcagtatatagagacgttgttttcactgtcttgtctgaaattcgaactgtaagcacatattgagactgagtttcgtaatctggcagggatgatcctttccaaactaagcccacttgaccgttgatgcatacagtcgattcaagaacccctgcggagactaatccatttactgtcgtgtcatccaccaactggaattgtgcgctagtcccgataatttgatagaacgagcacgatgcactgtctggatcagtagcgctaaacgtgcacgcggctgtgccttgttcgttcaaaatagtcggggtattctccactattacacaagtaacatcactcatcaaaggcatctcggggacatcagtaaccgttactgtgacgtagccacttccacataaacttgtctgcgctgcatcgcaaactcttatgttaagattgaagcgagtagggtccttctcaaagtctagagagcgtgttgtcttcaaagaccccgtctgtggatcaatttggaacggtgcaaagctatccacaagggcaaaaatattgtctgagctgcgatcaggatcaacataccttgctactaaaacactagtactagcagccatattctctgggacagacccattgatgccagtaacaacctcaggaggctcgttttcgtcgagtacggtgactgtaacgtttagataggaccaatactcaccgtcagaatacgctactttcagctcatactgagctttgctctcaaaattcaaagacacaagtgtttgcagctgaccatcacttcgaatcgaaaactctgattcgtttccactggaaataccttcttttatcaaaaacccataactgctccccacaactccatcaacgtccacgatttgaataggtcttccaactaaggaccctggagagagattttcttttatgacgaacatatctgccagcagagtggggggttcgttgacattttggaccgataaagtaataatagtagatgtaataattggcccacactgagactcctccagtagcatgtggcatcccactccattgtcttcgactgtaatttcaatatagagtactggcattgtttcatagtcaaaaggagccatcgaatacagcccaccgttcccggaaacgcgaatataatcactagcattcgttcgaaagaacagctcagaattcttatcaggatcagtggccactacagtcccgataaacgttccattaggttcgttttctagtatactttgagaactagagtgagcgaattgaggagcctcatttacatcaagtatcgtgacctccacgattgcttgcgactccagagcaacaggatatcctgaatcaataacacggacgccaaaggaatagcgattggtactttcgtagtctagattatcccactgtactaccagttgcccagagcagctttcgagccgaaatggcaaaccgccagcatttcgattcaccagctcatatcgtaaattctcggtctgaccacgatccggatcaatagcacgcatgggcgttccaaatggtagccccgatatggaattttcaggtacgctgaatgaatagtatgcttgctggaaaattggaggttcgttaacatccaagactttgatctgaatttcgcggtcactgtacagaccagctatatcagaaacctggatgcgaaccatgaccaatgaattcttctcgaaatcaaaacttgaactattgcgtacagacacatgtccagccggcgtcaataaaacgtctggtgatggaaatgctagttgaaatgctatccgatcgttgaagtcgggatcactaccaacaagctcaccaaccatggtaccaacagcgggattctcttgaatctcgaaataactatcagaaaggatgggtgcctcattcacatcatcaacaaaaacaaataatgtggcctctcccagtaactcctcgctatcgacaccaatcacaacgacttgcaatggctggtcgcgggaatcaatgtcttcgtaattgagcgtcgggttagtgagaaccagctctccagatgccgtgacaccaaaccaatcgctctttgaaccatggagaaaaaaatctaaagaagccggatacgcaccaaaaatgcacagcatgctcgttgatgttgtgtcaggtatctcaaacaccccgaggacgtttttttttatatatatatcgaaaataagcgcacttgcactactttgtgctattgcagtttgaccgccactctttatgaatgagttcttcgatacccactctggaacaaggtcagcatcctgctcgaccaaaacaaacaaactgcctcggctcgctaaaaacagcgcggtactcgacaaagacactgctgtaggtgtggatatcacgaaggcatttgccagctgaggtggaatgtctatccattcgaaatcagaagaaccagagaatacagatcctttttctttcagacttgacactatagcattgcctccagctggttcggtcaacgtcaaatagtattcatcgtcgtgctctggatcggaaacattaatatagcctaagctagttccaattactgcgttttcatacacgcggtacgctttaccagtgagaacaggtgcttcgttgatattcaaaatgaatatccagacggtggtaatcgcagttgcttggccatctgagcactgaacactgagttcatattgccgttgtgtctcgaaatcaagaaaagctgtttccgcatacacaattccacttttatgttctacactgaaggctgccgtatcgttaagaatgttgaaatataactcgtcaccgatgtcttcatcaaagcaaggaatcacccccagttgctctaagtttcgcgagtgtgtagatgcggctggtttcgatactaccgactctcttgctgtaaagttgacaggctgtagcactggcggatcgttcacattcaaaaccgtaaccagaagcgttgccgaaacatgaagtcctgcaacgtcggttgcaacaacgttaacggtgatcgacggtgtagtttcgaagtttatcttgtaatcgtcgactactgttagcaccccagttgctgcatcgatattaaactgtgaagagtccgcattttgcaacgaaaacttcaatatatcaccaaagtcttggtcgatggcttttacgtatccgacgatcgtaccatgtgcgctcatttctttcacaactagagactgctctccagaaattgctggagcttcattcacatcaataacactaatcacaatgtcgttgctgcaagcgagtgaggcaccagtacttggaacatcattcgcttgataacgaatgcgatacagactttgtgtttcaaaatccgcgaaagtaagttgattcgcagcaaactgaactacgccagtggcgtaatcgaccgcgaacgatcgactatcatttgtggaaatcaattgaaaaacgtgattttgcccagcatccttgtcttgcactactatctggcacaaaccttgcgtcccaggcagtcctctgttaatcaaaggagaattttccagtatccaacactgaaagggcgggcaaattggaggctcattgtcgtttacgaccgagataatcacattggtggaattcgttaaccctgatgaatcagtcacacgtgcactaagctgataagcttcattgttgtctgcatataaaaccccttgctttatcagggatattactcctgagtcagaagatatcgcaaagatactgctcacatctgaggttgcagtatcctccatattccatccatactgaattgtatcgttcaaatcgggatctgtagcttggacgacaccaataagcgagcctccaggggccagttcatgaacagatagctcatatgaagatagtctgaacacaggcgattccggtgcatccgataccataattgtaaaattgaaatttgtcgaggtgttatccgatgcggtcactgcccctcgtatggttagcattggctcaggcccaccgaaattcacctttctcatggcttcgtagtcaagagctactttcgttgttgctacccaaccatcagcatcaattgtgatggcattagcgataggacgcgaatcttgtagagaaaatacaaacttgtctccttccgggtccatgccctgaaggtggcccaatacatgaccacttgcagagttttcaaatagcacaagatctgacgctgacgcttgaagaagtactgggggctcgttgacgtttagaacaaatatgaatacggttacggtatctgaaagaaagcctttccccgtatctgtgacgattaactgcacccggtgcacccgcgcactctcgtagtcaaagagctcgcgactgtataacaggctacagtttcgatcggtatcttgaaccacccgagcgaaggaagctgaattttcgtcaatcgaataaaacagtcgctggtcactgtcctggtctatcgcttcgaagtgatgcaagatagatccaacttccacgtcctcatatacttggaggcagacgtgctggttctcttcgtagcggttatcagctgtgccgtcttcacgaactaagcagttccggtgtatctgtggtctctcatttatgtccacaaccctaatttcgacttgtgtcaccactgagtttaaagctgtgtcttcaacagcaacctcaaacgtatacagcgattgagtctcatagtccaaatctccggccacatgaatttgacccgtattgtaatcaattgtgaaaggtactccgttggcctcatcaggtagtattgagaagtgcaaaacgtcataaccatccggatcaaatgcatcgattggtacgccgaccaaagcatataggggcgtgttttcttcaacagactgttccaatacgtttgcgacgtggggtggttcattgacatccagcacatcaataattatgctgcacgtggaagacaacgacgataccgtaacttgtcccgcagagtcgtttctatagacaaaactatcaccaatctttacggttatcgtatacatggccacattttcatagtccaaagtgcctgagcgttgtaactttagccagtaggcattaccacggggctgaacctcaaaaatatcgaaagcaatgtcacctgtatcactgacaacttcaaacactgtaatcgtgtttttgatagatgaatccggatcattccacttaagaggaccgaacctctcggatgccgaggcattctcaaaaacagaaagtctcatgtcttcacagatcggcatgtcattgatgtcgatgatttctactggaattatatccgacaaagttttaagaccagtgcgatcggttgcacttactactagctcgtatcgatcgtttatttcatagtcgagagaattagatttagacaaccgcacttctccatcacacgagtgtatcataaaaatatcgacagatactccgctctgatccaatgcagtttcgatctggtatgaaataacgtcgtactcgtcaggatcataagcagctcccacgatacccacaactgtccctttctgtgaatcctcagcaatactcatttttgcctgcgatgtgaatactggtggttcgttgacgtccacaacatcgattcgcactatcgcgcttgagcgaaggttatggctgtccaccccagtaatatagagatcgtaatacgactttagctcgtagtcaacagggttcagcagcgatattaacccatcattcgaaatatgaaaaacattcgatgagttttgtgaaagtgacgtggaatcgttttcggggtcccagaatgtcacccgagcgtggctaccatcagttgctctcacccagtctagctcttcattgacagattctggcacccaaaaccggccaggtttgataaccggaggctcattaatgtcaagcacaagtagctggactgtggcagtgcatgtgaggccacctccgtccacagctccgaccttaaatgatatttggcgttgaacctcgtagtcaaaagggttgtttactacgatgtcacccatttctgaaacaacgacggtgatatctgtcattccgtcaccgatcatgtaataactcaacaacccccaagaaattgaggtatttgcatctggatcgatcgccacgactcgcccaacagtaccaatcgtgttctctgttattgaaaatgtcatgataggctgttcaaaggtgcacttctcgttctcgttcatcacatcaataatcaaagtaccagtagcttgcttggtcgggctgccgtcatcatgaacttccacggctagttgtatactacgtacgaactcatagtcaaaagcactgccgtcaagaactatgatggagcttgagaccatgcccacagttaccacgtcgttttttggcctcaaaacagtggagttgtcccgcagaaccaaagtagtttcaatcaaagtgaaaaaatggttctggttgaaatcctcgtcggtaatgcgtataacccccacgatagagctttgacttgggttctcaatgatcgaaaaatctgtagcgacaatggttggagcatcatttctgtctaaaacaacgatgtctaatgtcgctgtagtaaacagattgccactatcagatactaccactgaaaaagagtagtgcccctttttctcaaaatcaagctgttgtctcgtacgaataacccccgtagttaaatcgatactgaacgttggatcaacaatctcaaaaatcaatgtgtcactaaaatcggggtcgaatgcaaccatctgcccagtcaaggtttcactaagagctggtgacgtgaacacagaagacccaatctcagcattttcagcgacatacagcgtttgaggatatacctgaggtggttcgttaatatcttgaatacgaacagacaccgtagaaaagctccataatcctcctggcccattgtctgtcgcagcaactagcaccgtaaagagttgttgtctttcaaaatcaagggctagttggttacttacgaccaggctcccatctccaccaaatgataatattgaaaaagcgcctaagtcatttccatttgcgatggtatacgtaatggtgctgttttgatacttatcactgtcgacagcggtgatacgcccgactagtgtttgaactggtgcattctccacaacgctgaacatgtatcgatcagcttgaaaaacgggtggttcattgctgtccgtgacagtcacgattactgttgtatcagcatattccttctgttcatcaattacacgaataaataggcggtaatactgcttagtttcaaagtccagcagttttgtaagagtgattatacctgctgctgataccatgaatgcaccctcatctcctgggtcggtgtctaccatttgaaaacccaaaacagagttctcatcaacatcagaagctgatagaatgccaatcatgctacctttggttgccgtctcagcgaccgcaaactggtacaatttctgtgagaaaattgggggctcgttcacgtcaataacagctaaaataatcgttgccgtatccgcaaggtgcaatgatgaagtattggcattgtatgcgcttgtatcaggaaggacaacgtagacgacacacacccgcatggtttcatagtccaatccgccagttgcaagggtaagttgaccacatgagtcaagccaaaatgtagtacggcgaccacaagtcgtgtttgtaatgaccatctcaaccgagcatcccacaacagtattcaagttttcttcgtcaacaacaaaagcagtgagaggcttaccgatagcactaccaacatcaatattctcggttacatcaaattgaatcacttcatggtcgaagtatggcggttccaacacgttaataagattaacgctgtacctggatctcgtagtcagattcccttggtcttgtacacaaatatccacccaaagcgatggcacatattcgaaattgagagcagtggagttctgtacgataatctctgcgccgttgaagcctctattttgaacgcgaaatgtttgtgagtggctttgctcgcaaacccaaaactggtgcttgtccccgttgtcgggatcgtgaaccagtatgaataacacaactgtttcatctggcgaattctcacgtagaagtgtaggaagagatccctccagtataggaggctcattaacgtccgttatgtagatcacgctcttggaaaccacagtgaactctccatcagatgctcgcacaacgataggaaggctattcccgaaagaagactcaaaatctagtgagctgtcacttttaagcattaactgcccagaagatgaattgattctgatgcaggccaacacaggttcacttgaaccaacgagatcatactcaataatggaatcgatatcctcgtcgactgcagtcaccgggtctcctatggctgacccaggtgtaaggttttctggtacaaaaaaattgaaggaaccaatttttggtggctcattgacgtcgtcaatgtatacaacgaaatcggtttcatcagataagccgccatggtcaataacacgcacacgtaacttgtacacggacttggcttcataatctagacgacccgacagactcacttgacctgaccgtaaatcgatggcaaaggttgtctgattagtgatcacaagctcaaaggaaaacacttcatggccattttttaaatcgggatcgatagtgctgtcgacaaaatttcttcccacaagtgtactaataattgcatcttctcgtacagacgtactttggactcggattgttggcctctcgttaatatcgataacctttacagtaacattggtaatcgagaataatccaaacgcgtctcccgctcttaccagtaaagcgtattcgcttgtttcctcgtagtctagctgttccgcttgctgaaatattacgcctgtcagggtatcaatgccaaatgatgttggggcccgtggaactccactctccggtgactccgaaacgatgctaaaaaaaattgtgtcaccgtctggatctcgagctttcacagcaggtaaaacattcgttaaaacaggttggttttccgaaatattcagttgcatcgactcaacgatgggtggctcgttcaggttattgaccagaatgttcacgtttagaacgctgctttcggcccccgactcgtccgtcgcgataatggaaagcacgtaggttgacttgaattcaaaatcaagcccgtcatgcattaacacaactttgttacccacaacgttaaacatgtccagctggccaaccaagcggattttcacagcatctgggcggccatcattgcgagtgtcggggtcaaaaacagtaagatttcccgttgcttcagccccagaacgtgagttctcatctatgtcaaaagctgtagcgaggagctgaggcttttcgttaacatcacgtactacaatgtcaagtctctgcgggggactagtcaaacctcccaaatcgcttgcttgtaccattatcgaccaaatacgttgctcttcgtagttgatattctggggcatgttcacaacaatctgaccttgtggggtaatattaaaaggactcttttgatcattttccacgatatcccacgtgtgtatcttccaagatatgttcgctgctccatttttgtcttcatcttgaaccgtgagactgcacacaatcttcccaactagtagatttaattcctgttcatttaagtgataaggaaattcgtccatttcacatgtccgaggagacactattgtcggtgcatcattttcgtcagataaaattactgtagcagatccacatccttctaaaggtggattcccaccatcctttgcgcatacagtaattgtgagttgttggttaacttcgtaattgagggcagtggagttggcaactgataacattcctgaattcgaatcaatacggaaatttagtgcttctgtatctgaagtgcgtggaagataatacataaccctccctgaaaaaagggttacagtatccggatcagtcccatcaatgatacaaattgatgtcccgggtggtgcattttccgccagaaagcatgttgcatcgtgcacaactggaggctcgtttacgtcctcgatatccactgtgaccgtcgaaattgatgacagccctagacgatcagtaactgcaatcgagagggcgtatttactctcactttcaaaatcaagttgatcaatatttgccgtgagttcaccagtgtttttgtccacaaaaaatactggactaactggattgccctcgatgatagcaaagtgaacggtatcgctaacatcaggatccatgtatggaaccacaccgtcgacaaggataacacgtgagactgataaaagcaaatacgaaagttagaattaacaacaaaagctagagacctgatgaaatttaaatgaaagcagaccttgggtaacagtgatttcaagaacagaagccattgccgagctgcggtcgtatgaacttacttcccctattcctccccctgatattaaaaaggatacacgagtagtgttaatcatgtccgccataacaggttcgataattgacgctaagttaggtgaccaaatagtagttgctgaagcctcactttccacagtccactccacgaaggcaggattcccaaaactttccaagctatcagttgaaatccacgaattctgagagctcgtcatcacgcgaatctttagtgaaaatgggccaatctttccagatggaactgtaaggcgtaactgcgctaatgaaagtttagcgataaaagctggaagtataacaccctgaaattgaagaacaacacccactgcagttttgctaaaagggtcatatcccatttgaagagttgttcgcgaccagtccgtcgaatatacgccagccgcgttctttttgatgctataatcgtctcgagacgaaataatcggaaatacaaagatctcggtcgtgagcacacgcaaatgatcagaattaacacctaatgtcagtgcatacacacacacgttatactcactacacacatgacatcacatattagcggagtggcttctttcttaccttctggaatactaagatggcgattttctgtcaaaggactctcgttaatatctctggccgttatcgacaccacacagctcgaacttagtgtcccgaggccgttgtggtaagcaccgtcatcggcgacaacaatagtcgcaactgttgtgttgtgcgcggccgattcgaagtcaaggttgttctcgcgagttacaaatatctgtccattggtcggatatacccccagaaacgaatgttcaagtgcaaaagtgaagacagttccaatatctgtatcactcacgtacgtcgtaagtggcttcccaacaagcgctccaactggagagttttcatccatggcaaatacaatgtttcctctacagtttggtggggtattgtttacaagtacatccaccaaaactgtagtagtactaaaaagctgcttcatatcatacacgctggccatcagcataaatgaaaaaccttgcttaagagcttgtatgttctgtggtgaaaccacactgacttctcccgtttgcgggttcacagaaaacacaccaataccatttatagtggtagcagtcagtaaggtttcagatggctggagtgagaacgacaatgaatccccgtaatcaggatcaatagcttgaagtacgccaattacggatcccgtcgcggcgtaacgagatacggaaaactggataagtctttctgttgcaggtacaaaggatggaggctcgttgcagtcctcaacatttatacgaagaacgctagacgtacacagggcaaacgggtcgcatgctcgcagggtgacctcgtagagagacttctgttcgtagttcaacgaggcctccaaagtcagttcgggtgtggcgatattacggatggcaaaaacgccagcattattatctctgaccacttggtatgatagctgctgacctgcgtcttcgtcttgaacattccatacaagcactgtggttccaatagccgcatcttctctgacggaaacgcctatttcaacggcactagtaggtacttcattgacgtcttgaatcgcaataacgaaatgctcaattgaagacaaagtgggggttcctctatctgatacgcgcacatttatccaagcttgatggagctctaaaagctcaaaatttagtcgttttggatcctcggctattatgaacccgcttttagaatttacagaaaacaatccagctggagattgatcttcaatttcaaacacgagagtgctggtctggttgaagtctgggtcgtaagcgtggatggcataaaaagaagtgttacagttttcattcacttgaacttgctcactagctatgactggggcctcgttgacatccacaacacgaattgttattggacacgtactgcaagcaccgccagcgtcacagactgaaaatgcaagctgatacaactcacgcgattcataatcaagggacaagcgctctgaaacaatagagcggcccttgaactgaaatccagaagtctcctctttatcgattatagtgaaggtcggaatgtttccaagatcctccaaatcagggtcgtagctatcgaccgggggttttaaaaccattcctccgggactattctcttcgacatatctcgtttccgattgacaaatgggtggctcgttaatgtcgaacacgttgataataactaaaacacaagattccaacaaactgtcagtggcacacacttgaagtgagtaaacactctgggattcataatctaatgatgctgttgaagatacgataatttgtcctgtggttgatttaatctcgaaatcgtctgaaccacctgagccaccagatgactttatgaaaaagctcaggtggctatttgcatctggatcatatgccgtaagtgatcctacgacggtataacttgctgcattttcctgaattgccaaattaaattgactctgagcaaagctgggtggctcgttcacatcaacgatagagactgaaataggcacagccagtcgtgctttgttaggatcataaacagaaacagtcaaactgattgtgtttaggacttcttcgaaatctaatgggttctttgaacgtagctggcccgtacaaccatcaacagaaaaaaacggcgaaaactctgaggcatctaaaacatagctcagcgtattgttctcaggatcccaaatattagaagacaatgaagctccaatggccacccccgtgaccatattttcgaatacgttaaattgaataataccttgggaaatcaattcgggggcttcattcagatcaacgacgtcaataataatagttgcgttgacagtaaagctagtatccatcttgtccacttttactgtcagtgacattaattgatcaaattcataatccaaagggctgagcaggaagagctcgccagttatcccatcaattgcaaatttgttgtagttctcacccagcgagaaccttaatttttctcgagacgttcggtccggatcagaagcattaattagttctaatgatcgcgggacaactccattttcaaacacgcgtaaactgtgcgtttcaggaacgtgagggggctcctcttcgtttaaaacagatacaaaaatggtgctttccgctgtcaatagaccttcatctgtcacttgcacttgaagagcatactcggctgtggtttcaaaatctagcatacccggattcagaactgtaatatcccccgtatcggaattgatcgcaaatatgtcgctagccagcaagcgaaaatggagtaaagaccaatttaaattcgtatctgggtcaattgctttgactgtcataaccagctcccctttcactgcagattcgttgatatatccgcgttggccagttgaaatcacaggcacttcattcacatcggtgagctctataaatactgtagtatataatgttcgctggtttggagcgtcatccacaataagcacaacacagctaataatggccgtaccttcaaaatcaaacgctgacggatttccgacgagtaagttactggaaaggcgctcaattataaaagggcaatcacgcgtaagaatttgagcaccggatttctctccctcttcaacagactcgatttcgaattgtccgatcaaaaaacccttttggggattttcagcgattgtaaacttcctggttgtagttaaaggtgcatcgttcgcatcaaccacgtcaattccggttgccatattatcactgaggccaaattcatcataaacttcggcagtaaactgaagagacgactgcaattcaaagtcaatccatcgccggcgcactgaaatggatccggagcttgaattaatttgtatgcctgctggtcggtcagattctcgaagtttaaagcgcagcttgtttttcttatctgggtcagttgcagataggggatgctgcaagtaaaaatctaagggaagtaggaattagcaaccacattaaggtgtttcgaatcatcggaaatagaaaacgtaccagaatccgggccatacatctccaaacgcaggcaagtacttcctgaccagcctgtagggattattctgacttgtttcgcaatcacgttagggagcaccacgctagggttgaccgatgagtccatactagatccaaatgccacgaagtttatgctttttgtatcaccccccacaagaggtgtaaagcttgatgtttctgacagcctccatgctaactggaaagtttgtagatatccagtctggccatttttatcacttccagggtagacttggatgcgagatacgattgtcggtttaggaaaactaatttgaacccactcatttgccgaaccggatttagcgcaccaagatccatttgttgtgtcactacgaatgacatcgagcccaccatagccgtatccatgcatttcgctgttgtcaaggtaagaactagctgttatagaaccagcctttatcactcgtcgcgctgattcacctattaatctgctttcggcacctagtgcataaactcgttagaagtacgacatcacacttcctgaacgttacatgaacttggacaaatacgtacttttagagcagcatgcaaggtttgcattctcagcgttctgttctacctttccagcagaatttgagcaagcaggtgctgtcatagtgccttgggtggctgaaggaaacgctattgagctatagtgagggtcaacacagagcgaatcaaaatcctgcggatcacacaatcgtaaattttggctgctgcagtaagtttgccccccagcttgtgtgatttggtcctttgtccgcttaaagaaagtaggaactgtacacgtcacacacggcatggttaaacctcctgctgaacatagttgcagcaacacgatgtaggacaaacctgaataagagacgagttcctgaacttgaaaaagcgctattgctccagcaaagtagtgaaaccgtgatatccaccccgtaaacctagcactttgcgctgagagaactgcctctttagcgatagtaggtgctccaaggaacgccatcgatattgtgtccagctctccatcaatgaaaatacgaagatgttggtcgtttaccgtaaatatgatggctatataatgccagctatcgtccgatatatacttctgagagtcgatagcaaaggaattcgtcgaaacactcagttttccgtcactgttgatcgaaacattgaaatatgcggtacccattggatcacgcagaagcaaaatgttcgtgttagtgcttcgggtgcgaacacttgttgccgtactaaagtctaggtcgccaaacagaaagtcaaaattttcattgcttaatagatcgtgggtaatatttccaaaaaatattcctccgctggccgtatagtggacattcccattgaccccttcgctaagttgagctgcttcagtaaatgttgtgattgtcgacacaatatcaaaccgtggtggctcattaacgtcaactacagcaattgtaattttaccgcatcctttcattacagaaatataattatttctttccccatcatcgattgcgcaaagaacaaaggaccacaagggctgagtttcaaaatcgagcgaagttccccaaattgagatatctgctgcagtcctgttcactggacgaaatcgaaatttgtccgtatcagggatggtaaagaacgataaaacgcttcctgcataagtgtcaataccccagacacggccaatagtactgccaatctcaacgttctcaggcacgctaaacacaaattctcctaccacaggcggtgaatttacactgcttactgaaattatcaattgttgatttttctctgaggtgagtccactgccattgttatcgatcgcttcaatctcgatattgaatgtttccaacgcgtttacttttcggaccatagttatcactccagcgctgctcatagcaaacacgccattcgaattatcactgatgattttaaattgaagttcgtctacgacaccgtcaggatccaaagcgctcacgcgatgtactacgccgtccacattggcagattcgggaatatttagttgaacaatcaatttagtaaaaaatggcggttcgttaacatcggtgatgcccacgtgtaccataaaacttacgatattcccaccattgtcaataactcgacaagaaagatgagccattgtgacagcttcgtaattaagcccctctgagcttagaacataaatctgccctgtagaactcatgccaaacaatttatccgatggagcggaagacacaatttcaaaaagcgagctgtctccaacatcttggtctgaaaagtacatgctcataggatttccaaccgtcgatccattcggagtattttctggaatactcagaataacgggatcttcgaccagaagctttggaggctcatttatatcacttattagcacagtacacatctgtgatattgataggcggccctcactcatactagaagtatcagacaccgtgaccacaacgtcaaaacgtttttgctgctcgaaatccacttttgctcctcttacctccagttgcgcgttcgaggccgacgaatgcacaatgcgaaaagctgtgctattatcgtcagctgcaaagttcgaggtatacgtgaaagtaagagtctgattttcaggatctgacccttccaaatacgctacaatagtacccagtggtgcattttctgaaagggtaagcacattcagagatactgtaggtggctcgttcatatcaagcacattgattataatatcaacagtaaccgtaaaattatcgctgtcagttatcttcacttggaactgatactgagcaacacgttcataatcaatagcagcatttgcagtgacaaaaagttgacctgtcgttacggatattccaaatggaagttcttgtccgatggcaataattacaaattcaaaagaatcagcactgccttcaggatccaacggcctgagggcatcaccaacgagagtatacgcccctgaattttcattcacataaaaaacttgggggctcgacaagacaggtggttcattcacatcgagtacgttaatattcacccaaatactcgttgacagctccgtatccgttgcctcaatcagtactcgatataattgtttcgtttcgtaatcaagcaacttactctggcttagcacaaattggccagtcgttttttcaatagtaaatgcaccgggtgaatcttgctgcagtatcgtgtactgcagcatcgacgatgaatcctggtcgatagcacgaactggacgcccaacaggtgtgctttcgtcactgttctctgggattgatatatcgatttgtgcactgacaatcgagttaagattttgaacggagactggcatccattccccagactcaatctggaattctacagaaatagcgtggtttgatggtgcataaaggtatagcattatgtcgtgtcttccacgtcgtagatctccagcgtcaacttcgtcgctatactgcagatcaaaaccaggtcgagtcaacgccttccccgtaaacacgagcctgtcaatcatgaatacagcgtttaatttgacgctgctaaggatacgaaagactgtattggcttttgatcgcaaaagtaagttccatccgatgacagcagcatattcgccttcaaatccgttcgggcagaatgcaccattactgaattcggtaatactttcaatgttcccacaacaatacccaagtggctgactagctgatagaatcattgtcgcaagttccattccttccagcttggcaaaggtgtctgcgctagcccttttagttttaacagttagcgagtagtcaatcgtgaaaattggtgcttcgtttacatcgccaattacaactttaatcaatgctgatgcagttagcgatggcgtagcactgtctctaactgtaacttcgatctcgtacgaagctgtccactcatagtcgagagaattcgagcgtgtaacgtagatttgtccattacaagactggataccgaaaagttcgtgcccgtcatcctttgtgatcgtgaaactcaaacgctggccaatatctggatctgaggccaacataggagcacccactaaggcaccttctgaagcattctcatcaatgtttacttgcatgggaaaaataactggtggttcgttcacgtcaataattttgacagtaaacatagcactagaacacaacttcattgagtcgcaggcaagtagcgagaatgaatactctgctttgacctcataatccagatttgctctaaaaacgacaagttcattgttgtcaaaggcaaaaggtaacttgctggcattgcctccaatggaatatatcacggtatctccttccggatcgacagcaattacactcgcatcattcacgatgacagtatcaatggcgctattctcttcgatatcagcaccatacagtttgaaaatgggggcttcgttcgcgtcgacaacacgaatttcgatttgttcagttgtggaaagacctgcgaggtctgttgccgtaacgccaaggaaatacgatttacaaacctcaaaattaaatgcagatgtatcgatggcaaagattcttccagtatcaccatcgatacgaaacgatgaacttacgttgctactgatcaaagaaaagcttactcgatcgtttttgtcaggatcactagcagaaacgtttagcagagggacggaaggatcttgagccgattctttggtgaactctaaaattgacccaacttgaccaggtactataaaaggaggctcgtttatatctatgatatagacagtgacaacaccacttgacgtacacggctccacagcatcatcagtgacgtaaattgcgaggtcaactgacgatagtgtttcataattcaataccgacgggtcagcgactgtaacgactccggagtatttgctaattacaaatatatctgattccagcgagaatgtcagcctcccaaatgcatggagtccataatcttcatccattgcattaatgttatcaccaattgacgagccgacagcactgttctcattaatgtagcgtacctggtttggaacgcagggacaatcatttgtgtcgatcacattgatagctagcgggcaacgcacagtgttgactttgtcactgaccaagagatcaagcgcatagtagggttttgcttcaaaattaattaacgccaggtcttttacaaagacggacccagtagagtttgtataaaatgttccgttcgcctccattgcaaatgacagtggcttgttttcgggatctattgccccactttgcaataaccatttattataccctgctagctgttcttgaacatataatatttggatttcaccacaaacgggcggttcatcaacatcaagtactcgtactaccatcctacaaacaatgtagtttgcggatgaatagtaaacagcaaatgtcagagaataggtggactgttgctcatagtcgagcggcaccagattattcaccgaaatttctccagtcgttgtatttagtgtaaaaggcactagtggttcttgcagagaaaagctgttcggaacatacgcttcaggtaatacttttactcgacccacgatagcccccacggcattctcagatactgccaaattctgatctttttccaaacaagtaattgacggaggatccgaaagcatggtgaccacacacaacacttgaacgtggttgtagccgtcactcacaataatcgacattactacttggggtatagtggcgaatgcaccactacctgtcttattcacgcgtaatacttgatcatctgagatgacaaaacaatccagatcaacagacgaagtagtaattgaaaacgtaagagtcttcccttcagggtctctcatacccattgccaatattggatacccttggaacgcactgcgatgcatcgatccattgacaacggtcagtccgataggtggctcgtcgacatcaatcacctctactataacaacgcaatttccactgagtccacctggatcagtaaccagtacagcaacgaaatggtattgctgagcctcaaaatccatcagctgctgcaaaaccagtctgccggtattgtcaacagaaaaactgtcagtttcatttttgacgcgataggtaaacgttttatcagtagcgtcttcatcagtgacgagtagtgctccagagagtagcgtgtttactggagcattttcattgacatgaatacgcgcacacaccacgctcggggggtcatttgcatccagcactacaacgataattgaatatcttgcgactatatcagtgtcaacaatcgtaagattgatataatgatttggttttgcttcaaagtcaactccactgctatccacgtgtaagtatccacttgaagagtccaagtaaaaggtacctggcgtactttgagacatcaatgagaattgtaaacttgttgaaccctcagggtcatcaaatattaaagccattgaaggctcgatgggggagccaacaggcatgttctctaccacttcaagtattccgttcgccagtagcaaaggaggctcgttttggtcttgtacctcaatattaaattctttgactgttgataaagcttctagagatgaatcatgaactgcaaccaccacttgaaacgttttcttaaggctcgccacgtccaatagtcggtaccagttgtcatttctgagtttcaaatcaatcacatgccctgctcgaagacgaggaagctcaaatcttatagcccctatagcatatgcgtcgctctgcttctccgtcacattcacagctaaaaacaaattactagatacatccacgcgtaccagtagtctatcacgcaagggcagacttaatttgttcgcattttgctcaatccgggagtaaagaaactcgacaatggtagttgtttttgattcactaataaattgaactccggtaatggtcaccgtatcgtccagcccaagcgtagcactaacgagcacgttatcaaagagaaatgcctcatagtcaaagcacgatgccttcaaaacggaaagttgacctgatagcgatccaatcgccatgcaaccgcttatactttgcgaagcaatcgaaaacaacagcggtgtatcctggtccgggtcgtatgcagcaattgggagccccaccggagaaccgacgggcacattttcacgcaattgaaaagaagtgctgatgatgactgggggctcattaacatccagaacgtgaactgtcaaatttgtgaaagcactgagtgatccactatctatcacttgaaataggaggctgtatgaatcgcattgctcgaaatccagtgcctggcgaaggatcaaatcgttttcgctctcactgtattcaaatgggcaccctgaaggtatacaagatataaaagtgatcgttaaggtgtcattggtgtcttcgtctgctacggagagtacgttggtcaaactcaaacttgtgacacttccctcgttgatatataaatctgacgaaaaaattgttggaggctcattgacgtctgttatatcgatagtgacgtcaaaaagcgtcgattgaatcggatttccgtcgtcaaatgccctaactgtgagtacatactgtgtctccttttcatagtttaatgaagcgcccaactgataaactgcgtacgaattcgtcccaccacaactttttccaggtgtatcgttgcaagtcaaattgcacgtcgtttgatggagttggttttcttgttcgccaagtgccagcttactacaccagcactcagaattgacacccaacgccataaatggatacgcaaagcattgctgatagcacctgctgtaaggtgctgtggtatcgctagtaaaagaaattgacgattcactctgaagttgtgaaaaacaaccaatgaacagctcgggcccttcactaatcaaaagttgccctgttgccgcgtttaaagcgaaaaaattatcatcgtttccaccaacgatttcatacgaaacttgctgtcctacatcccgatcaattactgtcaggacatccccaattgtactaccaacccgagtattttcgcgtacttccctgctctggttactcccaaaggggggctcattttcatcaagtatgccgactctgacgtaccctgaagcagaaagtggattttcgatacgtaattctgtgactaaaagttgtagcgaatagaccgggtacgtttcgtagtccagcttttcacgcacaacaatcacggatttattctggatggagaatacatcgagataattgccatcaacgattgaaacctccaaatcatcgttttccggatcgtacagagcaatatctccaaccaacacgccaatttttgcggattcggaaacgtgaaggatggaactaggtaacagtgtcggcgcttcgttcacatttatgacccaaatggtgattgctgtcgttgtcaacaagttgccactgtcttgtactgatactcggagctcgtattgaccccgcgtttcgtaatcgagtccatcgacatttcgtctgatgtatcctgtcgacggattgatttcaaatgcaccactaaatggatcgctttgtaaaaaatacgtcaggttactagtacccgtgtcatcgtcatcagcatttacatactgtaccggggctgcatattgtttggttagtgttatggaacaaacatgtacgtcaacaaaacataaaacttaccgtaagatataaagcgtgagcacggcttgttggtgcttttcaggtacgaggacgatttccatgtctgagatgaggagtcccaatactgaatagcaacagttgaaatgtagtaattctgttggcaggcggtgtcgagaatcactttggatgcatcaactggagtcctaaaactcatttcaatccatatctttgaatcggctgttgtatccccagtgatgtatgcggcagtagttacatccccatcgattgctttgtcaatgctggacccgtcagatgatctagcagtcaataaatcagttattaagttaactcgggtgcgctctgaaaaccaagcaagctcagcaattccgaaagaagttgccccgccatcagataaaacagtgaggcgccagttgtgtgccacgtttccaattgaaccactaatcagtgggccatcgacatagatggttttgctcttctcggaactctccatattgtttccatgtgctgtagcgtctaccaaattgccgtcaaatctagaaaaatgcaatatttgaacaaattagaaaggttatgattggaatagctgcctagatgaatgataaacaataaaccaacctgtaataagcaagaatattctgctggtacgggcttaaagaccgggcaaaattggctcttatactctcaaaagacttaacctggctccacagtcgcagctcaaataatcaaaaacgtacagtacaattaaaaaaaatcatggacgtttaaatgaaggcaaaatacaaacctcatcgattgagaactgaaacggttttcctggacatcgaattccagaaatgcagtttccaacaatgagagttgatgataggccccattgaatgcccacgcaagaaataatttgccgtctgacaacaacaactccatccatatacaccatcagctggccttggatattatcgaaagtcaccgctacgtggtaccagacttgtggtgtaagtacaactttcgtgtcaattgttacaccgtgaagcgtcacaaggagcctatctcctgtatattccagtaacaaaccctttagtagtactgcactgtccctccatgccacaagcgatccctcctgagtgtctatgtatacccaactctccaaggtgaagtctataatggcagcttcaacctgttgttcttggtgtacgtacaacgtgtcgttcgaaatcagaagcgccccacttgatgaaatactagtgatgcgctcgaacccttcgcaatttgcacatgcgtcgcaagaaatgggcttgagagtgtataaggtgcactcagctgaggtgtagttgtacacgcctgactcgcaataggctttctggacacatcgctgcatacaacttccataagacactattggagatgattcgattgtcgctgtgtcatcattaatggtgcatgcgcctcttgaccgaaatctagatgtagcaattgtgtgttcaggaacattttcgtcaatgaaaaaatgctgatttgcttcaaagtagggagcttcgttaagatccgttacagcagcacgcaacgacagcagcgttccagcttcagcctgatccgatactgatatatctattggataagacgaccgagtctcataatccagaggctttaccagtttaacgttatagactgtggaatttatctgctccagtgcgaacgttgaagaggggtctgatatctgcactgtgcaaacatcaccagcgtcttcatcgaaaacaactacttcaccaattactgtacccaccgtagatgattcaggaatgtctacgttcatcgttgtcaggctaggaaatgaagcgaggaatgaagcctggggcggatcattggtatcgatcacctcaatgtttaacgccacaaatgagctcttcgccggccgaaacgcatttctgacaataagcgttaagttgtaggtgggacgttcttcaaaatcgagcacttcaattgctgaaatctgacctgtaaaggactcaacccgaaaagtaacactgctatctacaagttcgaagatgaatgatgtcgagtcactccgccctgtatctgggtctgtacagtatttccagatagcgtctcccacatacgtgtctctgattttattttcgggagcgctaacaaagatacctaacgagcaaacgggagactccaattcattcaaaatatcgataataaatgtgcacgtgccagcgagtggggttgcggcgtcatctcgaacccgaatcgttagcgaatatagcgcctttttctcataatcaggagccttcgtgcccactgtgacagccccactcgacgaatctatttgaaatacgtcatcatcatctccagctgcaatgtaaaatgaaaccgtatcgattgcatctgagtcacttcctatgacagtaccaacagctgtaccgtaaggcacattctcgatcaattgagtgtgcgcattggtgcacttaggtggatgattaatatttcgtgatagcacaccgattgtcacttcactatacgcagatgaattcactgtgtccgttacacgcaatgtgatgttaacgcgttgatcgtcaggtacatacgcgtctggccgcacagatagttcgcatgacgtggtgtcgctgctggttacaatcttgaaattatcagttgagtccgccgagatggagcactttcctccaatctgtcccggatctctgtcatgtattggtaaggacacggtagcataagaaccatcccctagttgactaatgtcaacatgataataactctgcgctgtaatagccccaacaaactgtgggggtgagggtatcattttgacagcgatatcaattggatgagagagcccactaagcccatctgggtcagtggcttgaataacgaatgagtattcgctgacaatttcagcattcagttgtgaattcacagtcagcttccccgtagattcagaaatagtgaattggtcaagcggattaccggagtgtattgagtacacaagttttgtcgattggccggtgtcttcatctgaaaacaaacatattgaatcaaggatcgccccaacaggtgtggtttcgggaatggtaacgccatctacgactcggttcacaacaggcgcatcgggaccatctgtaatttcgacaagatttggaccaatctcggcgcacatgcctaatggatcgcaggcactgatgttggccccaagcgaatgaatcgataacgcctcataatcaagttcagctattgtcaccaggaaactaccgttgataccaaaaagctccccaaatttgccagctgaatgaacaattgtatacctgatattatcgccttctgggtcgactgcgtcgaacgctaaaatcacagttccgacaggaacattctctgccacttgtttcgtaaagggtgatccagtaggagcttcatttacatcaacaacctgcacagtcattgttgcctcatcgctgctgcctttttcatcataaactgtaactgatatctgaattgcaggattcgtttcaaaatccatcggttttgaccataactgagaaccttgaacaaacagaaagtcgctcgtggacgttaattgaaatgtcaggactgcacctttgtcctcatcaacagcaaaagtacctagatttcctaccaagcacgtgttcccgatgcagttttcggaaacgcgaaaaacagcacttgttagaactggagcatcatttgagtctgtcacagcgatgcgataatcacacactgtttgaagccccgttttatccgtaaaacgaacagtgaagggcacggtgtcctggacttcgtagttgagatgttttttcaacgtcactttaccagtggaagaatccacatcaactgttcctatcgggatttgagataatacttcacatatcagatggtcgccggaatcggggtcgaatgcgtacccactcaactgtgcaactccagtctcaatcgaggtattttcagccacattaaatgaaaaggatacacaacttggtggctctggcatatcttggatcacgataaccacgttagcgtgagattccaagccaccaccgtcctttgcaacaacatccaatacgaattccgttgtactttcatagtcaatagcgttgatcgcaacgatattaccatcccgagtaatgacaaagttgccattcccaccactagatatagataaggtatgcgtgtcgtatgcatctggatcaatgacaccaacagcaggcatgagagttgcgggaggtagagtattttccttcaatgctcgagaaaattggtagccgctaatgatgggtgcctcattgacgtctaccacattcacatggattttacacgccgcaaacggtgcactcgctgaggtctcttggatcatgagtgttatttcgtatgcgttttgattctcgtagtctaatgccttcatcaggattaaatgtttctgctcgttcagtttgaaaaggtttgtagaagtggtcgatgtctggaggttatacgtcataccgattgaagaagatgcgaggctaaaactgacatccgagacaaatccagggtcattttcgtggatgctcagaatgtgatttctgtcgacacaaatcgaatctgggaacacaaacgacagcttacgagcaccacctgagcaactatagcccgtgggacaccttttgtattcgaatcggcgatcattcacagaaccctccggaatagaatagtaacccgctgggacattgatagcattgtaatacgagattgcttgtgatagcgggagtgccatctgcaagttgtaacgtgatcctggggtgactacctcgaacgtcgctggagtagtactgtcattgacgccaacgaacaaaaagcccgaggctgtagagttagtactaatctgagatatgccgtacccggttatccaggtactcaaactcgacggaattttcacgatacttgtaccaatgagaacagcaatatccccagcacccatcgcagataacgtgggcatataattattagacatcgtagcgtcaacagtagtgtcaaacatcttcgtgccaagtgccgagtacactaaaatatgtaagatatttgctgtatctgctatagttttcacagtttctccgttgacatagacactacccgtgcaagttggggcggcaattccgcataccgcgatatcgatatctaatgagatagactggccgtcgaaacgatcgcaacgcaagttacgaaagcttgcatcggtagattttcggaataaaattgttcttttgactacaaactgtgattcagctgcgataatggacaaatagtgttgggccccggacgatgacacaaacgcaaatgtgaatgtaccatttcgtgagcgaggaaccgcgataatcccatctagcagtacaacagcaccccaatcaaatccagcaacgggcagttcgaactcgtaggtgatatccgtctgtgaatgttcgaaataagaaatcgatatgttgtgtgcaaacccagaagaccaccctgtcttaaacgcagtcaggtttacatctttgaactcgaaaacgtctatagaaacagccgcagaactacttgtagcagcaaaaacaccttgcgtaagggcgtcgtccgttgaacctggttgtttcgataaaggtgcgagaagatatgagattgatgacgaagtcaaaactacttggtcagcgccactgggagaatctgggcatcggcttacgcttccagccttacaaacacaaccttgcgcacatcgaccagagcaaagtgagttgatgagacctacttgatcgccgaatcgagtaggtggacactgatatgctgtgcctcgtcggcaataataccctcttggacaggacagtattccagattgttgctcattcaccatcgatccgacagagtaataaccaggtggcacaatgatagggccagtgtttccttctgggcagtacacatttctagaggggcaaggcgctaacgcagtgtaggcacaaggggccccgctggaggggcagtagaaaccagcggggattgcgctacaattttgttgacctggcttcgaaaaagttccattcgtgcaaggaacgaaatctcttcgagccatctgatcaaatggacccgcataaccctctgggacattcaaacattcgcttgatccagcgtcagcaaaggtgccaacagaacacttagctggtgttgttgaaccgcttaagcatttgaaacctgatgggcacgatatgcacgctgcaacacctgacaacccaaccgacggcccatacgtcccggcagggcacatcgtacatccgttcgagtgtgattctgaataggtacccgccggacactgtgaacagtcgccactctgtgaactcgtgtagcctggtgggcatctgtccggccgtaaaagtaattttgaggagtcataaccatccacgttatcccatactatactccccgttttctgacaccagctttgcgcccatgcagcgtaggatgtgcaaatgggtagcatgtcaaatggcgcgtttaaatcggatctagttgcactaagacagtctgcataccagaatccgctccggcttcgcctcgcacaattttgactcgcggacagatcattatcctggtctggtgtgctgaaatacatattactaagcggggtaagagcatccgttgcataactcgccgtcgaattgtaccctcgcacagtcaataagtatttttccttttcgcttgccactcgaaaatcactataaaacgcggaagtgtccttataatctttggatacgacgatgattgcctcggtgactcgtgctgtgatagtagccagaatatcatttccaatccaccactcattggcagaaccaagaccaaacccgtcacgatactcattccaattgcgattgaaagttgtcttgccgtttacacggcgttgtgcaactatccatccgtcagagcaaggaagaaggactggatcaacatttgggtcgagttgaatatagtactggccatctggattcacatcaccgttggcagcacgctcgctcttcaggtgcatgcatgaaaccgcgggatttgctaacgatagtcctagaggtgtcttaaacgatgattccacctcaattgaaacttcgtcaatcccaaaggcactgatgttcaacgaaccaaaatccgtttcaaaacgtagttcgtttgctgctccgctaaatcctctcacagcaaatcgctcccttccacggtagaagtatgcctgttgcgctgaggtcgaactctgtccgtagcaagcataggtcactgaggtatcaacttgcagctgtcgaacaagttgggtaccaaggtaaacccgaatagtcctcggttcagagctcttgagaagaagtgctacccagtaaagtggttcaaacacaaaatagtatgtgaatttgacgcgcgcgttaaatggtttgtcgctcttgttgatggggctcaatgttttccttacaaaggatccgacccgtgagaatgatctacctccaagaattacgattccttgacaaagctggtaccacttgtctgcaccgctccaagaagttacccagagattgtcatcaccgcttccaataaaagagtcttggagagtgacactccaagaggttgatggactaacgtcgttggtagaacacaagtatctagtcgttgccagtccacgaggatcaaaactaaaagataagccgtctgacacactttggccattgaaagcagaatgcatgcctatccaccttcgttcgtctggtgcattcgcaggcgatacagtatcacaaatgctaccgttatcacgtaaccaccattttcctccatcggttgctgtccaatcgagcacagtttcgtcatcacttggtaggtcaatactatcgccaacgtaggcattggttactccgggaactgtcgagaaaaagagtgctccatattttcggtgcattgatataaaatgccccgaagaccgcggtaccacttgttgaagacccatgatcttacaactatcactgtcttttgggctagcactgctcagacaattgatgtcactgatgcccaaatcacacggcacaatcgtgtatccctcaccgctatcagcggtcatattgcaaaacgcatcatatattcctgctgacgtcgctatgacatatcgtccagttgccgcatctggggcatcgactttgatagcgtggcaagtctgaggcatattcaaatgaatagtagcggcacccgaggtattatatgtgcccaattggacactttggcgaaagtctgatgctgtgatagcataacatgttccatccgtatttgcgaagcctacacagtcactgcttgattggcaaatgcatcggcagcgctctttcaatgtgtacgtttgagaacaaataccagttccgcacgcgtcgcctttgcatggggttgcaagagatacattctgtccaagtgcccactttgtgttgtgaagaatcgtccacgcattccccgaaataaccacttcctgggttcttacaaacgccacgcgagttggatctgtcactaacgaggacgaattgcgagtcatataaaggttgcatgccttcgtagcaatgttgtatacgcctactcggcatagaccatgccgtgaacagcgtgactgacacatttcttgcgacagcaaggcacctctctcgaccgtgacaatttgtagttgcaccccttttaactggtacccagaactcgtcgatgttttaacgtcaactgttactgtgtgggagcctgcaacacaatcgataatagagtgcattgtcaaaggctcacacattcctgacaacactgtccccgccactgaagagaaatctccatcaaagatcgggtttccgtttttcgatatgcgcgcacgaaatacttcggtggctgcattgcggcagatggtaccaccaacacgaattatcaatgtgcttgaggaagagagtactaccattttggagaagatcgtgctattcaaggcagccgtgctgtcgctcaaggtttcagtagctaatgaaccccctggaacaaatgtggctgtcgttctgctaccaataatctttccagtcttcgttccttggtattgataactgagcaagatactcagtgcgctggctgttggaacgctagtaaagtaagaaaattcaagaggcacaccagaagggtcaataaatgattcatccaagcgaaactgagaggtgactgatgtagtgcttgtgccgaacccaaggtcgagacgaaacgttttcacagtacttttgtccacgaaaaatcttcccgtaaaagaacacatgagaaccccacttgttgcaggcgtcagggtcatacttaagccaggtattgcactccatagtgtgctaaccgctgtagcaaaagaaacttgagatgcccttgcactagcccacatcgaagttggaagaaacgagaaggacactaaggacgaggtatttagcttagcactcggggtatctgtcgtcgatagcgctgctaagtgcaatgcgaagcgtccgtcaacaggtgcactttggaactgtagtactgatacccctctgccaacattgctagctttgttagcttgttcagccgtaagcgtagctgatatcgtaaaatcaggagcagcaaagggagtccattttgtgagttccgtaatgagagcagcggtggaaacgagtaccgggttggatccttcgcgactgaaatatagactcgatgcttcaaactttgcagataatccgtaaaccttcaacacatgaaaaccgggtgtgattttcatgtcaacttgggtggttttcgtgaggacacctttccaatacccagtcttgcccatcaaatcttgcatcaccacaccatccaggaccacgacagcgccgttaccgagccgaaagggcagacctaaataccacgaccagcttgtattaactgggctagactccacaaagtccacttgcatcagaaaacccacgtttgtgtccacctgcttgaatctgccgcaaagatcgctctgagtgttcaataaaggtcccagtacagatagttctcgctcacaaaacccgtcgctttccacgggtgcaggggcagcgtcaaagtctttcagaaaagttggcccttgcccagctttgtacgctgttgctgagaatggttttgacgcataagagatctttgcgtatggactcagtgaagcttcaaaacggaaagcattcgagtgtgtcagttgctgttctacatttccaattgcaataaccagatacgtactggggtaatttagtcgcgattcgagtacattttccgtgtaatatcgagcatcctggtgtgagtggtaatggcaaacaacaatgcgttaggtgtttttaataccaatgccatggttggtcagcaaactgataacctacagttctgtagttgacgtgggcgacaagtactccaccgctcggagtcaccagactcgccgtcctgtccccatccaaattgtcatacacgaccgacggaagaactgcaattgaattagcctttatgaaaatgacgaatactggggggaggggcttcgcttacatggcgtgagctccacgttcgtgtctgtcggacgagagttgacgaaggtagtgaagctgggaggtgcgcatgccccagggtggaagagcagagcccatccggcgagcgtccacacgggtagcgagcggagaagcatgagcgggctctgggttctctcgcttggggtgtggtccgaagatccctcgaaagatataccggactgtattcttggaccaaaagtctagtaaaaaaaaagcctatattagaacaaatgtagctactttaacctgcacggaggcactgtgagcttggtgacagcatgactgctgcttctgccttgtcgtcagtctgatggtcttggcttgcctcttggtcagatcactactccgcctccttgcgactgccagtgacgtagccgtcatcggcagcctcagccgtacccccccccccccatccgggagggcgttaccccagcctgaccacgacacgactcagacaggaaggccttggcaatgccgagtcagcatctcccagcttgcgccgaacggtagactggaccatattaatccgacgggcaatctctctcgtcgagagagctggcgggggccccctgatcgctgctgcgaccgcagcaatagtgttctcggggggcgtggcggccggctagttcggtgcactaaaacctctggcggtcggtgtaaggcatccctcacgcactgctcgctcaggtggttctttagcgcaatggccttcacaatagtccccttgagctgcgtccccggtggtgcatcaccgtctgcgcgtcctcgaggatttgcgcgtgctgccccggcgtgatcgtagccttgctattcttcttggagctagacggcatgctggtgctggaagatcgaagtggccatgcgataaggctgatcgaagctcgagctattactgagttacgcatatggagttgcttctactaagttgcagtattgttactctacctcgacggccaccagttcgggctccagaggttgacgacgatcacagacaccttgaatgctgctacaccttcctctagtgccgcagcatcttcttctgcaaccgctgcatcggatcgagtaccacaccaattgtgacccctgcagtactttcagccgttagtgttgtctcggctgcttctcctttccttatgtaggcaggctcaaagtaagctcaatgcagttgttttataggcatgtattggcgtattccaccgcttacagttgctgaagaaggtgtgcttggaaccacgcagactccttacattggtgggtgtgccaatgcaattacgatgaagtgaggccctggcctcgactaatctggaaaggggtgtcgtcggctgtcacttggtgcatctgacagtgattgccctttcgtacagaccgggtgcagttctcagcctcgcaccgccgaccgccaccgtgagagatgcagtagccattgccacggtccttcttcttgcagccttccatgatgcagcggcgaattccgccgtgcaagtagcatagtccaagacgctgcgtccccttcgagcatcctggaacccggcacttcttaccacctccatgtgctcgacaaagccctccaccttgtgaactcttgtcgcaaccctgcacttggcatcgtgcgccgcccccgtgggtcttacaccggttgtttgattgcgctcgtcgagtgcatccttctgctatacactgcttcgctcgacgagttcctttcgatttggtagacgaagtggacgacactgtcgaagatgaagacgtgttcgaaactgtccgcagagtcggcgggggcagcggcactgatggtgcgaagtacggcacgttcttggcgaggagttgacttccaaatggcgacggaacgggaaggaggaaattgagtggtaacggattcacacgcccaggggtagtcgagattgtaaacgagcttggacaactgctggtcgtcttaaaagagggacgcgctgatcgtgtccccggaccgacgatgtcgtcccaggactcacgcaagaaggagtccatgaccggtgaaaagtcaggcagctcgctctcctgtttgatgaatacatctccagccatgggtgtacggccattaatgcggatcttcaagtcaggcgaaagacacgaagaaacaccggctggaaatccagatacaccagcagctagtgggtgctgcacattagcggcgagaggcgactccgtgtcttgcgggttgaaggcatcactcagtcccgtgaggttcaattcgagattgtcttggtcatctctttgaaatgccgatgtgggtgaggtggctgcggagacagcgcccgtgttatcgagcatcatcatcgtgctttcgaagtcgtcgtcgtccagcaggtccattgatgcagacagattcaaaatatccccaatgttgtacaacgacgcgttaccactggtgataggcgaagtgcgaggatccatggctggcggtatcgtctaggactgaagttgggatgcacttgcggtacagagcgaaattggatgtgagggagaagcctacctgttagaggcaaacgctgtcatagttggtagaatgaataaggtccgggatgttggcgtgtcctgatgagcagcgccttcgccatatgccccttgtatcttccctccaagccttattagcgattctggtgtagctgggtgagatggcgagtcacagcgtcaagcttcacgtcatgcacaccattttgtgttgagtataaatatccctcccgaccaggtcaacacccccagtgttgacaacgagaactgggtttagtactttgaagtcttctgtatgttagcaaaacattcaaaaggaactcaaatctgccaccaatgctgaattcatccgagcctcgtcaatcagaaaattatcagtacctcttaacaccattaagatgctctaaaggataaacgactgctggatgagctcaccttgagtcttcagagcgcacccgtacatcattttgctaagctcgttgagaacgaagtctcgtgtgcgttcgacatcctcaactcgacccgctagattaggctcgacctctgcataaatgcgcaggtagctacgcaccacggacgcaacgcgaggattcacctcggatgcagtgatgactgtccatgatggtgatcgaaattcaagcccttgtgtctgcagaatgattgagtccgcttttaccagaacaatccgctgggactcttcgaatttgcgagaaaaatggctcccgttaatctcaatgtcgctctgttctccccgtagtgtcatgacgaagctcttttcgagcgagttgggcttacttccgcgtagctgttttattagaggacacatgctagttaatgacaaaaagaaacagcgaagaaacactgcagaacacgtacgtagcgatatcgcttatcggggatgtcgtgcaccgtatttaggtccttccacaagatcgaagatgcttgttggatcgagcattcgactggagtcacagtctcgatctcaattaccttgccgtactgcgtgttattgttcacgttcattttggacgaaaccaggcaaggctggtcccacccgagtccggatttcgagtcgacgtacaagttgtccgcgtgtttctccaatatcttgaggagccctgggctgttgtccaaaggttgtggtggtcgataaatcggaggcgaaccaccaaacacgaggtcaataccctgcgtgagtaccaaagggattagtttcaccagggcacaaagctgcatcaagtcaaaagatacctgctaatacgtacgtcaagaacactgcgtctctgcaacaccccacgaagagactctaccacttgcctctggtttgccaggataactttcagctgcttgttggtgttttcggactgctgtc

SEQ ID NO: 17 - protein, 493 aa, Simmondsia chinensis
MEEMGSILEFLDNKAILVTGATGSLAKIFVEKVLRSQPNVKKLYLLLRATDDETAALRLQNEVFGKELFKVLKQNLGANFYSFVSEKVTVVPGDITGEDLCLKDVNLKEEMWREIDVVVNLAATINFIERYDVSLLINTYGAKYVLDFAKKCNKLKIFVHVSTAYVSGEKNGLILEKPYYMGESLNGRLGLDINVEKKLVEAKINELQAAGATEKSIKSTMKDMGIERARHWGWPNVYVFTKALGEMLLMQYKGDIPLTIIRPTIITSTFKEPFPGWVEGVRTIDNVPVYYGKGRLRCMLCGPSTIIDLIPADMVVNATIVAMVAHANQRYVEPVTYHVGSSAANPMKLSALPEMAHRYFTKNPWINPDRNPVHVGRAMVFSSFSTFHLYLTLNFLLPLKVLEIANTIFCQWFKGKYMDLKRKTRLLLRLVDIYKPYLFFQGIFDDMNTEKLRIAAKESIVEADMFYFDPRAINWEDYFLKTHFPGVVEHVLN

SEQ ID NO: 18 - protein, 460 aa, Bombyx mori
MSHNGTLDEHYQTVREFYDGKSVFITGATGFLGKAYVEKLAYSCPGIVSIYILIRDKKGSNTEERMRKYLDQPIFSRIKYEHPEYFKKIIPISGDITAPKLGLCDEERNILINEVSIVIHSAASVKLNDHLKFTLNTNVGGTMKVLELVKEMKNLAMFVYVSTAYSNTSQRILEEKLYPQSLNLNEIQKFAEEHYILGKDNDEMIKFIGNHPNTYAYTKALAENLVAEEHGEIPTIIIRPSIITASAEEPVRGFVDSWSGATAMAAFALKGWNNIMYSTGEENIDLIPLDYVVNLTLVAIAKYKPTKEVTVYHVTTSDLNPISIRRIFIKLSEFASKNPTSNAAPFAATTLLTKQKPLIKLVTFLMQTTPAFLADLWMKTQRKEAKFVKQHNLVVRSRDQLEFFTSQSWLLRCERARVLSAALSDSDRAVFRCDPSTIDWDQYLPIYFEGINKHLFKNKL

SEQ ID NO: 19 - DNA, 1491 bp, Arabidopsis thaliana
atggaattcagttgtgttcattttcttcaaaacaagacgattcttgtcacaggcgcaaccggtttccttgccaaagtttttgtggagaaaatattgagagtgcaaccaaatgtgaataagctgtacctcgtggtgagagcatccgacaatgaagccgcgacgaaacgcttacgcacagaggcattcgagaaagatcttttcaaggtgttaagagataatcttggcgatgagaaattgaatacattgttgtccgaaaaagttgtcccggtcgcaggtgatatcgcgatggatcatttaggcatgaaggactctaatctaagagaacgtatgcaaaaagaaatcgatattgttgtcaatgtcgcagctacaactaatttcgacgagagatatgatattggtcttggaataaacacatttggagctctcaatgtccttaacttcgccaaaaaatgtgttaaagctcaattgcttctccatgtttcaactgcttatgtttgcggagaaaaaccaggtctcctacctgaaaaaccgttcgtcatggaagagatttgtaatgagaatggtcttcaattggatataaaccttgaaagggagctgatgaaacaaagattgaaagaactcaatgaacaaggttgttcagaagaaggcactactttctacatgaaagaactcggcatggaaagggcaaagcttcatggatggccaaacacatatgttttcaccaaatcgatgggagagatgcttcttggtaaccataaagaaaatcttcctctcgtcattatccgtcccacgatgatcactagcactctttttgaaccatttcctggttggattgaaggactaagaactgtagacagtgtaattattgcgtacggaaagggagtgctcaagtgttttcttgtcgatgtaaactcggtttgcgatatgataccagcggatatggtggcaaacgcgatgatcgcagctgcagccacacatgctggaggttcaaaggttcacatggtgtaccaagttggttcatctcaccaaaacccaataatatatggagagatccgtgaaattttgttttgttacttcaccaaaaactcgttgcgcagtcgcaatggctcaatgataactgtctcgaaaatgaagctgataccaactctggctttgttcagcctctacatgaccatacgttacaaactacctgtccagttattaaaattggttgatataatatatccttcgagggaaggagacgaatacaaaaacaaaaaccgcaagatcgatatggtgatgagattggtaaagctttacgagccttacgtactcttcaagggcatattcgacgataggaatactaaaaacttatgcgccaagcagaaagaagaggacaacagaaattcagaaaattttatgtttgatttcgaccctaaaatcattaaatggaaagattatcttataaatgtacacattcctggcctcatcactcacgtgcttaagaagtga

SEQ ID NO: 20 - DNA, 1584 bp, Arabidopsis thaliana
atggctaccacaaatgtcctcgccacgagccacgccttcaaattgaatggtgtcagctacttctcctcttttccccgcaaacctaaccactacatgcctcgtcgtcgtttatcacatactactcgtagagtccaaacttcgtgtttttatggtgagacgtcttttgaagctgtaacgtcgttagttacgcctaagacagaaacaagtcgtaacagtgacggaattggaatcgtccgtttcttagaagggaaaagctatcttgttactggtgcaacagggtttcttgccaaagtgttgattgagaaactgttgagggaaagtcttgaaattgggaagatcttccttctgatgagatccaaggatcaagaatcagcaaacaagagactctacgatgagatcataagctcggatctgttcaagcttctgaagcaaatgcatgggagctcttacgaagctttcatgaagagaaagttgattccagtaattggagacattgaggaagacaatctagggatcaaatctgaaatagcaaacatgatcagtgaggagattgatgttattatcagttgtggtggtcgtacaacattcgacgacagatacgattctgccctaagtgtcaatgctcttggaccggcttacgtgactggtaagagagaggggacagtactagaaactcctctctgcattggagaaaacataacttctgacttgaacatcaaatccgagctgaaactagcttcagaagctgtaagaaagttccgtggcagagaagaaatcaagaaactcaaagaactcggttttgaaagagctcaacactatgggtgggaaaatagttacacattcacaaaagccataggtgaggctgtaattcacagcaagcgaggaaacttgcctgtagtgatcataaggcctagtattatcgaaagctcttacaatgagcctttccctggctggatccaagggacaagaatggctgatccaatcatcttggcttatgccaaaggccagatttctgacttctgggcagatcctcaatctttgatggacattatacctgttgacatggttgcaaacgcagcaatagcagccatggcaaagcatggttgtggtgtcccagagttcaaagtttacaatttaacttcttcatctcatgtgaaccccatgcgtgctggcaaattgatagacctctctcatcaacatctgtgtgactttccattggaagaaacagtgatagacttagagcatatgaaaatccacagttccttagagggtttcacttctgctttatcgaacacaataataaaacaggaaagagtgattgataatgaaggaggaggattgagcacgaagggaaagaggaagctaaactattttgtgtccttggcaaaaacatatgagccttacacattctttcaagctcggtttgacaacaccaatacaacaagtctgatacaggagatgtcaatggaagagaaaaaaacgtttgggttcgatatcaaaggcattgactgggagcattacattgtcaacgttcatcttccaggtctcaaaaaggaatttctttctaagaagaagactgagtaa

SEQ ID NO: 21 - DNA, 1628 bp, Ostrinia scapulalis
gaaaacagtctagaaaaatgtcagcaaataccatggaaactgatgaacaatttactgataattcaccaattgtgaatttttactctggaaaatctgtttttgttactggagctacaggatttctggggacggttttagtcgagaaactgctgttctcttgcaaaggaataaataatatttacattttgataaagcagacagaagacctgaccattgaagcgaggattttaaattatttgaattcgaaggcttttcatagagtgaaaaatacaaacccagagttgatgaaaaaaattataccgatatgtgggaatttggaagataaaaatcttggtatcagcgacagcgacatgaaaacgcttctagaggaggtatccatcgtttttcatgtagctgcaaaattgttatttaaaatgagcttgactgcagcagtcaatataaataccaaacccactgaacagctcatagcgatttgcaaaaaaatgcggcgtaatcccattttcatctatgtctctagcgcatacagtaatgtaaatgaacaaataatcgatgaaaaagtgtacaacactggagtacctttggaaactatttatgatacgctggatacagaaaatacacgaataacggatatttttttagataaaagaccaaatacgtatacctattcaaaagctcttgcagaagtagtagttgaaaaagaatttgatgaatcagcagccattgttcggccttcgataattgtgtcttcgattcgggaacccataccgggatggttgagcggttcgcacggattccctagggtagtaggagcagcatgcaaggggctcctcttgcggtggcatggggacggtacagttgtctgcgaccttatacctgtagaccacgttgcgaacctcatcattgcagcagcatgggaatccaatgaaagacggttaatgggcaacaaaggagtcaaggtatataactgttgttcaagcctacggaacccaatagacgtgatcaccgtagttaaaacttgcataaaatacaggaaatattttggaactcgcaccatgtccatatttaccccacgatttattatgaaaaagaattactttatctacaaattgttgtacttcacctgccacacaataccggcagctataatagacggcttcttctggctcactggacggactccaataatgctgaagaccctggacaaactcagcaaaatctcttctgtcctggagtacttcacgcaccaccaatttatattcctggacagcaacgtcagaggacttctcagaaggatggagggcacagacagacaaacgtttaattttgatgtcactgaaattgagtgggagccgtatctacaaaactttgtgcgcggcatcgcaaataattacgactatagtatgtaatatagtattgaggtttgatattgattgaggagaggtgaaatttagcctatgttactcgtgaataatgtagctttcgaatggtgaaagaattattaaaaacggtccagtagtttttgagcctataaattacaaccaaacaaacctaatattagtgtagatttgaatttgaaaagtgaggtataaaatcttgtcattactgtcaaaaaaaaaaaaaaaaaaaaa

(19) United States
(12) Patent Application Publication
(10) Pub. No.: US 2016/0052846 A1
(43) Pub. Date: Feb. 25, 2016

(54) METHOD OF DEMULSIFYING AND PURIFYING ORGANIC PRODUCTS FROM AN EMULSION

(71) Applicant: CODEXIS, INC., Redwood City, CA (US)

(72) Inventors: Owen Gooding, San Jose, CA (US); Gary Lee, Belmont, CA (US)

(73) Assignee: Codexis, Inc., Redwood City, CA (US)

(21) Appl. No.: 14/439,372

(22) PCT Filed: Sep. 27, 2013
(86) PCT No.: PCT/US2013/062295

§ 371 Date: Apr. 29, 2015

Related U.S. Application Data
(60) Provisional application No. 61/724,795, filed on Nov. 9, 2012.

(51) Int. Cl. C07C 29/86; B01D 17/04; C12P 7/04

(52) U.S. Cl. CPC C07C 29/86; C12P 7/04; B01D 17/047

(57) ABSTRACT

This invention provides methods to demulsify organic products from emulsions, using demulsifying solvents which act as a combination of demulsifier and low volume extraction solvent. The methods can be applied to purify organic products such as fatty alcohols from emulsions including those generated from fermentation broths.

### CROSS-REFERENCES TO RELATED APPLICATIONS

This application claims priority to U.S. Provisional Application No. 61/724,795, filed Nov. 9, 2012, which is hereby incorporated by reference in its entirety for all purposes.

### REFERENCE TO A “SEQUENCE LISTING,” A TABLE, OR A COMPUTER PROGRAM LISTING APPENDIX SUBMITTED ON A COMPACT DISK

The Sequence Listing written in file 90834-005910PC_ST25.TXT, created on Sep. 3, 2013, 391,272 bytes, machine format IBM-PC, MS-Windows operating system, is hereby incorporated by reference.

### FIELD OF THE INVENTION

This invention relates to methods of demulsifying and purifying organic products, such as fatty alcohols, fatty acids, fatty acid esters, terpenes, terpenols, triglycerides, carotene, carotenoids, β-lactams, sterols, statins, mycophenolic acid, aromatic odorants, lactones, antibiotics or antifungal compounds, pyrethoids, ketones and the like, from fermentation emulsions produced from microorganisms such as bacteria, cyanobacteria, fungi, yeast and algae.

### BACKGROUND OF THE INVENTION

The purification of organic products from emulsions can be difficult and expensive. This is particularly true in the case of fermentation broths which can form thick emulsions from the combination of water, cells, and the organic product, and which can be stable for weeks or months (see e.g. Li et al. Food Microbiology and Safety 66(4): 570-574 (2001) and Abbasnezhad et al. Colloids and Surfaces B: Biointerfaces 62: 36-41 (2008)). This makes typical liquid-liquid separation difficult. While demulsifying agents, such as peat moss and polymeric materials, are commercially available, they are expensive and have other disadvantages, especially where it is desired to recover the chemicals used in the production and purification process. Further, if the organic product to be recovered has polar functionality, the purification can be further complicated by several factors. Because polar functionality can make the compounds more soluble in water, they may form more stable emulsions and be more difficult to extract into non-aqueous solvents that must be lipophilic enough to be sufficiently immiscible in water to form distinct, separable phases.

In addition, traditional liquid-liquid extraction often requires several equivalent volumes of water-immiscible solvent in relation to the amount of aqueous solution being extracted, which increases production costs. Thus, there remains a need for efficient methods for demulsifying and purifying organic products from emulsions, such as those produced from fermentation processes. The present invention addresses this and other needs.

### BRIEF SUMMARY OF THE INVENTION

In one aspect the present invention provides methods of demulsifying an emulsion comprising contacting an emulsion with a demulsifying amount of a demulsifying solvent, whereby the emulsion transforms into at least two distinct phases. In other aspects the present invention provides methods for purifying an organic product from an emulsion, said method comprising adding a demulsifying amount of at least one demulsifying solvent to the emulsion, whereby the emulsion transforms into at least two distinct phases. In other aspects the present invention provides methods for producing an organic product from an emulsion of a fermentation mixture, said method comprising culturing a microorganism that produces an organic product under conditions wherein the organic product is produced and adding a demulsifying amount of at least one demulsifying solvent to the emulsion, whereby the emulsion transforms into at least two distinct phases. In some embodiments, the methods comprise a batch process, a fed-batch process or a continuous process. In other aspects the present invention provides an organic product recovered, purified or produced by the methods of the present invention. In other aspects the present invention provides compositions comprising a) an emulsion of a fermentation broth and b) a demulsifying solvent. These and other aspects are described in more detail below.

### BRIEF DESCRIPTION OF THE DRAWINGS

FIG. 1 shows a schematic diagram of a demulsification batch process as described herein.

FIG. 2 shows a schematic diagram of a demulsification continuous process as described herein.

### DETAILED DESCRIPTION OF THE INVENTION

### I. Definitions

Unless defined otherwise, all technical and scientific terms used herein generally have the same meaning as commonly understood by one of ordinary skill in the art to which this invention belongs. Generally, the nomenclature used herein and the laboratory procedures in analytical chemistry, cell culture, molecular genetics, organic chemistry, and nucleic acid chemistry and hybridization described below are those well-known and commonly employed in the art. It is noted that as used herein, “a,” “an,” and “the” include plural references unless the context clearly dictates otherwise. The term “or” is used herein to mean, and is used interchangeably with, the term “and/or,” unless context clearly indicates otherwise. The terms “comprising,” “having,” “including,” and “containing” are to be construed as open-ended terms (e.g., meaning “including, but not limited to,”) unless otherwise noted.

In the following description, all numbers disclosed herein are approximate values, regardless whether the word “about” or “approximate” is used in connection therewith. Numbers may vary by 1 percent, 2 percent, 5 percent or, sometimes, 10 to 20 percent. Moreover, any numerical range defined by two numbers as defined in the above is also specifically disclosed.

“Demulsification”, or emulsion breaking, refers to the process used to separate emulsions (e.g., oil in water or water in oil) into separate phases. The term “demulsifier” generally refers to a compound that breaks an emulsion formed when an oil or a hydrophobic substance (e.g., an organic product) is mixed with water or an aqueous substance. A demulsifier allows the oil and water phases to separate. Commercial demulsifiers are typically acid catalyzed phenol-formaldehyde resins; base catalyzed phenol-formaldehyde resins; polyamines; di-epoxides and polyols. These are usually ethoxylated (and/or propoxylated) to provide the desired degree of water/oil solubility. The addition of ethylene oxide increases water solubility, and propylene oxide decreases it. Commercially available demulsifier formulations are typically a mixture of two to four different chemicals, in carrier solvent(s).

The terms “demulsifying amount” refer to the amount of the demulsifying solvent that will elicit the demulsification of a media that is being sought by the researcher, or other of skill in the art. The term “demulsifying amount” includes that amount of a demulsifying solvent that, when added to a reaction mixture, is sufficient to cause the mixture to separate into at least two distinct phases. In some embodiments, the two distinct phases are separated by the aid of a centrifuge. The demulsifying amount will vary depending on the demulsifying solvent used and the volume and the type of media being demulsified.

“Distinct phases” refers to a multiphasic composition, wherein at least one phase can be distinguished from another phase either visually or spectroscopically.

An “emulsion” is a mixture of two or more liquids that are normally immiscible (nonmixable or unblendable). Emulsions are part of a more general class of two-phase systems of matter called colloids. Although the terms “colloid” and “emulsion” are sometimes used interchangeably, “emulsion” is used when both the dispersed and the continuous phase are liquids and not a solid. In an emulsion, one liquid (the dispersed phase) is dispersed in the other (the continuous phase). Examples of emulsions include vinaigrettes, milk, mayonnaise, cutting fluids for metal working, fermentation broths and the like. Examples of a colloid include the photo-sensitive side of photographic film. Two liquids can form different types of emulsions. As an example, oil and water can form, firstly, an oil-in-water emulsion, where the oil is the dispersed phase, and water is the dispersion medium. Secondly, they can form a water-in-oil emulsion, where water is the dispersed phase and oil is the external phase. Multiple emulsions are also possible, including a “water-in-oil-in-water” emulsion and an “oil-in-water-in-oil” emulsion. Emulsions contain both a dispersed and a continuous phase, with the boundary between the phases called the “interface”. Emulsions may be stable or unstable. Energy input, e.g., through shaking, stirring, homogenizing, or exposure, to power ultrasound can result in an emulsion. Over time, emulsions may revert to the stable state of the phases comprising the emulsion. Whether an emulsion of oil and water turns into a “water-in-oil” emulsion or it turns into an “oil-in-water” emulsion depends on the volume fraction of both phases.

“Emulsion stability” refers to the ability of an emulsion to resist change in its properties over time. There are three types of instability in emulsions: flocculation, creaming, and coalescence. Flocculation describes the process by which the dispersed phase comes out of suspension in the form of flakes. Coalescence is another form of instability—small droplets bump into each other within the media volume and continuously combine to form progressively larger droplets. Emulsions can also undergo creaming, where one of the substances migrates to the top (or the bottom, depending on the relative densities of the two phases) of the emulsion under the influence of buoyancy, or under the influence of the centripetal force induced when a centrifuge is used.

“Immiscible” refers to the relative inability of a compound to dissolve in another compound (such as but not limited to water) and is defined by the compound's partition coefficient.

“Liquid-liquid extraction”, also known as solvent extraction and partitioning, is a method to separate compounds based on their relative solubility in two different immiscible liquids, usually water and an organic solvent. It is an extraction of a substance from one liquid phase into another liquid phase. Liquid-liquid extraction is a basic technique in chemical laboratories, where it is performed using a separatory funnel. This type of process is commonly performed after a chemical reaction as part of the work-up.

As used herein, “partition coefficient” or “P,” is defined as the equilibrium concentration of a compound in a non-aqueous phase divided by the concentration at equilibrium in an aqueous phase (e.g., fermentation broth). In one embodiment of a bi-phasic system described herein, the non-aqueous phase is formed by the aldehyde or alkane during the production process. However, in some examples, a non-aqueous phase can be provided, such as by providing a layer of octane, to facilitate product separation.

When describing a two phase system, the partition characteristics of a compound can be described as log P. For example, a compound with a log P of 1 would partition 10:1 to the non-aqueous phase. A compound with a log P of −1 would partition 1:10 to the non-aqueous phase. By choosing an appropriate fermentation broth and non-aqueous phase, an organic fatty acid derivative or product with a high log P value can separate into the non-aqueous phase even at very low concentrations in the fermentation vessel.

“Process(es)” refers to a purification method(s) disclosed herein that is (are) useful for isolating an organic product. Modifications to the methods disclosed herein (e.g., starting materials, reagents) are also encompassed. “Partially processed” refers to a media which has been subject to a process which is useful for isolating an organic product.

“Organic products” as used herein (and sometimes referred to herein as “organic compounds”) are typically compounds produced (or which can be produced) in organisms such as bacteria, fungi, and algae. In general, organic compounds are aromatic or aliphatic nonpolar compounds that include carbon and hydrogen compounds. Exemplary examples of organic products that may be produced and/or purified according to the methods of the invention include fatty alcohols; fatty acids; fatty acid esters; terpenes (such as monocyclic terpenes (such as pinene and camphor), bicyclic terpenes, and sesquiterpenes (such as farnescene)); terpenols (such as sesquiterpenols); aromatic odorants (such as vanillin, cinnimate and eugenol); pyrethoids (such as chrysanthemate, allethrin, and permethrin); odorant lactones (such as aerangis lactone, cognac lactone and whisky lactone); odorant ketones (such as jasmonate and muscone); flavor and fragrance terpenoids (such as ambrox, ionones, and isopulego); triglycerides; carotene; carotenoids (such as lycopene); I-lactams (such as penicillins); sterols; statins (such as lovastatin, simvastatin, and atorvastatin); and antibiotics or antifungal compounds (such as mycophenolic acid). However, the invention is not limited to particular compounds, and it will be apparent to the skilled practitioner that may other organic compounds that may be purified using the methods of the invention.

A “fatty alcohol composition” refers to fatty alcohols produced from a recombinant microorganism (e.g., host cell such as E. coli). A fatty alcohol composition may comprise a plurality (e.g., combination) of fatty alcohols. As a non-limiting example, a fatty alcohol composition may comprise a plurality of fatty alcohols having carbon chain lengths, such as but not limited to fatty alcohols having a carbon chain length of C8 to C20. In some embodiments, a fatty alcohol composition may predominantly comprise fatty alcohols having a specific carbon chain length, such as but not limited to a fatty alcohol composition predominantly comprising C10, C12, C14, C16 and C18 fatty alcohols; a fatty alcohol composition predominantly comprising C12, C14, and C16 fatty alcohols; and/or a fatty alcohol composition predominantly comprising C12 and C14 fatty alcohols. The term “predominantly” as used herein refers to about least 90% of the total composition. For example, a fatty alcohol composition predominantly comprising C12 and C14 fatty alcohols will comprise at least 90% C12 and C14 fatty alcohols in the total fatty alcohol composition produced by a microorganism. The fatty alcohol composition may comprise saturated, unsaturated, and/or branched fatty alcohols. The phrase, such as but not limited to, (i) C12 to C14, (ii) C12 to C16, (iii) C14 to C16, or (iv) C12 to C18, is inclusive of the carbon chain length so denoted.

The term “fatty alcohol” as used herein refers to an aliphatic alcohol of the formula R—OH, where the R group is at least 4, 5, 6, 7, 8, 9, 10, 11, 12, 13, 14, 15, 16, 17, 18, 19, 20 or more carbons in length. R can be saturated, unsaturated, linear, branched or cyclic. Saturated or unsaturated fatty alcohols can also be described using the nomenclature “Ca:b” or, alternatively “Ca:b-OH”, wherein “a” is an integer that represents the total number of carbon atoms in the fatty alcohol and “b” is an integer that refers to the number of double bonds in the carbon chain. In some embodiments, a fatty alcohol produced according to the methods disclosed herein is a C8-C24 saturated or unsaturated fatty alcohol (i.e., a C8, C9, C10, C11, C12, C13, C14, C15, C16, C17, C18, C19, C20, C21, C22, C23, or C24 fatty alcohol). In some embodiments, multiple fatty alcohols are produced with varying saturation levels. In some embodiments, one or more of the following fatty alcohols are present: 1-decanol, 1-dodecanol, 1-tetradecanol, 1-hexadecanol, and 1-octadecanol. In some embodiments, the fatty alcohol may be a branched chain or a straight chain fatty alcohol.

Unsaturated fatty acids or fatty alcohols can be referred to as “cis Δ^{x}” or “trans Δ^{x}”, wherein “cis” and “trans” refer to the carbon chain configuration (geometry) around the double bond and “x” indicates the number of the first carbon of the double bond, wherein carbon 1 is the carboxylic acid carbon of the fatty acid or the carbon bound to the —OH group of the fatty alcohol.

The term “fatty acid” as used herein refers to a compound having the formula RCO₂H, or a salt thereof, wherein “R” is as defined above for a fatty alcohol. In some embodiments, the fatty acid salt is a potassium salt, a sodium salt, or an ammonium salt. Saturated or unsaturated fatty acids can be described as “Ca:b”, wherein “a” is an integer that represents the total number of carbon atoms and “b” is an integer that refers to the number of double bonds in the carbon chain.

As used herein, the term “aldehyde” means a hydrocarbon having the formula RCHO characterized by an unsaturated carbonyl group (C═O). In one embodiment, the R group is at least about 1, 2, 3, 4, 5, 6, 7, 8, 9, 10, 11, 12, 13, 14, 15, 16, 17, 18, 19, or 20 carbons in length. R can be straight or branched chain. The branched chains may have one or more points of branching. In addition, the branched chains may include cyclic branches. Furthermore, R can be saturated or unsaturated. If unsaturated, the R can have one or more points of unsaturation. In a preferred embodiment, the aldehyde is any aldehyde made from a fatty acid or fatty acid derivative.

“Fatty aldehyde” as used herein refers to a saturated or unsaturated aliphatic aldehyde. In one embodiment, the fatty aldehyde is produced biosynthetically. Fatty aldehydes have many uses. For example, fatty aldehydes can be used to produce many specialty chemicals. For example, fatty aldehydes are used to produce polymers, resins, dyes, flavorings, plasticizers, perfumes, pharmaceuticals, and other chemicals. Some are used as solvents, preservatives, or disinfectants. Some natural and synthetic compounds, such as vitamins and hormones, are aldehydes.

As used herein, the term “alkane” means a hydrocarbon containing only single carbon-carbon bonds.

“Fatty acyl-CoA reductase,” “fatty acyl reductase” and “FAR”, are used interchangeably herein to refer to an enzyme that catalyzes the conversion of a fatty acyl-CoA, fatty acyl-acyl carrier protein (“ACP”), or other fatty acyl thioester complex to a fatty alcohol, either directly or via two enzymatic conversion steps in which the fatty acyl complex is first reduced to a fatty aldehyde and the fatty aldehyde is reduced to a fatty alcohol. In one embodiment, a FAR enzyme is an FAR of EC 1.1.1.-, EC 1.2.1-, EC 1.2.1.50, 1.2.1.84, or 1.2.1.n2. “ACP” is a polypeptide or protein subunit of fatty acid synthase used in the synthesis of fatty acids. In some embodiments, the FAR is a fatty aldehyde forming FAR that catalyzes the reduction of a fatty acyl-CoA, a fatty acyl-ACP, or other fatty acyl thioester complex to a fatty aldehyde intermediate, which is reduced to a fatty alcohol by a second oxidoreductase enzyme.

The terms “fatty acyl-thioester” and “fatty acyl-thioester complex” refer to a compound having the formula R(C═O)SR and RCH₂CO—SR² (Formula I) respectively, in which a fatty acyl moiety is covalently linked via a thioester linkage to a carrier moiety. Fatty acyl-thioesters are substrates for wild-type FAR polypeptides and FAR variants.

The term “fatty acyl-CoA” refers to a compound of formula I, wherein the R group is as defined for “fatty alcohol” above, and “R¹” is CoA.

The term “fatty acyl-ACP” refers to a compound of formula I, wherein the R group is as defined for “fatty alcohol” above, and “R¹” is ACP.

The term “fatty acid synthase (FAS)” (EC 2.3.1.85) refers to an enzyme or enzyme complex that catalyzes the conversion of acetyl-CoA and malonyl-CoA to fatty acyl-ACP. In some embodiments, the separate polypeptides form one or more protein complexes.

The terms “fatty acyl-CoA synthetase” or “acyl-CoA synthetase” or “FACS” (EC 2.3.1.86) are used interchangeably herein to refer to an enzyme that catalyzes the formation of a covalent complex between the acyl portion of the fatty acid and CoA.

“Naturally-occurring” or “wild-type” refers to the form found in nature. For example, a naturally occurring or wild-type polypeptide or polynucleotide sequence is a sequence present in an organism that can be isolated from a source in nature and which has not been intentionally modified by human manipulation. A wild-type organism or cell refers to an organism or cell that has not been intentionally modified by human manipulation.

The term “wild-type fatty acyl-CoA reductase” or “wild-type FAR,” as used herein, refers to a naturally-occurring FAR polypeptide. In some embodiments, a wild-type FAR is produced by a gammaproteobacteria, including but not limited to strains of Marinobacter, Oceanobacter, and Hahella. Naturally occurring FAR polypeptides are described, for example and not limitation, in US patent publication 2011/0000125 (now U.S. Pat. No. 8,216,815), incorporated by reference herein. FARs that are not wild-type can be denoted “recombinant” FARs, whether prepared using recombinant techniques or by chemical synthesis.

The term “FAR variant” refers to a FAR polypeptide having substitutions at one or more positions relative to a wild type FAR polypeptide and to functional (or “biologically active”) fragments thereof. FAR fatty alcohol production and fatty alcohol profiles (i.e., chain length distribution) can be measured as described in WO2012/006114 and WO2013/096082. In one embodiment, “FAR variants” comprise at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to a specified reference sequence.

A “conservative substitution,” as used with respect to amino acids, refers to the substitution of an amino acid with a chemically similar amino acid. Amino acid substitutions which often preserve the structural and/or functional properties of the polypeptide in which the substitution is made are known in the art and are described, for example, by H. Neurath and R. L. Hill, 1979, in “The Proteins,” Academic Press, New York.

The most commonly occurring exchanges are isoleucine/valine, tyrosine/phenylalanine, aspartic acid/glutamic acid, lysine/arginine, methionine/leucine, aspartic acid/asparagine, glutamic acid/glutamine, leucine/isoleucine, methionine/isoleucine, threonine/serine, tryptopha n/phenylalanine, tyrosine/histidine, tyrosine/tryptophan, glutamine/arginine, histidine/asparagine, histidine/glutamine, lysine/asparagine, lysine/glutamine, lysine/glutamic acid, phenylalanine/leucine, phenylalanine/methionine, serine/alanine, serine/asparagine, valine/leucine, and valine/methionine.

In some embodiments, conservatively substituted variations of a polypeptide (e.g., a FAR polypeptide) include substitutions of one or more amino acids of the polypeptide with a conservatively selected amino acid of the same conservative substitution group. In some embodiments less than 10%, less than 5%, less than 2% and sometimes less than 1% of the amino acids of the polypeptide are replaced. In some embodiments, there may be at least 1, at least 2, at least 3, at least 4, at least 5, at least 6, at least 7, at least 8, at least 9, at least 10, at least 15, at least 20, at least 25, at least 30, at least 35, or at least 40 conservative substitutions in a polypeptide. In some embodiments, there is no more than 1, no more than 2, no more than 3, no more than 4, no more than 5, no more than 6, no more than 7, no more than 8, no more than 9, no more than 10, no more than 15, no more than 20, no more than 25, no more than 30, no more than 35, or no more than 40 conservative substitutions in a polypeptide. The addition of sequences which do not alter the encoded activity of a polynucleotide (e.g., a FAR polynucleotide), such as the addition of a non-functional or non-coding sequence, is considered a conservative variation of the polynucleotide.

The term “amino acid substitution set” or “substitution set” refers to a group of amino acid substitutions. A substitution set can have 1, 2, 3, 4, 5, 6, 7, 8, 9, 10, 11, 12, 13, 14, 15, or more amino acid substitutions. In some embodiments, a substitution set refers to the set of amino acid substitutions that is present in any of the variant FAR polypeptides listed herein.

A “host cell” refers to a cell (e.g., a prokaryotic cell or a eukaryotic cell) used to produce the organic products (e.g., fatty alcohols) described herein. In some embodiments, a host cell is recombinant, e.g., modified to express, overexpress, attenuate, or delete expression of one or more gene products (e.g., polypeptides).

The terms “fermentation broth” and “fermentation medium” used interchangeably herein refer to a medium that contains or contained a microorganism (e.g., a host cell) and supports microorganism life. Generally, a fermentation medium comprises a carbon source and the microorganism, in an aqueous environment. In one embodiment, most of the cells of the microorganism remain in the fermentation broth as part of the demulsification process. In another group of embodiments, some or essentially all of the cells of the microorganism are removed prior too or during the demulsification or purification process.

The terms “fermentation vessel”, “vessel”, “reaction vessel”, “bioreactor”, “chemstat”, “reactor”, “mixer”, “plug flow reactor (PFR)”, “continuous stirred tank reactor (CSTR)” and the like are terms used interchangeably herein to refer to a container designed or useful to hold the demulsification solvent and/or the product of fermentative bioconversion (such as the fermentation broth).

The term “contacting” as used herein refers to adding one substance to another. Contacting generally includes mixing, stirring and/or shaking.

As used herein, “conditions that permit product production” refers to any fermentation condition that allows a production host to produce a desired product, such as but not limited to acyl-CoA or fatty acid derivatives (e.g., fatty acids, hydrocarbons, fatty alcohols, waxes, or fatty esters).

The term “culturing” refers to growing a population of cells (e.g. microbial) under suitable conditions in a liquid or solid medium. Most often a liquid medium is used. In some embodiments, culturing refers to the fermentative bioconversion of a substrate to an end product (e.g., an organic product).

“Conversion” refers to the enzymatic conversion of the substrate to the corresponding organic product.

The terms “purify” and “purified” are used to refer to a molecule or component (e.g., an organic product, e.g., a fatty alcohol) that is substantially separated from other components (e.g., polypeptides, lipids, carbohydrates, other fermentation products, or contaminants that may be present following fermentation). In some embodiments, a component (e.g., an organic product, e.g., a fatty alcohol) is purified when at least about 50%, 60%, 70%, 80%, 85%, 90%, 91%, 92%, 93%, 94%, 95%, 96%, 97%, 98%, or 99% or more by weight of the sample is composed of the component (e.g., an organic product, e.g., a fatty alcohol).

The term “recoverable,” as used in reference to producing a composition (e.g., fatty alcohols) by a method of the present invention, refers to the amount of composition which can be isolated from the reaction mixture yielding the composition according to methods known in the art.

The techniques and procedures are generally performed according to conventional methods in the art and various general references. See, e.g., Sambrook et al., 2001, Molecular Cloning: A Laboratory Manual, 3rd ed.; Ausubel, ed., 1990-2008, Current Protocols in Molecular Biology; C. A. Reddy et al., 2007, Methods for General and Molecular Microbiology, 3rd Edition, ASM Press. Standard techniques, or modifications thereof, are used for nucleic acid and polypeptide synthesis and for chemical syntheses and chemical analyses. Generally, enzymatic reactions and purification steps are performed according to the manufacturer's specifications.

Throughout the specification, a reference may be made using an abbreviated gene name or polypeptide name, but it is understood that such an abbreviated gene or polypeptide name represents the genus of genes or polypeptides. Such gene names include all genes encoding the same polypeptide and homologous polypeptides having the same physiological function. Polypeptide names include all polypeptides that have the same activity (e.g., that catalyze the same fundamental chemical reaction).

Unless otherwise defined, all technical and scientific terms used herein have the same meaning as commonly understood by one of ordinary skill in the art to which this invention belongs.

Unless otherwise stated, amounts listed in percentage (%) are in volume percent, based on the total volume of the composition.

Recitation of ranges of values herein are merely intended to serve as a shorthand method of referring individually to each separate value falling within the range, unless otherwise indicated herein, and each separate value is incorporated into the specification as if it were individually recited herein.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The claimed subject matter can be understood more fully by reference to the following detailed description and illustrative examples, which are intended to exemplify non-limiting embodiments.

### II. Methods for Demulsifying and Purifying Organic Products

The present invention provides an efficient method to purify organic products from emulsions, such as those from fermentation broths. The methods of the present invention have advantages over prior methods, such as typical liquid-liquid extraction, by effectively demulsifying a broad range of different emulsions into at least two phases with low amounts of the demulsifying solvent which reduces purification costs. In addition, both the desired product and the demulsifying solvent can be easily recovered in high overall yields. The demulsifying solvent and the extraction solvents can be recovered and recycled from the non-aqueous phase (e.g. by distillation) and optionally from the aqueous phases (as a water azeotrope) and reused in subsequent purifications.

The present invention provides polar, partially or fully-water miscible organic solvents which act both as demulsifier and as a solvent to form a predominantly non-aqueous phase in which the organic product is miscible such that separation, purification and isolation of the desired organic compound can be easily achieved. In the process, it is not necessary to use large amounts of a separate non-aqueous extraction solvent and the demulsifying solvent can be recycled without the need for additional solvent or its costly disposal. For example, 3, 4, or 5-carbon primary, secondary and tertiary alcohols such as propanol (n-propanol), isopropyl alcohol (IPA), butanol (n-butanol), sec-butanol, isobutanol, tert-butanol, pentanol (n-pentanol), sec-pentanol(2-pentanol), isopentanol, 2-methyl butanol, neopentyl alcohol, sec-amyl alcohol, sec iso amyl alcohol and the like can be used at relatively low volumetric quantities to demulsify emulsions and act as a solvent for the organic product which separates from the aqueous phase as a distinct non-aqueous phase. Reference is also made to Table 1 herein.

In another aspect the demulsifying solvents of the present invention are compatible with liquid-liquid extraction.

### Demulsifying Solvents

The agents of the present invention are effective to demulsify emulsions. The agents of the present invention have other desirable properties, including at least partial miscibility in water to affect the demulsification. In some embodiments the emulsions may separate into a distinct, predominantly non-aqueous layer with boiling points which differ from the organic product. These properties facilitate the relatively easy separation from the product by distillation. Some of the agents useful in the present methods have the ability to form azeotropes so that they can be separated from water and be recycled.

Accordingly, demulsifying solvents of the present invention include low-molecular weight organic compounds that include functionality with hydrogen-bonding characteristics to promote partial water miscibility.

Demulsifying agents within the scope of this invention include alcohols, amines, carboxylic acids, esters, amides, ethers, ketones, aldehydes, and the like. In some embodiments, these functional groups are substituted on an alkanyl or aryl moiety. In some embodiments, the alkanyl moiety is a C2, or C3, or C4 or C5 or C6 alkanyl moiety. In another group of embodiments, the aryl moiety is a C4-C10 aryl group such as a benzene or furan moiety. In one group of embodiments, at least one functional group of the demulsifying solvent is the same as a functional group in the organic product. In another group of embodiments, at least one functional group of the demulsifying solvent is different from a functional group in the organic product.

The demulsifying solvents of the present invention are designed or chosen to have affinity for the organic product. In some embodiments, the demulsifying solvents are not overly miscible in water or hygroscopic such that they extract water into the predominantly non-aqueous phase. In other embodiments, the demulsifying solvents are overly miscible in water. The demulsifying solvent preferably has a boiling point which is sufficiently distinct from the organic product to facilitate subsequent purification by distillation. In some embodiments, the demulsifying solvent has a lower boiling point that the organic product. In one embodiment, the boiling point is for example lower than about 250° C., lower than about 200° C., lower than about 150° C., lower than about 100° C., or lower than about 50° C.

In other groups of embodiments, the demulsifying solvent has a higher boiling point that the organic product. This is useful for organic products that have a significantly lower boiling point that the demulsifying solvent. In one group of embodiments, the boiling point is for example higher than about 50° C., higher than about 150° C., higher than about 200° C., higher than about 250° C., higher than about 300° C., higher than about 350° C., higher than about 400° C. and higher than about 450° C. In some embodiments the boiling point is higher than between about 150° C. and 300° C.

Suitable demulsifying solvents for the methods described herein are not typical liquid-liquid extraction solvents or demulsifiers. They are distinguished by their properties of high water immiscibility, partial water immiscibility, distinct boiling point from the product to be purified, and their ability to demulsify emulsions comprising water, the organic products and other components.

In one group of embodiments, the demulsifying solvent is a C3, C4 or C5 primary, secondary or tertiary alcohol. In another group of embodiments, the demulsifying solvent is selected from the group in the Table 1, below.

TABLE 1

| | | | Azeotrope |
|---|---|---|---|
| | | Water sol (g | (wt/wt |
| Demulsifying solvent | Common name | per 100 g) | ROH:water) |
| 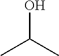 | Isopropanol, 2- propanol | soluble | 88:12 |
| propan-2-ol | | | |
| Chemical Formula: C₃H₈O | | | |
| Molecular Weight: 60.10 | | | |
| Boiling Point: 359.98 [K] | | | |
| CLogP: 0.0739999 | | | |
| 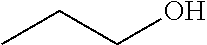 | Propanol, 1- | soluble | 72:28 |
| propan-1-ol | propanol, n- | | |
| Chemical Formula: C₃H₈O | propanol | | |
| Molecular Weight: 60.10 | | | |
| Boiling Point: 360.42 [K] | | | |
| CLogP: 0.294 | | | |
| 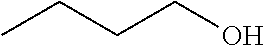 | Butanol, 1- | 9 at 15° C. | 62:38 |
| butan-1-ol | butanol, n- | | |
| Chemical Formula: C₄H₁₀O | butanol | | |
| Molecular Weight: 74.12 | | | |
| Boiling Point: 383.3 [K] | | | |
| CLogP: 0.823 | | | |
| 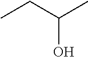 | 2-butanol, sec- butanol | 12.5 at 20° C. | 68:32 |
| butan-2-ol | | | |
| Chemical Formula: C₄H₁₀O | | | |
| Molecular Weight: 74.12 | | | |
| Boiling Point: 382.86 [K] | | | |
| CLogP: 0.603 | | | |
| 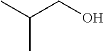 | Isobutanol, 2-methylpropanol | 10 at 15° C. | 67:33 |
| 2-methylpropan-1-ol | | | |
| Chemical Formula: C₄H₁₀O | | | |
| Molecular Weight: 74.12 | | | |
| Boiling Point: 382.86 [K] | | | |
| CLogP: 0.693 | | | |
| 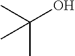 | Tert-butanol | Soluble | 88:12 |
| 2-methylpropan-2-ol | | | |
| Chemical Formula: C₄H₁₀O | | | |
| Molecular Weight: 74.12 | | | |
| Boiling Point: 380.07 [K] | | | |
| CLogP: 0.473 | | | |
| 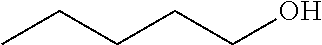 | Amyl alcohol, 1- | 2.7 at 22° C. | 46:54 |
| pentan-1-ol | pentanol, n- | | |
| Chemical Formula: C₅H₁₂O | pentanol | | |
| Molecular Weight: 88.15 | | | |
| Boiling Point: 406.18 [K] | | | |
| CLogP: 1.352 | | | |
| 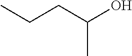 | 2-pentanol, sec- pentanol | 4 at 20° C. | 62:38 |
| pentan-2-ol | | | |
| Chemical Formula: C₅H₁₂O | | | |
| Molecular Weight: 88.15 | | | |
| Boiling Point: 405.74 [K] | | | |
| CLogP: 1.132 | | | |
| 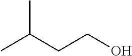 | Isopentanol, isoamyl | 2 at 14° C. | 50:50 |
| 3-methylbutan-1-ol | | | |
| Chemical Formula: C₅H₁₂O | | | |
| Molecular Weight: 88.15 | | | |
| Boiling Point: 405.74 [K] | | | |
| CLogP: 1.222 | | | |
| 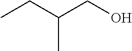 | 2-methyl butanol | 3.6 at 30° C. | |
| 3-methylbutan-1-ol | | | |
| Chemical Formula: C₅H₁₂O | | | |
| Molecular Weight: 88.15 | | | |
| Boiling Point: 405.74 [K] | | | |
| CLogP: 1.222 | | | |
| 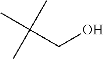 | Neopentyl alcohol | Slightly soluble | |
| 2,2-dimethylpropan-1-ol | | | |
| Chemical Formula: C₅H₁₂O | | | |
| Molecular Weight: 88.15 | | | |
| Boiling Point: 402.95 [K] | | | |
| CLogP: 1.092 | | | |
| 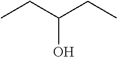 | Sec-amyl alcohol | 5.5 at 3° C. | 64:36 |
| pentan-3-ol | | | |
| Chemical Formula: C₅H₁₂O | | | |
| Molecular Weight: 88.15 | | | |
| Boiling Point: 405.74 [K] | | | |
| CLogP: 1.132 | | | |
| 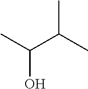 | Sec iso amyl alcohol | Slightly soluble | |
| 3-methylbutan-2-ol | | | |
| Chemical Formula: C₅H₁₂O | | | |
| Molecular Weight: 88.15 | | | |
| Boiling Point: 405.3 [K] | | | |
| CLogP: 1.002 | | | |
| 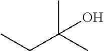 | Tert-amyl alcohol | Slightly soluble | 72:28 |
| 3-methylbutan-2-ol | | | |
| Chemical Formula: C₅H₁₂O | | | |
| Molecular Weight: 88.15 | | | |
| Boiling Point: 402.95 [K] | | | |
| CLogP: 1.002 | | | |

In one group of embodiments only a single type of demulsifying solvent is used, to facilitate recycling of the demulsifying solvent. In other embodiments, more than one demulsifying solvent can be used.

In one group of embodiments the partition coefficient is less than or equal to about 1.5. In one group of embodiments, the demulsifying solvent is propanol, isopropyl alcohol (IPA), butanol, sec-butanol, isobutanol, tert-butanol, pentanol, sec-pentanol, isopentanol, 2-methyl butanol, neopentyl alcohol, sec-amyl alcohol, sec iso amyl alcohol and the like. In one group of embodiments, the demulsifying solvent is isopropyl alcohol (IPA).

### Demulsification

The demulsification methods of the present invention comprise contacting an emulsion containing water (e.g. fermentation broth) and the desired organic compound to be purified with a demulsifying solvent as described above, whereby the emulsion transforms into at least two distinct phases: a predominantly aqueous phase and a predominantly non-aqueous phase. The mixture may be allowed to fully separate into at least two distinct phases or may be optionally mixed, blended or agitated by any convenient method to assist in the separation of the organic compound into the predominantly non-aqueous phase. In some embodiments, the two distinct phases will be separated by centrifugation. In some embodiments it is desirable to mix the mixture of water, organic compound and demulsifying solvent. In other embodiments, it is desirable to avoid mixing the mixture of water, organic compound and demulsifying solvent.

The organic products produced by the methods described herein generally will be relatively immiscible in the fermentation broth if they did not form emulsions. Bi-phasic separation is enhanced in part by the relative immiscibility of the combination of the organic product and demulsification solvent mixture in the predominantly aqueous mixture, which facilitates separation. One of ordinary skill in the art will now appreciate that choosing a demulsification solvent within a particular log P range, will enhance organic product separation even when the demulsification solvent and/or organic product are present in low concentrations in the fermentation vessel. Thus in one group of embodiments, the predominantly non-aqueous phase can be easily separated from the aqueous phase to purify the organic compound from aqueous and other aqueous soluble impurities.

The methods can be practiced at any suitable operating temperature that such reactions take place. Typically the temperature is between the freezing and boiling points of the reaction mixture. In one group of embodiments, the temperature is above about 0° C., or above about 10° C., or above about 20° C., or above about 30° C., or above about 40° C., or above about 50° C., or above about 60° C., or above about 70° C., or above about 80° C., or above about 90° C., or above about 95° C., or above about 100° C., or above about 105° C. or above about 110° C. In another group of embodiments the preferred temperature is between 20° C. and 100° C., between 20° C. and 90° C., between 20° C. and 70° C. and also between 25° C. and 65° C. when at ambient pressure. In another group of embodiments, the temperature is below about 100° C. but above 20° C. and also below about 70° C. but above 30° C.

The methods can be practiced for any suitable time that such reactions and separations result in an optimal yield of desired organic product. In some embodiments the reaction time will be between about 5 seconds and 72 hours, between about 30 seconds and 48 hours, between about 1 minute and about 48 hours, between about 1 minute and 36 hours, between about 1 minute and 24 hours, between about 1 minute and about 10 hours, between about 1 minute and about 5 hours, between about 1 minute and about 2 hours and also between about 1 minute and about 0.5 hours. Determination of the optimal time periods is routine in the art. In some embodiments and particularly in the continuous fermentation process the contact time of the reaction may be less than 1 minute, such as less than about 45 seconds, less than about 30 seconds and less than about 15 seconds.

The amount of demulsifying solvent used in the methods encompassed by the invention may vary depending on the components and total volume of the emulsion. In one group of embodiments, the volume of demulsifying solvent used will completely demulsify the emulsion. In another group of embodiments, the volume of demulsifying solvent will be sufficient to break up the emulsion such that product isolation is possible.

In one group of embodiments, the % volume of demulsifying solvent used will be at most about 100%, and at least about 1% of the volume of the emulsion. In another group of embodiments, the % volume of demulsifying solvent used will be at most 100% and at least 10%, at most 75% and at least about 10%, at most about 65% and at least about 10% of the volume of the emulsion, and at most about 60% and at least about 25% of the volume of the emulsion. In other groups of embodiments, the % volume of demulsifying solvent used will be at least about 10%, at least about 20%, at least about 30%, at least about 40%, at least about 50%, at least about 60%, at least about 70%, at least about 80%, at least about 90% and at most about 100%.

Thus the present method differs from typical liquid-liquid extraction. Typically in liquid-liquid extraction the emulsified component does not partition into the extraction solvent. Furthermore, in a typical extraction, one cannot use an extraction solvent that itself is completely miscible in water with the phase from which the compound of interest is present (for example, one cannot use IPA to extract an organic compound from water as IPA and water are fully miscible with each other). It is surprising that a completely water miscible solvent as disclosed herein (such as IPA) can produce a distinct organic-rich phase. Typical extractions are carried out with solvents that are not fully water miscible. Generally in the methods described herein less than one equivalent volume of solvent is used in relation to the amount of the emulsion.

In another group of embodiments, the demulsifying solvent and the organic compound (organic product) can be separated, and further purified by purification techniques well known in the art such as extraction, distillation, filtration or chromatography. If the organic compound or demulsifying solvents are suitably low boiling and thermally stable, distillation techniques may be used. If either the organic compound or demulsifying solvent contains acidic functionality, it may be purified by base extraction, or precipitated as a salt. If either the organic compound or demulsifying solvent contains a basic functionality, it may be purified by acid extraction, or precipitated as a salt. If there is a size difference between the demulsifying solvent and the organic product they can be purified by membrane separation.

Once the demulsifying solvent is purified from the organic compound it can be recycled and reused.

Similarly, the demulsifying solvents of the present invention may be partially miscible or fully miscible in the aqueous layer. Accordingly, in some embodiments, the demulsifying solvents of the present invention can be separated from the predominantly aqueous phase and purified by other purification techniques well known in the art such as extraction, distillation, filtration or chromatography. If the demulsifying solvents suitably different in boiling point from water, distillation techniques may be used. In some embodiments, the demulsifying solvent and water form an azeotrope, so a known amount of demulsifying solvent can be separated from the rest of the predominantly aqueous phase.

If the demulsifying solvent contains acidic functionality, it may be purified by acid extraction, or precipitated as a salt. If the demulsifying solvent contains a basic functionality, it may be purified by base extraction, or precipitated as a salt. If there is a size difference between the demulsifying solvent and water they can be purified by membrane separation.

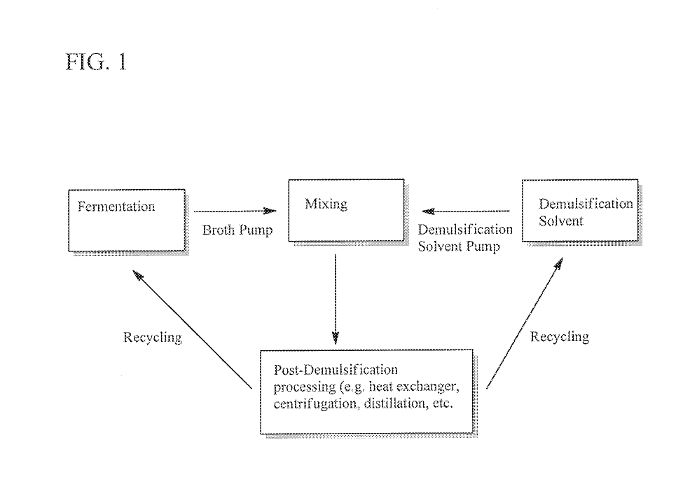

FIG. 1 is a non-limiting example of the general batch process of the present invention. The organic product is produced in a reaction vessel, such as by fermentation of a microorganism such as E. coli. The fermentation broth comprising the organic product is mixed with the demulsification solvent in a reaction vessel and allowed to proceed to completion. The fermentation mixture may optionally be heated or cooled prior to mixing with the demulsification solvent. In some embodiments, the fermentation broth comprising the organic product is subject to heat to kill the microorganism in the fermentor. Optionally the demulsification solvent may be added to the fermentation broth prior to heating. Typically the fermentation broth with or without the demulsification solvent comprising the microorganisms may be exposed to about 70° C. for about 1 hour.

Further any insoluble materials can be removed from the fermentation broth by filtration.

The demulsification solvent can be added to this mixture in the reaction vessel or alternatively, the reaction mixture and the demulsification solvent can be blended in a separate container. In some embodiments, more than one solvent may be used and in other embodiments the solvent may be recycled.

The mixture is then allowed to form distinct phases. This process can be assisted with centrifugation with a single centrifuge or a series of centrifuges. The speed of centrifugation can be optimized by those of ordinary skill in the art. Following centrifugation, the non-aqueous phase containing the demulsifying solvent and organic product may be removed or recovered by known means. The aqueous phase, may be sent, for example, to a distillation apparatus to recover any demulsification solvent present in the aqueous phase which can be recycled.

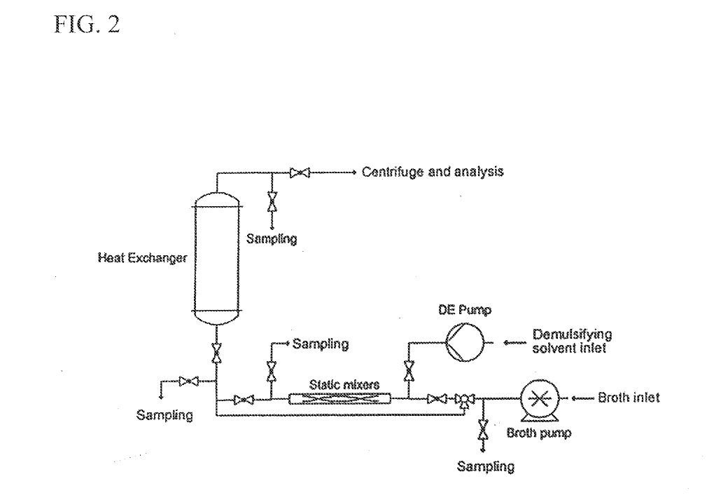

FIG. 2 is another non-limiting example of a general continuous process of the invention wherein the fermentation broth and demulsifying solvent are continually added to the mixing element. At the completion of a microbial fermentation, fermentation broth and the demulsification solvent may optionally be sent to a reaction vessel having mixing elements, such as static mixers, a plug flow reactor, a tubular reactor, a continuous stirred tank reactor and/or a heat exchanger before being centrifuged for organic product recovery. In this example, the fermentation mixture can be sampled and analyzed at any stage of the process.

In another embodiment, the fermentation broth can be optionally heat treated to kill the fermentation cells. Determination of the optimal contact residence time periods is routine in the art.

In some embodiments, the mixture of fermentation broth and organic product is pumped into a mixing tank and separately the demulsifying solvent is either simultaneously or currently pumped into the mixing tank. The emulsion is then allowed to separate into layers. This process can be assisted through the use of a centrifuge or the like. Separation times can vary from about 1 minute to about 4 hours depending on factors such as the flow rate, temperature of the mixture and the like. Upon separation into distinct layers, each layer is removed. Each layer can be further manipulated e.g. by centrifugation, extraction, distillation and/or hydrogenation, to produce the purified organic product.

### III. Organic Products

The organic products to be purified by the methods described herein include any suitable organic compound or combination of compounds which may be present in an aqueous mixture. As used herein, an “organic product” may be a single compound, such as a fatty alcohol octanol (C8:0), or a combination of compounds, such as a mixture of C8-C18 fatty alcohols. These include, but are not limited to fatty alcohols, fatty acids, fatty acid esters and sesquiterpenes and sesquiterpenols like farnescene and farnesol, triglycerides, carotene and carotenoids like lycopene, β-lactams like penicillins, sterols, statins like lovastatin and simvastatin, mycophenolic acid, and the like. In some embodiments, the organic products are produced from biological engineering (e.g., produced by recombinant organisms). In some embodiments, the purified organic product is derivatized, modified, used as a substrate for production of other compounds, and the like.

In some embodiments, the organic product comprises one or more fatty esters (e.g., methyl decanoate, methyl dodecanoate, methyl tetradecanoate, methyl 7-tetradeconoate, methyl hexadecanoate, methyl 9-hexadecenoate, methyl 11-octadecenoate (see, e.g., US 2010/0257778). In some embodiments, the organic product comprises one or more fatty alcohols (e.g., octanol (C8:0), decanol (C10:0), dodecanol (C12:0), tetradecanol (C14:0), hexadecanol (C16:0) and octadecanol (C18:0)) (see, e.g., US2012/0142979). In some embodiments, the organic product comprises one or more hydrocarbons (e.g., farnescene, zingiberene, bisabolene, farnescene expoxide, bisabolol, farnesol isomer, farnesol) (see, e.g., US 2011/0124071, WO2010/115097, and WO2010/115074). In some embodiments, the organic product comprises one or more fatty acids.

### Fatty Alcohols

In some embodiments, the organic product recovered according to the methods of the present invention is a fatty alcohol. In some embodiments, the fatty alcohol comprises a carbon chain that is at least 8, 10, 12, 14, 16, 18, 20, 22, 24, 26, 28, 30, 32, or 34 carbons long. In some embodiments, the fatty alcohol comprises a C8-C24 saturated or unsaturated fatty alcohol (i.e., a C8, C9, C10, C11, C12, C13, C14, C15, C16, C17, C18, C19, C20, C21, C22, C23 or C24 fatty alcohol).

In some embodiments, a composition comprising a fatty alcohol product recovered according to the methods described herein comprises one or more alcohols selected from 1-octanol (C8:0), 1-decanol (C10:0), 1-dodecanol (C12:0), 1-tetradecanol (C14:0), 1-hexadecanol (C16:0), 1-octadecanol (C18:0), 1-icosanol (C20:0), 1-docosanol (C22:0), 1-tetracosanol (C24:0), cis Δ⁹-1-hexadecenol (C16:1), and cis Δ¹¹-1-octadecenol (C18:1).

In some embodiments, a composition comprising a fatty alcohol product recovered according to the methods described herein comprises saturated fatty alcohols, unsaturated fatty alcohols, or both saturated and unsaturated fatty alcohols. In some embodiments, the fatty alcohol is a branched chain or a straight chain fatty alcohol. In some embodiments, the unsaturated fatty alcohols are monounsaturated fatty alcohols. In some embodiments, the fatty alcohol compositions comprise both saturated and unsaturated fatty alcohols, and the amount of unsaturated fatty alcohols is less than about 40%, such as less than about 30%, such as less than about 20%, such as less than about 10%, such as less than about 5%, such as less than about 1% of the fatty alcohols present in the composition. In other embodiments, the fatty alcohol compositions comprise both saturated and unsaturated fatty alcohols, and the amount of saturated fatty alcohols is at least about 40%, at least about 50%, at least about 60%, at least about 70%, at least about 80%, or at least about 90% of the fatty alcohols present in the composition. In some embodiments, the fatty alcohol compositions comprise one or more C10-C18 saturated or unsaturated fatty alcohols, and the amount of the C10-C18 saturated or unsaturated fatty alcohols is at least about 70%, at least about 75%, at least about 80%, at least about 85%, or at least about 90% of the fatty alcohols present in the composition. In some embodiments, the fatty alcohol compositions comprise one or more C10-C14 saturated or unsaturated fatty alcohols, and the amount of the C10-C14 saturated or unsaturated fatty alcohols is at least about 70%, at least about 75%, at least about 80%, at least about 85%, or at least about 90% of the fatty alcohols present in the composition. In some embodiments, the fatty alcohol compositions comprise one or more C12-C14 or C12-C16 saturated or unsaturated fatty alcohols, and the amount of the C12-C14 or C12-C16 saturated or unsaturated fatty alcohols is least about 80%, at least about 85%, at least about 90%, or at least about 95% of the fatty alcohols present in the composition.

### Fatty Alcohol Derivatives

Fatty alcohols produced using the methods and variants disclosed herein can be converted to a variety of commercially useful compounds, referred to as fatty alcohol derivatives. Without limitation, exemplary fatty alcohol derivatives include fatty acids, fatty aldehydes, fatty esters, wax esters, fatty acetates, ethoxylates, sulphates, phosphates, amines, alkanes, and alkenes. The fatty alcohol derivatives may be obtained from fatty alcohols using either enzymatic or chemical methods. In some embodiments, the fatty alcohols can be reacted with a sulfonic acid group to produce sulfate derivatives.

In some embodiments, total fatty alcohols produced in a fermentation are derivatized. In some embodiments, fatty alcohols produced in a fermentation are fractionated, and a fraction(s) is derivatized.

Alkane and/or Alkene Compositions

In some embodiments, the fatty alcohol compositions produced by the methods described herein can be reduced to yield alkanes and/or alkenes having the same carbon chain length as the fatty alcohol starting materials. Without being bound by any particular theory, the hydroxyl group of an alcohol is a poor leaving group, and therefore, in principle a chemical moiety that binds to the oxygen atom of the hydroxyl group to make it a better leaving group can be used to reduce the fatty alcohols described herein. In another embodiment, alkanes can be produced by hydrogenation of fatty alcohols or fatty acids.

Any method known in the art can be used to reduce the fatty alcohols produced according to the methods described herein. In some embodiments, reduction of fatty alcohols can be carried out chemically, for example, by a Barton deoxygenation (or Barton-McCombie deoxygenation), a two-step reaction in which the alcohol is first converted to a methyl xanthate or thioimidazoyl carbamate, and the xanthate or thioimidazoyl carbamate is reduced with a tin hydride or trialkylsilane reagent under radical conditions to produce the alkane and/or alkene. See J. J. Li, C. Limberakis, D. A. Pflum, Modern Organic Synthesis in the Laboratory (Oxford University Press, 2007) at pp. 81-83.

In some embodiments, reduction of fatty alcohols to the corresponding alkanes and/or alkenes can be accomplished using a microorganism that has a biosynthetic pathway for reducing fatty alcohols. In certain embodiments, the microorganism is a bacterium. In specific embodiments, the bacterium is Vibrio furnissii strain M1. In some embodiments, the fatty alcohol compositions produced by the methods described herein are contacted with the appropriate microorganism for reduction to alkanes and/or alkenes. In other embodiments, the fatty alcohol compositions produced by the methods described herein are contacted with membrane fractions from the appropriate microorganism so that the reduction is carried out in a cell free system. See, e.g., Park, 2005, J. Bacteriol. 187(4):1426-1429.

In certain embodiments, alkanes and/or alkenes produced by the reduction of fatty alcohols described herein are isolated from the reaction mixture and unreduced fatty alcohol starting materials to produce a composition that comprises substantially all alkanes and/or alkenes. In some embodiments, the alkanes and/or alkenes produced by the reduction of fatty alcohols described herein and the unreacted fatty alcohol starting materials are isolated from the reaction mixture to produce a composition comprising alkanes and/or alkenes and fatty alcohols.

In certain embodiments, the resulting compositions comprise at least about 60% alkanes and/or alkenes, such as at least about 70% alkanes and/or alkenes, such as at least about 80% alkanes and/or alkenes, such as at least about 85% alkanes and/or alkenes, such as at least about 90% alkanes and/or alkenes, such as at least about 92% alkanes and/or alkenes, such as at least about 95% alkanes and/or alkenes, such as at least about 96% alkanes and/or alkenes, such as at least about 97% alkanes and/or alkenes, such as at least about 98% alkanes and/or alkenes, such as at least about 99% alkanes and/or alkenes by weight of the composition after reduction.

In other embodiments, the resulting compositions comprise at least about 10% alkanes and/or alkenes, such as at least about 20% alkanes and/or alkenes, such as at least about 30% alkanes and/or alkenes, such as at least about 40% alkanes and/or alkenes, such as at least about 50% alkanes and/or alkenes by weight of the composition after reduction.

In some typical embodiments, the compositions produced by the methods described herein comprise one or more alkanes selected from octanes, decanes, dodecanes, tetradecanes, hexadecanes, octadecanes, icosanes, and docosanes. In other typical embodiments, the compositions produced by the methods described herein comprise one or more alkenes selected from octanes, decenes, dodecenes, tetradecenes, hexadecenes, octadecenes, icosenes, and docosenes.

In typical embodiments, C8 to C20 alkanes and/or alkenes comprise at least about 80%, such as at least about 85%, such as at least about 90%, such as at least about 92%, such as at least about 95%, such as at least about 97%, such as at least about 99% by weight of the total alkanes and/or alkenes in the composition. In certain embodiments, C10 to C18 alkanes and/or alkenes comprise about 80%, such as at least about 85%, such as at least about 90%, such as at least about 92%, such as at least about 95%, such as at least about 97%, such as at least about 99% by weight of the total alkanes and/or alkenes in the composition. In certain embodiments, C10 to C16 alkanes and/or alkenes comprise about 80%, such as at least about 85%, such as at least about 90%, such as at least about 92%, such as at least about 95%, such as at least about 97%, such as at least about 99% by weight of the total alkanes and/or alkenes in the composition. In certain embodiments, C10 to C14 alkanes and/or alkenes comprise about 80%, such as at least about 85%, such as at least about 90%, such as at least about 92%, such as at least about 95%, such as at least about 97%, such as at least about 99% by weight of the total alkanes and/or alkenes in the composition.

In certain embodiments, alkanes and/or alkenes having particular carbon chain lengths can be isolated from longer and/or shorter alkanes and/or alkenes, for example by HPLC. In certain embodiments, alkane and/or alkene compositions that are suitable, e.g., for use in jet fuels, comprise C10 to C14 alkanes and/or alkenes. In other embodiments, alkane and/or alkene compositions that are suitable, e.g., for use in diesel fuels comprise alkanes and/or alkenes that have 16 or more carbons (e.g., C16 or longer-chain alkanes and/or alkenes). For example, isoprenoids comprise a diverse class of compounds with over 50,000 members and have a variety of uses including as specialty chemicals, pharmaceuticals and fuels. Conventionally, isoprenoids can be synthesized from petroleum sources or extracted from plant sources. More recently, methods of making such compounds from microbial cells has been described in, for example, U.S. Pat. Nos. 7,399,323, 7,540,888, 7,671,245, 7,592,295, 7,589,243 and 7,655,739 and WO2010/115074.

### Ester Compositions

In certain embodiments, the fatty alcohols are further processed with a carboxylic acid to form acid esters. Esterification reactions of fatty alcohols are well-known in the art. In certain embodiments, the transesterification reaction is carried out in the presence of a strong catalyst, e.g., a strong alkaline such as sodium hydroxide. In other embodiments, the reaction is carried out enzymatically using an enzyme that catalyzes the conversion of fatty alcohols to acid esters, such as lipoprotein lipase. See, e.g., Tsujita et al., 1999, J. Biochem. 126(6):1074-1079.

### Sulfate Derivatives

In some embodiments, the fatty alcohols can be reacted with a sulfonic acid group to produce sulfate derivatives.

### Salt Forms

The organic products of the present invention are meant to include salts of the organic products which are prepared with acids or bases, depending on the particular substituents found on the organic products described herein. When organic products of the present invention contain relatively acidic functionalities, base addition salts can be obtained by contacting the neutral form of the organic products with a sufficient amount of the desired base, either neat or in a suitable inert solvent. Examples of salts derived from inorganic bases include aluminum, ammonium, calcium, copper, ferric, ferrous, lithium, magnesium, manganic, manganous, potassium, sodium, zinc and the like. Salts derived from organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When organic products of the present invention contain relatively basic functionalities, acid addition salts can be obtained by contacting the neutral form of the organic products with a sufficient amount of the desired acid, either neat or in a suitable inert solvent. Examples of acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific organic products of the present invention contain both basic and acidic functionalities that allow the organic products to be converted into either base or acid addition salts.

The neutral forms of the organic products may be regenerated by contacting the salt with a base or acid and isolating the parent organic product in the conventional manner. The parent form of the organic product differs from the various salt forms in certain physical properties, such as solubility in polar solvents, but otherwise the salts may be equivalent to the parent form of the organic product for the purposes of the present invention.

### IV. Emulsions for Production of Organic Products

The methods of the present invention are suitable for demulsification and purification of organic products from emulsions, for example those produced from fermentation broths. The demulsifying solvents enables the organic products to be purified without using high volumes of non-aqueous solvent typically used in liquid-liquid extraction. The methods of the invention can be applied to any emulsion. In some embodiments, the emulsion is an emulsion generated from a reaction mixture before or after the reaction has terminated and further processed, for example by filtration, centrifugation, decolorization, heat treatment or other manipulations. In other embodiments, the reaction mixture is an emulsion is generated from a fermentation broth producing the organic product.

In some embodiments, the methods of the present invention may be used to purify an organic product from a fermentation broth emulsion comprising a host cell that produces an organic product (e.g., a fatty alcohol) and a medium (e.g., an aqueous medium) for culturing the host cell.

### Host Cells for Fermentation

Host cells or strains which may be used to produce an organic product (e.g., a fatty alcohol) include, but are not limited to, bacteria, photosynthetic bacteria (cyanobacteria), yeast, filamentous fungi, and algae. In some embodiments, the host cell is a species of a genus of bacteria selected from the group consisting of Agrobacterium, Arthrobacter, Bacillus, Clostridium, Corynebacterium, Escherichia, Erwinia, Geobacillus, Klebsiello, Lactobacillus, Mycobacterium, Pantoeo, Rhodococcus, Streptomyces and Zymomonas. In some embodiments, the bacterial host cell is a species of Escherichia, e.g., E. coli. In some embodiments, the host cell is a species of a genus of yeast selected from the group consisting of Candida, Hansenula, Soccharomyces, Schizosaccharomyces, Pichia, Kluyveromyces, and Yarrowia. In some embodiments, the yeast host cell is a species of Sacchoromyces, e.g., S. cerevisiae, or a species of Yarrowia, e.g., Y. lipolytica.

In some embodiments, microorganisms useful as host cells are wild-type microorganisms. In particular embodiments, the host cell is a wild-type bacterium, e.g., a wild-type E. coli strain. In various embodiments, the wild-type E. coli bacterial strain useful in the processes described herein is selected from, but not limited to, strain W3110, strain MG16SS and strain BW25113. In some embodiments, the host cell is genetically modified. In particular embodiments, the microorganism is a genetically modified bacterium, e.g, a genetically modified E. coli strain. Examples of genetically modified E. coli useful as recombinant host cells include, but are not limited to, genetically modified E. coli found in the E. Coli Genetic Stock Center, Yale University, New Haven, Conn.; or genetically modified E. coli found in the Keio Collection, available from the National BioResource Project at NBRP E. coli, Microbial Genetics Laboratory, National Institute of Genetics 1111 Yata, Mishima, Shizuoka, 411-8540.

In some embodiments, the host cells are microorganisms that have been modified to have one or more improved properties, e.g., improved biomass utilization (e.g., improved sugar utilization), improved fatty alcohol production, improved thermostability, and/or improved thermoactivity. In some embodiments, a modified host cell is engineered to express an exogenous nucleic acid encoding a protein that, when expressed in the host cell, results in an improved property such as improved biomass utilization, improved fatty alcohol production, improved thermostability, and/or improved thermoactivity. In some embodiments, a modified host cell is engineered to delete or inactivate an endogenous gene and/or replace the endogenous gene with a heterologous gene. Examples of modified host cells having an improved property are described, for example, in US 2012/0003703; US 2012/0165562; and US 2012/0009640; the disclosure of each of which is incorporated by reference herein in its entirety.

It will be appreciated that, consistent with terminology standard in the art, reference to, for example, a “modified host cell” means that a cell or population of cells and their progeny are modified. For example, an exogenous gene can be introduced into a population (culture) of E. coli cells, subpopulations can be selected and cultured for many generations, and a progeny of the subpopulation can be described as a “modified host cell” for use in a fermentation reaction as described herein.

### Modified Host Cells Expressing FAR Polypeptides

Enzymes that convert acyl ACP substrates and/or acyl CoA substrates to fatty alcohols (collectively referred to as fatty alcohol reductases (FARs)) are known in the art (see, WO2011/008535; WO2011/019858; U.S. Patent Publication No. 2010/0203614; U.S. Patent Publication No. 2012/0184006, U.S. Pat. No. 7,332,311; U.S. Pat. No. 6,143,538, Metz et al., 2000, Plant Physiology 122:635-644; Reiser and Somerville, J. Bacterial. (1997) 179:2969; Kalscheuer et al., 2006, Appl. Environ. Microbiol 72:1373, each of which is incorporated herein by reference). In some embodiments, FAR enzymes reduce fatty acyl-CoA substrates to fatty alcohols in a two-step process wherein the acyl-CoA substrate is converted to fatty aldehyde and then the aldehyde is reduced by a NAD(P)H dependent alcohol dehydrogenase. Enzymes involved in the two-step process include Acr1 and YqhD. In some embodiments, FAR enzymes reduce fatty acyl CoA and/or fatty acyl ACP substrates to fatty alcohols in a single enzymatic step, wherein free fatty aldehydes are not produced or essentially not released as an intermediate. See, e.g., Hofvander et al., FEBS Letters (2011) 585:3538-3543; and Willis et al., Biochemistry (2011) 50:10550-10558.

Thus in some embodiments, the modified host cell expresses an exogenous nucleic acid encoding an improved fatty acyl-CoA reductase (FAR) polypeptide, wherein the modified host cell has increased fatty alcohol production compared to a cell expressing a wild-type FAR. Enzymes that convert acyl ACP substrates and/or acyl CoA substrates to fatty alcohols are known in the art (see, e.g., WO2011/008535; WO2011/019858; US2012/0184006, U.S. Pat. No. 7,332,311; U.S. Pat. No. 6,143,538, Metz et al., 2000, Plant Physiology 122:635-644; Reiser and Somerville, J. Bacteriol. (1997) 179:2969; and Kalscheuer et al., 2006, Appl. Environ. Microbiol 72:1373, each of which is incorporated herein by reference). In some embodiments, FAR enzymes reduce fatty acyl-CoA substrates to fatty alcohols in a two-step process wherein the acyl-CoA substrate is converted to fatty aldehyde and then the aldehyde is reduced by a NAD(P)H dependent alcohol dehydrogenase. Enzymes involved in the two-step process include Acr1 and YqhD. In some embodiments, FAR enzymes reduce fatty acyl CoA and/or fatty acyl ACP substrates to fatty alcohols in a single enzymatic step, wherein free fatty aldehydes are not produced or essentially not released as an intermediate.

While not meant to limit the invention, in some embodiments the FAR is a prokaryotic enzyme. In some embodiments, the FAR is derived from a species of Marinobacter including but not limited to M. algicola, M. alkaliphilus, M. aquaeolei, M. arcticus, M. bryozoorum, M. daepoensis, M. excellens, M. flavimaris, M. guodonensis, M. hydrocarbonoclasticus, M. koreenis, M. lipolyticus, M. litoralis, M. lutooensis, M. moritimus, M. sediminum, M. squalenivirans, and M. vinifirmus, and equivalent and synonymous species thereof.

In certain embodiments, the FAR is derived from M. algicolo strain DG893 (wild-type “FAR Maa,” see US 2011/0000125). In some embodiments the wild-type FAR Maa has an amino acid sequence that is at least about 30% identical, at least about 40% identical, at least about 50% identical, at least about 60% identical, at least about 70% identical, at least about 75% identical, at least about 80% identical, at least about 85% Identical, at least about 90% identical, at least about 93% identical, at least about 95% identical, at least about 97% identical, at least about 98% identical and/or at least about 99% identical to SEQ ID NO:1 and/or a functional fragment thereof. In another embodiment, the FAR enzyme has an amino acid sequence that is identical to SEQ ID NO:1.

In certain embodiments, the FAR is derived from Marinobacter aquaeolei strain VT8 (wild-type “FAR Maq,” see US 2011/0000125). In some embodiments the wild-type FAR Maq has an amino acid sequence that is at least about 30% identical, at least about 40%, at least about 50% identical, at least about 60% identical, at least about 70% identical, at least about 75%, at least about 80% identical, at least about 85% identical, at least about 90% identical, at least about 93% identical, at least about 95% identical, at least about 97% identical, at least about 98% identical and/or at least about 99% identical to SEQ ID NO:2 and/or a functional fragment thereof. In another specific embodiment, the isolated FAR enzyme has an amino acid sequence that is identical to SEQ ID NO:2.

In certain embodiments, the FAR is a variant of the wild-type FAR of SEQ ID NO:1 or SEQ ID NO:2 which has at least 1, at least 2, at least 3, at least 4, at least 5, at least 6, at least 7, at least 8, at least 9, at least 10, at least 12, at least 14, at least 16, at least 18, at least 20, at least 25, at least 30, at least 35, at least 40 or more amino acid alterations (e.g., substitutions, deletions and/or insertions) relative to SEQ ID NO:1 or SEQ ID NO:2, respectively. In certain embodiments, the FAR has an amino acid sequence of at least about 95% (such as at least about 96%, at least about 97%, at least about 98%, or at least about 99% sequence identity to any of SEQ ID NO:3, SEQ ID NO:4, or SEQ ID NO:5. In some embodiments, the variant FAR is “FAR-V1” which comprises the amino acid sequence of SEQ ID NO: 3. In some embodiments, the variant FAR is “FAR-V2” which comprises the amino acid sequence of SEQ ID NO: 4. In some embodiments, the variant FAR is “FAR-V3” which comprises the amino acid sequence of SEQ ID NO:5.

In some embodiments, the FAR has the amino acid sequence of any of the FAR polypeptides disclosed in WO2012/006114, and/or a functional fragment thereof. In some embodiments, the FAR has an amino acid sequence that is at least about 70% identical, at least about 75%, at least about 80% identical, at least about 85% identical, at least about 90% identical, at least about 93% identical, at least about 95% identical, at least about 97% identical, at least about 98% identical and/or at least about 99% identical to any of the FAR polypeptides disclosed in WO2012/006114 and/or a functional fragment thereof. In some embodiments, the FAR is encoded by a polynucleotide sequence having at least 85% (at least 90%, 91%, 92%, 93%, 94%, 95%, 96%, 97%, 98%, 99% sequence identity to any polynucleotide sequence disclosed in WO 2012/006114. The entire contents of WO2012/006114 are incorporated by reference herein.

In some embodiments, the FAR has the amino acid sequence of any of the FAR polypeptides disclosed in US 2012/0184006 and/or a functional fragment thereof. In some embodiments, the FAR has an amino acid sequence that is at least about 70% identical, at least about 75%, at least about 80% identical, at least about 85% identical, at least about 90% identical, at least about 93% identical, at least about 95% identical, at least about 97% identical, at least about 98% identical and/or at least about 99% identical to any of the FAR polypeptides disclosed in US 2012/0184006 and/or a functional fragment thereof. In some embodiments, the FAR is encoded by a polynucleotide sequence having at least 85% (at least 90%, 91%, 92%, 93%, 94%, 95%, 96%, 97%, 98%, 99% sequence identity to any polynucleotide sequence disclosed in US 2012/0184006. The entire contents of US 2012/0184006 are incorporated by reference herein.

In certain embodiments, the FAR is obtained from a marine bacterium selected from the group of Meptuniibacter caesoriensis strain MED92, Reinekea sp. strain MED297, Marinomonas sp. strain MED121, unnamed gamma proteobacterium strain HTCC2207, and Marinobacter sp. strain ELB17, as well as equivalents and synonymous species thereof. In certain embodiments, the FAR is obtained from the genus Oceanobacter. In some embodiments, the FAR is obtained from the Oceanobacter species strain RED65 and has an amino acid sequence that is at least about 30% identical, at least about 40% identical, at least about 50% identical, at least about 60% identical, at least about 65%, at least about 70% identical, at least about 75% identical, at least about 80% identical, at least about 85% identical, at least about 90% identical, at least about 93% identical, at least about 95% identical, at least about 97% identical, and/or at least about 98% identical to any polypeptide sequence disclosed in WO2011/008535. In another specific embodiment, the FAR comprises or consists of a sequence having about 100% identity to the polypeptide sequence SEQ ID NO:6 (“FAR_Ocs”) that is disclosed in WO2011/008535 (SEQ ID NO:6), and/or a functional fragment thereof. In other specific embodiments, the FAR polypeptide or functional fragment is obtained or derived from Oceanobacter kriegii. In still other specific embodiments, the isolated FAR enzyme or functional fragment is obtained or derived from Oceanobacter strain WH099. The entire contents of WO2011/008535 are incorporated by reference herein.

In various embodiments, the FAR has a sequence or is encoded by a polynucleotide selected from the group of FAR_Hch (Hahella chejuensis KCTC 2396 GenBank YP_{—}436183.1) (SEQ ID NO:7); FAR_Mac (from marine Actinobacterium strain PHSC20C1) (SEQ ID NO:8); FAR_JVC (JCVI_ORF_{—}1096697648832, GenBank Accession No. EDD40059.1) (SEQ ID NO:9); FAR_Fer (JCVI_SCAF_{—}1101670217388) (SEQ ID NO:10); FAR_Key (JCVI_SCAF_{—}1097205236585) (SEQ ID NO:11); FAR_Gal (JCVI_SCAF_{—}1101670289386) (SEQ ID NO:12); Vitis vinifera FAR (GenBank Accession No. CAO22305.1 [SEQ ID NO:13] or CAO67776.1 [SEQ ID NO:14]); Desulfatibacillum alkenivorans FAR (GenBank Accession No. NZ_ABII01000018.1); Stigmatella aurantiaca FAR (NZ_AAMD01000005.1) (SEQ ID NO:15); Phytophthoro ramorum FAR (GenBank Accession No.: AAQX01001105.1) (SEQ ID NO:16); Simmondsia chinensis acyl CoA reductase (GenBank Accession no. AAD38039.1 (SEQ ID NO:17); Bombyx mori fatty-acyl reductase (GenBank Accession no. BAC79425.1 (SEQ ID NO:18); GenBank Accession No. DQ446732.1 (SEQ ID NO:19) or NM_{—}115529.1 (SEQ ID NO:20); and Ostrinia scapulalis (GenBank Accession No. EU817405.1 (SEQ ID NO:21).

As used herein, a “functional fragment” refers to a polypeptide that has an amino-terminal deletion and/or carboxyl-terminal deletion and/or internal deletion, but where the remaining amino acid sequence is identical or substantially identical to the corresponding positions in the sequence to which it is being compared (e.g., a full-length wild-type FAR or FAR variant) and that retains substantially all of the activity of the full-length polypeptide. In various embodiments, a functional fragment of a full-length wild-type FAR or a variant FAR comprises at least about 70%, at least about 75%, at least about 80%, at least about 90%, at least about 91%, at least about 92%, at least about 93%, at least about 94%, at least about 95%, at least about 96%, at least about 97%, at least about 98%, or at least about 99% identity to the wild-type or reference amino acid sequence. In certain embodiments, a functional fragment comprises at least about 80%, at least about 85%, at least about 90%, at least about 91%, at least about 92%, at least about 93%, at least about 94%, at least about 95%, at least about 96%, at least about 97%, at least about 98%, or at least about 99% of the amino acid sequence of a full-length FAR polypeptide. In other embodiments, the functional fragment comprises at least 75%, at least 80%, at least 85%, at least 90%, and at least 95% of the activity of the full length FAR to which it is being compared.

In some embodiments, the preferred substrates for FAR are fatty acyl-ACP substrates comprising carbon chain lengths of C10 to C20. In certain embodiments, the fatty acyl-ACP substrates comprise carbon chain lengths of C12 to C18, and in other embodiments, the fatty acyl-ACP substrates comprise carbon chain lengths of C12 to C16 or C12 to C14. In certain embodiments, the substrate comprises a majority of saturated hydrocarbons. In certain embodiments, the substrate pool for FAR comprises over about 80% (e.g., 85%, 90%, 92%, 94%, 95%, 96%, 97%, and 98%) C12 to C18 fatty acyl-ACP substrates. In other embodiments, FAR catalyzes the reduction of fatty acyl CoA substrates to the corresponding fatty alcohol. In certain embodiments, the fatty acyl CoA substrate pool comprises over about 70% (e.g., about 75%, 80%, 85%, 88%, 90%, 92%, 94%, 95%, 96%, 97%, 98% and 99%) C10 to C18 fatty acyl CoA substrates; over about 70% (e.g., about 75%, 80%, 85%, 88%, 90%, 92%, 94%, 95%, 96%, 97%, 98% and 99%) C10 to C16 fatty acyl-CoA substrates; over about 70% (e.g., about 75%, 80%, 85%, 88%, 90%, 92%, 94%, 95%, 96%, 97%, 98% and 99%) C12 to C16 fatty acyl-CoA substrates, and also over about 70% (e.g., about 75%, 80%, 85%, 88%, 90%, 92%, 94%, 95%, 96%, 97%, 98% and 99%) C12 to C14 fatty acyl-CoA substrates. In certain embodiments, the substrate pool for FAR comprises over about 80% (e.g., about 85%, about 90%, or about 95%) C12 to C18 fatty acyl-CoA substrates. In certain embodiments, the C10 to C18 fatty acyl substrate (e.g., C12 to C14 fatty acyl substrate or C12 to C16 fatty acyl substrate) comprises a majority of saturated hydrocarbons. See, e.g., Rowland and Domergue, Plant Sci. (2012) 193-194: 28-38.

FAR variants, methods of generating FAR variants, and methods for modifying host cells to express one or more exogenous genes, are described in WO2012/006114, WO2013/096092, WO2013/096082, and U.S. Provisional Patent Application Nos. 61/636,044, filed Apr. 20, 2012; and 61/674,053, filed Jul. 20, 2012. Methods, reagents, and tools for transforming and culturing the host cells described herein are known in the art and can be readily determined by those skilled in the art. General methods, reagents and tools for transforming, e.g., bacteria can be found, for example, in Sambrook et al (2001) Molecular Cloning: A Laboratory Manual, 3^{rd} ed., Cold Spring Harbor Laboratory Press, New York. Many references are available for the culture and production of many cells, including cells of bacterial, plant, animal (especially mammalian) and archebacterial origin.

### Fermentation Conditions

Fermentation of a host cell is carried out under suitable conditions for a time sufficient to produce an organic product such as a fatty alcohol. Conditions for the culture and production of cells, including filamentous fungi, bacterial, and yeast cells, are readily available. See, e.g., Sambrook, Ausubel, and Berger (all supra), as well as Freshney (1994) Culture of Animal Cells, a Manual of Basic Technique, third edition, Wiley-Liss, New York and the references cited therein; Doyle and Griffiths (1997) Mammalian Cell Culture: Essential Techniques John Wiley and Sons, NY; Humason (1979) Animal Tissue Techniques, fourth edition W.H. Freeman and Company; and Ricciardelli, et al., (1989) In Vitro Cell Dev. Biol. 25:1016-1024, all of which are incorporated herein by reference. Culture conditions, such as temperature, pH and the like, will be apparent to those skilled in the art. Cell culture media in general are set forth in Atlas and Parks, eds., 1993, The Handbook of Microbiological Media. The individual components of such media are available from commercial sources, e.g., under the DIFCO™ and BBL™ trademarks. In some embodiments, the culture medium is an aqueous medium. In some embodiments, the aqueous nutrient medium is a “rich medium” comprising complex sources of nitrogen, salts, and carbon, such as YP medium, comprising 10 g/L of peptone and 10 g/L yeast extract of such a medium. In other embodiments, the aqueous nutrient medium is Yeast Nitrogen Base (DIFCO™) supplemented with an appropriate mixture of amino acids, e.g., SC medium. In particular embodiments, the amino acid mixtures lack one or more amino acids, thereby imposing selective pressure for maintenance of an expression vector within the recombinant host cell.

The culture medium can contain an assimilable carbon source. Assimilable carbon sources are available in many forms and include renewable carbon sources and the cellulosic and starch feedstock substrates obtained therefrom. Exemplary assimilable carbon sources include, but are not limited to, depolymerized cellulosic material, monosaccharides, disaccharides, oligosaccharides, saturated and unsaturated fatty acids, succinate, acetate and mixtures thereof. Further carbon sources include, without limitation, glucose, galactose, sucrose, xylose, fructose, glycerol, arabinose, mannose, raffinose, lactose, maltose, and mixtures thereof. In some embodiments, the term “fermentable sugars” is used interchangeably with the term “assimilable carbon source.” In some embodiments, fermentation is carried out with a mixture of fermentable sugars, e.g., a mixture of glucose and galactose or a mixture of glucose and xylose as the assimilable carbon source. In some embodiments, fermentation is carried out with glucose alone to accumulate biomass, after which the glucose is substantially removed and replaced with an inducer, e.g., galactose for induction of expression of one or more heterologous genes involved in fatty alcohol production. In some embodiments, fermentation is carried out with an assimilable carbon source that does not mediate glucose repression, e.g., raffinose, to accumulate biomass, after which the inducer, e.g., galactose, is added to induce expression of one or more heterologous genes involved in fatty alcohol production. In some embodiments, the assimilable carbon source is from cellulosic and starch feedstock derived from but not limited to, wood, wood pulp, paper pulp, grain, corn stover, corn fiber, rice, paper and pulp processing waste, woody or herbaceous plants, fruit or vegetable pulp, distillers grain, grasses, rice hulls, wheat straw, cotton, hemp, flax, sisal, corn cobs, sugar cane bagasse, switch grass, and mixtures thereof.

Fermentation conditions usually comprise many parameters. Exemplary conditions include, but are not limited to, temperature ranges, levels of aeration, and media composition. Each of these conditions, individually and/or in combination, allows the production host to grow. Exemplary media include broths and/or gels. Generally, a suitable medium includes a carbon source (e.g., glucose, fructose, cellulose, etc.) that can be metabolized by the microorganism directly. In addition, enzymes can be used in the medium to facilitate the mobilization (e.g., the depolymerization of starch or cellulose to fermentable sugars) and subsequent metabolism of the carbon source. To determine if the fermentation conditions permit product production, the production host can be cultured for about 4, 8, 12, 24, 36, 48, 60, or 72 hours. During culturing or after culturing, samples can be obtained and analyzed to determine if the fermentation conditions have permitted product production. For example, the production hosts in the sample or the medium in which the production hosts are grown can be tested for the presence of the desired product. Exemplary assays, such as TLC, HPLC, GC/FID, GC/MS, LC/MS, MS, as well as those provided herein, can be used identify and quantify the presence of a product.

The host cells can be grown under batch, fed-batch or continuous fermentation conditions. Classical batch fermentation is a closed system, wherein the compositions of the medium is set at the beginning of the fermentation and is not subject to artificial alternations during the fermentation. A variation of the batch system is a fed-batch fermentation which also finds use in the present invention. In this variation, the substrate is added in increments as the fermentation progresses. Fed-batch systems are useful when catabolite repression is likely to inhibit the metabolism of the cells and where it is desirable to have limited amounts of substrate in the medium. Batch and fed-batch fermentations are common and well known in the art. Continuous fermentation is an open system where a defined fermentation medium is added continuously to a bioreactor and an equal amount of conditioned medium is removed simultaneously for processing. Continuous fermentation generally maintains the cultures at a constant high density where cells are primarily in log phase growth. Continuous fermentation systems strive to maintain steady state growth conditions. Methods for modulating nutrients and growth factors for continuous fermentation processes as well as techniques for maximizing the rate of product formation are well known in the art of industrial microbiology.

In some embodiments, fermentations are carried out at a temperature of from about 10° C. to about 60° C., from about 15° C. to about 50° C., from about 20° C. to about 45° C., from about 20° C. to about 40° C., from about 20° C. to about 35° C. and from about 25° C. to about 45° C. In one embodiment, the fermentation is carried out at a temperature of about 28° C. and/or about 30° C. It will be understood that, in certain embodiments where thermostable host cells are used, fermentations may be carried out at higher temperatures.

In some embodiments, the fermentation is carried out for a time period of about 8 hours to 240 hours, about 8 hours to about 168 hours, about 8 hours to 144 hours, about 16 hours to about 120 hours, or about 24 hours to about 72 hours.

In some embodiments, the fermentation is carried out at a pH of about 4 to about 8, about 4.5 to about 7.5, about 5 to about 7, or about 5.5 to about 6.5.

### Post-Fermentation Treatments

After the fermentation process is deemed complete, the fermentation mixture may optionally be heated to about 100° C. The heating period may be for up to about 4 hours, (such as about 4 hours, 3 hours, 2 hours, and 1 hour). In some embodiments, about 2 hours is desirable. The heated fermentation mixture may optionally be cooled to between 0-95° C. (such as between 5-70° C.; between 10-60° C.; or between 10-45° C.) prior to the demulsification treatment.

### Production Levels

In some embodiments, the organic product (e.g., fatty alcohols) are produced in high yield. Routine culture conditions, e.g., culture of bacteria or yeast, may yield about 0.1 g to about 35 g hydrophobic products (such as about 0.5 g to about 35 g hydrophobic products), e.g., fatty alcohols, per liter of culture medium (e.g., nutrient medium). In some embodiments, the amount of hydrophobic products, e.g., fatty alcohols, produced by the methods described herein is at least 0.1 g/L, at least 0.5 g/L, at least 1 g/L, at least 1.5 g/L, at least 2 g/L, at least 2.5 g/L, at least 3 g/L, at least 3.5 g/L, at least 4 g/L, at least 4.5 g/L, at least about 5 g/L, or at least 10 g/L, at least 20 g/L, at least 30 g/L, at least 40 g/L, or at least 50 g/L of culture medium. In some embodiments, the organic product comprises C12-C16 fatty alcohols (e.g., C12-C14 fatty alcohols) wherein the C12-C16 fatty alcohols (e.g., the C12-C14 fatty alcohols) comprise at least about 50%, at least about 55%, at least about 60%, at least about 65%, at least about 70%, at least about 75%, at least about 80%, or at least about 85% of the amount of organic product produced. In some embodiments, for an organic product comprising C12-C16 fatty alcohols (e.g., C12-C14 fatty alcohols), the C12-C16 fatty alcohols (e.g., the C12-C14 fatty alcohols) comprise at least about 70%, at least about 75%, at least about 80%, or at least about 85% of the at least 0.1 g/L, at least 0.5 g/L, at least 1 g/L, at least 1.5 g/L, at least 2 g/L, at least 2.5 g/L, at least 3 g/L, at least 3.5 g/L, at least 4 g/L, at least 4.5 g/L, at least 5 g/L, at least 10 g/L, at least 20 g/L, at least 30 g/L, at least 40 g/L, at least 50 g/L, at least 60 g/L or at least 70 g/L produced.

In some embodiments, the amount of hydrophobic products, e.g., fatty alcohols, produced by the methods described herein is about 40 mg/g to about 1 g/g, about 40 mg/g to about 5 g/g, about 100 mg/g to about 1 g/g, about 100 mg/g to about 5 g/g, about 500 mg/g to about 2 g/g, about 1 g/g to about 4 g/g, or about 2 g/g to about 3 g/g of dry cell weight by routine modification of culturing conditions.

In some embodiments, the amount of hydrophobic products, e.g., fatty alcohols, produced by the methods described herein is about 4% to about 20%, about 10% to about 20%, about 20% to about 30%, about 30% to about 40%, about 40% to about 50%, about 50% to about 60%, about 60% to about 70%, or about 70% to about 80% of dry cell weight by routine modification of culturing conditions.

### Measuring Fatty Alcohol Production

FAR fatty alcohol production and fatty alcohol profiles (i.e., chain length distribution) can be determined using any other method known in the art. Fatty by methods described in the Examples section and/or alcohol production by an organism expressing a FAR polypeptide (e.g., a FAR variant) can be described as an absolute quantity (e.g., moles/liter of culture) or as a fold-improvement over production by an organism or culture expressing a reference FAR sequence (e.g., a wild-type FAR or a different FAR variant).

Fatty alcohol production and/or fatty alcohol profiles by a microorganism expressing a FAR polypeptide can be measured, for example, using gas chromatography. In general, cells expressing a FAR polypeptide are cultured, total or secreted fatty alcohols are isolated, and fatty alcohol amount and/or content is measured. Exemplary assays for measuring fatty alcohol production and fatty alcohol profiles (i.e., chain length distribution) are described in US 2012/0009640.

Fatty alcohol profiles (i.e., chain length distribution) can be determined, for example, using gas chromatography and/or mass spectroscopy. In an exemplary assay, fatty alcohols are produced as described above and the identification of individual fatty alcohols is performed by comparison to commercial standards (Sigma Chemical Company, 6050 Spruce St. Louis, Mo. 63103). The identity of the peaks can also be confirmed by running the samples through a gas chromatography (GC) equipped with mass spectrometer (MS) as needed.

### V. Compositions Comprising an Organic Product

The organic products that are recovered according to the methods of the present invention can be used as components of various compositions, including but not limited to, detergent compositions (e.g., laundry detergents in liquid and powder form, hard surface cleaners, dishwashing liquids, and the like); industrial compositions (e.g., lubricants, solvents; and industrial cleaners); personal care compositions (e.g., soaps, cosmetics, shampoos, and gels); and fuel compositions (e.g., biodiesels and petrodiesels).

In some embodiments, organic products (e.g., fatty alcohols) recovered according to the methods described herein and/or compounds derived therefrom can be used as components of fuel compositions. Fuel compositions include any compositions used in powering combustion engines, including but not limited to biodiesel fuels and petrodiesel fuels (e.g., jet fuels and rocket fuels).

In some embodiments, the fuel composition is diesel fuel. Diesel fuel is any fuel used in diesel engines and includes both petrodiesel and biodiesel. Petrodiesel is a specific fractional distillate of fossil fuel oil. It is comprised of about 75% saturated hydrocarbons and 25% aromatic hydrocarbons. Biodiesel is not derived from petroleum but from vegetable oil or animal fats and contains long chain alkyl esters. Biodiesel is made by the transesterification of lipids (e.g., spent vegetable oil from fryers or seed oils) with an alcohol and burns cleaner than petrodiesel. Biodiesel can be used alone or mixed with petrodiesel in any amount for use in modern engines.

In some embodiments, the fuel composition is kerosene. Kerosene is a combustible hydrocarbon that is also a specific fractional distillate of fossil fuel and contains hydrocarbons having 6 to 16 carbon atoms. Kerosene has a heat of combustion comparable to that of petrodiesel and is widely used in jet fuel to power jet engines and for heating in certain countries. In particular embodiments, the kerosene-like fuel compositions are included in various grades of jet fuel, including but not limited to, grades Avtur, Jet A, Jet A-1, Jet B, JP-4, JP-5, JP-7 and JP-8. In other embodiments, the kerosene-like fuel compositions are included in fuel compositions for heating. In still other embodiments, the kerosene-like fuel compositions derived from the fatty alcohol compositions described above are burned with liquid oxygen to provide rocket fuel, e.g., in RP-1 rocket fuel.

In various embodiments, fatty alcohols can be reacted with a carboxylic acid to produce acid esters. In particular embodiments, the acid esters are used as components of biodiesel fuel compositions. In other embodiments, fatty alcohols are reacted with a reducing agent to produce alkanes and/or alkenes. In some embodiments, alkanes and/or alkenes (e.g., C10 to C14) derived from the fatty alcohol compositions are used as components of jet fuel compositions. In other embodiments, alkanes and/or alkenes derived from fatty alcohol compositions are used as components of rocket fuel. In still other embodiments, alkanes and/or alkenes (e.g., C16 to C24) derived from the fatty alcohol compositions are used as components in petrodiesel-like fuel compositions.

In certain embodiments, fatty alcohols, or acid esters or alkanes and/or alkenes derived there from, are combined with other fuels or fuel additives to produce compositions having desired properties for their intended use. Exemplary fuels and fuel additives for particular applications are well-known in the art. Exemplary fuels which can be combined with the compositions described herein include, but are not limited to, traditional fuels such as ethanol and petroleum-based fuels. Exemplary fuel additives which can be combined with the compositions described herein include, but are not limited to, cloud point lowering additives, surfactants, antioxidants, metal deactivators, corrosion inhibitors, anti-icing additives, anti-wear additives, deposit-modifying additives and octane enhancers.

### Detergent Compositions

In certain embodiments, the organic products (e.g., fatty alcohols) compositions described herein and compounds derived there from can be used as components of detergent compositions. Detergent compositions containing fatty alcohols produced by the methods of the present invention include compositions used in cleaning applications, including, but not limited to, laundry detergents, hand-washing agents, dishwashing detergents, rinse-aid detergents, household detergents, and household cleaners, in liquid, gel, granular, powder, or tablet form. In some embodiments, the fatty alcohol compositions produced by the methods described above can be used directly in detergent compositions. In some embodiments, the fatty alcohols can be reacted with a sulfonic acid group to produce sulfate derivatives that can be used as components of detergent compositions. Detergent compositions that can be generated using the fatty alcohol compositions produced by the methods of the present invention include, but are not limited to, hair shampoos and conditioners, carpet shampoos, light-duty household cleaners, light-duty household detergents, heavy-duty household cleaners, and heavy-duty household detergents. Detergent compositions generally include, in addition to fatty alcohols, one or more or of builders (e.g., sodium carbonate, complexation agents, soap, and zeolites), enzymes (e.g., a protease, a lipase and an amylases); carboxymethyl cellulose, optical brighteners, fabric softeners, colorants and perfumes (e.g., cyclohexyl salicylate).

In some embodiments, sulfate derivatives derived from the fatty alcohol compositions are used in products such as hair shampoos, carpet shampoos, light-duty household cleaners, and light-duty household detergents. In some embodiments, fatty alcohol compositions (e.g., C16-C18) produced by the methods described herein are used in products such as hair shampoos and conditioners. In some embodiments, sulfate derivatives (e.g., C16-18) derived from the fatty alcohol compositions are used in products such as heavy-duty household cleaners and heavy-duty household detergents. Indeed, it is not intended that the present invention be limited to any particular detergent, detergent formulation nor detergent use.

### Personal Care Compositions

In certain embodiments, the organic products (e.g., fatty alcohols) described herein and compounds derived therefrom are used as components of personal care compositions. In some embodiments, the fatty alcohol compositions produced by the methods described above can be used directly in personal care compositions. Personal care compositions containing fatty alcohols produced by the methods of the present invention include compositions used for application to the body (e.g., for application to the skin, hair, nails, or oral cavity) for the purposes of grooming, cleaning, beautifying, or caring for the body, including but not limited to lotions, balms, creams, gels, serums, cleansers, toners, masks, sunscreens, soaps, shampoos, conditioners, body washes, styling aids, and cosmetic compositions (e.g., makeup in liquid, cream, solid, anhydrous, or pencil form). In some embodiments, the fatty alcohols can be reacted with a sulfonic acid group to produce sulfate derivatives that can be used as components of said compositions. Indeed, it is not intended that the present invention be limited to any particular formulation, nor use.

In some embodiments, organic products (e.g., fatty alcohols) produced by the methods described herein are used in products such as lubricating oils, pharmaceuticals, and as an emollient in cosmetics. In some embodiments, fatty alcohol compositions (e.g., C14) produced by the methods described herein are used in products such as cosmetics (e.g., cold creams) for its emollient properties. In some embodiments, fatty alcohol compositions (e.g., C16) produced by the methods described herein are used in products such as cosmetics (e.g., skin creams and lotions) as an emollient, emulsifier, or thickening agent. In some embodiments, fatty alcohol compositions (e.g., C18) produced by the methods described herein are used in products such as lubricants, resins, perfumes, and cosmetics, e.g., as an emollient, emulsifier, or thickening agent. In some embodiments, sulfate derivatives (e.g., C12 to C16 or C12 to 14) derived from the fatty alcohol compositions produced by the methods described herein are used in products such as toothpastes. Indeed, it is not intended that the present invention be limited to any particular formulation, nor use.

### Other Compositions

In some embodiments, organic products (e.g., fatty alcohols) produced by the methods described herein are used in products such as lubricating oils, pharmaceuticals, and as an emollient in cosmetics. In some embodiments, fatty alcohol compositions produced by the methods described herein are used in products such as cosmetics (e.g., cold creams) for its emollient properties. In some embodiments, fatty alcohol compositions produced by the methods described herein are used in products such as cosmetics (e.g., skin creams and lotions) as an emollient, emulsifier, or thickening agent. In some embodiments, fatty alcohol compositions produced by the methods described herein are used in products such as lubricants, resins, perfumes, and cosmetics, e.g., as an emollient, emulsifier, or thickening agent. In some embodiments, sulfate derivatives derived from the fatty alcohol compositions produced by the methods described herein are used in products such as toothpastes.

In some instances, organic products (e.g., fatty alcohols, especially cetyl alcohol, stearyl alcohol and myristyl alcohol) may be used as food additives (e.g., adjuvants and production aids).

While the processes and systems provided herein have been described with respect to a limited number of embodiments, the specific features of one embodiment should not be attributed to other embodiments of the processes or systems. No single embodiment is representative of all aspects of the methods or systems. In certain embodiments, the processes may include numerous steps not mentioned herein. In other embodiments, the processes do not include any steps not enumerated herein. Variations and modifications from the described embodiments exist.

It is noted that the purification methods are described with reference to a number of steps. In certain embodiments, these steps can be practiced in any sequence. In certain embodiments, one or more steps may be omitted or combined but still achieve substantially the same results. The appended claims intend to cover all such variations and modifications as falling within the scope of the claimed subject matter.

It will be understood that variations and combinations and subcombinations of the aspects and embodiments disclosed above comprise additional embodiments of the invention. Other aspects and embodiments of the invention are set forth below in the examples, from which a person of ordinary skill in the art can practice the invention described herein.

### VI. Examples

The following examples are offered to illustrate, but not to limit, the claimed invention.

### Example 1

### Preparation of Fermentation Broth Emulsions

Fatty alcohol reductase (FAR) variants were generated according to methods described in WO2012/006114 published Jan. 12, 2012 using the wild-type M. algicola FAR of SEQ ID NO:1 as a backbone. Other FAR variants described therein may also be used. The FAR variants were grown in 96-well shallow plates containing 180 μL Luria Bertani (LB) or M9YE medium supplemented with 1% glucose and 30 μg/mL chloramphenicol (CAM), for approximately 16-18 hours (overnight) in a shaker-incubator at 30° C., 200 rpm. A 5% inoculum was used in 96-deep-well plates to initiate fresh 380 μL culture containing 2×YT broth medium supplemented with 30 μg/mL CAM and 0.4% glucose. The culture was incubated for 2 hours at 30° C., 250 rpm to an OD₆₀₀ of 0.6-0.8, at which point expression of the heterologous FAR gene was induced with isopropyl-β-D-thio galactoside (IPTG) (1 mM final concentration). Incubation was continued for about 24 hours under the same conditions. Some variants were grown as stated above with M9YE medium but with an additional amount of glucose (0.5% w/v final conc.) added to the culture at 3 and 6 hours after induction by IPGT. In further rounds of screening M9YE medium containing 5% glucose was used to inoculate the cells and induction by IPTG. The culture was incubated at 30° C. for 48 hours to produce the emulsions.

For generating the data shown in the Tables below, the E. coli host cells were either strain W3110K, MG1655, W3110ΔfhuA, or W3110ΔfhuAΔfadE (available from E. coli Genetic Resources at Yale CGSC, The Coli Genetic Stock Center at website cgsc.biology.yale.edu/; see also the E. coli Genome Project at the University of Wisconsin, at website www.genetics.wisc.edu). The host cells were transformed to express or overexpress one or more exogenous genes, and were grown in a culture medium as described above or as in, for example U.S. Pat. No. 8,110,670. The fermentation emulsions listed below refer to a fermentation broth including fatty alcohols produced from a host strain transformed with a polynucleotide encoding a FAR variant and in some cases other overexpressed genes indicated as follows:
Fermentation Emulsion 1: strain W3110ΔfhuA expressing FAR variant V-1 (SEQ ID NO:3);
Fermentation Emulsion 2: strain W3110K or W3110ΔfhuAΔfadE expressing FAR variant V-2 (SEQ ID NO:4), overexpressing thioesterase (CaBayTES1) and overexpressing E. coli fadD (see U.S. Provisional Patent Application 61/636,044, filed Apr. 20, 2012, and PCT Application No. PCT/US2013/037472);
Fermentation Emulsion 3: strain W3110K or MG1655 expressing FAR variant V-3 (SEQ ID NO:5); the W3110K or MG1655 E. coli strain was transformed with a polynucleotide sequence encoding the FAR variant 8087 as disclosed in U.S. Provisional Patent Application 61/674,053, filed Jul. 20, 2012, and PCT Application No. PCT/US2013/051340.
Fermentation Emulsion 4: strain W3110K expressing FAR variant V-3 (SEQ ID NO:5) and overexpressing E. coli fabH (see U.S. Provisional Patent Application 61/674,053, filed Jul. 20, 2012, and PCT Application No. PCT/US2013/051340); and
Fermentation Emulsion 5: strain W3110K expressing FAR variant V-3 (SEQ ID NO:5) and overexpressing E. coli fabZ and fabI genes; an E. coli strain expressing a FAR variant and overexpressing E. coli fabZ and fabI genes is disclosed in U.S. Provisional Patent Application 61/577,756, filed Dec. 20, 2011, and WO2013/096092.

Table 2 provides the theoretical yield of C12 to C14 fatty alcohols produced for exemplary variants as indicated in the table.

TABLE 2

| Fermentation | Yield of Fatty |
|---|---|
| Emulsion # | Alcohol (g/L) |
| 1 | 24 |
| 2 | 41 |
| 3 | 60 |
| 4 | 50 |
| 5 | 29 |

### Example 2

### Demulsification of Fermentation Broths

To a 50 mL centrifuge tube (BD Falcon/352070/PPE) was placed 20.0 to 30.0 g of a Fermentation Emulsion as described in Example 1 followed by desired amount of demulsification solvent in grams. The tube was capped and placed in a shaking incubator at 50-60° C./300 cpm/rack at 45° angle for 30 minutes (Lab-Line Max Q 4000). The tube was removed, allowed to cool to ambient and centrifuged at 5000G for 5 minutes (Eppendorf Centrifuge 5430R). The efficiency of the separation was observed visually and optionally the upper layer was collected for analysis by Gas Chromatography. In controls where no solvent was employed there is little or no phase separation of product into a top layer. Results using various demulsification solvents are shown in Table 3 below. The amount fatty alcohol product recovered per liter of fermentation broth is also presented.

TABLE 3
Recovery of fatty alcohols utilizing demulsification solvents and
% recovery based on theoretical yield.

| Fermentation | | Amount of | % Theoretical |
|---|---|---|---|
| Emulsion # | Solvent | solvent/broth | Yield |
| 2 | 1-BuOH | 2 mL/10 mL | 68% |
| 2 | 1-BuOH | 3 mL/10 mL | 136% |
| 2 | 1-BuOH | 4 mL/10 mL | 85% |
| 2 | 1-BuOH | 12 g/30 g | 98% |
| 2 | 2-BuOH | 4 g/10 g | 106% |
| 2 | 2-BuOH | 5 mL/10 mL | 118% |
| 2 | dodecanol | 10 g/90 g | 45% |
| 3 and 4* | isoamy | 3 g/10 g | 3% |
| | alcohol | | |
| 3 and 4 | isoamyl | 4 g/10 g | 29% |
| | alcohol | | |
| 3 and 4 | isoamyl | 5 g/10 g | 71% |
| | alcohol | | |
| 3 and 4 | isoamyl | 6 g/10 g | 51% |
| | alcohol | | |
| 3 and 4 | lsobutyl | 3 g/10 g | 26% |
| | alcohol | | |
| 3 and 4 | isobutyl | 4 g/10 g | 74% |
| | alcohol | | |
| 3 and 4 | isobutyl | 5 g/10 g | 86% |
| | alcohol | | |
| 3 and 4 | isobutyl | 6 g/10 g | 86% |
| | alcohol | | |
| 3 and 4 | isobutyl | 20 g/25 g | 120% |
| | alcohol | | |

*A blend of the two fermentation emulsions was used

### Example 3

### Variations in Amounts of Demulsification Solvent

Using the method of Example 2 a series of tests was performed in which the volume of demulsifying solvent (isopropyl alcohol, IPA) was varied. The variance range of the solvent was typically from 0.2 to 0.6 weight percent based on the mass of the fermentation broth. For these studies 20 g of broth from Fermentation Emulsion 2 was used. After completing the series the samples were visually compared and optionally the upper layer was collected for analysis by Gas Chromatography and isolation of the product through evaporation of the solvent. Typically, the sample containing the most product, either by isolation or GC analysis, was judged have been produced with the optimal weight percent of demulsifying solvent for that particular broth lot with the most samples. The theoretical yield of fatty alcohol was determined to be 40.63 based on the results of Example 1, above. The results including variation of the actual % yield based on variation in amount of demulsification solvent used are shown below in Table 4.

TABLE 4
Recovery of Fatty Alcohols from 20 g of broth from Fermentation
Emulsion 2 Utilizing Isopropyl Alcohol (IPA).

| wt % IPA | Recovery of | % of Theoretical |
|---|---|---|
| added | fatty alcohol (g) | Yield |
| 25% | 0.55 | 68% |
| 50% | 0.68 | 84% |
| 20% | 0.21 | 26% |
| 30% | 0.43 | 53% |
| 40% | 0.6 | 74% |
| 50% | 0.4 | 49% |
| 60% | 0 | 0% |

### Example 4

### Variations in Amounts of Demulsification Solvent with Various Emulsion Compositions

Utilizing the method of Example 3 a series of tests was performed in which the volume of demulsifying solvent was varied with various emulsions. The variance range of the amount of demulsifying solvent was typically from 0.2 to 0.6 weight percent based on the mass of the fermentation broth sample. After completing the series the samples were visually compared and optionally the upper layer was collected for analysis by Gas Chromatography and isolation of the product through evaporation of the solvent. Typically, the sample containing the most product, either by isolation or GC analysis, was judged have been produced with the optimal weight percent of demulsifying solvent for that particular broth lot with the most samples. The results are shown below in Table 5.

TABLE 5
Recovery of fatty alcohols from fermentation batches utilizing various
amounts of Isopropyl Alcohol relative to 0% IPA control.

| Fermentation | wt % IPA | % Theoretical |
|---|---|---|
| Emulsion # | added | Yield |
| 1 | 0 | 0 |
| | 25 | 95 |
| 2 | 0 | 0 |
| | 40 | 65 |
| | 50 | 32 |
| 3 | 0 | 0 |
| | 40 | 97 |
| 4 | 0 | 0 |
| | 40 | 77 |
| | 45 | 74 |
| 4 | 0 | 0 |
| | 35 | 68 |
| | 40 | 100 |
| 3 and 4 | 0 | 0 |
| | 30 | 43 |
| | 35 | 80 |
| | 40 | 80 |
| | 45 | 91 |
| 5 | 0 | 0 |
| | 40 | 84 |
| | 45 | 79 |

*A blend of the two fermentation emulsionss was used.

### Example 5

### Demulsification of Fermentation Broth with Separation in a Continuous Centrifuge

Utilizing the method of Example 2, the optimal demulsification solvent amount is determined. For Fermentation Emulsion 4, the optimal demulsification solvent was determined to be 40 weight % and this was used in the following example. A 30-L fermentation vessel (Sartarious Biostat) containing 19.4 Kg of fermentation broth of Fermentation Emulsion 2 was treated with 7.76 Kg of isopropyl alcohol. The reactor was mixed and heated to 60° C. for 60 minutes, and then cooled to 20° C. The mixture was then transferred to a three-phase, disc-stack centrifuge (Westfalia, model OSD-2) where the top light liquid layer (A) was separated from the bottom heavy liquid layer (B) and the cells. Fraction A: The light liquid layer containing product was concentrated by distillation if isopropyl alcohol affording 0.568 Kg of fatty alcohol and 0.535 Kg of isopropyl alcohol. This recovered isopropyl alcohol could optionally be reused in the process. Fraction B: The bottom heavy liquid layer contained 0.043 Kg of fatty alcohol and 5.50 Kg of isopropyl alcohol. This isopropyl alcohol could optionally be recovered by distillation for reuse in the process.

As noted above, 1-butanol, 2-butanol, dodecanol, isoamyl alcohol and isobutyl alcohol give good recovery. In addition it was noted that isopropyl alcohol enhances the sedimentation of the cells which can further assist in purification.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

1. A method of demulsifying an emulsion comprising contacting an emulsion with a demulsifying amount of a demulsifying solvent, whereby the emulsion transforms into at least two distinct phases.

2. A method for purifying an organic product from an emulsion, said method comprising adding a demulsifying amount of at least one demulsifying solvent to the emulsion, whereby the emulsion transforms into at least two distinct phases.

3. A method for producing an organic product from an emulsion of a fermentation mixture, said method comprising culturing a microorganism that produces an organic product under conditions wherein the organic product is produced and adding a demulsifying amount of at least one demulsifying solvent to the emulsion, whereby the emulsion transforms into at least two distinct phases.

4. The method of claim 1, wherein the emulsion is a reaction mixture, a fermentation broth or partially processed fermentation broth.

5. The method of claim 4, wherein the fermentation broth or partially processed fermentation broth comprises a microorganism.

6. The method of claim 5, wherein the microorganism is engineered to produce the organic product.

7. The method of claim 1, wherein one of said two distinct phases is a predominantly non-aqueous phase and one of said two distinct phases is a predominantly aqueous phase.

8. The method of claim 1, further comprising separating the at least two distinct phases from each other.

9. The method of claim 2, wherein said organic product is selected from the group consisting of a fatty alcohol, a fatty acid, a fatty acid ester, a terpene, a terpenoid, a triglyceride, a carotene, a carotenoid, a β-lactam, a sterol, a statin, mycophenolic acid, and mixtures thereof.

10. The method of claim 9, wherein said organic product is a fatty alcohol or a mixture of fatty alcohols.

11. The method of claim 10, comprising saturated fatty alcohol and unsaturated fatty alcohol.

12. The method of claim 11, wherein greater than 50% of fatty alcohol(s) are saturated.

13. The method of claim 10, wherein the fatty alcohol or the mixture of fatty alcohols comprise branched fatty alcohols.

14. The method of claim 10, wherein the fatty alcohol or the mixture of fatty alcohols comprise straight chain fatty alcohols.

15. The method of claim 10, wherein the fatty alcohol comprises a) from 8 to 20 carbon atoms; b) from 8 to 18 carbon atoms; c) from 10 to 16 carbon atoms; or d) from 12 to 14 carbon atoms.

16. (canceled)

17. The method of claim 3, wherein the microorganism is E. coli.

18. The method of claim 3, wherein the microorganism is engineered to include at least one heterologous polynucleotide encoding a protein selected from the group consisting of a fatty acyl reductase, a fatty alcohol forming acyl-CoA reductase, a thioesterase, and a fatty acid biosynthetic enzyme.

19. The method of claim 18, wherein the heterologous polynucleotide encoding a protein is a fatty acyl reductase.

20. The method of claim 1, wherein the demulsifying solvent is a C3-C5 primary, secondary or tertiary alcohol.

21-35. (canceled)
